(19) United States
(12) Patent Application Publication
(10) Pub. No.: US 2008/0206161 A1
(43) Pub. Date: Aug. 28, 2008

(54) QUIESCENT FOAMABLE COMPOSITIONS, STEROIDS, KITS AND USES THEREOF

(76) Inventors: Dov TAMARKIN, Maccabim (IL); Meir EINI, Ness Ziona (IL); Doron FRIEDMAN, Karmei Yosef (IL); David SCHUZ, Moshav Gimzu (IL); Tal BERMAN, Rishon LeZiyyon (IL)

Correspondence Address: WILMERHALE/BOSTON, 60 STATE STREET, BOSTON, MA, 02109, US

(21) Appl. No.: 12/020,381

(22) Filed: Jan. 25, 2008

Related U.S. Application Data
(63) Continuation-in-part of application No. 10/911,367, filed on Aug. 4, 2004.
Application No. 10/911,367 is a continuation-in-part of application No. 11/114,410, filed on Apr. 26, 2005.
(60) Provisional application No. 60/492,385, filed on Aug. 4, 2003.
(60) Provisional application No. 60/897,638, filed on Jan. 26, 2007.

(30) Foreign Application Priority Data
Oct. 25, 2002 (IL) 152486

(51) Int. Cl. A61K 9/12; A61P 29/00

(52) U.S. Cl. 424/45

(57) ABSTRACT

The present invention relates to an emulsion steroid composition as a foamable vehicle in which the vehicle is stable or stabilized by the presence of at least one quiesence agent; therapeutical kit containing such composition; to methods of treatment using such composition; to methods of enhancing the stability of such composition.

### CROSS REFERENCE TO RELATED APPLICATIONS

This application claims the benefit of priority under 35 U.S.C. §119(e) to co-pending U.S. Provisional Application No. 60/897,638 filed Jan. 26, 2007, entitled “Quiescent Foamable Compositions, Steroids, Kits and Uses Thereof”, which is incorporated in its entirety by reference.

This is application is a continuation-in-part of and claims priority under 35 U.S.C. §120 to co-pending U.S. patent application Ser. No. 11/114,410, filed Apr. 26, 2005, entitled “Steroid Kit and Foamable Composition and Uses Thereof,” which is herein incorporated in its entirety by reference.

This application is a continuation-in-part of co-pending U.S. patent application Ser. No. 10/911,367, filed on Aug. 4, 2004, which claims the benefit of priority under 35 U.S.C. §119(e) to U.S. Provisional Patent Application No. 60/492,385, filed on Aug. 4, 2003, both entitled “Foam Carrier Containing Amphiphilic Copolymer Gelling Agent” and both herein incorporated in their entirety by reference.

This application is a continuation-in-part of co-pending International Patent Application No. IB03/005527, entitled “Cosmetic and Pharmaceutical Foam,” designating the United States and filed on Oct. 24, 2003, which claims the benefit of priority under 35 U.S.C. §119(e) to U.S. Provisional Patent Application Ser. No. 60/492,546, filed on Nov. 29, 2002 (same title) and under 35 USC §119(a) to Israeli Patent Application. No. 152486, filed Oct. 25, 2002, all of which are herein incorporated in their entirety by reference.

### BACKGROUND

Development of foamable pharmaceutical carriers and compositions of active agents requires a multi-disciplinary approach. The challenge of preparing emulsion carriers and compositions that are capable of forming stable breakable foams suitable for topical or mucosal application is beset with a multitude of challenges. The active agents are often sensitive and also may interact with other components in a composition. Agents may have limited biocompatibility or poor solubility. The agents may be difficult to deliver to a target site.

Additionally, the carriers may comprise emulsions which lack physical stability and may have a short, unstable shelf life.

Another issue with foamable compositions is that an appropriate rheology needs to be retained so that, on the one hand, the composition remains shakable so that it may be easily expelled through an aerosol system and valve over its period of use, yet, on the other hand have sufficient form that it will produce a stable foam of an acceptable quality. Some emulsions may be stable but do no meet the criteria of shakability or flowability as is required for a foamable formulation to be expelled under pressure through a canister valve using a propellant. Other emulsions albeit shakable may not produce stable breakable foams.

There is thus a need in the art to provide foamable pharmaceutical carriers and compositions of active agents, which are shakable, stable and retain the activity of one or more active agents. Moreover, there is a need that these formulations be resistant or resilient to aging and do not separate into two phases as can be determined by exposing the formulations to temperature changes, or by centrifugation. To the extent such formulations display creaming there is a need for them to be easily and effectively redispersible upon exposure to light shaking. “Mild agitation” or “lightly shaking” refers to the normal mild force applied to a canister containing a shakable foamable formulation with propellant, which is raised to about the level of head height and mildly brought to about waist height and back again a few times by an average adult without excessive or vigorous force.

Steroids are agents that are difficult to formulate for pharmaceutical use. Some steroids, such as betamethasone valerate, are known to be particularly unstable and tend to isomerize.

Foam products may, for example be pressurized preparations composed of liquid emulsion and propellant packed into a canister, such as an aluminum canister, mounted with a valve and anactuator. Upon actuation of the foam onto the skin, the propellant immediately vaporizes and the emulsion deposits on the skin surface.

There is a need for stable foams that are breakable under shear upon application. The foam should ideally spread easily on skin with minimal greasy or sticky feeling and should be absorbed quickly. These foam qualities are desirable for usability and compliance. Further desirable positive attributes are acceptable odor and color.

Topical application with a foam product is superior to treatments with cream or ointment dosage forms. Usage of foam provides a better skin moisturizing and protective capacity and is less irritable than the other dosage forms. Foams easily spread on large skin areas can be efficiently rubbed into the skin.

Body cavity application with a foam product is also superior to treatments with cream or ointment dosage forms. Usage of foam provides a better cavity moisturizing and protective capacity and may be less irritable than the other dosage forms. Breakable foams easily expand to occupy the body cavity and subsequently collapse on mechanical shear to spread over large internal areas to provide a localized or targeted effect as well as efficient delivery of one or more active agents/ingredients.

The usability of a pharmaceutical product is largely influenced by its physical properties, as manifested by its ease of application, spreadability and absorption. Stability is a primary feature that has to be ensured when a pharmaceutical formulation, and in particular a foam pharmaceutical product, is being developed.

Emulsions are inherently non-stable products, which are stabilized by the aid of surface-active agents, surfactants, and bulking agents that contribute to increased viscosity. Surfactants reduce the free energy between oil and aqueous phase and build a separation film between the two immiscible phases. Stronger and compact separation film contributes to enhanced stability and longer shelf life. The creaming phenomenon which may cause emulsion breaking and phase separation should be avoided since it may finally contribute to separation and a non-usable product.

Producing emulsion foamable compositions which are physically stable and resilient to creaming and produce stable breakable foams is a non obvious challenge. Producing such steroid foamable compositions which are chemically stable and retain the desired physical properties is a double challenge.

Steroids are currently available in topical dosage forms. Compositions containing steroids for topical treatment of dermatologic disorders are available primarily in cream, lotion, gel and ointment forms. While semi-solid compositions, such as creams, lotions, gels and ointments are commonly used by consumers, new forms are desirable, in order to achieve better control of the application, while maintaining or bestowing beneficial properties of such products on the skin. Thus, the development of new compositions having breakable foam consistency when released from a container and liquid properties when applied onto the skin are advantageous.

Foams and, in particular, foam emulsions are complicated systems which do not form under all circumstances. Slight shifts in foam emulsion composition, such as by the addition of active ingredients, may destabilize the foamable composition during storage, and/or impair the quality of the foam that is produced upon release from the aerosol container.

Foam compositions use aliphatic alcohols, which promote fast drying and thereby attempt to address the sticky feeling left by many topical formulations after application; however, alcohols, are defafting agents and may cause skin to become dry and cracked. Pharmaceutical foam compositions including (a) an active ingredient; (b) an occlusive agent; (c) an aqueous solvent; and (d) an organic cosolvent; wherein the active ingredient is insoluble in water and insoluble in both water and the occlusive agent; and wherein there is enough occlusive agent to form an occlusive layer on the skin are known.

Substantially lower alcohol-free foam compositions for topical treatment containing higher concentrations of oils that are robust and suitable for inclusion of a wide range of active ingredients are desired.

There remains an unmet need for improved, easy to use, stable and non-irritating foam formulations, intended for treatment of dermal and mucosal tissues, especially formulations which are stable for a substantial time period. Moreover, formulations that are able to remain physically stable and chemically stable or substantially so, over a sufficient period of time to allow for normal pharmaceutical use. Particularly, there remains an unmet need for improved, easy to use, stable and non-irritating steroid foam formulations, with unique therapeutic properties. There is more particularly a need to develop steroid carriers and compositions, steroid foamable carriers and foamable compositions and steroid foams with and without other active agents, which are stable, are non irritating, that facilitate penetration at a target, that are presentable in an easily applicable stable form, that can be handled with ease thereby facilitating compliance and that are adaptable for long term use where there is a need to minimize the amount of the potential isomerization of the steroids or the potential breakdown of ingredients/agents by oxidation/hydrolysis.

Some active agents such as certain steroid compounds are known to be generally unstable or susceptible to isomerisation or to breakdown, resulting in loss of activity and the use of stabilizers, anti-oxidants antimicrobials and buffers and the like in aqueous compositions to protect active or cosmetic agents is known. The problems of protecting active steroid pharmaceutical agents in emulsion compositions are multifold and can vary according to the type of emulsion environment and the nature of the agent and excipients being used. It has been surprisingly found that factors like small levels of acid residues in the raw materials can be significant in influencing agent stability. Similarly, the presence of low levels of metal ions can act to catalyze reactions or breakdown. There is therefore a need for simple and elegant solutions to stabilize active steroid ingredients in an emulsion environment. On one level it is far from simple or obvious to produce emulsion foamable compositions that when released produce foams of quality suitable for pharmaceutical or cosmetic application that are capable of satisfying the need for both physical stability of the foam and for chemical stability of the active steroid ingredient. On a further level having realized a composition that will produce an emulsion foam of quality there is an additional difficulty to be overcome, namely how to adapt the formula and achieve a formulation, which can accept a range of various other active pharmaceutical and cosmetic agents such that the composition, steroid and other active agents are compatible and capable of remaining stable for a prolonged period and the foam produced remains of quality. Specifically, one of the challenges in preparing such foamable compositions is ensuring that the active pharmaceutical or therapeutic agent does not react, isomerize or otherwise break down to any significant extent during is storage and use. Particularly, there remains an unmet need for improved, easy to use, stable and non-irritating foamable formulations, with unique therapeutic or beneficial properties containing a stable or stabilized active pharmaceutical or cosmetic agent which when stored in and subsequently expelled from a canister can be delivered as a breakable foam.

There is also a particular need for foamable compositions for mucosal and body cavity application especially, vaginal and uterine application.

The vaginal cavity, including the vagina and cervix, provides a unique site for delivery of therapeutic agents, both for systemic and local action.

There are multiple anatomical structures which comprise the internal and external female genital tract including the clitoris, labia minora and corpus spongiosum (vestibular) erectile tissue, vagina, peri-urethral glans, urethra, Halban's fascia, anterior formix erogenous zone, pubococcygeus muscle and cervix.

The vagina consists of a tube of autonomically-innervated smooth muscle (longitudinal outer, inner circular layer) lined by stratified squamous epithelium and a sub-dermal layer rich in capillaries. The vaginal wall consists of an inner glandular mucous type stratified squamous cell epithelium supported by a thick lamina propia. This epithelium undergoes hormone-related cyclical changes including slight keratinization of the superficial cells during the menstrual cycle. Deep in the epithelium lies the smooth muscles of the muscularis. There is a deeper surrounding fibrous layer above the muscularis which provides structural support to the vagina and is rich is collagen and elastin to allow for expansion of the cavity. Three sets of skeletal muscles surround the vagina including the ischiocavernosum, bulbocavernosus, transverse perinei and levator ani and pubococcygeus muscles.

Women are vulnerable to diseases of the genital tract as the lining of the vagina is a permeable mucous membrane. Intercourse, lack of lubrication during intercourse, changes in the cervix during the menstrual cycle, and asymptomatic infections facilitate the transmission of infection to women. Prepubertal girls and adolescents are particularly vulnerable because their vaginal and cervical tissues may be less mature and are more readily penetrated by organisms (e.g., chlamydia and gonococcus). Postmenopausal women are more likely than younger women to get small abrasions in the vagina during sexual activity as a result of thinning of the tissue and dryness. Women who already have an infection (particularly one that causes genital lesions) are more likely to acquire or transmit another sexually transmitted disease (STD), including HIV. Other biological risks include the use of vaginal douches, which increase the risk of pelvic inflammatory disease (PID), and the influence of hormonal contraceptives on acquiring or transmitting an STD (e.g., increased risk of chlamydial infection with use of oral contraceptives).

In particular, the cervix is prone to several diseases, such as cervicitis (an inflammation of the uterine cervix, usually caused by infection), cancer, inflammation, erosion, intraepithelial neoplasia (CIN), polyps, dysplasia, human papillomavirus (HPV) infections causing some tumors, condylomas or warts and abnormal pregnancy.

Several factors must be taken into consideration when developing therapeutic delivery systems for the female genital system. These factors include the vaginal anatomy, the mucosal surface, the presence and composition of vaginal fluids and secretions, cervical fluids (mucus), cyclic changes and endogenous microflora. Drug stability to enzyme activity, which is quite high in vaginal environment, and is again a function of menstrual cycle and lifecycle, should also be taken into account. Topical drug delivery through the cervix, as needed to treat disorders of the cervix and uterus also presents a challenge.

Vaginal topical formulation should be compatible with daily activities, be easy to administer and provide accurate dosing. Several types of formulations are known for delivery to the vaginal cavity. While semi-solid formulations, such as creams, lotions, gels and ointments are commonly used, they are often reported to be messy, require frequent application and can be difficult to remove after use. Furthermore, application of topical gels and creams require several steps of operation. Solid formulations such as tablets, suppositories and pessaries also require frequent application, show a poor retention in vagina, and exhibit insufficient spreadability.

Rectal drug administration can be directed to both local and systemic drug delivery. It has been effectively used to treat local diseases of the anorectal area as well as an alternative to oral administration in the systemic administration of drugs. Solid suppositories are the most common dosage form used for rectal drug administration and represent the majority of rectal dosage forms; however, creams ointments and foams are also being used.

Current formulations for rectal administration still have significant disadvantages. They are difficult to insert through the anal orifice; they are difficult to spread throughout the target cavity; and if spreadable, they tend to leak, causing major discomfort to the patient. Such negative attributes lead to their very limited use.

Thus, new forms are desirable in order to achieve better control and ease of application, while maintaining the beneficial properties of such products. A product for intravaginal and anorectal application would ideally exhibit the following properties: (1) easy insertion, thus leading to high patient compliance; (2) accurate dosing, to ensure effective treatment; (3) expandability, for increased coverage of the target cavity surface and cervix; and (4) drip free formulation with good adhesive properties, for prolonged drug residence. The duration of the drug inside the vagina or rectum is also important for ensuring extended activity.

### SUMMARY

The present invention relates to improved, easy to use, stable and non-irritating foamable compositions and foams comprising an active agent, intended for treatment of dermal and mucosal tissues.

The present invention relates to an emulsion composition as a vehicle in which the vehicle is stable or stabilized.

The present invention relates to an emulsion composition as a vehicle in which an active pharmaceutical or cosmetic agent, when added is stable or stabilized.

The present invention relates to an emulsion composition as a vehicle in which the vehicle is stable or stabilized by the presence of a quiesence agent.

The present invention relates to an emulsion composition as a vehicle in which an active pharmaceutical or cosmetic agent, when added is stable or stabilized in the presence of a quiesence agent.

The present invention more particularly relates to quiescence foamable carriers and compositions and quiesence agents.

The present invention more particularly relates to quiesence agents for use in quiescence foamable carriers and compositions including a quiescence agent selected from a stabilizing, a polymeric, a gelling, a viscoelastic, a pseuedoplastic or semi-pseudoplastic, a resilient agent and a modulating agent. A quiescence agent may have more than one of these characteristics. In certain compositions a combination of two or more agents is used to achieve a quiescence foamable composition having more than one of there characteristics.

The present invention more particularly relates to quiescence compositions which are physically and chemically stable or substantially so.

The present invention more particularly relates to quiescence compositions which are physically and chemically stable or substantially so for short, medium and or long term periods.

The present invention more particularly relates to quiescence compositions which are physically and chemically stable or substantially so for short, medium and or long term periods.

The present invention more particularly relates to quiescence compositions which are physically and chemically stable or substantially so at room temperature during the expected shelf life of a pharmaceutical product or for a lesser reasonable period.

The present invention more particularly relates to pseudoplastic or semi-pseudoplastic compositions which are physically stable or substantially so at room temperature during the expected shelf life of a pharmaceutical product or for a lesser reasonable period.

The present invention more particularly relates to quiescence compositions which are physically and chemically stable or substantially so for short, medium and or long term periods and is shakable or flowable; and is stored in an aerosol container, which upon release expands to form a breakable foam.

The present invention more particularly relates to quiescence compositions which are physically and chemically stable or substantially so at room temperature during the expected shelf life of a pharmaceutical product or for a lesser reasonable period and is shakable or flowable; and is stored in an aerosol container, which upon release expands to form a breakable foam.

The present invention more particularly relates to pseudoplastic or semi-pseudoplastic compositions which are physically stable or substantially so at room temperature during the expected shelf life of a pharmaceutical product or for a lesser reasonable period and is shakable or flowable; and is stored in an aerosol container, which upon release expands to form a breakable foam.

The present invention more particularly relates to modulating agents that are able to be effective in relatively low concentrations and which have little or no significant effect on the foam compositions.

According to one or more embodiments, the emulsion foamable carrier includes: a hydrophobic solvent, a stabilizing surfactant, at least one quiesence agent, water and a propellant.

According to one or more embodiments, the emulsion foamable carrier includes: a hydrophobic solvent, a stabilizing surfactant, water and a propellant and one or more active pharmaceutical agents.

According to one or more embodiments, the foamable emulsion composition, includes: a hydrophobic solvent, a stabilizing surfactant, a quiesence agent, water and a propellant and one or more active pharmaceutical agents.

According to one or more embodiments, the foamable emulsion composition, includes: a hydrophobic solvent, a stabilizing surfactant, a resilient agent, a modulating agent, water and a propellant and one or more active pharmaceutical agents.

According to one or more embodiments, the foamable emulsion composition, includes: a solvent, a stabilizing surfactant, a pseudoplastic or semi-pseudoplastic agent, a modulating agent, water a propellant and one or more active pharmaceutical agents.

According to one or more embodiments, at least one of the active pharmaceutical agents is a steroid.

According to one or more embodiments, at least one of the resilient agents is a viscoelatic, which in turn is capable of forming a pseudoplastic or semi-pseudoplastic composition.

According to one or more embodiments, the viscoelatic comprises an effective amount of at least one polymeric additive selected to form a foamable carrier or composition which is resilient to creaming/phase separation

According to one or more embodiments, the viscoelatic comprises an effective amount of at least one polymeric additive selected to form a foamable carrier or composition which is shakable and flowable to a sufficient amount to be expelled by the propellant from a canister to form a foam of appropriate quality for topical or mucosal application.

According to one or more embodiments, the viscoelatic comprising at least one polymeric additive selected from the group consisting of a bioadhesive agent, a gelling agent, a film forming agent and a phase change agent.

In the process of trying to overcome the challenge of formulating the steroid-like betamethasone valerate or calcipitriol, it has been discovered that it is possible to improve and increase the physical stability of emulsion foamable compositions and therefore any resultant foam produced therefrom. It has been further found that it is possible to improve the chemical stability of the steroid or other active agent in the composition.

In some embodiments, the compositions described herein are emulsions adapted to provide excellent foam upon actuation from a pressurized canister. The emulsion foam platform is made of oily phase and water phase emulsified and stabilized with surfactants and polymers and co-stabilizers that impart better foam properties.

Whilst the quiescence, the creaming resilience, the pseudoplastic and semi-pseudoplastic formulations and long term physical and chemical stability of the carriers, formulations, foamable compositions and foams have been primarily demonstrated with steroids, which are known to be unstable or in combination with other active agents or with compounds with particular solubility issues, the concepts, carriers, formulations, foamable compositions and foams disclosed are likewise applicable to active agents other than steroids.

In one or more embodiments for example the carriers, formulations, foamable compositions and foams disclosed herein are likewise applicable to coal tar formulations. One of the advantages, is that they can be formulated to exhibit bioadhesive properties and can be used for body cavity applications with minimal or no leakage. They are also of pleasant appearance and skin feeling and do not sting, making them suitable for delivery to sensitive areas. Also by storing them in sealed canisters with propellant, they can provide a safer medium for active agents that tend to break down upon exposure to light or air.

In some aspects, the present invention provides an easy to use steroid containing a vaginal delivery system that will be simple to operate with minimal preparation, will be very tolerable without having a feeling of foreign matter, will provide accurate dose administration, will evenly spread throughout the vaginal cavity surface, will effectively reach the cervix, will not leak and will retain intravaginally an active agent for a significant period of time. In other aspects, the present invention provides a steroid and a lubricating vaginal drug vehicle for moisture replenishing or moisturizing vaginal vehicles. In other aspects, the invention provides an improved delivery system for steroids alone or in combination with other active agents to other body cavities, such as the rectum, penile urethra, nasal cavity and ear cavity and to mucosal surfaces.

The present invention relates to foam compositions for intra-vaginal and body cavity application of a wide range of steroids on their own or in combination with a very wide variety of ingredients. The compositions contain at least one active steroid agent in a biocompatible alcohol-free foamable carrier, including, oil-in-water foamable emulsions and water-in-oil foamable emulsions, liposome-based foams and nanoparticle-based foams. These compositions provide long lasting, drip-free, expandable formulations for drug delivery into body cavities.

In one or more embodiments it has been possible to achieve formulations suited for body cavity use by incorporating into the breakable foam formulation a bioadhesive character where force is required to dislodge from the foam. In an embodiment the foam has a bioadhesive value of about −3 g to about −30 g, preferably between about −8 g to about −25 g.

The present invention further provides a therapeutic kit including a foamable steroid composition. The kit includes an aerosol packaging assembly having a container accommodating a pressurized product and an outlet capable of releasing the pressurized product as a foam.

The therapeutic kit includes an aerosol packaging assembly including (a) a container accommodating a pressurized product and (b) outlet capable of releasing the pressurized product as a foam; wherein the pressurized product contains a foamable composition including: (i) a steroid; (ii) at least one organic carrier selected from the group consisting of a hydrophobic organic carrier, an organic polar solvent, an emollient and mixtures thereof, at a concentration of about 2% to about 50% by weight; (iii) a surface-active agent; (iv) about 0.01% to about 10% by weight of at least one quiesence agent; (v) water; and (vi) liquefied or compressed gas propellant at a concentration of about 3% to about 25% by weight of the total composition.

The therapeutic kit includes an aerosol packaging assembly including (a) a container accommodating a pressurized product and (b) outlet capable of releasing the pressurized product as a foam; wherein the pressurized product contains a foamable composition including: (i) a steroid; (ii) at least one organic carrier selected from the group consisting of a hydrophobic organic carrier, an organic polar solvent, an emollient and mixtures thereof, at a concentration of about 2% to about 50% by weight; (iii) a surface-active agent; (iv) about 0.01% to about 5% by weight of at least one resilient agent; (v) a modulating agent (vi) water; and (vii) liquefied or compressed gas propellant at a concentration of about 3% to about 25% by weight of the total composition.

The steroid kit further contains an applicator of appropriate length and cross section adapted for body cavity application. For example, an applicator ideal for intravaginal use for example to deliver a steroid hormone such as esradiol or prgestron will be of an appropriate length to facilitate delivery to the area of the uterine entrance.

In preferred embodiments, the composition further contains a therapeutically effective foam adjuvant to increase the foaming capacity of surfactants and/or to stabilize the foam. In one or more embodiments, the foam adjuvant includes fatty alcohols having 15 or more carbons in their carbon chain, fatty acids having 16 or more carbons in their carbon chain, and combinations or mixtures thereof.

In one or more embodiments, a combination of a fatty acid and a fatty ester is employed.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

In one or more embodiments, at least a portion of the steroid is suspended in the composition, yet, in other embodiments, the steroid is dissolved in the composition.

In one or more embodiments, the foam composition is formulated as an oil-in-water emulsion or oil-in-water microemulsion.

In some embodiments, the steroid is selected from the group consisting of:
a steroid compound containing a cyclopenta[a]phenanthrene skeleton;
a steroid compound containing a cyclopenta[a]phenanthrene skeleton carrying one or more functional groups selected from halogens, alkyl groups, aryl groups, benzyl groups, carboxy groups and alkoxy groups;
a steroid compound selected from the families of (a) cardanolides, (b) bufanolides, (c) spirostans, (d) furostans, (e) steroid alkaloids, (f) steroid lactones, (g) oxo-steroids, (h) steroid-alcohols and (i) steroid-amines;
a steroid compound, where one or more of the cyclopenta[a]phenanthrene rings is contracted by loss of an unsubstituted methylene group;
a steroid compound, where one or more of the cyclopenta[a]phenanthrene rings is expanded by inclusion of a methylene group;
a steroid compound containing a cyclopenta[a]phenanthrene skeleton and a carbocyclic or heterocyclic ring component fused to it;
a compound, wherein two or more steroid molecules are linked together covalently;

a compound selected from the group consisting of 5α-pregnane, 5β-pregnane, 5α-cholane (allocholane), 5β-cholane, 5-cholestane, 5β-cholestane, 5α-ergostane, 5β-ergostane, 5α-campestane, 5β-campestane, 5α-poriferastane, 5β-poriferastane, 5α-stigmastane, 5β-stigmastane, 50-gorgostaneacrihellin, actodigin, alfacalcidol, aldosterone, androsterone, betamethasone, brassinolide, calcidiol, calciol, calcitriol, canrenone, clomegestone, cholesterol, cholic acid, corticosterone, cortisol, cortisol acetate, cortisone, cortisone acetate, cyproterone, deoxycorticosterone, dexamethasone, disogluside, ecdysone, ercalciol, ergosterol, estradiol, estriol, estrone, ethinylestradiol, fluazacort, fluocortin, fusidic acid, gestrinone, gonane, halometasone, hydrocortisone, lanosterol, lithocholic acid, mebolazine, medroxyprogesterone, meproscillarin, mespirenone, mestranol, naflocort, norenthisterone, norgesterone, norgestrel, oxandrolone, oxymetholone, pancuronium bromide, prednisolone, prednisone, progesterone, proscillardin, pseudotigogenin, roxibolone, sarsasapogenin, smilagenin, spironolactone, timobesone, testosterone, tigogenin triamcinolone, ursodeoxycholic acid;
an anti-inflammatory steroid;
a steroid possessing immunomodulating and/or anti-inflammatory properties;
a steroid, selected from the group of low-potency anti-inflammatory steroids, medium potency anti-inflammatory steroids and high potency anti-inflammatory steroids;
an anti-inflammatory steroid, selected from the group consisting of hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol, clobetasol-17-propionate;
a steroid that positively affects the McKenzie vasoconstrictor assay;
a steroid hormone;
a steroid hormone, selected from the group consisting of an androgen, an estrogen and a progestogen;
an androgen, selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone;
an estrogen selected from the group consisting of estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin;
a progestogen, selected from the group consisting of progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate;
an inhibitor of a steroid hormone;
an inhibitor of a steroid hormone selected from the group consisting of finasteride, dutasteride and spironolactone;
a vitamin D;
a steroid that exhibits qualitatively the biological activity of calciol;
a vitamin D selected from the group consisting of cholecalciferol, 25-hydroxycholecalciferol, 1α,25-dihydroxycholecalciferol, ergocalciferol, 1α,25-dihydroxyergocalciferol, 22,23-dihydroergocalciferol, 1,24,25-trihydroxycholecalciferol, previtamin D₃, tachysterol₃ (also termed tacalciol);
a vitamin D3 analogue;
isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol;
a vitamin D₃ analogue selected from calcipotriol, tacalcitol, maxacalcitol, and calcitriol;
a phytosteroid or a phytosterol;
a steroid derived or extracted from one of the families of phytosteroids, phytosterols, phytostanols, ecdysones, withanolids, sterines, steroid saponins and soflavonoids;
a steroid selected from the group consisting of alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol;
a plant oil or a plant extract, which contains a steroid;
a plant oil or a plant extract, selected from the group consisting of nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, and calendula officinalis;
any one of the compounds exemplified in the present specification;
and salts thereof.

According to further embodiments, there is provided a method of treating, alleviating or preventing disorders of the skin, a body cavity or mucosal surface, where the disorder involves inflammation as one of its etiological factors. The method includes topically administering a foamed composition to a subject having the disorder, the foamed composition including a steroid, at least one organic carrier selected from a hydrophobic organic carrier, a polar solvent, an emollient and mixtures thereof, at a concentration of about 2% to about 50% by weight, about 0.1% to about 5% by weight of a surface-active agent, about 0.01% to about 5% by weight of a polymeric additive selected from a bioadhesive agent, a gelling agent, a film forming agent and a phase change agent and water, wherein the steroid is administered in a therapeutically effective amount.

Disorders suitable for treatment include vaginal disorders, vulvar disorders, anal disorders, disorders of a body cavity, ear disorders, disorders of the nose, disorders of the respiratory system, bacterial infections, fungal infections, viral infections, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, cancroids, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, infertility, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; sexual dysfunction in men and women, androgen deficiency; estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, hirsutism, virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

There is thus provided according to some embodiments, a storable foamable emulsion composition adapted for delivery of an active pharmaceutical ingredient (API) to a delivery site in a subject, the composition including:
at least one organic carrier selected from the group consisting of a hydrophobic organic carrier, an organic polar solvent, an emollient and mixtures thereof, at a concentration of about 2% to about 50% by weight;
at least one surface-active agent at a concentration of about 0.01% to about 5% by weight;
at least one polymeric agent selected from the group consisting of a bioadhesive agent, a gelling agent, a film forming agent and a phase change agent, each in a concentration of about 0.01% to about 5% by weight;
water;
an effective amount of at least one API selected from the group consisting of a steroid, a steroid derivative, a further active agent together with the steroid or derivative thereof and combinations thereof; and
a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least about one month;
wherein the at least one API remains chemically stable for at least about one month; and
wherein the composition is stored in an aerosol container and upon release expands to form a breakable foam having an average bubble size range of about 30 to about 250 micron.

In one embodiment, when the foam or foamable emulsion composition is compressed between two surfaces, a negative adhesive force is required to effect their separation. A negative force could be about −1 g or less. By less it is meant say for example about −3 g to about −30 g, and preferably about −8 g to about −25 g.

There is also provided a storable foamable emulsion composition adapted for delivery of an active pharmaceutical ingredient (API) to a delivery site in a subject, the composition comprising:
at least one fatty alcohol, at a concentration of about 2% to about 50% by weight;
at least one surface-active agent at a concentration of about 0.01% to about 5%
by weight;
at least one polymeric agent selected from the group consisting of a bioadhesive agent, a gelling agent, a film forming agent and a phase change agent, each in a concentration of about 0.01% to about 5% by weight;
water;
an effective amount of at least one API selected from the group consisting of a steroid, a steroid derivative, a further active agent together with the steroid or derivative thereof and combinations thereof; and
a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least about one month;
wherein the at least one API remains chemically stable for about at least about one month; and
wherein the composition is stored in an aerosol container and upon release expands to form a breakable foam having an average bubble size range of about 30 to about 250 micron.

In one or more embodiments the composition is physically and chemically stable within acceptable pharmaceutical limits for a foamable composition for about at least three months. In one or more embodiments the composition is physically and chemically stable within acceptable pharmaceutical limits for a foamable composition for about six months, for about a year or for about 2 years.

The carrier compositions described herein may also be suitable for storing and delivering API's other than steroids. Thus, there is also provided according to specific embodiments a foamable carrier composition containing an active agent without steroid. In one embodiment the active agent is coal tar or a coal tar extract.

In some embodiments, the compositions include at least one quiescence agent. In some embodiments, the API is stable or is stabilized in the presence of at least one quiescence agent. In some embodiments, the API and at least one quiescence agent function synergistically to achieve improved chemical or physical characteristics for the composition. In some embodiments, the API demonstrates improved characteristics when combined with at least one quiescence agent. In some embodiments, the improved chemical or physical characteristics is improved stability over time, whether chemical or physical stability. In some embodiments, the improved chemical or physical characteristics include, without limitation, one or more of the following properties: foam quality, bioadhesivity, color, odor, and collapse time. Moreover, in some embodiments, the improved chemical or physical characteristics are demonstrated under various storage conditions (e.g., temperature ranging from about 25° C. to about 50° C. or any specific temperature between and including these temperatures; and humidity ranging from about 0% to about 100% relative humidity or any specific humidity between and including these values). In some embodiments, the compositions demonstrate improvement in at least one property. In some embodiments, the compositions demonstrate improvement at least in two properties. In some embodiments, the compositions demonstrate improvement in at least three properties. In some embodiments, the compositions demonstrate improvement in at least four properties. In some embodiments, the compositions demonstrate improvement in at least five properties. In some embodiments, the compositions demonstrate improvement in at least six properties. In some embodiments, the compositions demonstrate improvement in at least seven properties. In some embodiments, the compositions demonstrate improvement in at least eight properties. In some embodiments, the compositions demonstrate improvement in at least nine properties. In some embodiments, the compositions demonstrate improvement in at least ten properties. In some embodiments, the compositions demonstrate improvement in at least eleven properties. In some embodiments, the compositions demonstrate improvement in at least twelve properties.

In some embodiments the bioadesivity of the composition to the delivery site requires a force of about −3 g to about −30 g to dislodge the foam from the delivery site. In some embodiments, the bioadesivity of the composition to the delivery site requires a force of about −8 g to about −25 g to dislodge the foam from the delivery site.

In some embodiments, the color of the composition or the foam resulting from the composition is white, white to off-white, yellowish, or yellow.

In some embodiments, the foam quality of the composition or the foam resulting from the composition is 5-6 after at least one freeze-thaw cycle. In some embodiments, the foam quality of the composition or the foam resulting from the composition is 5-6 after two freeze-thaw cycles, three freeze-thaw cycles, four freeze-thaw cycles, five freeze-thaw cycles or ten freeze-thaw cycles. In some embodiments, the foam quality of the composition or the foam resulting from the composition is 5-6 after about 1 week, 2 weeks, 3 weeks, 4 weeks, 1 month, 2 months or 3 months. Storage conditions include, without limitation, storage at room temperature, 25° C., 30° C., 40° C., 50° C., or 60° C. or combinations thereof.

In some embodiments, the collapse time of the composition or the foam resulting from the composition is, without limitation, at least 30 seconds, at least 40 seconds, at least 50 seconds, at least 60 seconds, at least 70 seconds, at least 80 seconds, at least 100 seconds, at least 120 seconds, at least 140 seconds, at least 160 seconds, at least 180 seconds, at least 200 seconds, at least 220 seconds, at least 240 seconds, at least 260 seconds, at least 280 seconds or at least 300 seconds.

There is thus provided according to some further embodiments, a method for forming a breakable foam adapted for delivery of an active pharmaceutical ingredient (API) to a delivery site in a subject, the method including:
forming an emulsion from at least one organic carrier, at least one surface-active agent and at least one polymeric additive in water,
wherein the emulsion includes the at least one surface active agent in a suitable concentration relative to the at least one organic carrier such that the at least one surface active agent is matched with the at least one organic carrier;
mixing the emulsion with an effective amount of at least one API to form the storable foamable emulsion composition; and
propelling the storable foamable emulsion composition with a propellant to form a breakable bioadhesive foam having an average bubble size range of about 30 to about 250 micron.

There is thus provided according to some additional embodiments, a method of treating, alleviating or preventing a disorder of the skin, a body cavity or a mucosal surface, wherein the disorder involves inflammation as one of its etiological factors, which method includes administering topically to a subject having the disorder in need thereof a therapeutically effective amount of a storable foamable emulsion composition including:
at least one organic carrier selected from the group consisting of a hydrophobic organic carrier, an organic polar solvent, an emollient and mixtures thereof, at a concentration of about 2% to about 50% by weight;
at least one surface-active agent at a concentration of about 0.01% to about 5% by weight;
at least one polymeric additive selected from the group consisting of a bioadhesive agent, a gelling agent, a film forming agent and a phase change agent, each in a concentration of about 0.01% to about 5% by weight;
water;
an effective amount of at least one API selected from the group consisting of a steroid, a steroid derivative, a further active agent together with the steroid or derivative thereof and combinations thereof; and
a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least one month;
wherein the at least one API remains chemically stable for about at least one month; and
wherein the composition is stored in an aerosol container and upon release expands to form a breakable bioadhesive foam having an average bubble size range of about 30 to about 250 micron.

### BRIEF DESCRIPTION OF THE DRAWINGS

The invention is described with reference to the figures which are presented for the purpose of illustration and are not intended to be limiting.

FIG. 1 is a schematic illustration of an aerosol valve suitable for use in the aerosol packaging assembly according to in one or more embodiments.

FIG. 2A-2F are photomicrographs (x400, polarized light) of (A) the emulsion composition of Example 4 demonstrating that no crystals are observable; (B) the water phase of the composition of Example 4 following addition of a surfactant demonstrating that no crystals are observable; (C and D) the water phase of the composition of Example 4 prior to the addition of surfactant in which crystals are clearly visible; (E) betamethasone valerate powder used in the preparation of the composition of Example 4 and (F) a commercial betamethasone valerate 0.12% cream.

FIG. 3 is a photomicrograph illustrating a steroid (bmv) emulsion.

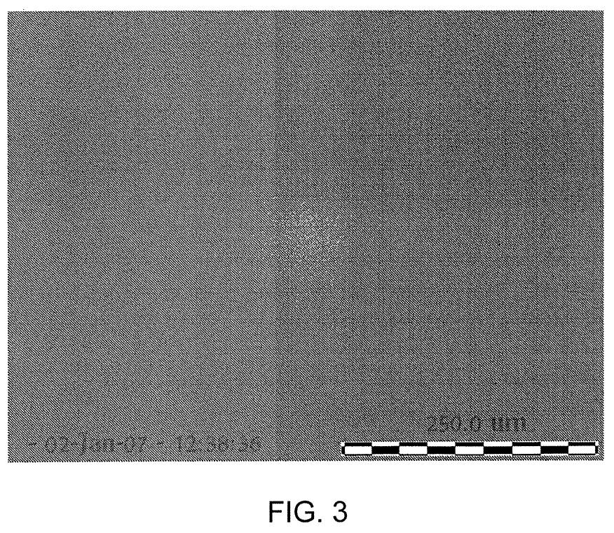

### DETAILED DESCRIPTION

The present invention relates to improved, easy to use, stable and non-irritating steroid foamable compositions and foams, intended for treatment of dermal and mucosal tissues. In the process of investigating steroid foamable compositions and carriers it has been noted that it is not sufficient to produce foams of quality but the problem is how produce formulations which are able to display characteristics of physical and chemical stability, which are biocompatible and which are capable of remaining sufficiently stable physically and chemically for a significant or even prolonged period of time.

Formulation of an oil-in-water emulsion foam is a very delicate balance between the functional inactive ingredients, excipients, which contribute to droplet size, separating film, viscosity and stability. In order to assure accurate and continuous foam actuation, the “Pre Foam Formulation” (PFF) should be liquid and shakable in the canister, otherwise the PFF will not flow easily and completely towards and through the valve.

Stability of emulsions and resilience to creaming is desired. In the context of foamable emulsion compositions it has been discovered that improved physical stability is obtained by an appropriate increase in product viscosity through use of viscoelastic agents, which can provide suitable rheology whilst retaining the requirements of shakability and by controlling droplet size. Smaller droplets will migrate in a slower manner and appropriate increased viscosity minimizes droplets migration and creaming and reduces the occurrences of droplets contact and fusion. However, the formulation should have sufficient viscosity to support emulsion stability, inhibit droplets migration and separation phenomenon and yet remain flowable.

By creaming, is meant that particles of the disperse phase concentrate in the upper layer, form a cream-like concentrated emulsion. The creaming value is defined as the relative volume of the creamed phase and the total volume the sample. The expression used for calculation of the creaming volume is as follows:

$$\% Creaming = \frac{{V}_{Creamed Phase}}{{V}_{total}} \times 100$$

Creaming values are between 1% and 99%, accordingly. 100% means “no creaming” which is the desirable best score.0% (Zero value) indicates phase separation and is the worst score.

In the present invention it has been discovered that it is possible to meet and balance these contradictory aspects to improve the physical properties and increase the physical stability period of the foamable composition and therefore any resultant foam produced there from by incorporating into the composition an effective amount of a stabilizing agent. In parallel it has likewise been discovered that it is possible to improve the chemical stability of the steroid or other active agent in the composition by use of a modulating agent in the composition. The modulating agent acts to create or maintain an environment within the formulation suitable for maintaining chemical stability of the steroid in its active form.

It has further been discovered that it is possible to make foamable compositions that will produce foam of a reasonable quality without the addition of a polymeric or a gelling agent. However, in order to produce a physically rugged or durable foamable composition that will produce a foam of quality it is necessary to introduce a suitable stabilizing agent, which can be polymeric or gelling agent. Additionally the polymeric or gelling agent may contribute to the elegance or quality of foam produced from the composition. By physically rugged or durable it is intended that the formulation is capable of physically withstanding to a substantial degree one, two or possibly more freeze thaw cycles; or a period of time at an elevated temperature of say 30° C. or say 40° C. for say one or two or possibly three months; or a prolonged period of time at room temperature for say three to six months or possibly longer.

It has been discovered that in the context of foamable formulations it is possible to improve the properties of the foamable composition and thereby the resultant foam produced there from by selecting carefully a suitable stabilizing agent, which in turn can be a suitable polymeric agent or gelling agent that has appropriate rheological properties suitable for a foamable composition. On the one hand the composition should be shakable to a sufficient or minimal degree. By shakable it indicates that some motion or movement of the formulation can be sensed when the canister containing the formulation is shaken or is strongly shaken. When the formulation is like a block or excessively viscous like an ointment it will not be shakable. On the other hand the foamable formulations have to be capable of being extruded through a valve and tube in an aerosol canister under pressure of a propellant and therefore the compositions should be flowable to a sufficient extent that allows the formulation to pass through a tube and valve to form a foam of sufficient quality upon being expelled from the aerosol. It has been discovered that in the context of the foamable compositions that in a preferred embodiment the stabilizing agent should exhibit pseudoplastic rheological behavior. In other words, to exhibit physical behavior that provides a flowable or shakable viscous system that at rest is capable of introducing to or restoring in the composition a sufficient viscosity to retard the motion or migration of emulsion droplets but whose viscosity decreases when subjected to shear stress, which in this case can be vigorous shaking.

By selective use of appropriate stabilizing surfactant, co-surfactants and stabilizing polymers the compositions can be stabilized. One method to overcome the contradicting physical requirements is to base the stabilizing agent on viscoelastic polymers which can form pseudo-plastic or semi pseudoplastic compositions.

Thus in one or more embodiments there is provided a pseudoplastic viscoelasic or semi-pseudoplastic viscoelastic steroid composition.

It has been further discovered that by creating an appropriate rheological emulsion flow behavior it has been possible to develop longer lasting compositions. By appropriate selection of agents and excipients to facilitate biocompatibility and to achieve the appropriate balance of physical properties, including rheology, emulsion droplet size, the density of each phase, and the viscosity of the continuous phase, it has been discovered in the context of foamable compositions that it is possible to prepare formulations that are resilient to creaming/phase separation when subjected to centrifugation. It has been further discovered that it is possible to prepare resilient formulations that are resilient to creaming/phase separation even under harsh centrifugation conditions such that could be extrapolated to reflect a stable shelf life of years.

It has been further discovered that polymers such as cellulose derivatives, Xanthan gum, Sodium CMC, methylcellulose, hydroxylpropyl methyl cellulose and the like, are suitable to provide viscoelastic compositions that have pseudoplastic or semi-pseudoplastic viscoelastic properties and which are able to contribute sufficiently to viscosity stabilizing the PFF emulsion, but are able to temporarily loose this property upon stress such as human hand shaking. The result is a stable emulsion and product with low or very low creaming (droplets migration) on shelf and liquid and shakable and dispensing foam upon shaking and actuation.

Thus, in one or more embodiments, there is provided a resilient formulation. In certain embodiments the resilient formulation is a viscoelastic formulation. In certain embodiments the viscoelastic formulation is a pseudoplastic viscoelastic composition. Thus in certain formulations the composition comprises a resilient agent, which can in turn be a viscoelastic or a pseudoplastic or a semi-pseudoplastic agent.

It has been further discovered that for certain formulations where the active agent is susceptible tone or more of isomerization, chemical breakdown, chemical reaction, oxidation and the like and the compound is to be formulated in a composition suitable for being stored for a period of time, which could be a short, medium or longer period of time within the context of a pharmaceutical composition in order to achieve a composition that is capable of being physically and chemically stable the formulation should comprise one or more quiescence agents. By chemically stable reference is intended to the chemical stability of an active agent in the pharmaceutical compositions and foams such that the active agent should be capable of remaining chemically stable or substantially so in the composition for a sufficient reasonable period of time to allow for pharmaceutical application of an effective amount of active agent to a subject. By quiescence agents is meant agents which on their own or in combination or within the context of the formulation have a calming or quieting or stabilizing or modulating effect or combinations thereof on the composition and includes the ability to calm or restore viscosity and or may include the ability to protect the composition or one or more ingredients from a form of chemical change and or may include the ability to resist or be resilient to creaming/phase separation.

Thus, in one or more embodiments there is provided a foamable composition comprising at least one quiescence agent.

Thus, in one or more embodiments there is provided a quiescence foamable composition including a quiescence agent selected from a stabilizing, a polymeric, a gelling, a viscoelastic, a pseuedoplastic or semi-pseudoplastic, a resilient or a modulating agent. In certain embodiments an agent may have more than one of these characteristics. In certain other compositions a combination of two or more agents is used to achieve a quiescence foamable composition having more than one of their characteristics.

In some embodiments, a therapeutic kit including a foamable steroid composition is provided. The kit includes an aerosol packaging assembly having a container accommodating a pressurized product and an outlet capable of releasing the pressurized product as a foam.

### Aerosol Packaging Assembly

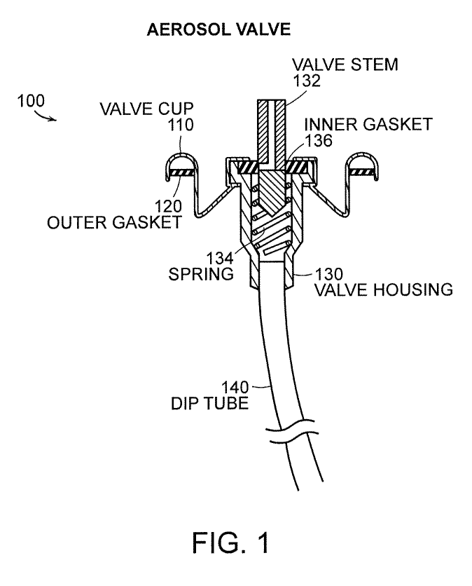

The aerosol packaging assembly typically includes a container suitable for accommodating a pressurized product and an outlet capable of releasing a foam. The outlet is typically a valve. FIG. 1 illustrates a typical aerosol valve 100. The valve is made up of the valve cup 110 typically constructed from tinplated steel, or aluminum, an outer gasket 120, which is the seal between the valve cup and the aerosol can (not shown), a valve housing 130, which contains the valve stem 132, spring 134 and inner gasket 136, and a dip tube 140, which allows the liquid to enter valve. The valve stem is the tap through which the product flows. The inner gasket 136 covers the aperture 150 (hole) in the valve stem. The valve spring 134 is usually made of stainless steel.

The valve stem is fitted with small apertures 150 (also termed “orifices” and “holes”), through which the product flows. Valves may contain one, two, three, four or more apertures, depending on the nature of the product to be dispensed. In the closed position, the aperture(s) is covered by the inner gasket. When the actuator is depressed it pushes the valve stem through the inner gasket, and the aperture(s) is uncovered, allowing liquid to pass through the valve and into the actuator.

The valve can have a stem with 1 to 4 apertures, or 1 to 2 apertures. Each aperture can have a diameter of about 0.2 mm to about 1 mm, or a diameter of about 0.3 mm to about 0.8 mm. The total aperture area, i.e., the sum of areas of all apertures in a given stem, is between about 0.01 mm² and 1 mm² or the total aperture area is between about 0.04 mm² and 0.5 mm².

In order to provide proper therapy, precise dosing is desired. According to one or more embodiments, the valve is attached, directly or through a tube, to a metered dose device for dispensing an accurate dose of drug in the form of a foam. The metered dose valve is selected to release a foam in a volume that provides an adequate therapeutic dose to the target site of the skin, a body surface, a body cavity or mucosal surface, e.g., the mucosa of the nose, mouth, eye, ear, respiratory system, vagina or rectum.

In one or more embodiments, the meter dose valve provides a unit dose of between about 10 μL and about 1000 μL of liquid. Assuming a representative foam density (specific gravity) of 0.06 g/mL, a 10 μL valve provides a foamed volume of about 0.17 mL, and a 1000 μL metered dose valve provides a foamed volume of about 17 mL. Thus, by selecting a specific metered dosing valve, adjusting the foam density by fine-tuning formulation parameters and adjusting the ratio between the liquid components of the composition and the propellant, one can design an adequate dosage form for a specific target site.

### Pharmaceutical Composition

All % values are provided on a weight (w/w) basis.

According to one or more embodiments, the foamable therapeutic composition for administration to the skin, a body surface, a body cavity or mucosal surface, e.g., the mucosa of the nose, mouth, eye, ear, respiratory system, vagina or rectum, e.g., the “target site” includes:
(1) a steroid, wherein the amount of the amount of the steroid is effective in the treatment of a disorder of the target site;
(2) at least one organic carrier selected from a hydrophobic organic carrier, a polar solvent, an emollient and mixtures thereof, at various concentrations, e.g., about 2% to about 5%; or about 5% to about 10%; or about 10% to about 20%; or about 20% to about 50% by weight;
(3) a surface-active agent;
(4) about 0.01% to about 5% by weight of at least one polymeric agent selected from a bioadhesive agent, a gelling agent, a film forming agent and a phase change agent; and
(5) a liquefied or compressed gas propellant at a concentration of about 3% to about 25% by weight of the total composition.

Water and optional ingredients are added to complete the total mass to 100%. Upon release from an aerosol container, the foamable composition expands to form foam that is suitable for topical administration.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

In one or more specific embodiments where the active ingredient is coal tar, which may be formulated for inclusion in one or more of the carrier compositions of the present invention as an alcoholic extract. In an embodiment the alcoholic component of the coal tar extract can amount to about 8% of the total composition. Thus, when the active agent is coal tar the formulation is regarded as substantially alcohol free if it includes less than about 10% final concentration of lower alcohols.

In one or more embodiments, the alcoholic content of the coal tar extract is reduced or substantially eliminated by gentle warming to distil off the lower alcohols.

In one or more embodiments, at least a portion of the steroid is suspended in the composition, yet, in other embodiments, the steroid is dissolved in the composition.

In one or more embodiments, the foam composition is formulated as an oil-in-water emulsion or oil-in-water microemulsion.

The present invention provides foamable therapeutic compositions useful for delivery of a steroid agent to a mucosal body. The composition is dispensed as a foam providing a stable product that is pleasant and easy to use for high patient compliance.

The foamable therapeutic composition is suitable for facile administration into the rectum, bladder, the cavity between the uterus and the fallopian tubes, the ovaries and other body areas, which may accept topically-applied products.

In one or more embodiments, a foamable composition includes water in one phase and at least one solvent selected from a hydrophobic organic carrier, a polar solvent, an emollient and mixtures thereof in the second phase. The compositions may be water-in-oil or oil-in-water emulsions.

Despite the commonly accepted understanding that hydrophobic organic carriers, polar solvents and emollients are difficult to formulate into a foam-producing product and that addition of such solvents interferes with the foam forming ability of a surfactant, the present invention has surprisingly identified a series of foam compositions, which, upon admixing with a liquefied gas propellant in an aerosol container, produce a stable foam composition that is suitable for topical and mucosal administration to body cavities, such as the vagina, rectum, colon, penile, urethra, nasal cavity and ear cavity. Upon discharge from an aerosol container, the composition forms an expanded foam, which does not break down immediately upon discharge, and remains in the body cavity for an extended time.

Such compositions have been developed so that they are physically stable and resilient and that the steroid remains chemically stable when the composition is placed in an aerosol container and combined with a liquefied gas propellant, to provide an emulsion, which, upon release from the aerosol container, provides a therapeutically beneficial foam product.

### Steroids

Steroids are compounds possessing the skeleton of cyclopenta[a]phenanthrene or a skeleton derived therefrom by one or more bond scissions or ring expansions or contractions. Methyl groups are normally present at C-10 and C-13. An alkyl side chain may also be present at C-17. Sterols are steroids carrying a hydroxyl group at C-3 and most of the skeleton of cholestane. Additional carbon atoms may be present in the side chain.

Steroids are numbered and rings are lettered as in formula 1. If one of the two methyl groups attached to C-25 is substituted it is assigned the lower number (26); if both are substituted, that carrying the substituent cited first in the alphabetical order is assigned the lower number.

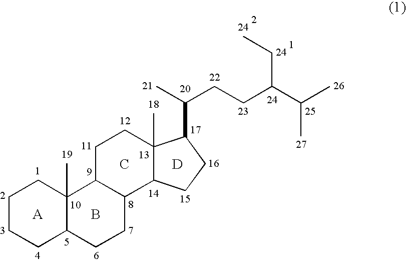

The steroids can have substituents on the steroid side chain as exemplified in formula 4-7:

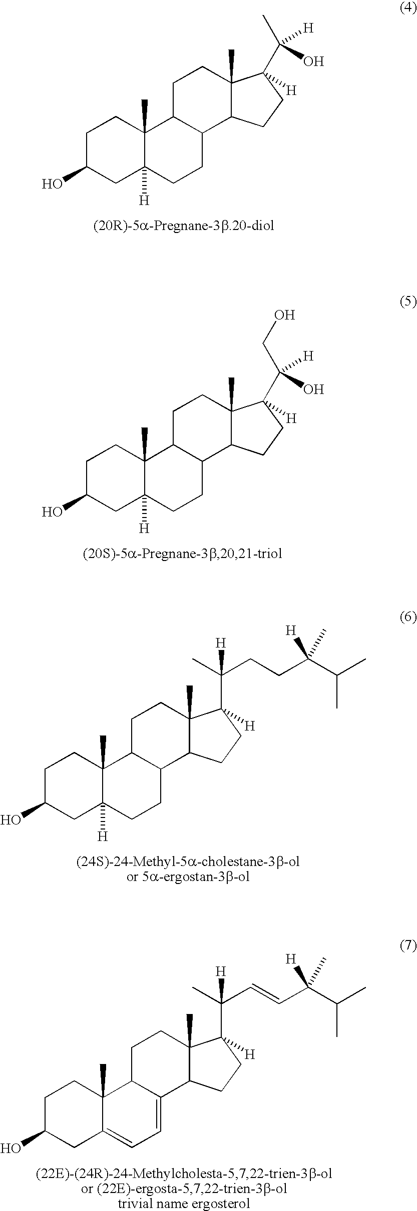

The steroids can have the formalae as exemplified in formula 9-18. In one or more embodiments, the steroid or sterol has no substitution at C-17, as exemplified by gonane, e.g., formulae 9 and 10, estrange (also termed oestrane), e.g. Formulae 11 and 12, and androstane, e.g., formulae 13 and 14. In one or more embodiments, the steroid or sterol has methyl groups at both C-10 and C-13 and a side chain R at C-17 (formulae 15 and 16), as exemplified in Table 1.

TABLE 1
Hydrocarbons with side chain at C-17

| | |
|---|---|
| | (9) |
| 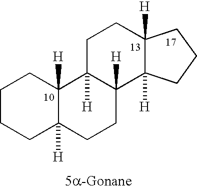 | |

| | |
|---|---|
| | (10) |
| 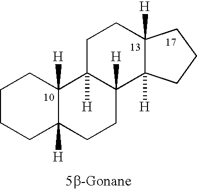 | |
| | (11) |
| 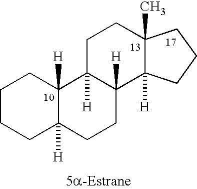 | |
| | (12) |
| 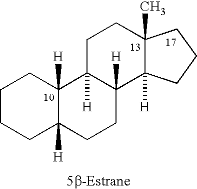 | |
| | (13) |
| 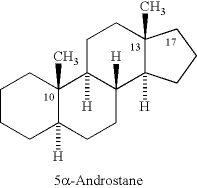 | |
| | (14) |
| 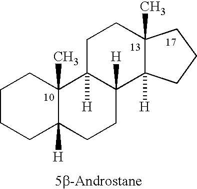 | |
| | (15) |
| 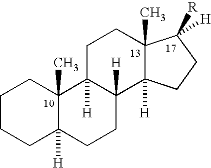 | |
| | (16) |
| 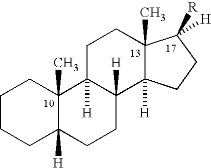 | |
| | (17) |
| 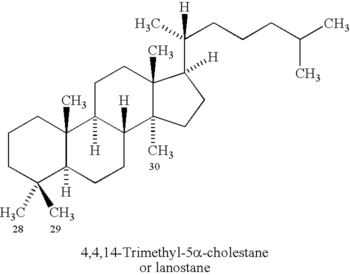 | |
| | (18) |
| 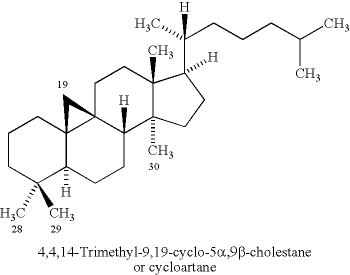 | |

| Side chain | 5α-Series (15) | 5β-Series (16) |
|---|---|---|
| 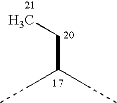 | 5α-pregnane(allopregnane) | 5β-pregnane |
| 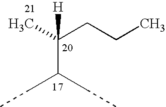 | 5α-cholane(allocholane) | 5β-cholane |
| 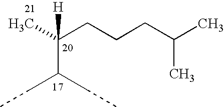 | 5α-cholestane | 5β-cholestane(coprostane) |
| 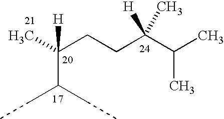 | 5α-ergostane | 5β-ergostane |
| 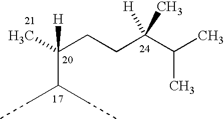 | 5α-campestane | 5β-campestane |
| 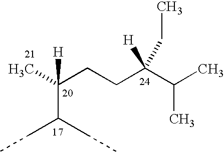 | 5α-poriferastane | 5β-poriferastane |
| 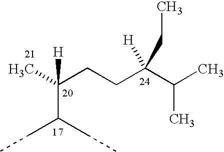 | 5α-stigmastane | 5β-stigmastane |
| 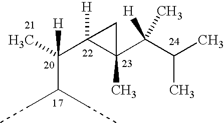 | 5α-gorgostane | 5β-gorgostane |

Examples of unsaturated steroids and sterols are provided in formulae 19-22:

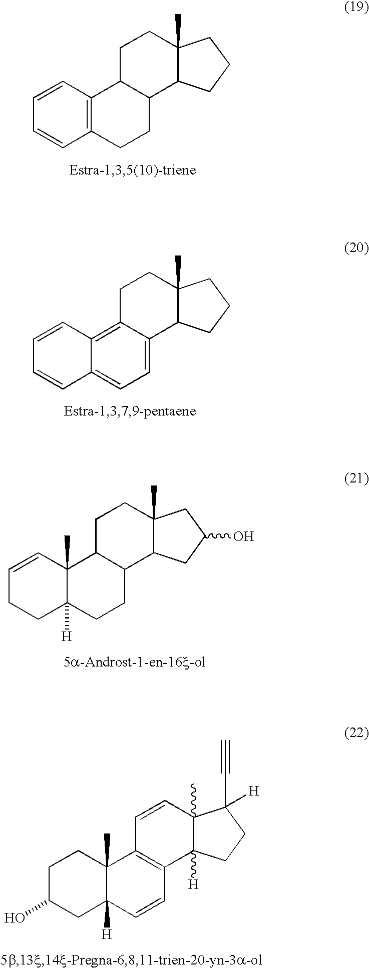

The stereochemistry of double bonds in the side chain is indicated using the E,Z convention. The same applies to the seco compounds of the vitamin D series (example in formula 23). In certain cases, the steroid has two carbon chains attached at position 17, e.g. 17-methyl-5α-pregnane 24,17-methyl-5α, 17β-pregnane 25, and 17-ethyl-5-cholestane and 17-(2-bromoethyl)-5α, 17-cholestane 26. Other examples of a steroid that has two carbon chains attached at position 17, are 17,17-dimethyl-5α-androstane 27 and 17β-methyl-17α-propyl-5α-androstane 28. In certain embodiments, the carbon skeleton of a steroid a carbon atom is replaced by a hetero atom, as exemplified by 17β-hydroxy-4-oxaandrost-5-en-3-one 29. Yet, in additional embodiments, an additional ring is formed by means of a direct link between any two carbon atoms of the steroid ring system or the attached side chain, as exemplified by formulae 30, 31 and 32.

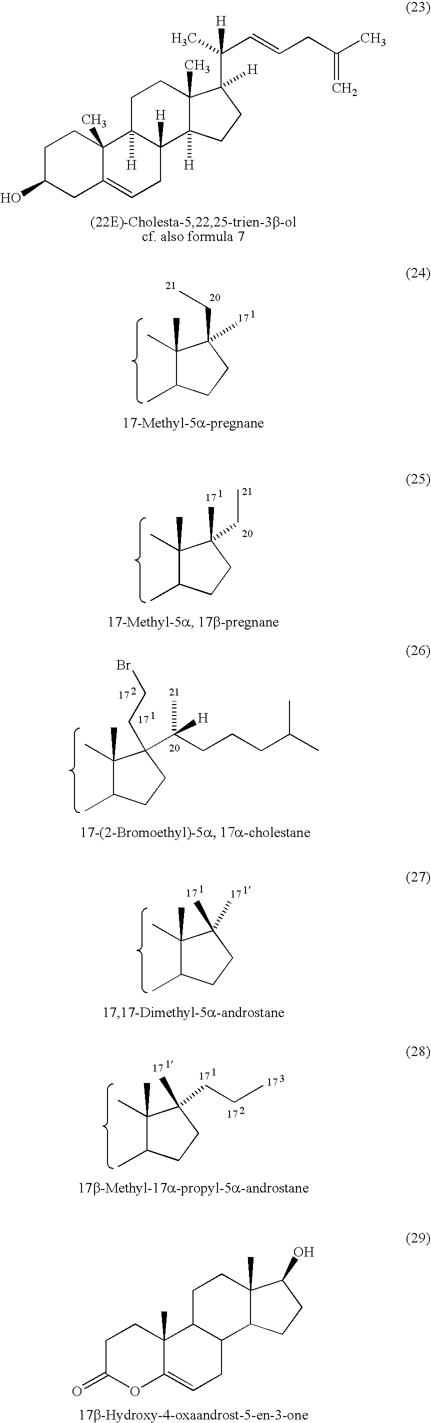

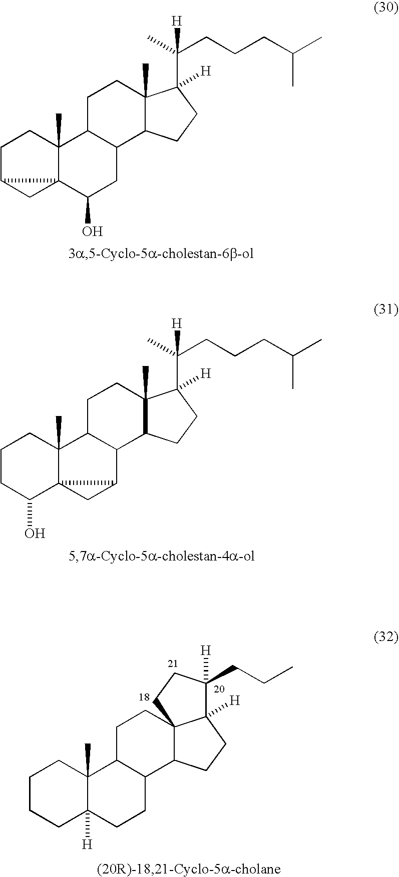

Many important naturally occurring steroids contain one or more additional heterocyclic ring(s), fused or attached to ring D, formed by modifications of the side chain. These steroids can be grouped into the following families: (a) cardanolides, e.g., 5β-cardanolide 33, 3β,14-dihydroxy-5β-card-20(22)-enolide (digitoxigenin) 34 and 3β,5,14-trihydroxy-19-oxo-5β-card-20(22)-enolide (strophanthidin) 35, as well as epoxycardanolides, containing a 14,21- or a 16,21-oxygen bridge, as shown in 36, (b) bufanolides, e.g., structures 37-39, (c) spirostans, e.g., structures 40-43, (d) furostans, e.g., structures 44-45, and (e) steroid alkaloids.

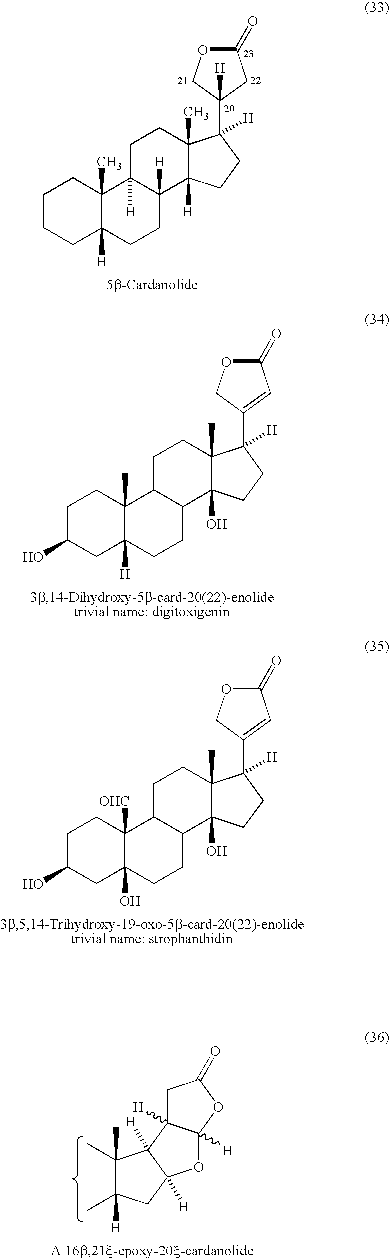

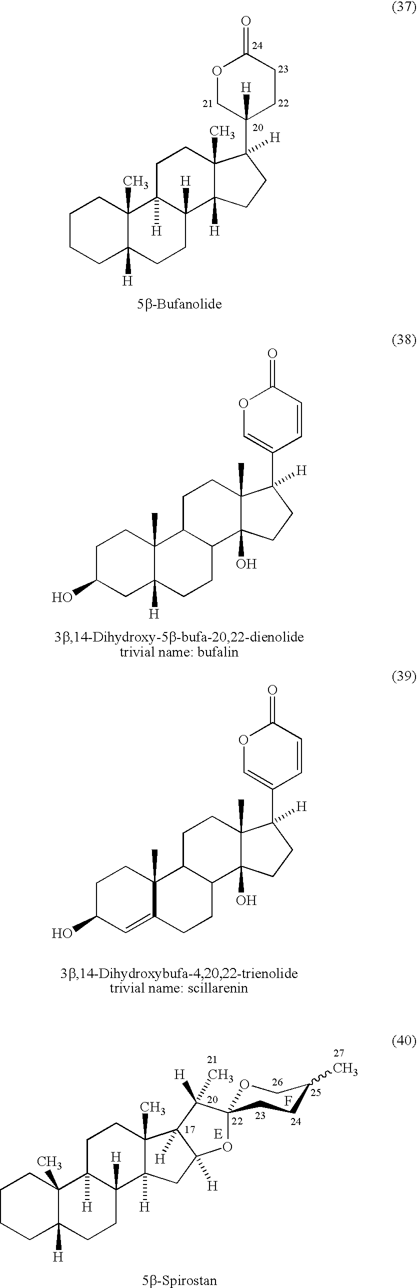

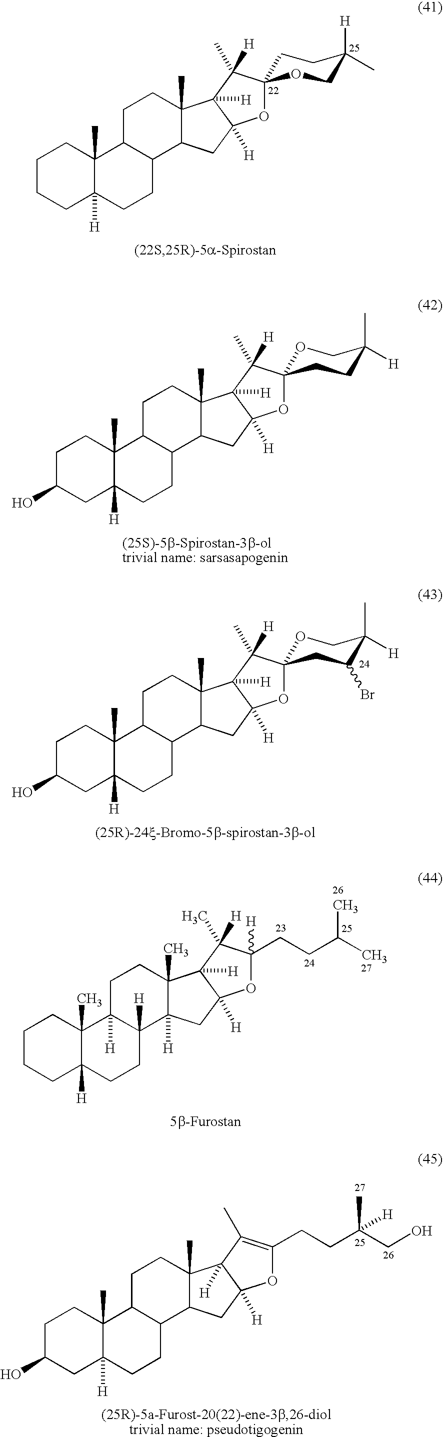

Several biologically important steroids are derivatives of the parent hydrocarbons carrying various functional groups. Some of the common functional groups include but are not limited to halogens, alkyl groups, aryl groups, benzyl groups, carboxy groups and alkoxy groups.

In one or more embodiments, the steroid is selected from the group consisting of an acid, a salt of an acid, as exemplified in formulae 46-49, and esters, as exemplified in formulae 50 and 51. In one or more embodiments, the steroid is a lactone, as exemplified in formulae 52-54.

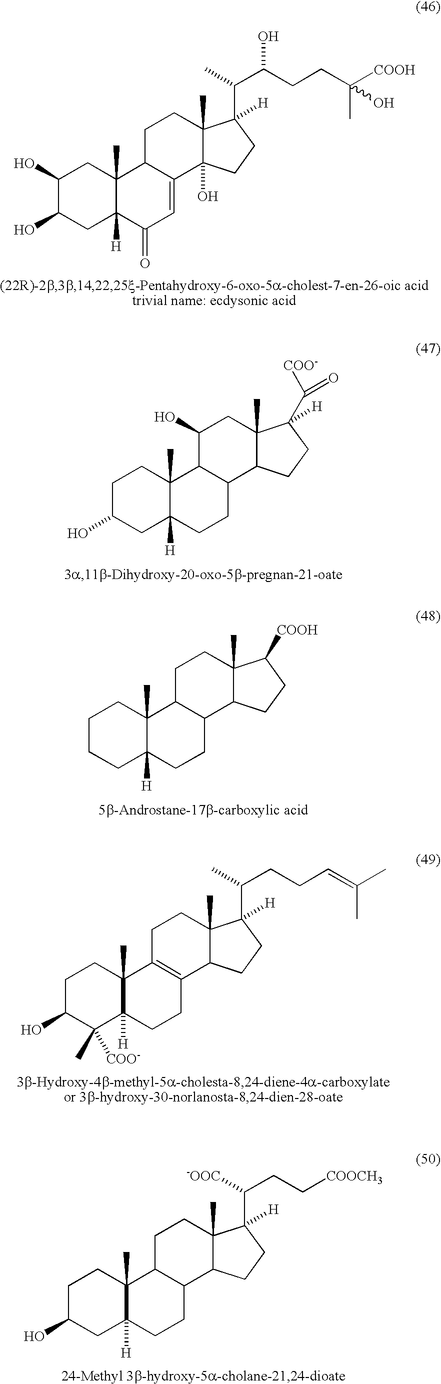

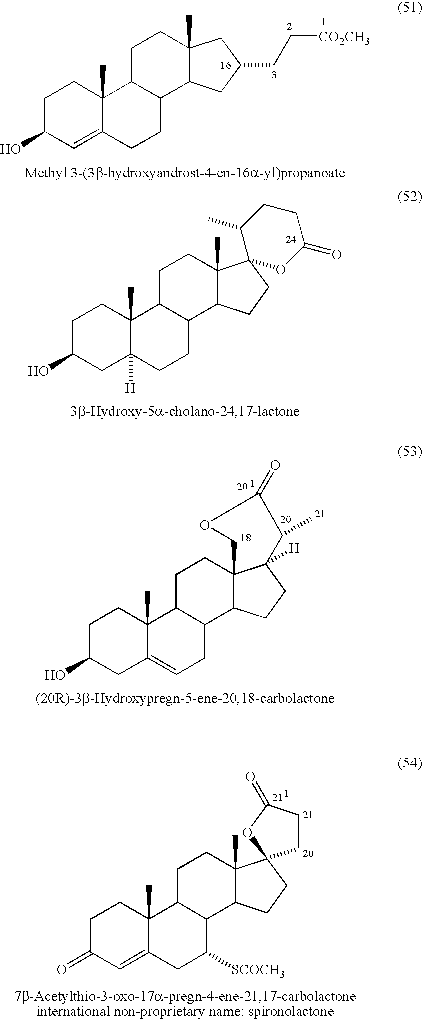

In one or more embodiments, the steroid is an ester of a steroid alcohol, as exemplified by 5-cholestan-3-yl acetate, 5-cholestane-3,12-diyl diacetate, 3-oxoandrost-4-en-17-yl acetate (trivial name testosterone acetate), 17-hydroxy-20-oxopregn-5-en-3-yl sulfate, 3-acetoxy-5-cardanolide, 3-benzoyloxy-11-hydroxy-20-oxo-5-pregnan-21-oate (monobenzoate of 47), 3-acetoxy-5-cholano-24,17-lactone (acetate of 52), 3-O-acetylcholic acid, 17-O-benzoylestradiol-17, 3-O-linolenoylcholesterol, as well as in formulae 55 and 56.

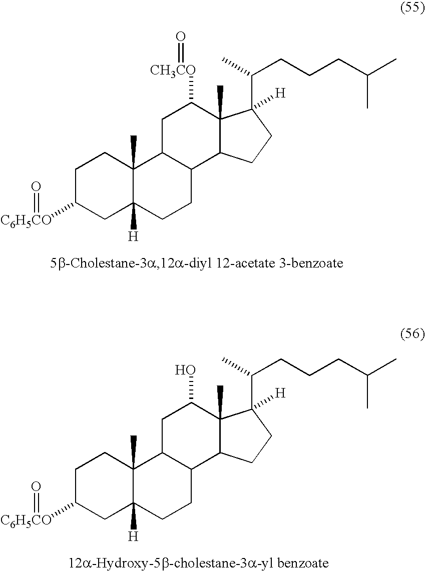

In one or more embodiments, the steroid is an oxo compound. The oxo compound can be an aldehde, as exemplified by 5-androstan-19-al, 5-cholan-24-al, 3-formyl-5-cholan-24-oic acid and by formulae 57 and 58, or a ketone, as exemplified by 5-androstan-3-one, pregn-5-ene-3,20-dione and 11-oxo-5-cholan-24-oic acid.

In one or more embodiment, the steroid is an alcohol as exemplified by 5-cholestane-3,11-diol, 3-hydroxy-5-androstan-17-one (trivial name: androsterone) and by formulae 59.

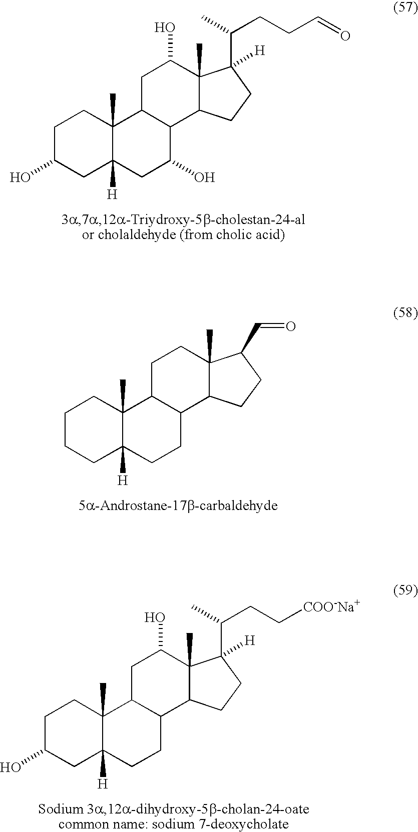

In additional embodiments, the steroid is an amine as exemplified by androst-5-en-3-amine and formula 60, an ether as exemplified by 17-methoxyandrost-4-en-3-one, (20S)-3,17,20-trimethoxy-5-pregnane, (20S)-3,17-dimethoxy-5-pregnan-20-ol, 21-O-methylcortisol and formula 61, an acetal or a ketal of an oxo steroid (also named as dialkoxy steroids) as exemplified by 3,3-dimethoxycholest-4-ene, 2,3-(methylenedioxy)pregn-5-ene and formula 62.

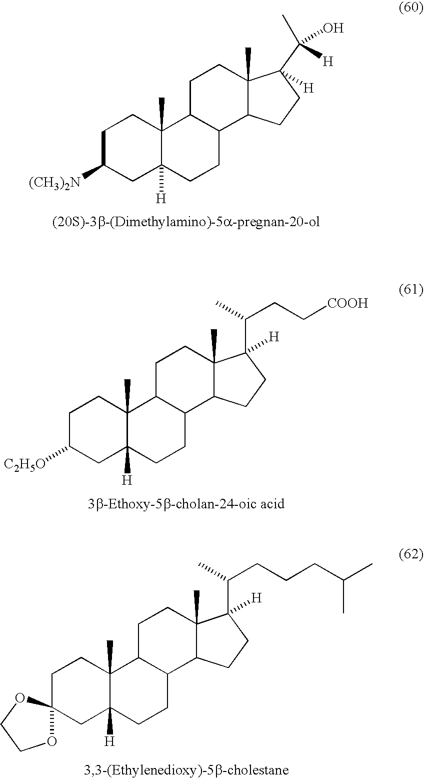

Examples of trivial names retained for important steroid derivatives, these being mostly natural compounds of significant biological activity, are given in Table 2.

TABLE 2
Trivial names of some important steroid derivatives

| Trivial name | Systematic steroid name |
|---|---|
| Aldosterone | 18,11-hemiacetal of 11^{β},21-dihydroxy-3,20- |
| | dioxopregn-4-en-18-al or 11^{β},18-epoxy-18ξ,21- |
| | dihydroxypregn-4-ene-3,20-dione |
| Androsterone | 3α-hydroxy-5α-androstan-17-one |
| Brassinolide | (22R,23R)-2α,3α,22,23-tetrahydroxy-6,7-seco- |
| | 5α-cmpestano-6,7-lactone |
| Calcidiol (93) | (5Z,7E)-(3S)-9,10-secocholesta-5,7,10(19)-triene- |
| | 3,25-diol |
| Calciol = | (5Z,7E)-(3S)-9,10-secocholesta-5,7,10(19)-trien- |
| cholecalciferol (92) | 3-ol |
| Calcitriol (94) | (5Z,7E)-(1S,3R)-9,10-secocholesta-5,7,10(19)- |
| | triene-1,3,25-triol |
| Cholesterol | cholest-5-en-3^{β}-ol |
| Cholic acid | 3α,7α,12α-trihydroxy-5^{β}-cholan-24-oic acid |
| Corticosterone | 11^{β},21-dihydroxypregn-4-ene-3,20-dione |
| Cortisol | 11^{β},17,21-trihydroxypregn-4-ene-3,20-dione |
| Cortisol acetate | 21-O-acetylcortisol |
| Cortisone | 17,21-dihydroxypregn-4-ene-3,11,20-trione |
| Cortisone acetate | 21-O-acetylcortisone |
| Deoxycorticosterone | 21-hydroxypregn-4-ene-3,20-dione (i.e. The 11- |
| | deoxy derivative of corticosterone) |
| Ecdysone | (22R)-2^{β},3^{β},14^{α},22,25-pentahydroxy-5^{β}-cholest- |
| | 7-en-6-one |
| Ercalciol = | (5Z,7E,22E)-(3S)-9,10-secoergosta-5,7,10(19),22- |
| ergocalciferol | tetren-3-ol |
| Ergosterol (7) | (22E)-ergosta-5,7,22-trien-3^{β}-ol |
| Estradiol-17α | estra-1,3,5(10)-triene-3,17α-diol |
| Estradiol-17^{β} | estra-1,3,5(10)-triene-3,17^{β}-diol |
| Estriol | estra-1,3,5(10)-triene-3,16α,17^{β}-triol |
| Estrone | 3-hydroxyestra-1,3,5(10)-trien-17-one |
| Lanosterol | lanosta-8,24-dien-3^{β}-ol |
| Lithocholic acid | 3α-hydroxy-5^{β}-cholan-24-oic acid |
| Progesterone | pregn-4-ene-3,20-dione |
| Pseudotigogenin | (25R)-5α-furost-20(22)-ene-3^{β},26-diol |
| Sarsasapogenin | (25S)-5^{β}-spirostan-3^{β}-ol |
| Smilagenin | (25R)-5^{β}-spirostan-3^{β}-ol |
| Testosterone (63) | 17^{β}-hydroxyandrost-4-en-3-one |
| Tigogenin | (25R)-5α-spirostan-3^{β}-ol |

Additional non-limiting examples of steroids that are applicable according to the present invention are provided in formulae 63-79.

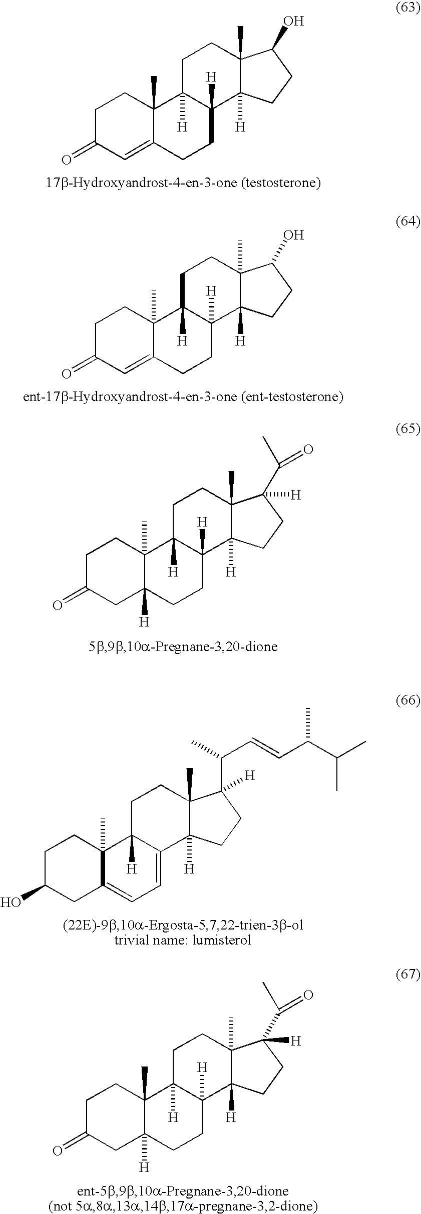

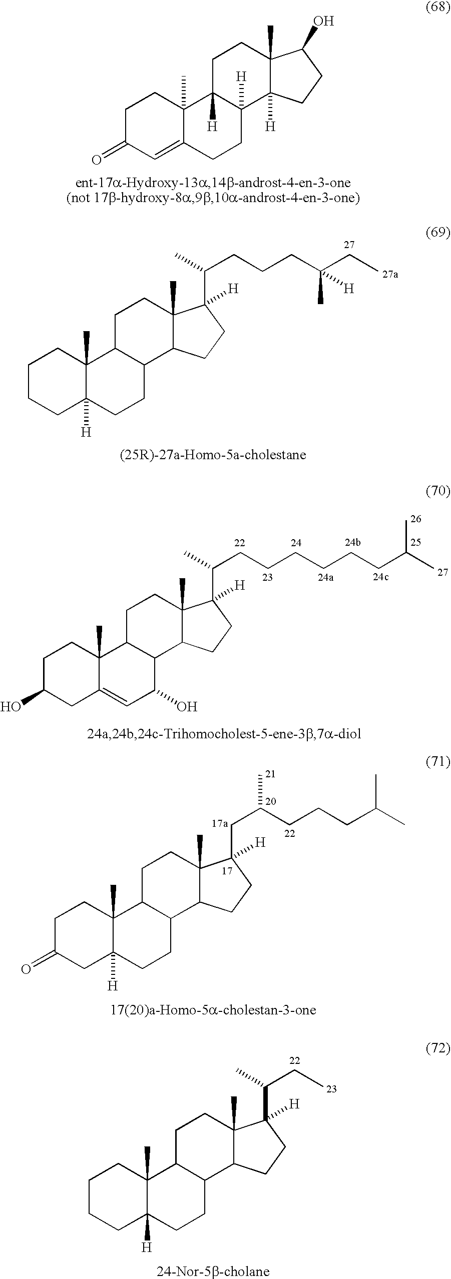

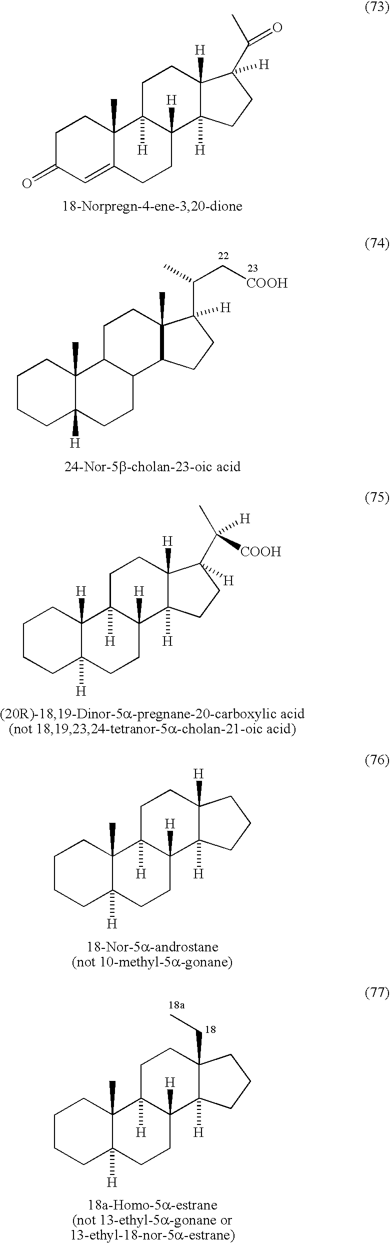

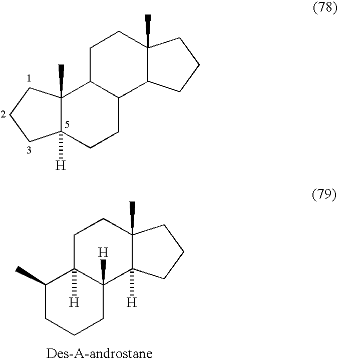

In one or more embodiments, the steroid is a compound, in which one or more of the cyclopenta[a]phenanthrene rings is contracted by loss of an unsubstituted methylene group, as exemplified by 4-nor-5-androstane (78), where C-4 is missing. In other embodiments one or more of the cyclopenta[a]phenanthrene rings is expanded by inclusion of a methylene group, as exemplified by formulae 80-86.

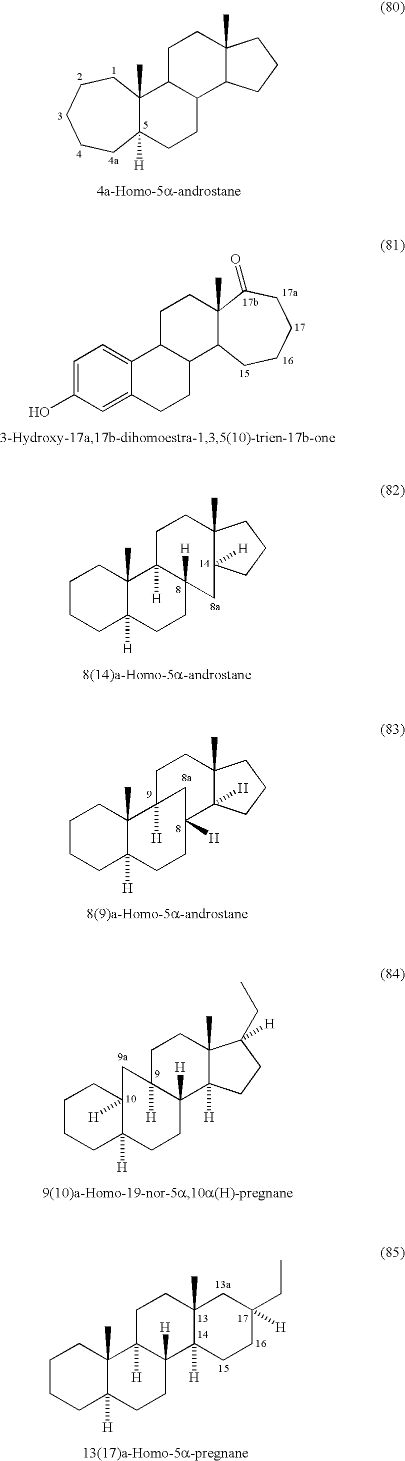

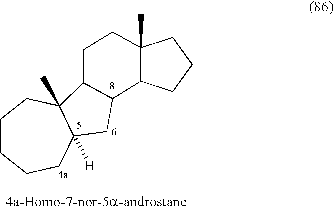

In one or more embodiments, the steroid contains additional rings that are formed within, or on, a steroid nucleus. In additional embodiments, the steroids contains a bivalent bridge such as —O—O—, —[CH₂]ₙ—, linking non-adjacent ring positions as exemplified by formulae 99-102.

In one or more embodiments, the steroid contains a cyclopenta[a]phenanthrene skeleton and a carbocyclic or heterocyclic ring component fused to it, as exemplified by formulae 103-111, and in other embodiments, an additional ring is linked to the cyclopenta[a]phenanthrene skeleton through a spiro system, as exemplified by formula 112.

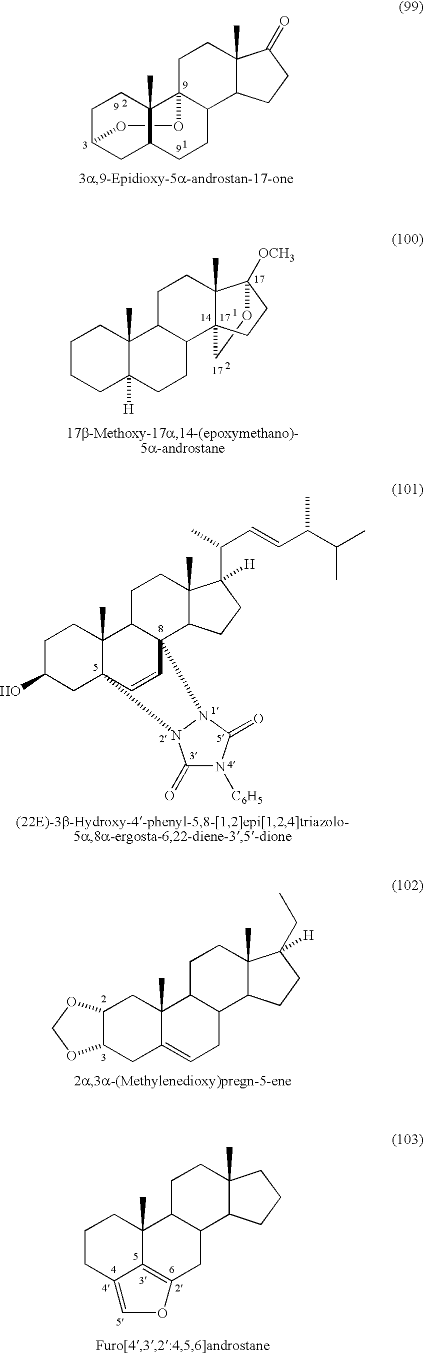

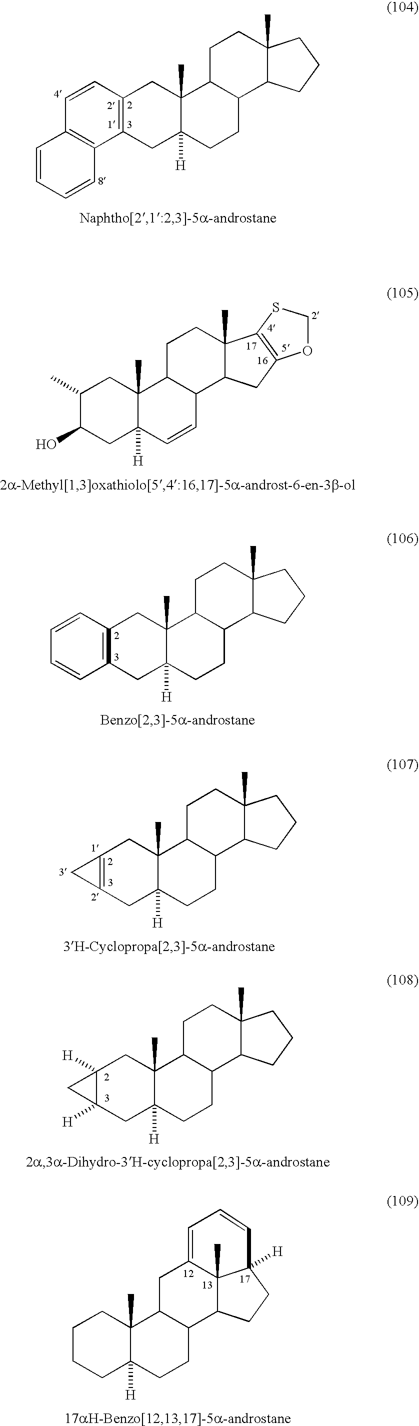

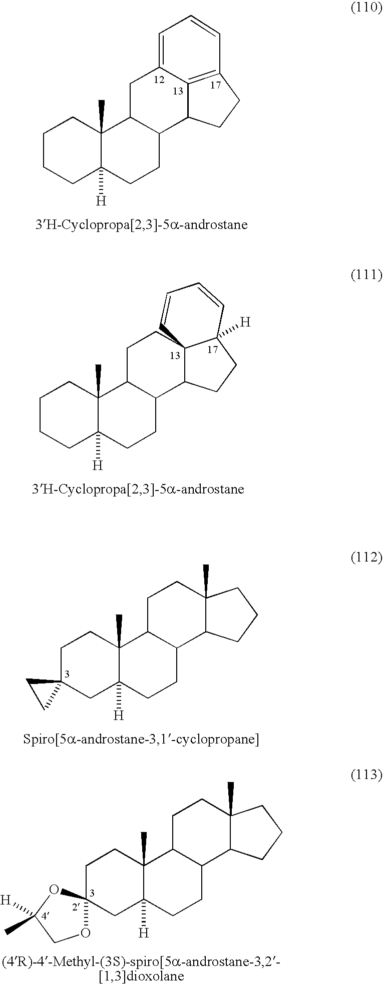

Yet, in certain embodiments, two or more steroid molecules are linked together covalently, as exemplified by formulae 3a and 3b.

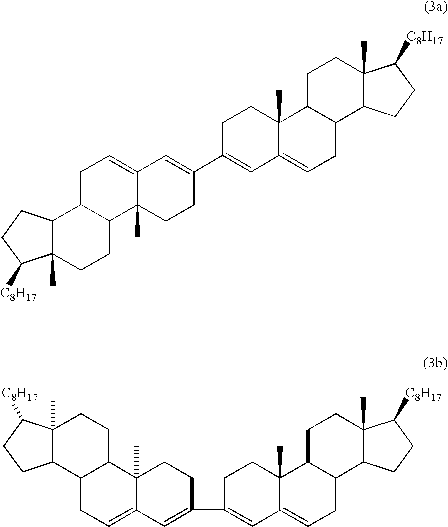

Table 3 provides examples of steroids that are useful according to the present invention.

TABLE 3
Exemplary steroids, which are useful according to the present invention.

| | | Molecular |
|---|---|---|
| Trivial name | Chemical name | formula |
| Acrihellin | 5,14-dihydroxy-3^{β}-[(3-methylcrotonoyl)oxy]- | C₂₉H₃₈O₇ |
| | 19-oxo-5^{β}-bufa-20,22-dienolide | |
| Actodigin | 3^{β}-(^{β}-D-glucopyranosyloxy)-14-hydroxy-24- | C₂₉H₄₄O₉ |
| | nor-5^{β},14^{β}-chol-20(2)-eno-21,23-lactone | |
| Alfacalcidol | (5Z,7E)-(1S,3R)-9,10-secocholesta-5,7,10(19)- | C₂₇H₄₄O₂ |
| | triene-,3-diol | |
| Betamethasone | 9-fluoro-11^{β},17,21-trihydroxy-16^{β}- | C₂₂H₂₉FO₅ |
| | methylpregna-1,4-diene-3,20-dione | |
| Canrenone | 3-oxo-17α-pregna-4,6-diene-21,17- | C₂₂H₂₈O₃ |
| | carbolactone | |
| Clomegestone | 6-chloro-17-hydroxy-16α-methylpregna-4,6- | C₂₂H₂₉ClO₃ |
| | diene-3,20-dione | |
| Cyproterone | 6-chloro-1^{β},2^{β}-dihydro-17-hydroxy-3′H- | C₂₂H₂₇ClO₃ |
| | cyclopropa[1,2]pregna-4,6diene-3,20-dione | |
| Dexamethasone | 9-fluoro-11^{β},17,21-trihydroxy-16α- | C₂₂H₂₉FO₅ |
| | methylpregna-1,4-diene-3,20-dione | |
| Disogluside | (25R)-3^{β}-(^{β}-D-glucopyranosyloxy)spirost-5- | C₃₃H₅₂O₈ |
| | ene | |
| Ethinylestradiol | 19-nor-17α-pregna-1,3,5(10)-trien-20-yne- | C₂₀H₂₄O₂ |
| | 3,17-diol | |
| Fluazacort | 21-acetoxy-9-fluoro-11^{β}-hydroxy-2′-methyl- | C₂₅H₃₀FNO₆ |
| | 16bH-oxazolo[5′,4′:16,17]pegna--1,4-diene- | |
| | 3,20-dione | |
| Fluocortin | 6α-fluoro-11^{β}-hydroxy-16α-methyl-3,20- | C₂₂H₂₇FO₅ |
| | dioxopregna-1,4-dien-21-oic acid | |
| Fusidic Acid | (17Z)-ent-16α-acetoxy-3^{β},11^{β}-dihydroxy- | C₃₁H₄₈O₆ |
| | 4^{β},8,14-trimethyl-18-nor-5^{β},10α-cholesta-- | |
| | 17(20),24-dien-21-oic acid | |
| Gestrinone | 17-hydroxy-18α-homo-19-nor-17α-pregna- | C₂₁H₂₄O₂ |
| | 4,9,11-trien-20-yn-3-one | |
| Halometasone | | |
| | 2-chloro-6α,9-difluoro-11^{β},17,21-trihydroxy- | C₂₂H₂₇ClF₂O₅ |
| | 16α-methylpregna-1,4-diene-3,20-dione | |
| Hydrocortisone | 11^{β},17,21-trihydroxypregn-4-ene-3,20-dione | C₂₁H₃₀O₅ |
| Mebolazine | 17^{β}-hydroxy-2α,17-dimethyl-5α-androstan-3- | C₄₂H₆₈N₂O₂ |
| | one azine | |
| Medroxy- | 17-hydroxy-6α-methylpregn-4-ene-3,20-dione | C₂₂H₃₂O₃ |
| progesterone | | |
| Meproscillarin | 3^{β}-(6-deoxy-4-O-methyl-α-L- | C₃₁H₄₄O₈ |
| | mannopyranosyloxy)-14-hydroxybufa-4,20,22- | |
| | rienolide | |
| Mespirenone | 7α-acetylthio-15α,16α-dihydro-3-oxo-3′H- | C₂₅H₃₀O₄S |
| | cyclopropa[15,1]-17α-pregna--1,4-diene- | |
| | 21,17-carbolactone | |
| Mestranol | 3-methoxy-19-nor-17α-pregna-1,3,5(10)-trien- | C₂₁H₂₆O₂ |
| | 20-yn-17-ol | |
| Naflocort | 9-fluoro-1′,4′-dihydro-11^{β},21-dihydroxy-16bH- | C₂₉H₃₃FO₄ |
| | naphtho[2′,3′:16,17]prena--1,4-diene-3,20- | |
| | dione | |
| Norenthisterone | 17-hydroxy-19-nor-17α-pregn-4-en-20-yn-3- | C₂₀H₂₆O₂ |
| | one | |
| Norgesterone | 17-hydroxy-19-nor-17α-pregna-5(10),20-dien- | C₂₀H₂₈O₂ |
| | 3-one | |
| Norgestrel | rac-17-hydroxy-18α-homo-19-nor-17α-pregn- | C₂₁H₂₈O₂ |
| | 4-en-20-yn-3-one | |
| Oxandrolone | 17^{β}-hydroxy-17α-methyl-2-oxa-5α-androstan-3-one | C₁₉H₃₀O₃ |
| Oxymetholone | 17^{β}-hydroxy-2-(hydroxymethylene)-17α- | C₁₉H₂₈O₃ |
| | methyl-5α-androstan-3-one | |
| Pancuronium | 1,1′-(3α,17^{β}-diacetoxy-5α-androstane- | C₃₅H₆₀Br₂N₂O₄ |
| bromide | 2^{β},16^{β}-diyl)bis(-methylpiperidinium) dibromide | |
| Prednisolone | 11^{β},17,21-trihydroxypregna-1,4-diene-3,20- | C₂₁H₂₈O₅ |
| | dione | |
| Prednisone | 17,21-dihydroxypregna-1,4-diene-3,11,20- | C₂₁H₂₆O₅ |
| | trione | |
| Proscillardin | 3^{β}-(6-deoxy-α-L-mannopyranosyloxy)-14- | C₃₀H₄₂O₈ |
| | hydroxybufa-4,20,22-trienolide | |
| Roxibolone | 11^{β},17^{β}-dihydroxy-17α-methyl-3- | C₂₁H₂₈O₅ |
| | oxoandrosta-1,4-diene-2-carboxylic acid | |
| Spironolactone | 7α-acetylthio-3-oxo-17α-pregn-4-ene-21,17- | C₂₄H₃₂O₄S |
| | carbolactone | |
| Timobesone | S-methyl 9-fluoro-11^{β},17α-dihydroxy-16^{β}- | C₂₂H₂₉FO₄S |
| | methyl-3-oxoandrosta--1,4-diene-17^{β}- | |
| | carbothioate | |
| Triamcinolone | 9-fluoro-11^{β},16α,17,21-tetrahydroxypregna- | C₂₁H₂₇FO₆ |
| | 1,4-diene-3,20-dione | |
| Ursodeoxycholic | 3α,7^{β}-dihydroxy-5^{β}-cholan-24-oic acid | C₂₄H₄₀O₄ |
| acid | | |

Mixtures of these steroids may also be employed according to the present invention.

Solubility of the steroid is an important factor in the development of a stable foamable composition according to the present invention.

For definition purposes in context herein, the descriptive terminology for solubility according to the US Pharmacopoeia (USP 23, 1995, p. 10), the European Pharmacopoeia (EP, 5^{th} Edition (2004), page 7) and several other textbooks used in the art of pharmaceutical sciences (see for example, Martindale, The Extra Pharmacopoeia, 30^{th} Edition (1993), page xiv of the Preface; and Remington's Pharmaceutical Sciences, 18^{th} Edition (1990), page 208) is adapted:

| | Parts of Solvent Required for 1 |
|---|---|
| Descriptive Term | Part of Solute |
| Very soluble | Less than 1 |
| Freely soluble | From 1 to 10 |
| Soluble | From 10 to 30 |
| Sparingly soluble | From 30 to 100 |
| Slightly soluble | From 100 to 1,000 |
| Very slightly soluble | From 1,000 to 10,000 |
| Practically insoluble or Insoluble | 10,000 and over |

Thus, in one or more embodiments, the steroid is “soluble”, “freely soluble” or “very soluble” (as defined above) in the aqueous phase of the emulsion. In other embodiments, the agent possesses hydrophobic characteristics and the steroid is “soluble”, “freely soluble” or “very soluble” in the oil phase of the emulsion. Yet, in certain cases, the steroid is “very slightly soluble”, “slightly soluble” or “sparingly soluble” in either the water phase or oil phase of the emulsion.

Yet, in one or more embodiments, the steroid is insoluble i.e., “requires 10,000 parts or more of a solvent to be solubilized”, in either the water phase of the composition, or the oil phase of the composition, but not in both.

It has been surprisingly discovered herein, that while insoluble in water, a steroid can be solubilized in the aqueous phase of the emulsion (prior to combining the oil and aqueous phases to form an emulsion), by adding a surfactant and optionally, a polymeric agent, without the need of an “organic co-solvent”. It has been further surprisingly discovered that including a steroid in a foamable emulsion that is insoluble both in water and in the oil phase of the composition can result in a composition in which the steroid is dissolved, with no relation to the addition of an “organic co-solvent”.

Thus, in further embodiments, the steroid is solubilized in the emulsion, although it is insoluble both in water and in the oil phase of the composition. In more specific embodiments, the composition contains a solubilized steroid, although the composition does not contain an “organic co-solvent”. An “organic co-solvent”, is one of the group consisting of an ester of a fatty acid for example a C12-C₁₋₅ alkyl benzoate, a medium to long chain alcohol, an aromatic and/or alkyl pyrollidinone, an aromatic and/or alkyl, and/or cyclic ketone, an aromatic and/or alkyl, and/or cyclic ether, substituted and/or unsubstituted single or multiple ring aromatic, straight chain and/or branched chain and/or cyclic alkane or silicone.

In yet additional embodiments, the steroid is not fully dissolved in either the aqueous phase or the oil phase of the emulsion concurrently, and thus, it is suspended in the emulsion, i.e., at least a portion of the steroid portion remains in solid state in the final composition. In such a case, the polymeric agents that are listed herein serve as suspension-stabilizing agents to stabilize the composition.

In certain embodiments, the composition and properties of the aqueous phase of the emulsion (e.g., pH, electrolyte concentration and chelating agents) and/or the composition of the oil phase of the emulsion are adjusted to attain a desirable solubility profile of the active agent.

The steroid is included in the composition in a concentration that provides a desirable ratio between the efficacy and safety. Typically, steroids are included in the composition in a concentration between about 0.005% and about 12%. However, in some embodiments, the concentration is between about 0.005% and about 0.5%, in other embodiment between about 0.5% and about 2%, and in additional embodiments between about 2% and about 5% or between about 5% and about 12%.

In one or more embodiments, the steroid possesses immunomodulating and/or anti-inflammatory properties. Without being bound to a specific theory, immunomodulating and/or anti-inflammatory steroids act, among other mechanisms, through inhibition of the activity of phospholipase A₂. They also may have anti-proliferative effects on keratinocytes and other cell types. They can suppress collagen synthesis by fibroblasts, but this may lead to adverse effects. Anti-inflammatory steroids are roughly grouped according to relative anti-inflammatory activity, but activity may vary considerably depending upon the vehicle, the site of application, disease, the individual patient and whether or not an occlusive dressing is used, as exemplified in Table 4.

TABLE 4
Exemplary anti-inflammatory steroids that are useful according to the
present invention.

| Relative | | Typical concentration in |
|---|---|---|
| Potency | Generic Name | topical products |
| Low | Hydrocortisone | 0.5%-1% |
| Potency | hydrocortisone acetate | 0.5-1.0% |
| | Desonide | 0.02-0.2% |
| Medium | Betamethasone valerate | 0.05%-0.1% |
| Potency | Prednicarbate | 0.02-0.2% |
| | Clobetasone-17-butyrate | 0.05% |
| | Flucinonide | 0.01%-0.05% |
| | Fluocinolone acetonide | 0.01-0.01% |
| | Alcometasone dipropionate | 0.01% |
| | Mometasone furoate | 0.1% |
| | Triamcinolone acetonide | 0.025%-0.1% |
| High | Betamethasone-17-benzoate | 0.025% |
| Potency | Methylprednisolone aceponate | 0.1% |
| | Betamethasone dipropionate | 0.025%, 0.05% |
| | Halcinonide | 0.1% |
| | Triamcinolone acetonide | 0.5% |
| Highest | Halobetasol | 0.05% |
| Potency | Clobetasol-17-propionate | 0.05% |

In one or more embodiments, the steroid is selected from the group of low-potency anti-inflammatory steroids, medium potency anti-inflammatory steroids and high potency anti-inflammatory steroids.

In one or more embodiments, the anti-inflammatory steroid is included in the composition at a concentration between about 0.005% and about 1%.

The McKenzie vasoconstrictor assay, as described, for example, in the British Journal of Dermatology 1975; 93:563-71 and versions thereof, has been the primary method used for classifying the potency of a product, containing an anti-inflammatory steroids. Thus, in one or more embodiments, the anti-inflammatory steroid is a steroid that positively affects the vasoconstrictor assay.

In one or more embodiments, the steroid is a hormone. Hormones are known to affect a variety of biological processes in any organism, and thus, their inclusion in the composition, which is intended for local treatment of the skin, the vagina, the rectum as well as other body surfaces and cavities provided an advantageous treatment modality. Such compositions containing hormones can be further administered systemically, via the transdermal or transmucosal route, in order to alleviate a disorder that is affected by the specific hormone, or in order to tune the hormonal balance of the body in order to attain certain effects controlled by hormones, such as contraception and birth induction.

In one or more embodiments, the steroid hormone is a male hormone or an androgen. Non-limiting examples of male hormones/androgens include testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone.

In one or more embodiments, the steroid hormone is a female hormone or an estrogen. Non-limiting examples of female hormones/estrogens include estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin.

In one or more embodiments, the steroid hormone is a progestogen. Non-limiting examples of progestogens include progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate.

Yet, in additional embodiments, the steroid an inhibitor of a steroid hormone. Non-limiting examples of such inhibitors are finasteride, dutasteride and spironolactone.

In one or more embodiments, the steroid is a vitamin D. The term vitamin D is used to describe all steroids that exhibit qualitatively the biological activity of calciol (vitamin D₃). Non limiting examples of vitamin D compounds are provided in Table 5.

Yet, in additional embodiments, the steroid is a vitamin D₃ analogue. Exemplary vitamin D₃ analogs include calcipotriol, tacalcitol, maxacalcitol, and calcitriol, with calcipotriol being especially preferred. Vitamin D₃ analogues and derivatives are known to degrade at low pH levels. Therefore, in certain preferred embodiments, the steroid is a vitamin D₃ or an analogue or a derivative thereof, the pH is adjusted to the range between about 7 and about 10, or between about 7.5 and about 9. In one or more embodiments, the pH is adjusted using a buffering agent, suitable of maintaining a pH level between about 7 and about 10, or between about 7.5 and about 9.

TABLE 5
Examples of vitamin D compounds

| Vitamin D name | Systematic steroid name |
|---|---|
| Cholecalciferol (also termed calciol, | (5Z,7E)-(3S)-9,10-seco-5,7,10(19)- |
| cholecalciferol, vitamin D₃ and | cholestatrien-3-ol |
| colecalciferol) | |
| 25-Hydroxycholecalciferol (also termed | (5Z,7E)-(3S)-9,10-seco-5,7,10(19)- |
| calcidiol) | cholestatriene-3,25-diol |
| 1α,25-Dihydroxycholecalciferol (also | (5Z,7E)-(1S,3R)-9,10-seco-5,7,10(19)- |
| termed calcitriol) | cholestatriene-1,3,25-triol |
| Ergocalciferol (also termed ercalciol | (5Z,7E,22E)-(3S)-9,10-seco-5,7,10(19),22- |
| and ergocalciferol) | ergostatetraen-3-ol |
| 1α,25-Dihydroxyergocalciferol (also | (5Z,7E,22E)-(1S,3R)-9,10-seco- |
| termed ercalcitriol) | 5,7,10(19),22-ergostatetraen-1,3,25-triol |
| 22,23-Dihydroergocalciferol (also | (5Z,7E)-(3S)-9,10-seco-5,7,10(19)- |
| termed (24S)-methylcalciol and 22,23- | ergostatrien-3-ol |
| dihydroercalciol) | |
| 1α,24R,25-Trihydroxycholecalciferol | (5Z,7E)-(1S,3R,24R)-9,10-seco-5,7,10(19)- |
| (also termed calcitetrol) | cholestatriene-1,3,24,25-tetrol |
| Previtamin D₃ (also termed | (6Z)-(3S)-9,10-seco-5(10),6,8-cholestatrien- |
| precalciferol and (6Z)-tacalciol) | 3-ol |
| Tachysterol₃ (also termed tacalciol) | (6E)-(3S)-9,10-seco-5(10),6,8-cholestatrien- |
| | 3-ol |
| Isovitamin D₃ (also termed (5E)- | (5E,7E)-(3S)-9,10-seco-1(10)5,7- |
| isocalciol) | cholestatrien-3-ol |
| Dihydrotachysterol₃ (also termed | (5E,7E)-(3S,10S)-9,10-seco-5,7- |
| dihydroercalciol) | cholestadien-3-ol |

Further examples of vitamin D compounds include, but are not limited to (1S)-Hydroxycalciol (also termed 1(c-hydroxycholecalciferol and alfacaleidol), (24R)-Hydroxycalcidiol (also termed 24(R),25-dihydroxycholecalciferol), 25-Fluorocalciol (also termed 25-fluorocholecalciferol), Ercalcidiol (also termed 25-hydroxyergocalciferol), Ertacalciol (also termed tachysterol₂, (5E)-Isocalciol (also termed isovitamin D₃, 22,23-Dihydroercalciol), (24S)-methylcalciol (also termed vitamin D₄), (5E)-(10S)-10,19-Dihydroercalciol, (also termed dihydrotachysterol₂, hytakerol, and dihydrotachysterol), (24S)-Ethylcalciol (also termed vitamin D₅) and (22E)-(24R)-Ethyl-22,23-didehydrocalciol, (also termed vitamin D₆).

In one or more embodiments, the steroid is a phytosteroid or a phytosterol. As used herein, the term “phytosteroid” or “phytosterol” includes all steroids that are obtained, derived or extracted from plant sources. Non-limiting examples of families of phytosteroids and phytosterols include ecdysones, withanolids, sterines, steroid saponins and soflavonoids. Non-limiting examples of phytosteroid and phytosterol compounds include alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol, as well as all natural or synthesized forms and derivatives thereof, such as fatty acid esters, such as ferulic acid esters, oleoyl esters, and cinnamic acid esters, including isomers.

Plant oils and extracts which contain steroids are also useful. Non limiting examples of plants that contain steroids include nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, calendula officinalis

In one or more embodiments, the steroid is a compound that is positively identified using a laboratory method, suitable of detecting a steroid.

### Additional Active Agents

Whenever there is reference to an additional active agent herein it will be appreciated by a man of the art that each of the additional active agents described herein can be an active agent in there own right on their own or in combination with other active agents in any of the carriers, foamable compositions and foams. Likewise whenever there is reference to an active or therapeutic agent herein it will be appreciated by a man of the art that each of the active or therapeutic agents described herein can be an additional therapeutic agent in combination with other active agents in any of the carriers, foamable compositions and foams

Several disorders of the skin, a body cavity or mucosal surface (e.g., the mucosa of the nose, mouth, eye, ear, vagina or rectum) involve a combination of inflammation, cell proliferation and differentiation abnormalities, and other biological abnormalities that can be effected by a steroid; and other etiological factors that require an additional therapeutic modality. For example, psoriasis involves inflammation as well as excessive cell proliferation and inadequate cell differentiation. Atopic dermatitis involves inflammation, skin dryness and keratinocyte growth abnormality. Bacterial, fungal and viral infections involve pathogen colonization at the affected site and inflammation. Likewise, hair growth disorders and other pilosebaceous disorders involve an impaired hormonal balance (which can be affected by a steroid hormone or a steroid hormone antagonist), together with other etiological factors, that can be affected a non-steroidal active agent. Hence, in many cases, the inclusion of an additional therapeutic agent in the foamable pharmaceutical composition, contributes to the clinical activity of the steroid. Thus, in one or more embodiments, the foamable composition further includes at least one additional therapeutic agent, in a therapeutically effective concentration.

In one or more embodiments, the at least one additional non-steroidal therapeutic agent is selected from the group consisting of an anti-infective, an antibiotic, an antibacterial agent, an antifungal agent, an antiviral agent, an antiparasitic agent, a nonsteroidal anti-inflammatory drug, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

In certain cases, the disorder to be treated involves unaesthetic lesions that need to be masked. For example, rosacea involves papules and pustules, which can be treated with a steroid, as well as erythema, telangiectasia and redness, which do not respond to treatment with a steroid. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

### Coal Tar Solution—(LCD)

Coal tar or crude coal tar is obtained by the destructive distillation of bituminous coal at very high temperatures. The crude coal tar can be further refined with alcohol extraction to yield liquor carbonis detergens (LCD). It is believed that over 10,000 different compounds make up coal tar but only 400 or so have been identified. The main groups of compounds making up crude coal tar are 48% hydrocarbons, 42% carbon and 10% water.

In its natural form coal tar is a thick, nearly black, viscous liquid with a characteristic smell. It is most often obtained in solution form (0.1 to 20%) and mixed with other ingredients. Crude coal tar and LCD have long been used to treat psoriasis, eczema, seborrhea and other skin diseases. Coal tar has antiseptic, anti-itching and photosensitizing properties. After an initial but short proliferation of epidermal cells, coal tar treatment slows epidermal cell production and results in an overall reduction in epidermal thickness. Coal tar may not clear psoriasis as quickly as some other agents, but longer periods of remission occur with its use. In one or more embodiments LCD is used in combination with one or more steroids.

In an embodiment, the active agent or additional active agent is coal tar. In an embodiment, active agent or the additional active agent is an biologically active agent which is isolated, distilled, extracted or otherwise derived from coal tar.

Active agents can be used on their own or in combination with other agents. The vehicles and compositions can be used to provide a physically and or chemically stable or stabilizing environment for the active agent(s). The creaming resistant, viscoelastic compositions provide a physically stable carrier for the active agents which can be a chemically stable or stabilizing environment for the active agent. Chemical stability is improved by appropriate use of modulating agents.

Suitable therapeutic agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers. As is known to one skilled in the art, in some instances a specific active agent may have more than one activity, function or effect.

In an embodiment, the therapeutic agent is an active herbal extract. Suitable active herbal extracts include but are not limited to angelica, anise oil, astragali radix, azalea, benzyl acetate, birch tar oil, bornyl acetate, cacumen biotae, camphor, cantharidin, capsicum, cineole, cinnamon bark, cinnamon leaf, citronella, citroneliol, citronellyl acetate, citronellyl formate, eucalyptus, eugenyl acetate, flos carthami, fructus mori, garlic, geraniol, geranium, geranyl acetate, habanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, .alpha.-pinene, .beta.-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof. In an embodiment, the active agent is an acaricide. Suitable acaricides include but are not limited to amitraz, flumethrin, fluvalinate and derivatives, esters, salts and mixtures thereof.

In an embodiment, the therapeutic agent is an age spot and keratoses removing agent. Suitable age spot and keratoses removing agent include but are not limited to hydroxy acids, azelaic acid and other related dicarboxylic acids, retinoids, kojic acid, arbutin, nicotinic, ascorbic acid, hydroquinone and derivatives, esters, salts and mixtures thereof. Certain nonsteroidal anti-inflammatory agents, such as diclofenac are also useful for the treatment of keratoses.

In an embodiment, the therapeutic agent is an analgesic. Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

In an embodiment, the therapeutic agent is a local anesthetic. Suitable local anesthetics include but are not limited to benzocaine, benzyl alcohol, bupivacaine, butamben picrate, chlorprocaine, cocaine, dibucaine, dimethisoquin, dyclonine, etidocaine, hexylcaine, ketamine, lidocaine, mepivacaine, phenol, pramoxine, procaine, tetracaine, salicylates and derivatives, esters, salts and mixtures thereof.

In an embodiment, the therapeutic agent is an antiacne agent. Suitable antiacne agents include but are not limited to N-acetylcysteine, adapalene, azelaic acid, benzoyl peroxide, cholate, clindamycin, deoxycholate, erythromycin, flavinoids, glycolic acid, meclocycline, metronidazol, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

In an embodiment, the therapeutic agent is an antiaging agent. Suitable antiaging agents include but are not limited to sulfur-containing D and L amino acids, alpha-hydroxy acids s, beta-hydroxy acids (e.g. salicylic acid), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate) skin barrier forming agents, melatonin and derivatives, esters, salts and mixtures thereof.

### Dicarboxylic Acid and Esters Thereof

In an embodiment, the organic carrier comprises an ester of a dicarboxylic acid. In the context herein, a dicarboxylic acid is an organic material, having two carboxylic acid moieties on its carbon atom skeleton. They have the general molecular formula HOOC—(CH₂)ₙ—COOH.

In an embodiment, the dicarboxylic acid is a short-chain dicarboxylic acid. The simplest Short-chain dicarboxylic acid are oxalic acid (n=0), malonic acid (n=1), succinic acid (n=2) and glutaric acid (n=3).

Additional members of dicarboxylic acid group are derived from natural products or from synthesis, having “n” value from 4 up to 21. In one or more embodiments, the dicarboxylic acid is selected from the group consisting of adipic acid (hexanedioic acid; n=4), pimelic acid (heptanedioic acid; n=5), suberic acid (octanedioic acid; n=6), azelaic acid (nonanedioic acid; n=7), sebacic acid (decanedioic acid; n=8) and dodecanedioic acid (n=10).

In an embodiment, the additional active agent is azelaic acid. Azelaic acid is a naturally occurring saturated dicarboxylic acid. (Structural Formula: HOOC—(CH₂)₇-COOH; Chemical Name: 1,7-heptanedicarboxylic acid; Empirical Formula: C₉H16O4; Molecular Weight: 188.22)

The exact mechanism of action of azelaic acid is not known. Azelaic acid has been shown to possess antimicrobial activity against Propionibacterium acnes and Staphylococcus epidermidis. The antimicrobial action may be attributable to inhibition of microbial cellular protein synthesis.

A normalization of keratinization leading to an anticomedonal effect of azelaic acid may also contribute to its clinical activity. Electron microscopic and immunohistochemical evaluation of skin biopsies from human subjects treated with AZELEX® (20% azelaic acid cream) demonstrated a reduction in the thickness of the stratum corneum, a reduction in number and size of keratohyalin granules, and a reduction in the amount and distribution of filaggrin (a protein component of keratohyalin) in epidermal layers. This is suggestive of the ability to decrease microcomedo formation.

In an additional embodiment, the dicarboxylic acid contains 10 to 32 carbon atoms in their carbon atom skeleton, such as brassylic acid (n=11), thapsic acid (n=14), 14-methylnonacosanedioic acid (C29) and 14,15-dimethyltriacontanedioic acid (C30).

The carbon atom skeleton of the dicarboxylic acid can be saturated or unsaturated, such as in the case of maleic acid and fumaric acid.

An ester of a dicarboxylic acid is a chemical compound produced by the reaction between a dicarboxylic acid and at least one alcohol, with the elimination of a molecule of water. The reaction of a dicarboxylic acid with one alcohol molecule results in a mono ester of said dicarboxylic acid, and the reaction of a dicarboxylic acid with two alcohol molecules results in a diester of the dicarboxylic acid.

The alcohol molecule, to be linked to the dicarboxylic acid, can be selected from the group of an alkyl an aryl alcohol. Exemplary alcohol, suitable according to the present invention include methyl alcohol, ethyl alcohol, propyl alcohol, isopropyl alcohol, butyl alcohol, isobutyl alcohol, t-butyl alcohol, pentyl alcohol, hexyl alcohol, octyl alcohol, decyl alcohol, capryl alcohol, phenol, benzyl alcohol and the like.

In one or more embodiments, the alcohol is a biologically active alcohol. In an embodiment, biologically active alcohol possesses keratolytic activities. Examples of keratolytically active alcohol suitable according to the present invention include ortho-, meta- and para-hydroxyalkylbenzoate, salicylic acid, ortho-, meta-, and para-dihydroxybenzene, ortho-, meta-, and para-hydroxytoluene, alpha-hydroxy acid, retinol, and derivatives thereof such as provided in U.S. Pat. No. 6,180,669. In an embodiment, the biologically active alcohol is selected from the group consisting of steroidal hormones, steroidal anti-inflammatory agents, vitamin E and vitamin D, such as provided in U.S. Pat. Appl. 20040191196.

### Antibiotics

In an embodiment, the therapeutic agent is an antibiotic. The terms “antibiotic” as used herein shall include, but is not limited to, any substance being destructive to or inhibiting the growth of bacteria or any substance having the capacity to inhibit the growth of or to destroy bacteria.

In one or more embodiments, the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

Suitable antibiotics include but are not limited to amanfadine hydrochloride, amanfadine sulfate, amikacin, arnikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocyldline, iodate, iodine, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, microcrystalline and nanocrystalline particles of silver, copper, zinc, mercury, tin, lead, bismuth, cadmium and chromium, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

In one or more embodiments, the antibiotic agent is selected from the classes consisting of beta-lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroides, sulfonamides, tetracycline, dicarboxylic acids, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Beta-lactam antibiotics include, but are not limited to, 2-(3-alanyl)clavam, 2-hydroxymethylclavam, 8-epi-thienamycin, acetyl-thienamycin, amoxicillin, amoxicillin sodium, amoxicillin trihydrate, amoxicillin-potassium clavulanate combination, ampicillin, ampicillin sodium, ampicillin trihydrate, ampicillin-sulbactam, apalcillin, aspoxicillin, azidocillin, azlocillin, aztreonam, bacampicillin, biapenem, carbenicillin, carbenicillin disodium, carfecillin, carindacillin, carpetimycin, cefacetril, cefaclor, cefadroxil, cefalexin, cefaloridine, cefalotin, cefamandole, cefamandole, cefapirin, cefatrizine, cefatrizine propylene glycol, cefazedone, cefazolin, cefbuperazone, cefcapene, cefcapene pivoxil hydrochloride, cefdinir, cefditoren, cefditoren pivoxil, cefepime, cefetamet, cefetamet pivoxil, cefixime, cefmenoxime, cefmetazole, cefminox, cefminox, cefmolexin, cefodizime, cefonicid, cefoperazone, ceforanide, cefoselis, cefotaxime, cefotetan, cefotiam, cefoxitin, cefozopran, cefpiramide, cefpirome, cefpodoxime, cefpodoxime proxetil, cefprozil, cefquinome, cefradine, cefroxadine, cefsulodin, ceftazidime, cefteram, cefteram pivoxil, ceftezole, ceftibuten, ceftizoxime, ceftriaxone, cefuroxime, cefuroxime axetil, cephalosporin, cephamycin, chitinovorin, ciclacillin, clavulanic acid, clometocillin, cloxacillin, cycloserine, deoxy pluracidomycin, dicloxacillin, dihydro pluracidomycin, epicillin, epithienamycin, ertapenem, faropenem, flomoxef, flucloxacillin, hetacillin, imipenem, lenampicillin, loracarbef, mecillinam, meropenem, metampicillin, meticillin, mezlocillin, moxalactam, nafcillin, northienamycin, oxacillin, panipenem, penamecillin, penicillin, phenethicillin, piperacillin, tazobactam, pivampicillin, pivcefalexin, pivmecillinam, pivmecillinam hydrochloride, pluracidomycin, propicillin, sarmoxicillin, sulbactam, sulbenicillin, talampicillin, temocillin, terconazole, thienamycin, ticarcillin and analogs, salts and derivatives thereof.

Aminoglycosides include, but are not limited to, 1,2′-N-DL-isoseryl-3′,4′-dideoxykanamycin B, 1,2′-N-DL-isoseryl-kanamycin B, 1,2′-N—[(S)-4-amino-2-hydroxybutyryl]-3′,4′-dideoxykanamycin B, 1,2′-N—[(S)-4-amino-2-hydroxybutyryl]-kanamycin B, 1-N-(2-Aminobutanesulfonyl) kanamycin A, 1-N-(2-aminoethanesulfonyl]-3′,4′-dideoxyribostamycin, 1-N-(2-Aminoethanesulfonyl)3′-deoxyribostamycin, 1-N-(2-aminoethanesulfonyl)3′4′-dideoxykanamycin B, 1-N-(2-aminoethanesulfonyl)kanamycin A, 1-N-(2-aminoethanesulfonyl)kanamycin B, 1-N-(2-aminoethanesulfonyl)ribostamycin, 1-N-(2-aminopropanesulfonyl)3′-deoxykanamycin B, 1-N-(2-aminopropanesulfonyl)3′4′-dideoxykanamycin B, 1-N-(2-aminopropanesulfonyl)kanamycin A, 1-N-(2-aminopropanesulfonyl)kanamycin B, 1-N-(L-4-amino-2-hydroxy-butyryl)2,′3′-dideoxy-2′-fluorokanamycin A, 1-N-(L-4-amino-2-hydroxy-propionyl)2,′3′-dideoxy-2′-fluorokanamycin A, 1-N-DL-3′,4′-dideoxy-isoserylkanamycin B,1-N-DL-isoserylkanamycin, 1-N-DL-isoserylkanamycin B, 1-N—[L-(−)-(alpha-hydroxy-gamma-aminobutyryl)]-XK-62-2,2′,3′-dideoxy-2′-fluorokanamycin A,2-hydroxygentamycin A3,2-hydroxygentamycin B, 2-hydroxygentamycin B1, 2-hydroxygentamycin JI-20A, 2-hydroxygentamycin JI-20B, 3″-N-methyl-4″-C-methyl-3′,4′-dodeoxy kanamycin A, 3″-N-methyl-4″-C-methyl-3′,4′-dodeoxy kanamycin B, 3″-N-methyl-4″-C-methyl-3′,4′-dodeoxy-6′-methyl kanamycin B, 3′,4′-Dideoxy-3′-eno-ribostamycin,3′,4′-dideoxyneamine,3′,4′-dideoxyribostamycin, 3′-deoxy-6′-N-methyl-kanamycin B,3′-deoxyneamine,3′-deoxyribostamycin, 3′-oxysaccharocin,3,3′-nepotrehalosadiamine, 3-demethoxy-2″-N-formimidoylistamycin B disulfate tetrahydrate, 3-demethoxyistamycin B,3-O-demethyl-2-N-formimidoylistamycin B, 3-O-demethylistamycin B,3-trehalosamine,4″, 6″-dideoxydibekacin, 4-N-glycyl-KA-6606VI, 5″-Amino-3′,4′,5″-trideoxy-butirosin A, 6″-deoxydibekacin,6′-epifortimicin A, 6-deoxy-neomycin (structure 6-deoxy-neomycin B),6-deoxy-neomycin B, 6-deoxy-neomycin C, 6-deoxy-paromomycin, acmimycin, AHB-3′,4′-dideoxyribostamycin,AHB-3′-deoxykanamycin B, AHB-3′-deoxyneamine,AHB-3′-deoxyribostamycin,AHB-4″-6″-dideoxydibekacin, AHB-6″-deoxydibekacin, AHB-dideoxyneamine, AHB-kanamycin B, AHB-methyl-3′-deoxykanamycin B, amikacin, amikacin sulfate, apramycin, arbekacin, astromicin, astromicin sulfate, bekanamycin, bluensomycin, boholmycin, butirosin, butirosin B, catenulin, coumamidine gammal, coumamidine gamma2, D,L-1-N-(alpha-hydroxy-beta-aminopropionyl)-XK-62-2, dactimicin, de-O-methyl-4-N-glycyl-KA-6606VI,de-O-methyl-KA-66061, de-O-methyl-KA-70381,destomycin A, destomycin B, di-N6′,O3-demethylistamycin A, dibekacin, dibekacin sulfate, dihydrostreptomycin, dihydrostreptomycin sulfate, epi-formamidoylglycidylfortimicin B, epihygromycin, formimidoyl-istamycin A, formimidoyl-istamycin B, fortimicin B, fortimicin C, fortimicin D, fortimicin KE, fortimicin KF, fortimicin KG, fortimicin KG1 (stereoisomer KG1/KG2), fortimicin KG2 (stereoisomer KG1/KG2), fortimicin KG3, framycetin, framycetin sulphate, gentamicin, gentamycin sulfate, globeomycin, hybrimycin A1, hybrimycin A2, hybrimycin B1, hybrimycin B2, hybrimycin C1, hybrimycin C2, hydroxystreptomycin, hygromycin, hygromycin B, isepamicin, isepamicin sulfate, istamycin, kanamycin, kanamycin sulphate, kasugamycin, lividomycin, marcomycin, micronomicin, micronomicin sulfate, mutamicin, myomycin, N-demethyl-7-O-demethylcelesticetin, demethylcelesticetin, methanesulfonic acid derivative of istamycin, nebramycin, nebramycin, neomycin, netilmicin, oligostatin, paromomycin, quintomycin, ribostamycin, saccharocin, seldomycin, sisomicin, sorbistin, spectinomycin, streptomycin, tobramycin, trehalosmaine, trestatin, validamycin, verdamycin, xylostasin, zygomycin and analogs, salts and derivatives thereof.

Ansa-type antibiotics include, but are not limited to, 21-hydroxy-25-demethyl-25-methylthioprotostreptovaricin, 3-methylthiorifamycin, ansamitocin, atropisostreptovaricin, awamycin, halomicin, maytansine, naphthomycin, rifabutin, rifamide, rifampicin, rifamycin, rifapentine, rifaximin, rubradirin, streptovaricin, tolypomycin and analogs, salts and derivatives thereof.

Antibiotic anthraquinones include, but are not limited to, auramycin, cinerubin, ditrisarubicin, ditrisarubicin C, figaroic acid fragilomycin, minomycin, rabelomycin, rudolfomycin, sulfurmycin and analogs, salts and derivatives thereof.

Antibiotic azoles include, but are not limited to, azanidazole, bifonazole, butoconazol, chlormidazole, chlormidazole hydrochloride, cloconazole, cloconazole monohydrochloride, clotrimazol, dimetridazole, econazole, econazole nitrate, enilconazole, fenticonazole, fenticonazole nitrate, fezatione, fluconazole, flutrimazole, isoconazole, isoconazole nitrate, itraconazole, ketoconazole, lanoconazole, metronidazole, metronidazole benzoate, miconazole, miconazole nitrate, neticonazole, nimorazole, niridazole, omoconazol, ornidazole, oxiconazole, oxiconazole nitrate, propenidazole, secnidazol, sertaconazole, sertaconazole nitrate, sulconazole, sulconazole nitrate, tinidazole, tioconazole, voriconazol and analogs, salts and derivatives thereof.

Antibiotic glycopeptides include, but are not limited to, acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, zorbamycin and analogs, salts and derivatives thereof.

Macrolides include, but are not limited to, acetylleucomycin, acetylkitasamycin, angolamycin, azithromycin, bafilomycin, brefeldin, carbomycin, chalcomycin, cirramycin, clarithromycin, concanamycin, deisovaleryl-niddamycin, demycinosyl-mycinamycin, Di-O-methyltiacumicidin, dirithromycin, erythromycin, erythromycin estolate, erythromycin ethyl succinate, erythromycin lactobionate, erythromycin stearate, flurithromycin, focusin, foromacidin, haterumalide, haterumalide, josamycin, josamycin ropionate, juvenimycin, juvenimycin, kitasamycin, ketotiacumicin, lankavacidin, lankavamycin, leucomycin, machecin, maridomycin, megalomicin, methylleucomycin, methymycin, midecamycin, miocamycin, mycaminosyltylactone, mycinomycin, neutramycin, niddamycin, nonactin, oleandomycin, phenylacetyldeltamycin, pamamycin, picromycin, rokitamycin, rosaramicin, roxithromycin, sedecamycin, shincomycin, spiramycin, swalpamycin, tacrolimus, telithromycin, tiacumicin, tilmicosin, treponemycin, troleandomycin, tylosin, venturicidin and analogs, salts and derivatives thereof.

Antibiotic nucleosides include, but are not limited to, amicetin, angustmycin, azathymidine, blasticidin S, epiroprim, flucytosine, gougerotin, mildiomycin, nikkomycin, nucleocidin, oxanosine, oxanosine, puromycin, pyrazomycin, showdomycin, sinefungin, sparsogenin, spicamycin, tunicamycin, uracil polyoxin, vengicide and analogs, salts and derivatives thereof.

Antibiotic peptides include, but are not limited to, actinomycin, aculeacin, alazopeptin, amfomycin, amythiamycin, antifungal from Zalerion arboricola, antrimycin, apid, apidaecin, aspartocin, auromomycin, bacileucin, bacillomycin, bacillopeptin, bacitracin, bagacidin, berninamycin, beta-alanyl-L-tyrosine, bottromycin, capreomycin, caspofungine, cepacidine, cerexin, cilofungin, circulin, colistin, cyclodepsipeptide, cytophagin, dactinomycin, daptomycin, decapeptide, desoxymulundocandin, echanomycin, echinocandin B, echinomycin, ecomycin, enniatin, etamycin, fabatin, ferrimycin, ferrimycin, ficellomycin, fluoronocathiacin, fusaricidin, gardimycin, gatavalin, globopeptin, glyphomycin, gramicidin, herbicolin, iomycin, iturin, iyomycin, izupeptin, janiemycin, janthinocin, jolipeptin, katanosin, killertoxin, lipopeptide antibiotic, lipopeptide from Zalerion sp., lysobactin, lysozyme, macromomycin, magainin, meliftin, mersacidin, mikamycin, mureidomycin, mycoplanecin, mycosubtilin, neopeptifluorin, neoviridogrisein, netropsin, nisin, nocathiacin, nocathiacin 6-deoxyglycoside, nosiheptide, octapeptin, pacidamycin, pentadecapeptide, peptifluorin, permetin, phytoactin, phytostreptin, planothiocin, plusbacin, polcillin, polymyxin antibiotic complex, polymyxin B, polymyxin B1, polymyxin F, preneocarzinostatin, quinomycin, quinupristin-dalfopristin, safracin, salmycin, salmycin, salmycin, sandramycin, saramycetin, siomycin, sperabillin, sporamycin, a streptomyces compound, subtilin, teicoplanin aglycone, telomycin, thermothiocin, thiopeptin, thiostrepton, tridecaptin, tsushimycin, tuberactinomycin, tuberactinomycin, tyrothricin, valinomycin, viomycin, virginiamycin, zervacin and analogs, salts and derivatives thereof.

In one or more embodiments, the antibiotic peptide is a naturally-occurring peptide that possesses an antibacterial and/or an antifungal activity. Such peptide can be obtained from a herbal or a vertebrate source.

Polyenes include, but are not limited to, amphotericin, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, nystatin methyl ester, oxypricin, partricin, pentamycin, perimycin, pimaricin, primycin, proticin, rimocidin, sistomycosin, sorangicin, trichomycin and analogs, salts and derivatives thereof.

Polyethers include, but are not limited to, 20-deoxy-epi-narasin, 20-deoxysalinomycin, carriomycin, dianemycin, dihydrolonomycin, etheromycin, ionomycin, iso-lasalocid, lasalocid, lenoremycin, lonomycin, lysocellin, monensin, narasin, oxolonomycin, a polycyclic ether antibiotic, salinomycin and analogs, salts and derivatives thereof.

Quinolones include, but are not limited to, an alkyl-methylendioxy-4(1H)-oxocinnoline-3-carboxylic acid, alatrofloxacin, cinoxacin, ciprofloxacin, ciprofloxacin hydrochloride, danofloxacin, dermofongin A, enoxacin, enrofloxacin, fleroxacin, flumequine, gatifloxacin, gemifloxacin, grepafloxacin, levofloxacin, lomefloxacin, lomefloxacin, hydrochloride, miloxacin, moxifloxacin, nadifloxacin, nalidixic acid, nifuroquine, norfloxacin, ofloxacin, orbifloxacin, oxolinic acid, pazufloxacine, pefloxacin, pefloxacin mesylate, pipemidic acid, piromidic acid, premafloxacin, rosoxacin, rufloxacin, sparfloxacin, temafloxacin, tosufloxacin, trovafloxacin and analogs, salts and derivatives thereof.

Antibiotic steroids include, but are not limited to, aminosterol, ascosteroside, cladosporide A, dihydrofusidic acid, dehydro-dihydrofusidic acid, dehydrofusidic acid, fusidic acid, squalamine and analogs, salts and derivatives thereof.

Sulfonamides include, but are not limited to, chloramine, dapsone, mafenide, phthalylsulfathiazole, succinylsulfathiazole, sulfabenzamide, sulfacetamide, sulfachlorpyridazine, sulfadiazine, sulfadiazine silver, sulfadicramide, sulfadimethoxine, sulfadoxine, sulfaguanidine, sulfalene, sulfamazone, sulfamerazine, sulfamethazine, sulfamethizole, sulfamethoxazole, sulfamethoxypyridazine, sulfamonomethoxine, sulfamoxol, sulfanilamide, sulfaperine, sulfaphenazol, sulfapyridine, sulfaquinoxaline, sulfasuccinamide, sulfathiazole, sulfathiourea, sulfatolamide, sulfatriazin, sulfisomidine, sulfisoxazole, sulfisoxazole acetyl, sulfacarbamide and analogs, salts and derivatives thereof.

Tetracyclines include, but are not limited to, dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, museftamycin, oxytetracycline, rhodirubin, rolitetracycline, rubomycin, serirubicin, steffimycin, tetracycline and analogs, salts and derivatives thereof.

Dicarboxylic acids, having between about 6 and about 14 carbon atoms in their carbon atom skeleton are particularly useful in the treatment of disorders of the skin and mucosal membranes that involve microbial. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred. It has antibacterial effects on both aerobic and anaerobic organisms, particularly propionibacterium acnes and staphylococcus epidermidis, normalizes keratinization, and has a cytotoxic effect on malignant or hyperactive melanocytes. In a preferred embodiment, the dicarboxylic acid is azelaic acid in a concentration greater than 10%. Preferably, the concentration of azelaic acid is between about 10% and about 25%. In such concentrates, azelaic acid is suitable for the treatment of a variety of skin disorders, such as acne, rosacea and hyperpigmentation.

In one or more embodiments, the antibiotic agent is an antibiotic metal. A number of metals ions been shown to possess antibiotic activity, including silver, copper, zinc, mercury, tin, lead, bismutin, cadmium, chromium and ions thereof. It has been theorized that these antibiotic metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the antibiotic metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The antibiotic metal can further be intercalated in a chelating substrate.

In further embodiments, the antibiotic metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

In one or more embodiments, the antibiotic metal or metal ion is embedded into a substrate, such as a polymer, a mineral (such as zeolite, clay and silica).

In further embodiments, the antibiotics comprise oxidizing agents and substances that release free radicals and/or active oxygen.

In one or more embodiments, the antibiotic agent comprises strong oxidants and free radical liberating compounds, such as oxygen, hydrogen peroxide, benzoyl peroxide, elemental halogen species, as well as oxygenated halogen species, bleaching agents (e.g., sodium, calcium or magnesium hypochloride and the like), perchlorite species, iodine, iodate, and benzoyl peroxide. Organic oxidizing agents are also included in the definition of “oxidizing agent” according to the present invention, such as quinones. Such agents possess a potent broad spectrum activity.

In one or more embodiments the antibiotic agent is a cationic antimicrobial agent. The outermost surface of bacterial cells universally carries a net negative charge, making them sensitive to cationic substances. Examples of cationic antibiotic agents include: quaternary ammonium compounds (QAC's)—QAC's are surfactants, generally containing one quaternary nitrogen associated with at least one major hydrophobic moiety; alkyltrimethyl ammonium bromides are mixtures of where the alkyl group is between 8 and 18 carbons long, such as cetrimide (tetradecyltrimethylammonium bromide); benzalkonium chloride, which is a mixture of n-alkyldimethylbenzyl ammonium chloride where the alkyl groups (the hydrophobic moiety) can be of variable length; dialkylmethyl ammonium halides; dialkylbenzyl ammonium halides; and QAC dimmers, which bear bi-polar positive charges in conjunction with interstitial hydrophobic regions.

In one or more embodiments, the antibiotic agent is selected from the group of biguanides, triguanides, bisbiguanides and analogs thereof.

Guanides, biguanides, biguanidines and triguanides are unsaturated nitrogen containing molecules that readily obtain one or more positive charges, which make them effective antimicrobial agents. The basic structures a guanide, a biguanide, a biguanidine and a triguanide are provided below.

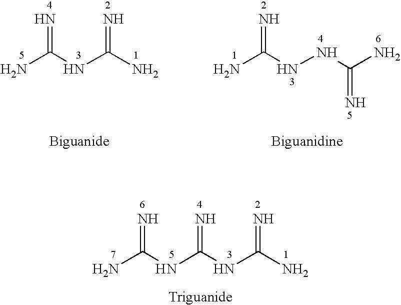

In one or more preferred embodiments, the guanide, biguanide, biguanidine or triguanide, provide bi-polar configurations of cationic and hydrophobic domains within a single molecule.

Examples of guanides, biguanides, biguanidines and triguanides that are currently been used as antibacterial agents include chlorhexidine and chlorohexidine salts, analogs and derivatives, such as chlorhexidine acetate, chlorhexidine gluconate and chlorhexidine hydrochloride, picloxydine, alexidine and polihexanide. Other examples of guanides, biguanides, biguanidines and triguanides that can conceivably be used according to the present invention are chlorproguanil hydrochloride, proguanil hydrochloride (currently used as antimalarial agents), metformin hydrochloride, phenformin and buformin hydrochloride (currently used as antidiabetic agents).

In one or more embodiments, the cationic antimicrobial agent is a polymer.

Cationic antimicrobial polymers include, for example, guanide polymers, biguanide polymers, or polymers having side chains containing biguanide moieties or other cationic functional groups, such as benzalkonium groups or quarternium groups (e.g., quaternary amine groups). It is understood that the term “polymer” as used herein includes any organic material comprising three or more repeating units, and includes oligomers, polymers, copolymers, block copolymers, terpolymers, etc. The polymer backbone may be, for example a polyethylene, ploypropylene or polysilane polymer.

In one or more embodiments, the cationic antimicrobial polymer is a polymeric biguanide compound. When applied to a substrate, such a polymer is known to form a barrier film that can engage and disrupt a microorganism. An exemplary polymeric biguanide compound is polyhexamethylene biguanide (PHMB) salts. Other exemplary biguanide polymers include, but are not limited to poly(hexamethylenebiguanide), poly(hexamethylenebiguanide) hydrochloride, poly(hexamethylenebiguanide) gluconate, poly(hexamethylenebiguanide) stearate, or a derivative thereof. In one or more embodiments, the antimicrobial material is substantially water-insoluble.

Yet, in one or more embodiment, the antibiotic is a non-classified antibiotic agent, including, without limitation, aabomycin, acetomycin, acetoxycycloheximide, acetylnanaomycin, an actinoplanes sp. Compound, actinopyrone, aflastatin, albacarcin, albacarcin, albofungin, albofungin, alisamycin, alpha-R,S-methoxycarbonylbenzylmonate, altromycin, amicetin, amycin, amycin demanoyl compound, amycine, amycomycin, anandimycin, anisomycin, anthramycin, anti-syphilis imune substance, anti-tuberculosis imune substance, antibiotic from Eschericia coli, antibiotics from Streptomyces refuineus, anticapsin, antimycin, aplasmomycin, aranorosin, aranorosinol, arugomycin, ascofuranone, ascomycin, ascosin, Aspergillus flavus antibiotic, asukamycin, aurantinin, an Aureolic acid antibiotic substance, aurodox, avilamycin, azidamfenicol, azidimycin, bacillaene, a Bacillus larvae antibiotic, bactobolin, benanomycin, benzanthrin, benzylmonate, bicozamycin, bravomicin, brodimoprim, butalactin, calcimycin, calvatic acid, candiplanecin, carumonam, carzinophilin, celesticetin, cepacin, cerulenin, cervinomycin, chartreusin, chloramphenicol, chloramphenicol palmitate, chloramphenicol succinate sodium, chlorflavonin, chlorobiocin, chlorocarcin, chromomycin, ciclopirox, ciclopirox olamine, citreamicin, cladosporin, clazamycin, clecarmycin, clindamycin, coliformin, collinomycin, copiamycin, corallopyronin, corynecandin, coumermycin, culpin, cuprimyxin, cyclamidomycin, cycloheximide, dactylomycin, danomycin, danubomycin, delaminomycin, demethoxyrapamycin, demethylscytophycin, dermadin, desdamethine, dexylosyl-benanomycin, pseudoaglycone, dihydromocimycin, dihydronancimycin, diumycin, dnacin, dorrigocin, dynemycin, dynemycin triacetate, ecteinascidin, efrotomycin, endomycin, ensanchomycin, equisetin, ericamycin, esperamicin, ethylmonate, everninomicin, feldamycin, flambamycin, flavensomycin, florfenicol, fluvomycin, fosfomycin, fosfonochlorin, fredericamycin, frenolicin, fumagillin, fumifungin, funginon, fusacandin, fusafungin, gelbecidine, glidobactin, grahamimycin, granaticin, griseofulvin, griseoviridin, grisonomycin, hayumicin, hayumicin, hazymicin, hedamycin, heneicomycin, heptelicid acid, holomycin, humidin, isohematinic acid, karnatakin, kazusamycin, kristenin, L-dihydrophenylalanine, a L-isoleucyl-L-2-amino-4-(4′-amino-2′, 5′-cyclohexadienyl) derivative, lanomycin, leinamycin, leptomycin, libanomycin, lincomycin, lomofungin, lysolipin, magnesidin, manumycin, melanomycin, methoxycarbonyl methyl monate, methoxycarbonylethylmonate, methoxycarbonylphenylmonate, methyl pseudomonate, methylmonate, microcin, mitomalcin, mocimycin, moenomycin, monoacetyl cladosporin, monomethyl cladosporin, mupirocin, mupirocin calcium, mycobacidin, myriocin, myxopyronin, pseudoaglycone, nanaomycin, nancimycin, nargenicin, neocarcinostatin, neoenactin, neothramycin, nifurtoinol, nocardicin, nogalamycin, novobiocin, octylmonate, olivomycin, orthosomycin, oudemansin, oxirapentyn, oxoglaucine methiodide, pactacin, pactamycin, papulacandin, paulomycin, phaeoramularia fungicide, phenelfamycin, phenyl, cerulenin, phenylmonate, pholipomycin, pirlimycin, pleuromutilin, a polylactone derivative, polynitroxin, polyoxin, porfiromycin, pradimicin, prenomycin, prop-2-enylmonate, protomycin, pseudomonas antibiotic, pseudomonic acid, purpuromycin, pyrinodemin, pyrrolnitrin, pyrrolomycin, amino, chloro pentenedioic acid, rapamycin, rebeccamycin, resistomycin, reuterin, reveromycin, rhizocticin, roridin, rubiflavin, naphthyridinomycin, saframycin, saphenamycin, sarkomycin, sarkomycin, sclopularin, selenomycin, siccanin, spartanamicin, spectinomycin, spongistatin, stravidin, streptolydigin, streptomyces arenae antibiotic complex, streptonigrin, streptothricins, streptovitacin, streptozotocine, a strobilurin derivative, stubomycin, sulfamethoxazol-trimethoprim, sakamycin, tejeramycin, terpentecin, tetrocarcin, thermorubin, thermozymocidin, thiamphenicol, thioaurin, thiolutin, thiomarinol, thiomarinol, tirandamycin, tolytoxin, trichodermin, trienomycin, trimethoprim, trioxacarcin, tyrissamycin, umbrinomycin, unphenelfamycin, urauchimycin, usnic acid, uredolysin, variotin, vermisporin, verrucarin and analogs, salts and derivatives thereof.

In one or more embodiments, the antibiotic agent is a naturally occurring antibiotic compound. As used herein, the term “naturally-occurring antibiotic agent” includes all antibiotic that are obtained, derived or extracted from plant or vertebrate sources. Non-limiting examples of families of naturally-occurring antibiotic agents include phenol, resorcinol, antibiotic aminoglycosides, anamycin, quinines, anthraquinones, antibiotic glycopeptides, azoles, macrolides, avilamycin, agropyrene, cnicin, aucubin antibioticsaponin fractions, berberine (isoquinoline alkaloid), arctiopicrin (sesquiterpene lactone), lupulone, humulone (bitter acids), allicin, hyperforin, echinacoside, coniosetin, tetramic acid, imanine and novoimanine.

Ciclopirox and ciclopiroxolamine possess fungicidal, fungistatic and sporicidal activity. They are active against a broad spectrum of dermatophytes, yeasts, moulds and other fungi, such as trichophyton species, microsporum species, epidermophyton species and yeasts (candida albicans, candida glabrata, other candida species and cryptococcus neoformans). Some aspergillus species are sensitive to ciclopirox as are some penicillium. Likewise, ciclopirox is effective against many gram-positive and gram-negative bacteria (e.g., escherichia coli, proteus mirabilis, pseudomonas aeruginosa, staphylococcus and streptococcus species), as well as mycoplasma species, trichomonas vaginalis and actinomyces.

Plant oils and extracts which contain antibiotic agents are also useful. Non limiting examples of plants that contain agents include thyme, perilla, lavender, tea tree, terfezia clayeryi, Micromonospora, pufterlickia verrucosa, putterlickia pyracantha, putterlickia retrospinosa, Maytenus ilicifolia, maytenus evonymoides, maytenus aquifolia, faenia interjecta, cordyceps sinensis, couchgrass, holy thistle, plantain, burdock, hops, echinacea, buchu, chaparral, myrrh, red clover and yellow dock, garlic and St. John's wort.

Mixtures of these antibiotic agents may also be employed according to the present invention.

In an embodiment, the therapeutic agent is an antidandruff agent. Suitable antidandruff agents include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide,climbazole-, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione and derivatives, esters, salts and mixtures thereof.

In an embodiment, the therapeutic agent is an antihistamine. Suitable antihistamines include but are not limited to chlorcyclizine, diphenhydramine, mepyramine, methapyrilene, tripelennamine and derivatives, esters, salts and mixtures thereof.

### Antifungal

In an embodiment, the therapeutic agent is an antimycotic, also termed antifungal agent. The terms “antimycotic” and “antifungal” as used herein include, but are not limited to, any substance being destructive to or inhibiting the growth of fungi and yeast or any substance having the capacity to inhibit the growth of or to destroy fungi and/or yeast.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina. Thus, in one or more embodiments, the antifungal agent is selected from the group including but not limited to, azoles, diazoles, triazoles, miconazole, fluconazole, ketoconazole, clotrimazole, itraconazole, Climbazole, griseofulvin, ciclopirox, ciclopirox-olamine, amorolfine, terbinafine, Amphotericin B, potassium iodide and flucytosine (5FC) at a therapeutically effective concentration.

Azoles are pharmaceutically active compounds that are unsaturated five member ring heterocyclic compound, wherein one, two or three members of the ring are nitrogen atoms, as exemplified in a non-limiting way and illustrated in the following schemes:

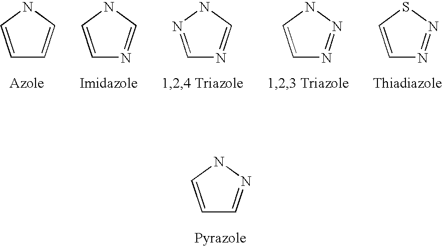

Azole is a compound including an unsaturated five member ring heterocyclic compound, wherein one, two or three members of the ring are nitrogen atoms.

Examples of therapeutic azoles include, but are not limited to, azanidazole, bifonazole, butoconazol, chlormidazole, climbazole, cloconazole, clotrimazole, dimetridazole, econazole, enilconazole, fenticonazole, fezatione, fluconazole, flutrimazole, isoconazole, itraconazole, ketoconazole, lanoconazole, metronidazole, metronidazole benzoate, miconazole, neticonazole, nimorazole, niridazole, omoconazol, ornidazole, oxiconazole, posaconazole, propenidazole, ravuconazole, secnidazol, sertaconazole, sulconazole, thiabendazole, tinidazole, tioconazole, voriconazol and salts and derivatives thereof. Such azoles are mainly used as antifungal agents, yet several of them also possess other therapeutic benefits, such as anti-inflammatory, antibacterial and antiviral effects.

Additional non-limiting exemplary classes of azoles include oxazoles, thiazoles, thiadiazoles and thiatriazoles, benzimidazoles, and salts and derivatives thereof.

In an embodiment, the azole is metronidazole.

In one or more embodiments, the antifungal agent is a peptide.

In certain embodiments, antifungal agent is a naturally-occurring peptide that possesses an antibacterial and/or an antifungal activity. Such peptide can be obtained from a herbal or a vertebrate source.

In an embodiment, the antifungal agent is a polyene. Polyene compounds are so named because of the alternating conjugated double bonds that constitute a part of their macrolide ring structure. Polyenes include, but are not limited to, amphotericin, aureofungin, ayfactin, azalomycin, blasticidin, candicidin, candicidin methyl ester, candimycin, candimycin methyl ester, chinopricin, filipin, flavofungin, fradicin, hamycin, hydropricin, levorin, lucensomycin, lucknomycin, mediocidin, mediocidin methyl ester, mepartricin, methylamphotericin, natamycin, niphimycin, nystatin, oxypricin, partricin, pentamycin, perimycin, pimaricin, primycin, proticin, rimocidin, sistomycosin, sorangicin, trichomycin and analogs, salts and derivatives thereof.

In an embodiment, the antifungal agent is a pyrimidine, such as Flucytosine.

In an embodiment, the antifungal agent is an allylamine, such as terbinafine and naftifine.

In an embodiment, the antifungal agent is a morpholine derivative, such as amorolfine.

In an embodiment, the antifungal agent is selected from the group consisting of C₁₋clopirox, ciclopiroxolmine, griseofulvin.

In an embodiment, the antifungal agent is a Thiocarbamate, such as tolnaftate.

In an embodiment, the antifungal agent is a Sulfonamide, such as Mafenide and Dapsone.

In an embodiment, the antifungal agent consists of a plant oil or a plant extract possessing antifungal activity; or a plant oil or extract which contains antifungal agents. Non-limiting examples of plants containing agents include, but are not limited to, anise, basil, bergemont, burdock, buchu, chaparral, camphor, cardamom, carrot, canola, cassia, catnip, cedarwood, citronella, clove, couchgrass, cypress, echinacea, eucalyptus, faenia interjecta, garlic, ginger, grapefruit, holy thistle, hops, hyssop, jasmine, jojova, lavender, lavandin, lemon, lime, mandarin, marigold, marjoram, maytenus ilicifolia, maytenus evonymoides, maytenus aquifolia, micromonospora, myrrh, neroli, nutmeg, orange, ordyceps sinensis, peppermint, perilla, petitgrain, plantain, putterlickia verrucosa, putterlickia pyracantha, putterlickia retrospinosa, rosemary, sage, spearmint, star anise, St. John's wort, red clover, tangerine, tea tree, terfezia clayeryi, thyme vanilla, verbena, white clover and yellow dock.

In an embodiment, the antifungal agent is an anti-microbial metal. A number of metals ions been shown to possess antibiotic activity, including silver, copper, zinc, mercury, tin, lead, bismutin, cadmium, chromium and ions thereof. It has been theorized that these anti-microbial metal ions exert their effects by disrupting respiration and electron transport systems upon absorption into bacterial or fungal cells. Anti-microbial metal ions of silver, copper, zinc, and gold, in particular, are considered safe for in vivo use. Anti-microbial silver and silver ions are particularly useful due to the fact that they are not substantially absorbed into the body.

Thus, in one or more embodiment, the anti-microbial metal consists of an elemental metal, selected from the group consisting of silver, copper, zinc, mercury, tin, lead, bismutin, cadmium, chromium and gold, which is suspended in the composition as particles, microparticles, nanoparticles or colloidal particles. The anti-microbial metal can further be intercalated in a chelating substrate.

In further embodiments, the anti-microbial metal is ionic. The ionic antibiotic metal can be presented as an inorganic or organic salt (coupled with a counterion), an organometallic complex or an intercalate. Non binding examples of counter inorganic and organic ions are sulfadiazine, acetate, benzoate, carbonate, iodate, iodide, lactate, laurate, nitrate, oxide, palmitate, a negatively charged protein. In preferred embodiments, the antibiotic metal salt is a silver salt, such as silver acetate, silver benzoate, silver carbonate, silver iodate, silver iodide, silver lactate, silver laurate, silver nitrate, silver oxide, silver palmitate, silver protein, and silver sulfadiazine.

Yet, in another embodiment, the antifungal agent is an oxidizing agent or a substance that releases free radicals and/or active oxygen. Exemplary oxidizing agents are hydrogen peroxide, benzoyl peroxide, elemental halogen species (compounds), as well as oxygenated halogen species (compounds), bleaching agents (e.g., sodium, calcium or magnesium hypochloride and the like), perchlorite species (compounds), iodine and iodate compounds. Organic oxidizing agents are also included in the definition of “oxidizing agent” according to the present invention, such as quinones. Such agents possess a potent broad spectrum activity

In further embodiments, the antibiotic agent is a cationic antimicrobial agent. The outermost surface of bacterial and fungal cells universally carries a net negative charge, making them sensitive to cationic substances. Examples of cationic antibiotic agents include: quaternary ammonium compounds, such as alkyltrimethyl ammonium bromides, benzalkonium chloride, dialkylbenzyl ammonium halides, and dimers thereof, which bear bi-polar positive charges in conjunction with interstitial hydrophobic regions.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina.

Suitable antimycotics include but are not limited to allylamines, amorolfine, amphotericin B, azole compounds, bifonazole, butoconazole, chloroxine, clotrimazole, ciclopirox olamine, clotrimazole, econazole, elubiol, fenticonazole, fluconazole, flucytosine (5FC), griseofulvin, itraconazole, ketoconazole, mafenide acetate, miconazole, naftifine, natamycin, tolnaftate, nystatin, polyenes, oxiconazole, sulbentine, sulconazole, terbinafine, terconazole, tioconazole, undecylenic acid and derivatives, esters, salts and mixtures thereof.

### Vasoactive, Calcium Channel Blocker and Cholinergic Agent

### Vasoactive

A vasoactive agent is a substance that changes the diameter of a blood vessel.

In one or more embodiments, the vasoactive agent is a vasodilator. A vasodilator is any of various agents that relax or widen blood vessels and thereby maintain or lower blood pressure.

Alteration in the release and action of endothelium-derived vasoactive factors is responsible for changes in vascular reactivity early in the course of vascular disease. These factors include nitric oxide, eicosanoids, endothelium-derived hyperpolarizing factor, endothelin, and angiotensin 11.

Nitric oxide (NO) has been recognized as an important messenger molecule having a broad spectrum of functions in many biological systems ranging from physiological control to pathological cytotoxic effect1-3. Along with prostacyclin, NO is responsible for endothelium derived tonic relaxation of all types of blood vessels. NO is formed from L-arginine through the action of a family of isoenzymes, the nitric oxide synthases (NOS). Thus, in one or more embodiments, the vasoactive agent is selected from the group of therapeutic agents that modulate the production of nitric oxide or otherwise modulate or activate the effect of nitric oxide. In one or more embodiments, the vasoactive agent is selected from the group of therapeutic agents that modulate the activity of the enzyme nitric oxide synthase. In one or more embodiments, the vasoactive agent is selected from the group of therapeutic agents that enhance the effect of NO by inhibiting enzymes from the phosphodiesterase group, such as phosphodiesterase type 5 (PDE5).

In one or more embodiments, the vasoactive agent is selected from the group including nitrites, nitrates and their analogs, esters and salts. In one or more embodiments the vasoactive agent possesses a moiety selected from the group consisting of ONO, and ONO2.

Exemplary vasodilators include, but are not limited to, amyl nitrite, amyl nitrate, ethyl nitrite, butyl nitrite, isobutyl nitrite, glyceryl trinitrate, also known as nitroglycerin, octyl nitrite, sodium nitrite, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone (6,7-dimethoxycoumarin) and salts, isomers, analogs and derivatives thereof.

In one or more embodiments, the vasoactive agent belongs to a class of drugs that are known of possess vasodilator properties. Non limiting examples of drug classes that possess vasodilator properties include, but are not limited to, beta-adrenergic blockers, alpha-adrenoceptor blockers, prostaglandin and prostaglandin-like compounds, inhibitors of type 5 phosphodiesterase (PDE-5), angiotensin converting enzyme inhibitors, calcium antagonists, angiotensin II receptor antagonists, direct acting smooth muscle vasodilators, adrenergic inhibitors, endothelin antagonists, mineralocorticoid receptor antagonists, vasopeptidase inhibitors and renin inhibitors. Active agents belonging to such drug classes, as well as active agents belonging to other classes, which cause a vasodilator effect are also included in the scope of vasoactive agents according to the present invention.

Non-nitrate vasodilators from different classes include, but are not limited to sildenafil, dipyridamole, catecholamine, isoproternol, furosemide, prostaglandin, prostacyclin, enalaprilat (ACE-inhibitor), morphine (opiate), acepromazine (α-blocker), prazosin (α-blocker), enalapril(ACE-inhibitor), captopril (ACE-inhibitor), amlodipine (Ca channel blocker), minoxidil, tadalafil, vardenafil, phenylephrin, etilefein, caffeine, capsaicin and salts, isomers, analogs and derivatives thereof.

In one or more embodiments, the vasoactive agent is selected from the group of vasodilator peptides and proteins. Non-limiting examples of vasodilator paprides include, but are not limited to bradykinin, bradykinin-like peptide 1, bradykinin-like peptide III Phyllokinin (bradykinyl-isoleucyl-tyrosine O-sulfate), megascoliakinin ([Thr6]bradykinin-Lys-Ala), lysyl-bradykinin-like waspkinin, lysyl-bradykinin, maximakinin (Bombinakinin M), bombinakinin-GAP, kininogen-1 associated peptides, kininogen-2 associated peptides, T-kinin, thiostatin, prolixin-S, vespulakinin 2, vespakinin X, relaxin, adrenomedullin, ghrelin, maxadilan, substance P, calcitonin gene-related peptide (CGRP), Natriuretic peptides (NPs), e.g., atrial natriuretic peptide (ANP), C-type natriuretic peptide (CNP), and adrenomedullin (ADM), adrenomedullin, ovine corticotropin-releasing factor, sauvagine, urotensin and salts, isomers, analogs and derivatives thereof.

In one or more embodiments, the vasoactive agent is selected from the group of therapeutic agents that induce the production of a vasodilator peptide or otherwise enhance or activate the effect of a vasodilator peptide.

In one or more embodiments, the vasoactive agent is a substance derived or extracted from herbs having a vasodilator effect. Non limiting examples of herbs that contain vasoactive agents include achillea millefolium (Yarrow), allium sativum (garlic), amoracia rusticana (horseradish), berberis vulgaris (barberry), cimicifuga racemosa (black cohosh), coleus forskholii (coleus), coptis (Goldenthread), crataegus (hawthorn), eleutherococcus senticosus (siberian ginseng), ginkgo biloba(ginkgo), melissa offiicnalis (lemon balm), olea europaea (olive leaf), panax ginseng (Chinese ginseng), petroselinum crispum (parsley), scutellaria baicalensis (baical skullcap), tilia europaea (linden flower), trigonella foenum-graecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut, walnut and analogs and derivatives thereof.

According to one or more embodiments, the foamable composition includes a vasodilator and a vasoactive agent such that the vasodilator can have a synergistic effect by readily facilitating facile penetration of the vasoactive agent.

In one or more embodiments, the vasoactive agent is a vasoconstrictor. A vasoconstrictor is any of various agents that narrow blood vessels and thereby maintain or increase blood pressure, and/or decrease blood flow. There are many disorders that can benefit from treatment using a vasoconstrictor. For example, redness of the skin (e.g., erythema or cuperose), which typically involves dilated blood vessels, benefit from treatment with a vasoconstrictor, which shrinks the capillaries thereby decreasing the untoward redness.

Other descriptive names of the vasoconstrictor group include vasoactive agonists, vasopressor agents and vasoconstrictor drugs. Certain vasoconstrictors act on specific receptors, such as vasopressin receptors or adrenoreceptors.

In one or more embodiments, the vasoconstrictor is a calcium channel agonist. Calcium channel agonists are agents that increase calcium influx into calcium channels of excitable tissues, thereby causing vasoconstriction.

Non limiting examples of vasoconstrictors include ephedrine, epinephrine, phenylephrine, angiotensin, vasopressin, and analogs and derivatives thereof.

In one or more embodiments, the vasoactive agent is a substance derived or extracted from herbs, having a vasoconstrictor effect.

Thus, in one or more embodiments, the vasoactive agent is a substance derived or extracted from a herbal source, selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom), cypress and salts, isomers, analogs and derivatives thereof.

Yet, in additional embodiments, the vasoactive agent is a metal oxide or a mineral, such as zinc oxide and bismuth subgallate.

The McKenzie vasoconstrictor assay, as described, for example, in the British Journal of Dermatology 1975; 93:563-71 and versions thereof, has been the primary method used for classifying the strength of a vasoconstrictor clinical efficacy. Thus, in one or more embodiments, the vasoactive agent is an agent that positively affects the vasoconstrictor assay.

Mixtures of these vasoactive agents may also be employed according to the present invention.

Solubility of the vasoactive agent is an important factor in the development of a stable foamable composition according to the present invention.

### Calcium Channel Blockers

Calcium channel blockers are a chemically and pharmacologically heterogeneous group of drugs, but physiologically they all share the ability to selectively antagonize the calcium ion movements that are responsible for the excitation-contraction coupling in the cardiovascular system. Beyond their cardiovascular effects, calcium channel blockers are known to possess other effects, such as inhibition of the growth and proliferation of vascular smooth muscle cells and fibroblasts, inhibition of the synthesis of extracellular matrix proteins, immunomodulation, inhibition of mast cell degranulation and platelet aggregation and suppression of neutrophil adhesion and superoxide anion (O-2) production. Some calcium channel blockers also have analgesic effects.

Current therapeutic uses of calcium channel blockers include (but are not limited to) hypertension, angina, arrhythmia and subarachnoid hemorrhage. Calcium channel blockers may further relieve or prevent reactive vasodilation of migraine sufferers by inhibiting the vasoconstriction during the prodromal phase.

There are two main classes of calcium channel blockers: dihydropyridines (e.g., nifedipine, nicardipine, amlodipine, felodipine and nimodipine) and nondihydropyridines which include diltiazem (a benzothiazepine) and verapamil (a phenylalkylamine). Flunarizine is an antihistamine with calcium channel blocking activity.

In an embodiment, the calcium channel blocker can be selected from the group consisting of an amlodipine, anipamil, barnidipine, benidipine, bepridil, darodipine, diltiazem, efonidipine, felodipine, isradipine, lacidipine, lercanidipine, lidoflazine, manidipine, mepirodipine, nicardipine, nifedipine, niludipine, nilvadipine, nimodipine, nisoldipine, nitrendipine, perhexyline, tiapamil, verapamil, pharmaceutically acceptable salts, isomers, analogs and derivatives thereof.

### Cholinergic Drugs

Cholinergic drugs produce the same effects as acetylcholine. Acetylcholine is the most common neurohormone of the parasympathetic nervous system, the part of the peripheral nervous system responsible for the every day work of the body. A cholinergic agent, also known as a parasympathomimetic agent, is a chemical which functions to enhance the effects mediated by acetylcholine in the central nervous system, the peripheral nervous system, or both. These include acetylcholine receptor agonists muscarine and nicotine, as well as anticholinesterases.

Suitable cholinergic drugs in accordance with the present invention are selected from a cholinergic agonist of acetylcholine, bethanechol, carbachol, methacholine, and pilocarpine, or an anticholinesterase of ambenonium, neostigmine, physostigmine, pyridostigmine, dyflos, and ecothinopate, and pharmaceutically acceptable salts, isomers, analogs and derivatives thereof.

### Nitric Oxide Donors

Nitric oxide is an inorganic free radical, which has the chemical formula of N═O and abbreviated to NO, and is a remarkably versatile biological messenger. The chemical properties of NO are crucial in defining its biological roles, both as a transcellular signal in the cardiovascular and nervous systems and as a cytotoxic antipathogenic agent released during an inflammatory response. Endogenous NO is synthesized from the amino acid L-arginine by three isoforms of the enzyme NO synthase (NOS). The endothelial (eNOS) and neuronal (nNOS) isoforms that synthesize NO for transcellular signaling are constitutively expressed tightly regulated by a number of cofactors. These NOS isoforms typically synthesize small amounts of NO and require activation by Ca²⁺-calmodulin, making them sensitive to agents and processes that increase intracellular calcium levels. The NO generated diffuses to neighboring target cells where it acts primarily through activation of soluble guanylate cyclase (sGC) to generate cGMP from GTP, and bring about the cellular response through a reduction in intracellular calcium levels.

In an embodiment, the nitric oxide donors can be selected from several classes, including, but not limited to inorganic nitrites and nitrates (e.g., sodium nitrite), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil, FK-409 (4-Ethyl-2-[(Z)-hydroxyiminol]-5-nitro-3(E)-hexeneamide) and derivatives thereof and hybrid NO donor drugs.

In an embodiment, the organic nitric oxide donor includes at least one organic nitrate, which includes esters of nitric acid and may be an acyclic or cyclic compound. For instance, the organic nitrate may be ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, nitroglycerin, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and pharmaceutically acceptable salts, isomers, analogs and derivatives thereof.

In one embodiment, vasoactive drugs that act via eNOS activity enhancement, such as sildenafil, vardenafil and tadalafil are also regarded “nitric oxide donors.”

In an embodiment, the active agent is an antipruritic. Suitable antipruritics include but are not limited to menthol, methdilazine, trimeprazine, urea and derivatives, esters, salts and mixtures thereof.

In an embodiment, the therapeutic agent is an additional antipsoriatic agent. Suitable additional antipsoriatic agents include but are not limited to 6-aminonicotinamide, 6-aminonicotinic acid, 2-aminopyrazinamide, anthralin, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

In an embodiment, the active agent is an antirosacea agent. Suitable antirosacea agents include but are not limited to azelaic acid, metronidazole, sulfacetamide and derivatives, esters, salts and mixtures thereof. Certain nonsteroidal anti-inflammatory agents, such as salicylic acid, salycilates, piroxicam and diclofenac are also useful for the treatment of Rosacea.

In an embodiment, the therapeutic agent is an antiseborrheic agent. Suitable antiseborrheic agents include but are not limited to glycolic acid, salicylic acid, selenium sulfide, zinc pyrithione, a dicarboxylic acid, such as azelaic acid and derivatives, esters, salts and mixtures thereof.

In an embodiment, the therapeutic agent is an antiviral agent. Suitable antiviral agents include but are not limited to acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

In an embodiment, the therapeutic agent is a chemotherapeutic agent. Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

Several disorders of the skin, a body cavity or mucosal surface (e.g., the mucosa of the nose, mouth, eye, ear, vagina or rectum) involve a combination of inflammation, cell proliferation and differentiation abnormalities, and other biological abnormalities that can be effected by a steroid; and other etiological factors that require an additional therapeutic modality. For example, psoriasis involves inflammation as well as excessive cell proliferation and inadequate cell differentiation. Atopic dermatitis involves inflammation, skin dryness and keratinocyte growth abnormality. Bacterial, fungal and viral infections involve pathogen colonization at the affected site and inflammation. Likewise, hair growth disorders and other pilosebaceous disorders involve an impaired hormonal balance (which can be affected by a steroid hormone or a steroid hormone antagonist), together with other etiological factors, that can be affected a non-steroidal active agent. Hence, in many cases, the inclusion of an additional therapeutic agent in the foamable pharmaceutical composition, contributes to the clinical activity of the steroid. Thus, in one or more embodiments, the foamable composition further includes at least one additional therapeutic agent, in a therapeutically effective concentration.

In one or more embodiments, the at least one additional non-steroidal therapeutic agent is selected from the group consisting of an anti-infective, an antibiotic, an antibacterial agent, an antifungal agent, an antiviral agent, an antiparasitic agent, a nonsteroidal anti-inflammatory drug, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

In certain cases, the disorder to be treated involves unaesthetic lesions that need to be masked. For example, rosacea involves papules and pustules, which can be treated with a steroid, as well as erythema, telangiectasia and redness, which do not respond to treatment with a steroid. Thus, in one or more embodiments, the additional active agent is a masking agent, i.e., a pigment. Non limiting examples of suitable pigments include brown, yellow or red iron oxide or hydroxides, chromium oxides or hydroxides, titanium oxides or hydroxides, zinc oxide, FD&C Blue No. 1 aluminum lake, FD&C Blue No. 2 aluminum lake and FD&C Yellow No. 6 aluminum lake.

In an embodiment, the active agent is a hair growth regulator. Suitable hair growth regulators include but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chiorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione and derivatives, esters, salts and mixtures thereof.

In an embodiment, the therapeutic agent is a hormone. Suitable hormones include but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, androsteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b-ol-20-one, 16,5a-pregnen-3b-ol-20-one, 4-pregnen-20b-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone, progestins and derivatives, esters, salts and mixtures thereof.

In an embodiment, the therapeutic agent is a hydroxyacid. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

### Keratolytic Agents

In an embodiment, the active agent is a keratolytic agent. The term “keratolytic agent” is used herein to mean a compound which loosens and removes the stratum corneum of the skin, or alters the structure of the keratin layers of skin. Keratolytic agents are used in the treatment of many dermatological disorders, which involve dry skin, hyperkeratiinization (such as psoriasis), skin itching (such as xerosis), acne and rosacea. Suitable keratolytic agents include but are not limited to N-acetylcysteine, azelaic acid, cresols, dihydroxy benzene compounds, such as resorcinol and hydroquinone, alpha-hydroxy acids, such as lactic acid and glycolic acid, phenol, pyruvic acid, resorcinol, sulfur, salicylic acid, retinoic acid, isoretinoic acid, retinol, retinal, urea and derivatives, esters, salts and mixtures thereof.

The term “keratolytic agent” refers herein to a compound which loosens and removes the stratum corneum of the skin, or alters the structure of the keratin layers of skin.

Suitable keratolytic agents also include alpha-hydroxy acids. Alfa hydroxy acids are keratolytic, and they are also capable of trapping moisture in the skin and initiating the formation of collagen. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

Yet, another preferred keratolytic agent is urea, as well as derivatives thereof. Urea possesses both keratolytic and skin-hydration properties which are beneficial to the damaged tissue of the skin.

Another preferred group of keratolytic agents, suitable for inclusion in the therapeutic composition according to the present invention is beta-hydroxy acids, such as salicylic acid (o-hydroxybenzoic acid). Beta hydroxyl acids are keratolytic, and they are also have anti-inflammatory and antibacterial properties.

Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). In the context of the present invention, di-carboxylic acids having up to 6 carbon atoms in their skeleton are also suitable under the definition of short chain carboxylic acids having up to 6 carbon atoms in their skeleton. Non-limiting examples of suitable dicarboxylic acids are malonic acid (propanedioic acid), succinic acid (butanedioic acid), glutaric acid (Pentanedioic acid) and adipic acid (Hexanedioic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH2FCO2H), chloroethanoic acid (CH2ClCO2H) and dichloroethanoic acid (CHCl2CO2H). Dicarboxylic acids, having between about 6 and about 14 carbon atoms in their carbon atom skeleton also possess leratolytic properties. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid.

Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinol, retinal, retinoic acid, retinyl acetate, retinyl palmitate, retinyl ascorbate, isotretinoin, tazarotene, adapalene, 13-cis-retinoic acid, acitretin all-trans beta carotene, alpha carotene, lycopene, 9-cis-beta-carotene, lutein and zeaxanthin are another class of keratolytic agents, which alter the structure of the skin and promote peeling.

In certain embodiments, the keratolytic agent includes at least two keratolytic agents. At least two or more keratolytic agents in the therapeutic composition, a safe and effective peeling agent is attained, which breaks down the keratin layer of the skin, where the microorganisms reside. As a result of such breaking down of the keratin layer, the microorganisms cannot further survive in the infected area. The combination of at least two keratolytic agents enables a selective breaking down of keratin in infected skin areas, while non-infected skin areas are not affected. This phenomenon is explained by the fact that the keratin layer in infected skin areas is deformed and thus it is more vulnerable to keratolytic disintegration. Furthermore, combining at least two keratolytic agents facilitates use of each agent in a substantially minimally-irritating concentration, thus decreasing the overall irritation of the therapeutic composition.

In one or more embodiments, the keratolytic agent includes at least two keratolytic agents, from different families of chemicals. Thus, in preferred embodiments, the keratolytic agent includes at east two agents, from different chemical families, selected from the group consisting of: (1) an alpha-hydroxy acid; (2) a beta-hydroxy acid; (3) a short-chain carboxylic acid; (4) a hydroxylbenzene; (5) a vitamin A derivative; and (6) urea. As detailed above, each of these keratolytic agent families possess, in addition to their keratolytic property, additional therapeutically-beneficial feature, such as anti-inflammatory, skin hydration and antibacterial properties for readily contributing to the overall therapeutic benefit of the therapeutic composition.

In an embodiment, the active agent is a lactam. Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-I-actam, 2-acetamido-2-deoxygalactono-1,5-lactam, D-glucaro-1,4:6,3-dilactam-, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

### Nonsteroidal Anti-Inflammatory Agent

In an embodiment, the therapeutic agent is a non-steroidal anti-inflammatory agent.

Inflammation is defined as “redness, swelling, and fever in a local area of the body, often with pain and disturbed function, in reaction to an infection or to a physical or chemical injury” (Random House Webster's Dictionary). Typical symptoms of disorders of the skin, body surfaces, body cavities and mucosal surfaces (e.g., the mucosa of the nose, mouth, eye, ear, respiratory system, vagina or rectum) that involve inflammation, as at least one of their etiological factors, include redness (rash, erythema), tissue thickening and/or swelling (oedema), itch (pruritus), blistering and exudate. Inflammatory disorders can by short term or long term (chronic). Inflammation typically involves overproduction of pro-inflammatory cytokines, such as TNF-alpha, TNF-beta, interleukin-1, interleukin-4, interleukin-6, interleukin-10, interleukin-12, IFN-gamma from T cells, or increased release of cytokines and pro-inflammatory mediators from mast cells.

In the context herein, a nonsteroidal immunomodulating agent (also termed herein “nonsteroidal anti-inflammatory agent” and “NSAID”) is a pharmaceutically active compound, other than a corticosteroid, which affects the immune system in a fashion that results in a reduction, inhibition, prevention, amelioration or prevention of an inflammatory process and/or the symptoms of inflammation and or the production pro-inflammatory cytokines and other pro-inflammatory mediators, thereby treating or preventing a disease that involves inflammation.

In one or more embodiments, the NSAID is an inhibitor of the cyclooxygenase (COX) enzyme. Two forms of cyclooxygenase are known today: the constitutive cyclooxygenase (COX-1); and the inducible cyclooxygenase (COX-2), which is proinflammatory. Thus, in one or more embodiments, the NSAID is selected from the group consisting of a COX-1 inhibitor, a COX-2 inhibitor or a non-selective NSAID, which simultaneously inhibits both COX-1 and COX-2.

The term “selective COX-2 inhibitor” relates o a compound able to inhibit cyclooxygenase-2 without significant inhibition of COXe-1. Typically, it includes compounds that have a COX-2IC₅₀ of less than about 0.2 micro molar, and also have a selectivity ratio of COX-2 inhibition over COX-1 inhibition of at least 50, and more typically, of at least 100. Inhibitors of the cyclooxygenase pathway in the metabolism of arachidonic acid used in the present invention may inhibit enzyme activity through a variety of mechanisms. By the way of example, and without limitation, the inhibitors used in the methods described herein may block the enzyme activity directly by acting as a substrate for the enzyme.

Selective COX-2 Inhibitors include, in an exemplary manner diaryl-substituted furanones (e.g., Rofecoxib); diaryl-substituted pyrazoles (e.g., Celecoxib); indole acetic acids (e.g., Etodolac); and sulfonanilides (e.g., Nimesulide) and salts and derivatives thereof.

In one or more embodiments, the selective COX-2 inhibitor is selected from the group consisting of celecoxib, deracoxib, valdecoxib, rofecoxib, lumiracoxib, etoricoxib, meloxicam, parecoxib, 4-(4-cyclohexyl-2-methyloxazol-5-yl)-2-fluorobenzenesulfonamide, 2-(3,5-difluorophenyl)-3-(4-(methylsulfonyl)phenyl)-2-cyclopenten-1-one, N-[2-(cyclohexyloxy)-4-nitrophenyl]methanesulfonamide, 2-(3,4-difluorophenyl)-4-(3-hydroxy-3-methylbutoxy)-5-[4-(methylsulfonyl)-phenyl]-3(2H)-pyridazinone, 2-[(2,4-dichloro-6-methylphenyl)amino]-5-ethyl-1-benzeneacetic acid, (3Z)-3-[(4-chlorophenyl)[4-(methylsulfonyl)phenyl]met-hylene]dihydro-2(3H)-furanone, and (S)-6,8-dichloro-2-(trifluoromethyl)-2H-1-benzopyran-3-carboxylic acid.

In additional embodiments, the selective COX-2 inhibitor is selected from the group consisting of ibuprofen, naproxen, benoxaprofen, flurbiprofen, fenoprofen, fenbufen, ketoprofen, indoprofen, pirprofen, carprofen, oxaprozin, prapoprofen, miroprofen, tioxaprofen, suprofen, alminoprofen, tiaprofenic acid, fluprofen, bucloxic acid, indomethacin, sulindac, tolmetin, zomepirac, diclofenac, fenclofenec, alclofenac, ibufenac, isoxepac, furofenac, tiopinac, zidometacin, acetyl salicylic acid, indometacin, piroxicam, tenoxicam, nabumetone, ketorolac, azapropazone, mefenamic acid, tolfenamic acid, diflunisal, podophyllotoxin derivatives, acemetacin, droxicam, floctafenine, oxyphenbutazone, phenylbutazone, proglumetacin, acemetacin, fentiazac, clidanac, oxipinac, mefenamic acid, meclofenamic acid, flufenamic acid, niflumic acid, flufenisal, sudoxicam, etodolac, piprofen, salicylic acid, choline magnesium trisalicylate, salicylate, benorylate, fentiazac, clopinac, feprazone, isoxicam, and 2-fluoro-a-methyl[1,1′-biphenyl]-4-ace-tic acid, 4-(nitrooxy)butyl ester.

In one or more embodiments, the NSAID is salicylic acid a salicylic acid derivatives. Exemplary salicylic acid derivative include, in a non limiting fashion, aspirin, sodium salicylate, choline magnesium trislicylate, salsalate, diflunisal, salicylsalicylic acid, sulfasalazine, olsalazine, esters of salicylic acid with a carboxylic acid, esters of salicylic acid with a dicarboxylic acid, esters of salicylic acid with a fatty acid, esters of salicylic acid with a hydroxyl fatty acid, esters of salicylic acid with an essential fatty acid, esters of salicylic acid with a polycarboxylic acid, and any compound wherein salicylic acid is linked to an organic moiety through a covalent bond.

In one or more embodiments, the NSAID is para-aminophenol (e.g., acetaminophen) and salts and derivatives thereof.

In one or more embodiments, the NSAID is an indole or an indole—acetic acid derivative (e.g., indomethacin, sulindac, etodolac) and salts and derivatives thereof.

In one or more embodiments, the NSAID is an aryl acetic acids (e.g., tolmetin, diclofenac, ketorolac) and salts and derivatives thereof.

In one or more embodiments, the NSAID is an arylpropionic acid and salts and derivatives thereof. Exemplary arylpropionic acid derivative include, in a non limiting fashion, are ibuprofen, naproxen, flubiprofen, ketoprofen, fenoprofen, oxaprozin.

In one or more embodiments, the NSAID is anthranilic acids or an anthranilic acid derivative, also termed “fenamates” (e.g., mefenamic acid, meclofenamic acid) and salts and derivatives thereof.

In one or more embodiments, the NSAID is selected from the group of enolic acids, enolic acid salts, enolic acid esters, amides, anhydrides and salts and derivatives thereof. Non-limiting examples of enolic acid derivatives include oxicams (piroxicam, tenoxicam) and pyrazolidinediones (phenylbutazone, oxyphenthratrazone)

Yet, in additional embodiments, the NSAID is an alkanone (e.g., nabumetone).

Certain imidazole drugs (e.g., ketoconazole) also possess anti-inflammatory properties, (See: J Am Acad. Dermatol. 1991 August; 25(2 Pt 1):257-61).

Another group of nonsteroidal immunomodulating agents includes agents, which inhibit pro-inflammatory cytokines, such as TNF-alpha, TNF-beta, interleukin-1, interleukin-4, interleukin-6, interleukin-10, interleukin-12 and IFN-gamma from T cells, which are especially important in the induction of inflammation or inhibit the release of cytokines and pro-inflammatory mediators from mast cells.

Agents that are used to affect the untoward influence of pro-inflammatory cytokines are chemically or biologically-originated materials that suppress the pro-inflammatory effect of a pro-inflammatory cytokine via various mechanisms, including, but not limited to (a) inhibiting the formation of a pro-inflammatory cytokine; (b) suppressing the interaction of a pro-inflammatory cytokine with its receptors; or (c) neutralization the proinflammatory cytokine by direct or indirect interaction.

Examples of chemical anti TNF-α agents are known pharmaceutical materials, such as pentoxifylline, propentofylline, torbafylline (and other related xanthines), amiloride, chloroquine, thalidomide and structural analogs thereof. Examples for biological anti-TNF-α agents are anti-TNF-α antibodies and soluble TNF-β receptors. Additional compounds are those that impair the signal transduction cascade from the receptor to other functional organs of the living cell. Such active agents, as well additional compounds, which are capable of inhibiting the production or otherwise suppressing the pro-inflammatory effects of TNF-α can be used in the composition.

Immunosuppressant agents, immunoregulating agents and immunomodulators constitute an additional class of nonsteroidal anti-inflammatory agents, which are used according to the present invention. Such agents are chemically or biologically-derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod. In one or more embodiments, the non steroidal immunomodulating agent is a calcineurin Inhibitor.

In one or more embodiments, the NSAID is a nitric oxide inhibitor. Nitric oxide (NO) is a potent secondary messenger that is both highly reactive and highly diffusible. It is generated physiologically by a family of enzymes, referred to as NO synthases (NOS). Overproduction of NO plays a key role in the pathology of a wide range of disorders including disorders that involve inflammation, and NOS inhibitors have been suggested as anti-inflammatory agents. Agents that neutralize NO (also called “NO scavengers”) are considered as potential anti-inflammatory agents as well.

Also useful are compounds that inhibit or slow down the migration of leucocytes (white blood cell), e.g., macrophages, neutrophils, and monocytes towards an afflicted skin surface or mucosal membrane, which is known to accelerate the inflammatory process.

Among other inhibitors of leucocyte chemoaxis, dicarboxylic acids, having between about 6 and about 14 carbon atoms in their carbon atom skeleton are particularly useful in the treatment of disorders of the skin and mucosal membranes that involve inflammation. Suitable dicarboxylic acid moieties include, but are not limited to, adipic acid, pimelic acid, suberic acid, azelaic acid, sebacic acid, 1,11-undecanedioic acid, 1,12-dodecanedioic acid, 1,13-tridecanedioic acid and 1,14-tetradecanedioic acid. Thus, in one or more embodiments, dicarboxylic acids, having between about 6 and about 14 carbon atoms in their carbon atom skeleton, as well as their salts and derivatives (e.g., esters, amides, mercapto-derivatives, anhydraides), are useful immunomodulators in the treatment of disorders of the skin and mucosal membranes that involve inflammation. Azelaic acid and its salts and derivatives are preferred.

Certain preferred dicarboxylic acid derivatives include a dicarboxylic acid wherein at least one ester moiety of the compound comprises a keratolytic agent, selected from the group consisting of alpha-hydroxy acids and derivatives thereof, beta-hydroxy acids and derivatives thereof, hydroxybenzoic acid and their ester, anhydride and amine derivatives, alkylhydroxybenzoate, dihydroxy benzene and their ester, anhydride and amide derivatives, cresols and their ester, anhydride and amide derivatives. Keratolytic agents also include alcohol derivatives of Vitamin A (retinoic acid), e.g., retinol and derivatives thereof, as provided in U.S. Pat. No. 6,180,669. Additional preferred dicarboxylic acid derivatives comprise at least one ester of a active alcohol moiety, selected from the groups of steroid hormones, corticosteroids, vitamin E and vitamin D, as provided in US Patent Application 20040191196.

In one or more embodiments, the NSAID is an ion channel modulator. Ion channels are protein macromolecules located in the cell membranes that enable the selective movement of sodium, potassium, and calcium from outside the cell to inside the cell and vice-versa.

In one or more embodiments, the NSAID is a potassium ion channel modulator. It has been shown that the potassium ion channel modulator play important roles in controlling T-cell activation and thus, they can be used to control inflammation.

In one or more embodiments, the potassium ion channel modulator is selected from the group consisting of dendrotoxin, dendrotoxin I, dendrotoxin K, alpha-dendrotoxin, beta-dendrotoxin, gamma-dendrotoxin, margatoxin, stichodactyla toxin, tityustoxin K, apamin, charylotoxin, clotrimazole, dequalinium chloride, iberiotoxin, kaliotoxin, neuropeptide Y, noxiustoxin, tolbutamide, chlorpropamide, glibenclamide, glipizide, nategliniide, repagliniide, glyburide, tolazamide, nicorandil, fampridine and penitrem A, or is a pharmaceutically acceptable salt or prodrug thereof.

In an embodiment, the potassium ion channel modulator is selected from the list of potassium ion channel modulators, provided in WO 20041093895.

In one or more embodiments, the NSAID is a sodium ion channel modulator. In one or more embodiments, the sodium ion channel blocker is selected from the group consisting of disopyramide, procainimide, quinidine, tocamide, mexiletene, lidocane, phenyloin, fosphenyloin, flecamide, propafenone, morcizine, lubeluzole, carbamazepine, sipatrigine, riluzole, tetrodotoxin, spheroidine, maculotoxin, vinpocetine, anthopleurin-c, lamotrigine, crobenetine, lifarizine, lanodipine, lomerizine, encamide, and flunarizine or is an isomer, a pharmaceutically acceptable salt, ester, or prodrug thereof.

In an embodiment, the potassium ion channel modulator is selected from the list of potassium ion channel modulators, provided in U.S. Pat. Appl. 20040224940 and 20040220187.

In one or more embodiments, the NSAID is a modulator of serotonin (5-hydroxytryptamine, 5-HT) activity. 5-HT is known to affect inflammation through its modulation effect on cytokine production (Cloëz-Tayarani et al. Int. Immunol. 2003, 15 233). In certain embodiments, the serotonin activity modulator is a serotonin reuptake inhibitor. It has been shown that serotonin reduces inflammation and assists healing of experimental skin wounds, and thus, serotonin reuptake inhibitor can be used to control inflammation and associated disorders.

In one or more embodiments, the serotonin reuptake inhibitor is selected from the group consisting of citalopram, fluoxetine, fluvoxamine, paroxetine, escitalopram oxalate, sertraline, norfluoxetine and N-demethylsertraline.

In an embodiment, the serotonin reuptake inhibitor is selected from the list of potassium ion channel modulators, provided in US Pat Appl. 20040171664.

In one or more embodiments, the NSAID is an antioxidant. Reactive oxygen species play an important role in mediating skin inflammation, and antioxidants may provide protection.

Non-limiting examples of antioxidant agents include 21-[4-[2-amino-6-(diethylamino)-4-pyrimidinyl]-1-piperazinyl]-17α-hydroxypregna-4,9(11)-diene-3,20-dione, 17α-hydroxy-21-[4-[2,6-bis(dimethylamino)-4-pyrimidinyl]-1-piperazinyl]pregna-4,9(11)-diene-3,20-dione, 21-[4-[2-(diethylamino)-6-(1-pyrrolidinyl)-4-pyrimidinyl]-1-piperazinyl]-17α-hydroxypregna-4,9(11)-diene-3,20-dione, 17α-hydroxy-21-[4-[2-(diethylamino)-6-(4-methyl-1-piperazinyl(4-pyrimidinyl)]-1-piperazinyl]pregna-4,9(11)-diene-3,20-dione, 17α-hydroxy-21-[4-[2,6-bis(diethylamino)-4-pyrimidinyl]1-piperazinyl]pregna-4,9(1)-diene-3,20-dione, 1α-hydroxy-21-[4-[2-(diethylamino-)-6-(1-piperidinyl)-4-pyrimidinyl]-1-piperazinyl]pregna-4,9(11)-diene-3,20-dione, 21-[4-[2,6-bis(diethylamino)-4-pyrimidinyl)-4-pyrimidinyl]-1-piperazinyl]-1-piperazinyl]-17α-hydroxy-16α-methylpregna-1,4,9(11)-triene-3,20-dione, 17α-hydroxy-21-[4-[2,6-bis(4-methyl-1-piperazinyl]pregna-4,9(11)-diene-3,20-dione, 17α-hydroxy-6α-methyl-21 [4-2,6-bis-(1-pyrrolidinyl-4-pyrimidinyl]-1-piperazinyl]pregna-1,4,9(11)-triene-3,20-dione, 21-[4-2,6-bis(diethylamino)-4-pyrimidinyl]-1-piperazinyl]-1-1α,17α-dihydroxypregn-4-ene-3,20-dione, 21-[4-[2,6-bis(diethylamino)-4-pyrimidinyl]-1 piperazinyl]-17α-hydroxypregn-4-ene-3,20-dione, 21-[4-[2,6-bis(diethylamino)-4-pyrimidinyl]-1-piperazinyl]-17α-hydroxy-6α-methylpregna-1,4,9(11)-triene-3,20-dione, 17α-hydroxy-21-[4-[2,6-bis(-pyrrolidinyl)-4-ppyrimidinyl]-1-piperazinyl]pregna-4,9(11)-diene-3,20-dione, 21-[4-[2,6-bis(diethylamino)-4-pyrimidinyl]-1-piperazinyl]-11α-hydroxypregn-4-ene-3,20-dione, 21-[4-[2,6-bis(diethylamino)-4-pyrimidinyl]-1-piperazinyl]-11α,17α-dihydroxypregn-4-ene-3,20-dione, 17α-hydroxy-16α-methyl-21-[4-[2,6-bis-(1-pyrrolidinyl)-4-pyrimidinyl]-1-piperazinyl]pregna-1,4,9(−11)-triene-3,20-dione, 17α-hydroxy-21-[4-[2,6-bis(1-pyrrolidinyl)-4-pyrimidinyl]-1-piperazinyl]pregna-1,4,9(11)-triene-3,20-dione, 21-[4-[2,6-bis(diethylamino)-4-pyrimidinyl]-1-piperazinyl]-17 α-hydroxypregna-1,4,9(1)-triene-3,20-dione, 21-[4-[4,6-bis(diethylamino)-2-pyrimidinyl]-1-piperazinyl]-17α-hydroxypregna-1,4,9(11)-triene-3,20-dione, 21-[4-[2,6-bis(diethylamino)-4-pyrimidinyl]-1-piperazinyl]-16α-methylpregna-1,4,9(11)-triene-3,20-dione, 21-[4-[2,6-bis(diethylamino)-4-pyrimidinyl]-1-piperazinyl]-11.alpha.-hydroxy-16α-methylpregna-1,4-dien-e-3,20-dione, 21-[4-[2,6-bis(diethylamino)-4-pyrimidinyl]-1-piperazinyl]-1-6α-methylpregna-1,4-diene,3,20-dione, 16α-methyl-21-[4-[2,6-bi-s(1-pyrrolidinyl)-4-pyrimidinyl]-1-piperazinyl]pregna-1,4,9(11)-triene-3,2-O-dione, 11α-hydroxy-16α21-[4-[2,6-bis(1-pyrrolidinyl)-4-pyrimidinyl]piperazinyl]pregna-1,4-diene-3,20-dione, 16α-methyl-21-[4-[2,6-bis(1-pyrrolidinyl)-4-pyrimidinyl]-1-piperazinyl]pregna-1,4-diene-3,20-dione, 16.alpha.-methyl-21-[4-[2,6-bis(4-morpholino)-4-pyrimidinyl]-1-piperazinyl]pregna-1,4,9(11)-triene-3,20-dione, 11α-hydroxy-16α-methyl-21-[4-[2,6-bis(4-morpholino)-4-pyrimidinyl]-1-piperazinyl]pregna-1,4-diene-3,20-dione, 16.alpha.-methyl-21-[4-[2-,6-bis(4-morpholino(4-pyrimidinyl]-1-piperazinyl]pregna-1,4-diene-3,20-dione, 21-[4-[2,6-bis(allylamino)-4-pyrimidinyl]-1-piperazinyl[-16α-methylpregna-1,4,9(11)-triene-3,20-dione, 21-[4-[2,6-bis(allylamino)-4-pyrimidinyl]-1-piperazinyl]-11α-hydroxy-16α-methylpregna-1,4-ene-3,2-O-dione, 21-[4-[2,6-bis(allylamino)-4-pyrimidinyl]-1-piperazinyl]-16α-methylpregna-1,4-ene-3,20-dione, 21-[4-[2,6-bis(1-pyrrolidinyl)-4-pyrimidinyl]-1-piperazinyl]pregn-4-ene-3,11,20-trione, 21-[4-[2,6-bis(1-pyrrolidinyl)-4-pyrimidinyl]-1-piperazinyl]pregna-4,9(11)-diene-3,20-dione, 21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]pregna-4,9(11)-diene-3,20-dione, 21-[4-(2,6-bis(4-morpholino)-4-pyrimidinyl)-1-piperazinyl]-17α-hydroxypregna-4,9(11)-diene-3,20-dione, 21-[4-(2,6-bis(1-pyrrolidinyl)-4-pyrimidinyl)-1-piperazinyl]pregna-4-en-3-one, 21-[4-(2,6-bis(1-pyrrolidinyl)-4-pyrimidinyl)-1-piperazinyl]pregn-4-en-3-one, 16α-methyl-21-[4-[2,6-bis(1-pyrrolidinyl)-4-pyrimidinyl]-1-piperazinyl]pregna-1,4,9(11)-triene-3,20-dione, 21-[4-(2,6-bis(1-pyrrolidinyl)-4-pyrimidinyl)-1-piperazinyl]pregna-1,4,9(11)-triene-3,20-dione, 21-[4-(2,6-bis(1-pyrrolidinyl)-4-pyrimidinyl)-1-piperazinyl]-20-methylpregna-1,4-dien-3-one, 21-[4-(2,6-bis(1-pyrrolidinyl)-4-pyrimidinyl)-1-piperazinyl]pregna-1,4,9(11),16-tetraene-3,20-dione, 21-[4-[2,6-bis(4-morpholino)-4-pyrimidinyl]-1-piperazinyl]pregna-1,4-diene-3,20-dione, 21-[4-[2,6-bis(diethylamino)-4-pyrimidinyl]-1-piperazinyl]-6α-fluoro-17α-hydroxy-16β-methylpregna-4,9(11)-diene-3,20-dione, 6α-fluoro-17α-hydroxy-16β-methyl-21-[4-[2,6-bis(1-pyrrolidinyl)-4-pyrimidinyl]-1-piperazinyl]pregna-4,9(11)-diene-3,20-dione, 16 α-methyl-21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]-16α,17α-dimethylpregna-1,4,9(11)-riene-3,2-O-dione, 3β-hydroxy-16α-methyl-21-[4-[2,6-bis(1-pyrrolidinyl)-4-1-pyrimidinyl]-1-piperazinyl]-pregn-5-en-20-one, 16α-methyl-21-[4-[2,-6-bis-(1-pyrrolidinyl)-4-pyrimidinyl]-1-piperazinyl]pregna-1,4,6,9(11)-tetraene-3,20-dione, 3β-hydroxy-16α-methyl-21-[4-[2,6-bis(1-pyrrolidinyl)-4-pyrimidinyl]-1-piperazinyl]pregn-5-en-20-one, 16α-methyl-17β-(1-oxo-4-[4-[2,6-bis(1-pyrrolidinyl)-4-pyrimidinyl]-1-piperazinyl]butyl)-androsta-4,9(11)-dien-3-one, tocopherol, vitamin C, beta-carotene, lycopene, coenzyme Q, idebenone, lipoic acid, and ginkgo biloba; or is an isomer, a pharmaceutically acceptable salt, ester, or prodrug thereof.

In one or more embodiments, the NSAID is a cannabinoid. Cannabnoids are known to affect inflammation through suppression of runaway inflammation and other untoward effects of immune system activation, as well as pain.

In certain embodiments, the cannabinoid agent is selected from the group consisting of: 2-arachidonylglycerol; N-arachidonyl-1-(2,3-dichlorobenzoyl)-2-methyl-3-(2-[1-morpholino]ethyl)—5-methoxyindole; 2-methyl-1-propyl-3-(1-naphthoyl)indole; 1-methoxy-N,N-dimethylmethanamide; 1-methoxy-endo-4-hydroxy-9-oxabicyclo[3.3.1)nonane; dronabinol; (2-methyl-1-propyl-1H-indol-3-yl)-1-naphthalenylmethanone; 3-(1,1-dimethylbutyl)-6a,7,10,10a-tetrahydro-6,6,9-6h-dibenzo[b,d]pyran; [2,3-dihydro-5-methyl-3(4-morpholinylmethyl)pyrrolo[1,2,3-de]methane; 5-(1,1-dimethylheptyl)-2-[(1R,2R,5R)-5-hydroxy-2-(3-hydroxypropyl)cyclohexyl]phenol; 5-(4-chlorophenyl)-1-(2,4-dichlorophenyl)-4-methyl-N-1-piperidinyl-1H-pyr-azole-3-caroxamide; [6-methoxy-2-(4-methoxyphenyl)benzo[b]furan-3-yl](4-cyanophenyl)methanone; [6-iodo-2-methyl-1-[2-(4-morpholinyl)ethyl]-1H-indol-3-yl](4-methoxy phenyl)methanone; 5-(4-chloro-3-methylphenyl)-1-[(4-methylphenyl)methyl]-N-(1,3,3-trimethyl-bicyclo[2.2.1]hept-2-yl)-(1S-endo)-1H-pyrazole-3-carboxamide; 1-(2,4-dichlorophenyl)-5-(4-iodophenyl)-4-methyl-n-1-piperidinyl-1H-pyraz-ole-3-carboxamide; 1-(2,4-dichlorophenyl)-5-(4-iodophenyl)-4-methyl-N4-morpholinyl-1H-pyraz-ole-3-carboxamide; 3-(6-azido-2-hexynyl)-6a,7,10,10a-tetrahydro-6,6,9-trimethyl-(6aR, 10aR)-6-H-dibenzo[b,d]pyran-1-ol; 3-[(2Z)-6-azido-2-hexynyl]-6a,7,10,10a-tetrahydro-6,6,9-trimethyl-(6aR,10-aR)-6H-dibenzo[b,d]pyran-1-ol; (−)-6,7-dichloro-1,4-dihydro-5-[3-(methoxymethyl)-5-(3-pyridinyl)-4H-1,2,-4-triazol-4-yl]-2,3-quinoxalinedione; (2R,4S)-rel-5,7-dichloro-1,2,3,4-tetrahydro-4-[[(phenylamino)carbonyl]ami-no]-2-quinolinecarboxylic acid; (2R,6S)-1,2,3,4,5,6-hexahydro-3-[(2S)-2-methoxypropyl]-6,11,11-trimethyl-2,6-methano-3-benzazocin-9-ol; (3E)-2-amino-4-(phosphonomethyl)-3-heptenoic acid; (3R,4S)-rel-3,4-dihydro-3-[4-hydroxy-4-(phenylmethyl)-1-piperidinyl]-2H-1-benzopyran-4,7-diol; (3S,4aR,6S,8aR)-decahydro-6-(phosphonomethyl)-3-isoquinoline carboxylic acid; (R)-9-bromo-2,3,6,7-tetrahydro-2,3-dioxo-N-phenyl-1H,5H-pyrido[1,2,-3-de]quinoxaline-5-acetamide; (.alpha.R)-.alpha.-amino-5-chloro-1-(phosphonomethyl)-1H-benzimidazole-2-propanoic acid; [2-(8,9-dioxo-2,6-diazabicyclo[5.2.0]non-1 (7)-en-2-yl)ethyl]-phosphonic acid; [5-(aminomethyl)-2-[[[(5S)-9-chloro-2,3,6,7-tetrahydro-2,3-dioxo-1H,5H-py-rido[1,2,3-de]quinoxalin-5-yl]acetyl]amino]phenoxy]-acetic acid; 1,4-dihydro-6-methyl-5-[(methylamino)methyl]-7-nitro-2,3-quinoxaline-dion-e monohydrochloride; 1-[2-(4-hydroxyphenoxy)ethyl]-4-[(4-methylphenyl)methyl]-4-piperidinol hydrochloride; 1-[4-(1H-imidazol-4-yl)-3-butynyl]-4-(phenylmethyl)-piperidine; 1-aminocyclopentane-carboxylic acid (ACPC); 2-[(2,3-dihydro-1H-inden-2-yl)amino]-acetamide monohydrochloride; 2-hydroxy-5-[[(pentafluorophenyl)methyl]amino]-benzoic acid (PBAS); 2-methyl-6-(phenylethynyl)-pyridine (MPEP); 3-(phosphonomethyl)-L-phenylalanine; 3-[(1E)-2-carboxy-2-phenylethenyl]-4,6-dichloro-1H-indole-2-carboxylic acid; 4,6-dichloro-3-[(E)-(2-oxo-1-phenyl-3-pyrrolidinylidene)methyl]-1H-indole-2-carboxylic acid; 6-chloro-2,3,4,9-tetrahydro-9-methyl-2,3-dioxo-1H-indeno[1,2-b]pyrazine-9-acetic acid; 7-chlorothiokynurenic acid; 8-chloro-2,3-dihydropyridazino[4,5-b]quinoline-1,4-dione 5-oxide salt with 2-hydroxy-N,N,N-trimethyl-ethanaminium; aptiganel; besonprodil; budipine; conantokin G; delucemine; dexanabinol; felbamate; fluorofelbamate; gacyclidine; glycine; ipenoxazone; kaitocephalin; lanicemine; licostinel; midafotel; milnacipran; N′-[2-chloro-5-(methylthio)phenyl]-N-methyl-N-[3-(methylthio)phenyl]-guan-idine; N′-[2-chloro-5-(methylthio)phenyl]-N-methyl-N-[3-[(R)-methylsulfiny-I]phenyl]-guanidine; neramexane; orphenadrine; remacemide; topiramate; alpha.-amino-2-(2-phosphonoethyl)-cyclohexanepropanoic acid; .alpha.-amino-4-(phosphonomethyl)-benzeneacetic acid; 8-[4-(1,1-dimethylheptyl)-2-hydroxyphenyl]decahydro-2-naphthalene methanol; 5,6,6a,7,8,9,10,10a-octahydro-6-methyl-3-[(1R)-1-methyl-4-pheny-I butoxy]-1,9-phenanthridinediol; Desacetyl-L-nantradol; R-(±)-methanandamide; 11-hydroxy-9,15-dioxoprosta-8,12,13-dienoic acid; 2-[3-methyl-6-(1-methylethenyl)-2-cyclohexen-1-yl]-5-pentyl-(1R-trans)-1,-3-benzenediol (cannabidiol); 3-amyl-1-hydroxy-6,6,9-trimethyl-6H-dibenzo[b,d]pyran (cannabinol); 3-(1,1-dimethylheptyl)-6a,7,8,9,10,10a-hexahydro-1-hydroxy-6,6-dimethyl-(−6aR,9R, 10aR)-6H-dibenzo[b,d]pyran-9-methanol; 7-(1,1-dimethylheptyl)-1,2,3,4,4a,9b-hexahydro-2,2-dimethyl-4-methylene-1,3-methanodibenzofuran-9-ol; 7-(1,1-dimethylheptyl)-1,2,3,4,4a,9b-hexahydro-2,2-dimethyl-4-methylene-1-(s),3-methanodibenzofuran-9-ol; 2-[4-[(acetyloxy)methyl]-6,6-dimethylbicyclo[3.1.1]hept-3-en-2-yl]-5-(1,1′-1-dimethylheptyl)-diacetate[1R-(1a,2a,5a)]-1,3-benzenediol; 2-[4-[(acetyloxy)methyl]-6,6-dimethylbicyclo[3.1.1]hept-3-en-2-yl]-5-(1,1-dimethylheptyl)-diacetate[1S-(1a,2a,5a)]-1,3-benzenediol; 5-(1,1-dimethylheptyl)-2-[4-(hydroxymethyl)-6,6-dimethylbicyclo[3.1.1]hep-t-3-en-2-yl]-[1S-(1a,2a,5a)]-1,3-benzenediol; and 5-(1,1-dimethylheptyl)-2-[4-(hydroxymethyl)-6,6-dimethylbicyclo[3.1.1]hep-t-3-en-2-yl]-[1R-(1a,2a,5a)]-1,3-benzenediol; or is an isomer, a pharmaceutically acceptable salt, ester, or prodrug thereof.

In one or more embodiments, the NSAID is an angiotensin 11 receptor antagonist. Angiotensin II receptor antagonists are known to affect inflammation and pain, as shown, for example in J Pharmacol Exp Ther. 2003 October; 307(1):17-23. Epub 2003 Aug. 27.

In certain embodiments, the angiotensin II receptor antagonist is selected from the group consisting of candesartan, eprosartan, irbesartan, losartan, olmesartan, tasosartan, telmisartan, valsartan, saralasin, and 1-[[4-(dimethylamino)-3-methylphenyl]methyl]-5-(diphenylac-etyl)-4,5,6,7-tetrahydro-1H-imidazo[4,5-c]pyridine-6-carboxylic acid ditrifluoroacetate, or an isomer, a pharmaceutically acceptable salt, ester, or prodrug thereof.

In one or more embodiments, the NSAID is an UDP-glucuronosyltransferase inhibitor (UGT inhibitor).

In certain embodiments, the UGT inhibitor is selected from the group consisting of epicatechin gallate, epigallocatechin gallate, octyl gallate, propyl gallate, quercetin, tannic acid, benzoin gum, capsaicin, dihydrocapsaicin, eugenol, gallocatechin gallate, geraniol, menthol, menthyl acetate, naringenin, allspice berry oil, N-vanillylnonanamide, clovebud oil, peppermint oil, silibinin and silymarin.

Mixtures of these non-steroidal immunomodulators may also be employed according to the present invention.

Suitable non-steroidal anti-inflammatory agent include but are not limited to azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

In an embodiment, the therapeutic agent is insecticide. The term “insecticide”, is used herein to mean a compound which kills, inhibits the growth of, impeded the proliferation of or repels insects. Insecticides include, for example, agents that can kill lice, flees, ticks, mites, scabies and mousquitos, as well as agents that repel such insects. Suitable insecticides include but are not limited to DDT, lindane, malathion, permethrin, allethrin, biopermethrin, transpermethrin, phenothrin, diethyl-m-toluamide, dimethyl phthalate, piperonyl butoxide, pyrethroids and derivatives, esters, salts and mixtures thereof.

In an embodiment, the therapeutic agent is a vasodilator. Suitable vasodilators include but are not limited to agents that modulate the activity of the enzyme nitric oxide synthase, nicotinic acid, ethyl nicotinate, amyl nitrite, amyl nitrate, ethyl nitrite, butyl nitrite, isobutyl nitrite, glyceryl trinitrate, octyl nitrite, sodium nitrite, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone, a beta-adrenergic blocker, an alpha-adrenoceptor blocker, a prostaglandin, sildenafil, dipyridamole, catecholamine, isoproternol, furosemide, prostaglandin, prostacyclin, enalaprilat, morphine, acepromazine, prazosin (α-blocker), enalapril, Captopril, amlodipine, minoxidil, tadalafil, vardenafil, phenylephrin, etilefein, caffeine, capsaicin, an extract capsicum, achillea millefolium (Yarrow), allium sativum (garlic), amoracia rusticana (horseradish), berberis vulgaris (barberry), cimicifuga racemosa (black cohosh), coleus forskholii (coleus), coptis (goldenthread), crataegus (hawthorn), eleutherococcus senticosus (siberian ginseng), ginkgo biloba(ginkgo), melissa offiicnalis (lemon balm), olea europaea (olive leaf), panax ginseng (Chinese ginseng), petroselinum crispum (parsley), scutellaria baicalensis (baical skullcap), tilia europaea (linden flower), trigonella foenum-graecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut and walnut, and derivatives, esters, salts and mixtures thereof.

In an embodiment, the therapeutic agent is a vasoconstrictor. Suitable vasodilators include but are not limited to ephedrine, epinephrine, phenylephrine, angiotensin, vasopressin; an extract ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom) and cypressand and derivatives, esters, salts and mixtures thereof.

### Retinoid Agents

In an embodiment, the active agent is a retinoid.

In the context of the present invention, a retinoid is a compound a class of compounds consisting of four isoprenoid units joined in a head-to-tail manner, and derivatives, salts, structural analogs and functional analogs thereof, as reviewed herein in a non-limiting fashion. Typically, retinoids may be formally derived from a monocyclic parent compound containing five carbon-carbon double bonds and a functional group at the terminus of the acyclic portion.

Suitable, but non-limiting, retinoids for use in the present invention are listed below.

It is convenient to omit the explicit representation of C and H atoms in the parent skeletal structure of retinoids as follows:

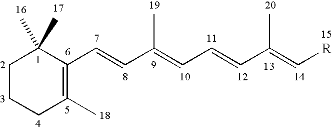

| | |
|---|---|
| (I) R = CH₂OH | (VI) R = CH₂NH₂ |
| (II) R = CHO | (VII) R = CH═NOH |
| (III) R = CO₂H | (VIII) R = CH═N[CH₂]₄CHNH₂CO₂H |
| (IV) R = CH₃ | (IX) R = CO₂C₂H₅ |
| (V) R = CH₂OCOCH₃ | 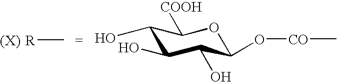 |

Compound (I) (2E,4E,6E,8E)-3,7-dimethyl-9-(2,6,6-trimethylcyclohex-1-en-1-yl)nona-2,4,6,8-tetraen-1-ol is also known as vitamin A, vitamin A alcohol, retinal, vitamin A₁, vitamin A₁ alcohol, axerophthol or axerol. Compound (II) also known as vitamin A aldehyde, vitamin A₁ aldehyde, retinene or retinene₁ and retinal or, if liable to be confused with the adjective retinal (pertaining to the retina), retinaldehyde. Compound (III) also known as tretinoin (see note), vitamin A acid or vitamin A₁ acid should be designated retinoic acid. Compound (IV), is known as axerophthene. Functional substitution at the 15 position of the basic hydrocarbon is denoted by the use of the group names retinyl (R is CH₂—) or retinylidene (R is CH═), with retention of the original numbering of the basic hydrocarbon. For example (V) is retinyl acetate and (VI) is retinylamine. Derivatives of retinal include for example Compound (VII)— retinal oxime and Compound (VII)—N⁶-retinylidene-L-lysine. Other derivatives of retinoic acid, named as carboxylic acid derivatives Compound (IX)— ethyl retinoate and Compound (X)-1-O-retinoyl-b-D-glucopyranuronic acid.

Retinoids that differ in hydrogenation level from the parent structure (displayed above) are named by use of the prefixes ‘hydro’ and ‘dehydro’ together with locants specifying the carbon atoms at which hydrogen atoms have been added or removed. Examples of such retinoid compounds are Compound (XI)-3,4-Didehydroretinol (also known as dehydroretinol or vitamin A₂) and Compound (XII)-4,5-Didehydro-5,6-dihydroretinol (also known as alpha-vitamin A).

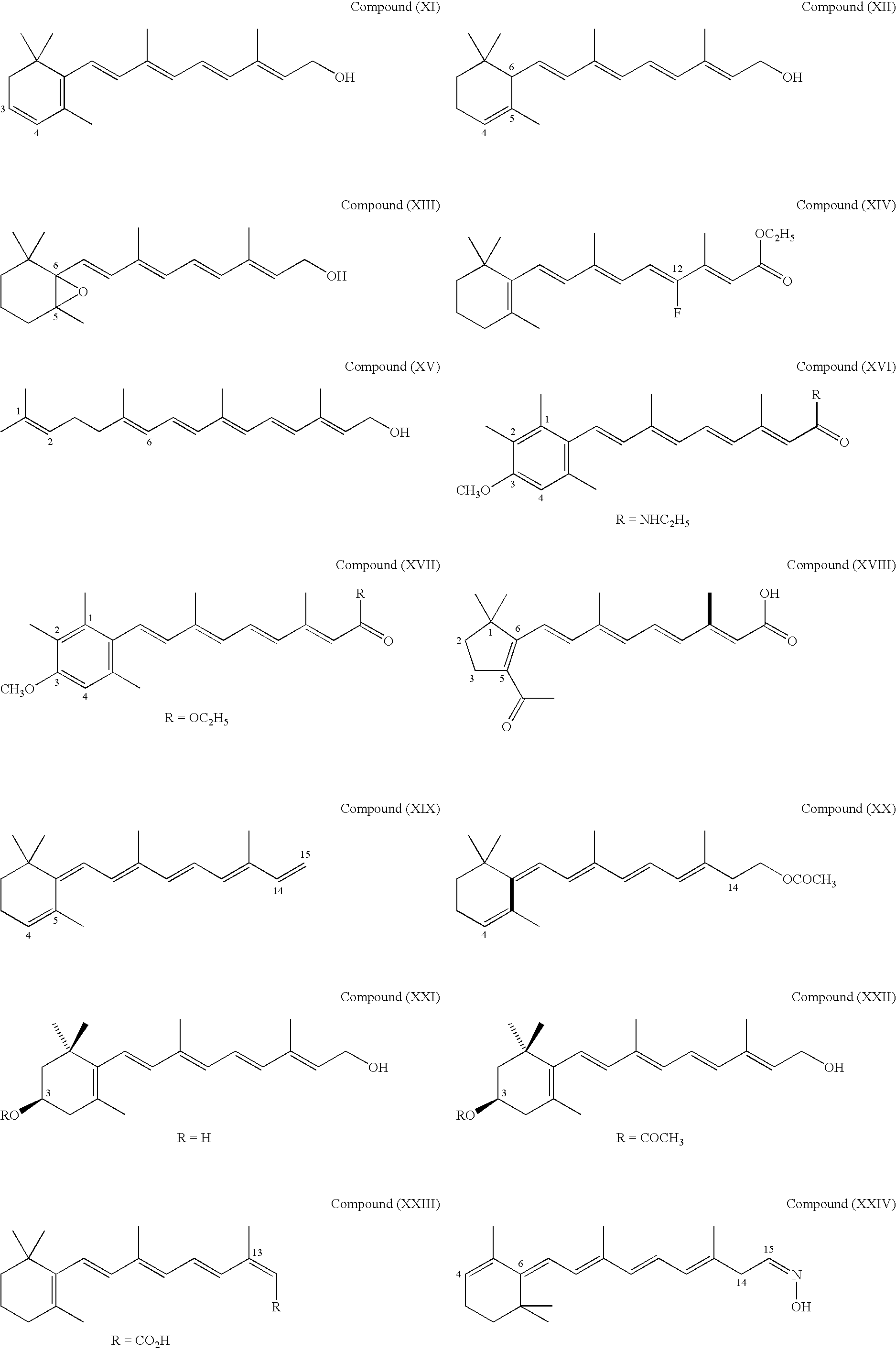

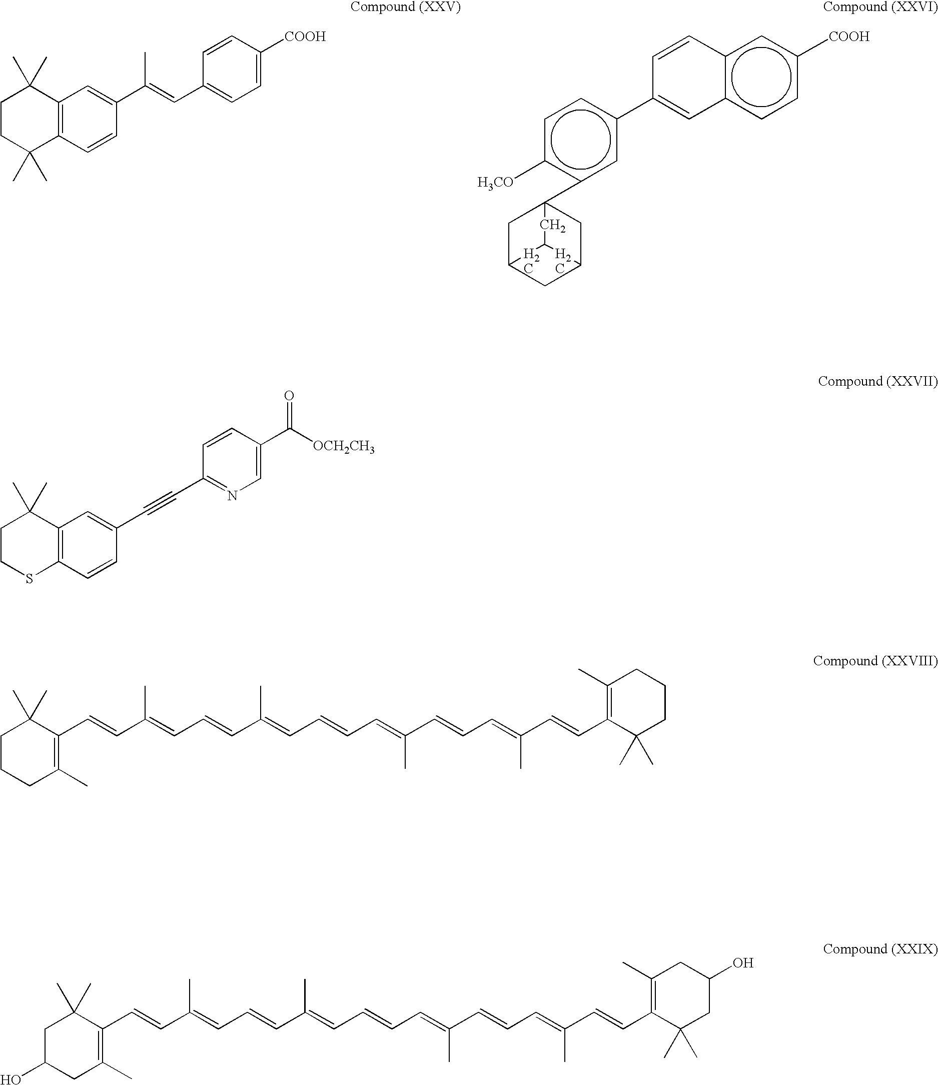

Substituted derivatives of retinoids are exemplified by Compound (Xiii)-5,6-Epoxy-5,6-dihydroretinol (also known as hepaxanthin) and Compound (XIV)—Ethyl 12-fluororetinoate. Seco Retinoids are exemplified by Compound (XV)—1,6-Seco-1,2-didehydroretinol, also known as g-vitamin A, and Nor Retinoids, which result from the elimination of a CH₃, CH₂, CH or C group from a retinoid are exemplified by Compound (XVI)-N-Ethyl-3-methoxy-2-methyl-17-nor-1,2,3,4-tetradehydroretinamide (also known as motretinide), Compound (XVII)— Ethyl 3-methoxy-2-methyl-17-nor-1,2,3,4-tetradehydroretinoate (also known as etretinate), acitretin (Compound (XVII), wherein R═H) and Compound (XVIII)-5-Acetyl-4,18-dinor-retinoic acid. Retro Retinoids are exemplified by Compound (XIX)-4,5-Didehydro-15,5-retro-deoxyretinol (also known as anhydro vitamin A and Compound (XX)-4,14-retro-Retinyl acetate. Stereoisomers of retinoids are exemplified by Compound (XXI)-(3R)-3-Hydroxyretinol and Compound (XXII)-(3R)-3-Acetoxyretinol. Other stereochemical isomers can are exemplified by Compound (XXIII)-13-cis-Retinoic acid or (7E,9E,11E,13Z)-retinoic acid (also known as isotretinoin) and Compound (XXIV)-(6E,8E, 10E, 12E, 15Z)-4,14-retro-Retinaloxime.

‘Arotinoids’ or ‘retinoidal benzoic acid derivatives’ contain, aromatic rings replacing either the basic β-ionone type ring structure or unsaturated bonds of the tetraene side chain of the parent retinoid skeleton, as exemplified by Compound (XXV) and Compound (XXVI)—6-[3-(1-adamantyl)-4-methoxyphenyl]-2-naphthoic acid, also known as adapalene. Several artinoids, possessing potent retinoid properties, including but not limited to short retinoids, short heterocyclic retinoids, isoxazole-containing retinoids, heterocyclic isoxazole-containing retinoids, isoxazoline-containing retinoids, stilbene retinoid analogs, are disclosed in Pure Appl. Chem., Vol. 73, No. 9, pp. 1437-1444, 2001.

Tazarotene (Ethyl 6-[2-(4,4-dimethylthiochroman-6-yl)ethynyl]nicotinate) is exemplary to a retinoid precursor—Compound (XXVII), suitable as retinoid for use in the present invention.

Yet, other non-limiting exemplary retinoid precursors are carotenes, such as all-trans beta carotene—Compound (XXVIII), alpha carotene, lycopene and 9-cis-beta-carotene, as well as xanthophils (also termed “oxicarotenoids”), such as lutein and zeaxanthin—Compound (XXIX).

Salts and derivatives of retinoid compounds are also suitable as “retinoid” for use in the present invention.

Retinoid compounds can be ascertained recognized and identified by methods known in the art. One method involves the use of competitive nuclear retinoic acid (RA and RX) receptor binding assays for identifying compounds which bind directly to the receptors. For instance, J. J. Repa et al., “All-trans-retinol is a ligand for the retinoic acid receptors”, Proc. Natl. Acad. Sci. USA, Vol. 90, pp. 7293-7297, 1993, discloses a competitive RA receptor binding assay based on human neuroblastoma cell nuclear extracts. H. Torma et al. ((1994) “Biologic activities of retinoic acid and 3,4-dehydroretinoic acid in human keratinoacytes are similar and correlate with receptor affinities and transactivation properties,” J. Invest. Dermatology, Vol. 102, pp. 49-54) discloses assays for measuring binding affinities for the nuclear retinoic acid receptors and for measuring transcriptional activation induction. M. F. Boehm et al. ((1994) “Synthesis of high specific activity [.sup.3H]-9-cis-retinoic acid and its application for identifying retinoids with unusual binding properties,” J. Med. Chem., Vol. 37, pp. 408-414) discloses a ligand-binding assay and a receptor/reporter cotransfection assay for monitor regulation of gene expression. EP 0 552 612 A2, published Jul. 28, 1993, describes ligand-binding trapping assays based on incubation of radiolabeled compounds with transfected COS-1 cells which express RA and RX receptors.

Mixtures of these retinoids may also be employed according to the present invention.

Suitable retinoids include but are not limited to retinol, retinal, retinoic acid, all-trans retinoic acid, isotretinoin, tazarotene, adapalene, 13-cis-retinoic acid, acitretin all-trans beta carotene, alpha carotene, lycopene, 9-cis-beta-carotene, lutein and zeaxanthin.

In an embodiment, the therapeutic agent is selected from the group consisting of an immunosuppressants and immunoregulating agents. Suitable immunosuppressants and immunoregulating agents include but are not limited to cyclic peptides, such as cyclosporine, tacrolimus, tresperimus, pimecrolimus, sirolimus (rapamycin), verolimus, laflunimus, laquinimod, imiquimod derivatives, esters, salts and mixtures thereof. In one or more embodiments, the immunomodulator is a calcineurin Inhibitor.

In an embodiment, the therapeutic agent is a wart remover. Suitable wart removers include but are not limited to imiquimod, podophyllotoxin and derivatives, esters, salts and mixtures thereof.

### Vitamin

The term vitamin includes those vitamins and derivatives thereof (including salts) which are officially recognized as vitamins, and those vitamins which were once recognized or designated as vitamins but are now classified in another way (e.g. vitamin F) and pseudo vitamins including those substances which are a member of a group or complex but are not formally recognized (e.g. para-amino benzoic acid (PABA), which is claimed to prevent greying hair and to be useful as an anti aging supplement) and also vitamin mimetics, which have vitamin like properties or effects.

Suitable vitamins include vitamin A, vitamins of the B complex B1, B2, B3, B5, B6, B7, B9, B12, vitamin C, vitamins D1-D4, vitamin E, vitamin K and so called vitamin F and a derivative thereof and combinations thereof.

Vitamin A is a fat-soluble vitamin and describes compounds that exhibit the biological activity of retinol. The two main components in foods are retinol and the carotenoids. ‘Retinoid’ refers to the chemical entity retinol or other closely related naturally occurring derivatives. These include: retinal (retinaldehyde); retinoic acid; and retinyl esters (e.g. retinyl acetate, retinyl palmitate, retinyl propionate). Retinoids also include structurally related synthetic analogues which may or may not have retinol-like activity. Vitamin A (in the form of retinal) is essential for normal function of the retina and particularly for visual adaptation to darkness. Other forms (retinol, retinoic acid) are necessary for maintenance of the structural and functional integrity of epithelial tissue and the immune system, cellular differentiation and proliferation, bone growth, testicular and ovarian function and embryonic development. It may act also as a co-factor in biochemical reactions. Deficiency can amongst other things result in skin dryness and papular eruptions. Vitamin A and its derivatives have the ability to normalize keratinization. Note that vitamin C may ameliorate the toxic effects of vitamin A; that large doses increase the need for vitamin E; and that vitamin E protects against the oxidative destruction of vitamin A. Retinol is susceptible to breakdown from oxygen and light. Synthetic retinoids may be used for skin problems (e.g. Acne).

According to certain embodiments the retinoid is selected from the group consisting of: (1) a compound consisting of four isoprenoid units joined in a head-to-tail manner, a compound having the formula:

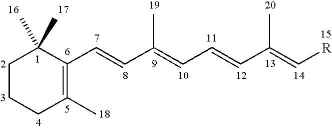

where R is selected from the group consisting of H, alkyl, aryl, alkenyl, benzyl, CH₂OH, CH₂NH₂, CHO, CH═NOH, CO₂H, CH═N[CH₂]₄CHNH₂CO₂H, CH₃, CO₂C₂H₅, CH₂OCOCH₃, a heteroatom, a saccharide and a polysaccharide; (2) a compound selected from the group consisting of a hydro retinoid, a dehydro retinoid, 3,4-Didehydroretinol, 4,5-Didehydro-5,6-dihydroretinol, a substituted derivative of a retinoid, 5,6-epoxy-5,6-dihydroretinol, ethyl 12-fluororetinoate, a seco retinoid, 1,6-Seco-1,2-didehydroretinol, a nor retinoid, (3) a compound which results from the elimination of a CH₃, CH₂, CH or C group from a retinoid, N-ethyl-3-methoxy-2-methyl-17-nor-1,2,3,4-tetradehydroretinamide, ethyl 3-methoxy-2-methyl-17-nor-1,2,3,4-tetradehydroretinoate, 5-acetyl-4,18-dinor-retinoic acid, a retro retinoid, 4,5-didehydro-15,5-retro-deoxyretinol, 4,14-retro-retinyl acetate, a stereoisomer of a retinoid, (3R)-3-hydroxyretinol, (3R)-3-Acetoxyretinol, (7E,9E, 11E, 13Z)-retinoic acid, (6E,8E, 10E, 12E, 15Z)-4,14-retro-retinaloxime, an arotinoids, a retinoidal benzoic acid derivative, 6-[3-(1-adamantyl)-4-methoxyphenyl]-2-naphthoic acid, a short retinoid, a short heterocyclic retinoid, an isoxazole-containing retinoids, a heterocyclic isoxazole-containing retinoid, an isoxazoline-containing retinoid, a stilbene retinoid analog, a retinoid precursor, (ethyl 6-[2-(4,4-dimethylthiochroman-6-yl)ethynyl]nicotinate, a carotene, a xanthophil and an oxicarotenoid; (4) a compound selected from the group consisting of retinol, retinal, retinoic acid, all-trans retinoic acid, isotretinoin, tazarotene, adapalene, 13-cis-retinoic acid, acitretin, all-trans beta carotene, alpha carotene, lycopene, 9-cis-beta-carotene, lutein and zeaxanthin; (5) a compound that is positively identified using a laboratory method, suitable of detecting a retinoid, and salts and derivatives thereof.

Vitamin B is known as the vitamin B complex and comprises B₁ (thiamine), B₂ (riboflavin), B₃ (niacin), B₅ (pantothenic acid), B₆ (pyridoxine), B₇ (biotin), B₉ (folic acid) and B₁₂ (cyanocobalamin). Adequate amounts of all B vitamins are required for optimal functioning; deficiency or excess of one B may lead to abnormalities in the metabolism of another.

Thiamine is a water soluble vitamin and is also known as aneurine and functions as a co-enzyme in the oxidative decarboxylation of alpha ketoacids (involved in energy production) and in the transketolase reaction of the pentose phosphate pathway (involved in carbohydrate metabolism). Thiamine is also important in nerve transmission (independently of co-enzyme function). It may also act as an insect repellant.

Riboflavin is a water soluble vitamin and functions as a component of two flavin co-enzymes—flavin mononucleotide (FMN) and flavin adenine dinucleotide (FAD). It participates in oxidation-reduction reactions in numerous metabolic pathways and in energy production. Examples include: the oxidation of glucose, certain amino acids and fatty acids; reactions with several intermediaries of the Krebs cycle; conversion of pyridoxine to its active co-enzyme; and conversion of tryptophan to niacin. Riboflavin has a role as an antioxidant. It may be involved in maintaining the integrity of erythrocytes. Common forms are riboflavin, riboflavin butyrate and flavin adenine dinucleotide.

Niacin is a water-soluble vitamin and describes the compounds that exhibit the biological properties of nicotinamide. It occurs as nicotinamide and nicotinic acid. It is sometimes known as niacinamide. An example of a derivative is benzyl nicotinate. Niacin functions as a component of two co-enzymes, nicotinamide adenine dinucleotide (NAD) and nicotinamide adenine dinucleotide diphosphate (NADP). These co-enzymes participate in many metabolic processes including glycolysis, tissue respiration, lipid, amino acid and purine metabolism. It has been shown to have ant-inflammatory properties that result in the improvement of acne. Topically it has showed benefit for various skin conditions including psoriasis and rosacea. It has also been said to have a photo protection role, perhaps through anti-oxidant activity and reduces or prevents UV damage to cells and UV induced disorders.

Pantothenic acid is also a water soluble vitamin and functions mainly as a component of co-enzyme A and acyl carrier protein. Co-enzyme A has a central role as a co-factor for enzymes involved in the metabolism of lipids, carbohydrates and proteins; it is also required for the synthesis of cholesterol, steroid hormones, acetylcholine and porphyrins. As a component of acyl carrier protein, pantothenic acid is involved in various transfer reactions and in the assembly of acetate units into longer-chain fatty acids. Pantothenic acid has been used for a wide range for disorders such as acne, alopecia, allergies, burning feet, asthma, grey hair, dandruff, and cholesterol lowering. Panthenol the alcoholic form functions as a humetic. Examples of pantothenic acid derivatives are calcium pantothenate, D-pantothenyl alcohol, pantothenyl ethyl ether, and acetylpentothenyl ethyl ether.

Vitamin B₆ is water soluble vitamin. Vitamin B₆ a generic term used to describe the compounds that exhibit the biological activity of pyridoxine. It occurs in food as pyridoxine, pyridoxal and pyridoxamine. Vitamin B₆ is converted in erythrocytes to pyridoxal phosphate and, to a lesser extent, pyridoxamine phosphate. It acts as a co-factor for enzymes which are involved in more than 100 reactions that affect protein, lipid and carbohydrate metabolism. Pyridoxal phosphate is also involved in: the synthesis of several neurotransmitters; the metabolism of several vitamins (e.g. The conversion of tryptophan to niacin); haemoglobin and sphingosine formation. Lack of Vitamin B₆ may affect vitamin C. Examples are pyridoxine hydrochloride and pyridoxine dioctanate.

Biotin is a water soluble vitamin which was formerly known as vitamin H or co-enzyme R. Biotin functions as an integral part of the enzymes that transport carboxyl units and fix carbon dioxide. Biotin enzymes are important in carbohydrate and lipid metabolism, and are involved in gluconeogenesis, fatty acid synthesis, propionate metabolism and the catabolism of amino acids. Biotin has been claimed to be of value in the treatment of brittle finger nails, acne, seborrhoeic dermatitis, hair fragility and alopecia.

Folic acid (pteroylglutamic acid) is a water soluble vitamin and is the parent compound for a large number of derivatives collectively known as folates. Folate is the generic term used to describe the compounds that exhibit the biological activity of folic acid; it is the preferred term for the vitamin present in foods which represents a mixture of related compounds (folates). Folates are involved in a number of single carbon transfer reactions, especially in the synthesis of purines and pyrimidines (and hence the synthesis of DNA), glycine and methionine. They are also involved in some amino acid conversions and the formation and utilization of formate. Deficiency leads to impaired cell division (effects most noticeable in rapidly regenerating tissues).

Vitamin B₁₂ is a water-soluble vitamin and it is the generic term used to describe the compounds that exhibit the biological activity of cyanocobalamin. It includes a range of cobalt-containing compounds, known as cobalamins. Cyanocobalamin and hydroxocobalamin are the two principal forms in clinical use. Vitamin B₁₂ is involved in the recycling of folate co-enzymes and the degradation of valine. It is also required for nerve myelination, cell replication, haematopoiesis and nucleoprotein synthesis.

Vitamin C is a water-soluble vitamin and describes the compounds that exhibit the biological activity of ascorbic acid. These include L-ascorbic acid (ascorbic acid) and L-dehydroascorbic acid (dehydroascorbic acid). The functions of vitamin C are based mainly on its properties as a reducing agent. It is required for: the formation of collagen and other organic constituents of the intercellular matrix in bone, teeth and capillaries; and the optimal activity of several enzymes—it activates certain liver-detoxifying enzyme systems (including drug-metabolizing enzymes) and is involved in the synthesis of carnitine and norepinephrine (noradrenaline) and in the metabolism of folic acid, histamine, phenylalanine, tryptophan and tyrosine. Vitamin C also acts: as an antioxidant (reacting directly with aqueous free radicals)—which is important in the protection of cellular function; and to enhance the absorption of non-haem iron. It can function as a whitening agent. Vitamin C may assist with wound healing. Vitamin C can spare vitamin E and vice versa and it may reduce toxic effects of vitamin A. Vitamin C is unstable in solution especially alkaline solution and readily undergoes oxidation on exposure to air. Oxidation is accelorated by light and heat. Cosmetic forms include calcium ascorbate, magnesium ascorbate, sodium ascorbate, sodium ascorbyl phosphate, ascorbyl palmitate, magnesium ascorbyl phosphate, L-ascorbic acid and magnesium-L-ascorbyl-2-phosphate, L-ascorbic acid palmitate, L-ascorbic acid 2-sulfate, L-ascorbic acid phosphate, and DL-.alpha.-tocopherol-L-ascorbic acid phosphate diester dipotassium. L-ascorbic acid is the most bioactive form and has been found to have many skin benefits but it is unstable in the presence of water and oxygen. Inclusion of ascorbic acid in the vitamin carrier, wherein the composition does not contain or is essentially free of water or wherein water is not freely available due to the hygroscopic properties of the composition r and or is not exposed to air during storage makes it possible to derive stable products with the most bioactive form of vitamin C.

Vitamin D is a fat-soluble vitamin and describes all sterols that exhibit the biological activity of cholecalciferol. These include: vitamin D₁ (calciferol), vitamin D₂ (ergocalciferol) vitamin D₃ (cholecalciferol), 1 (OH)D₃ (1 Hydroxycholecalciferol; alfacalcidol), 25(OH)D₃ (25 Hydroxycholecalciferol; calcifediol), 1,25(OH)₂D₃ (1,25, Dihydroxycholecalciferol; calcitriol), 24,25(OH)₂D₃ (24,25, Dihydroxycholecalciferol) and dihydrotachysterol, calcipotriene, 25-hydroxycholecalciferol, 1α,25-dihydroxycholecalciferol, 11,25-dihydroxyergocalciferol, 22,23-dihydroergocalciferol, 1,24,25-trihydroxycholecalciferol, previtamin D₃, tachysterol₃ (also termed tacalciol), isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol. Vitamin D is essential for promoting the absorption and utilisation of calcium and phosphorus, and normal calcification of the skeleton. Along with parathyroid hormone and calcitonin, it regulates serum calcium concentration by altering serum calcium and phosphate blood levels, as needed, and mobilizing calcium from bone. It maintains neuromuscular function and various other cellular processes, including the immune system. Calcipotriene, as well as other vitamin C forms is useful in the treatment of psoriasis.

Vitamin E is a fat-soluble vitamin and describes all tocopherol and tocotrienol derivatives that exhibit the biological activity of alpha tocopherol. Those used commercially are d-alpha tocopherol (natural vitamin E), d-alpha tocopherol acetate, d-alpha tocopherol succinate, d,l-alpha tocopherol (synthetic vitamin E), d,l-alpha tocopherol acetate and d,l-alpha tocopherol succinate. Vitamin E is an antioxidant, protecting polyunsaturated fatty acids in membranes and other critical cellular structures from free radicals and products of oxidation. It works in conjunction with dietary selenium (a co-factor for glutathione peroxidase), and also with vitamin C and other enzymes, including superoxide dismutase and catalase. Vitamin E is not very stable. It may have an anti-inflammatory effect and some studies state that it improves immune function in the elderly. It is also said to reduce oxidative damage and to improve lung function. Vitamin E can spare vitamin C and vice versa. It is said to be photo protective and to have an anti aging effect on skin showing reduced wrinkles and tumors

Vitamin K is a fat soluble vitamin and describes 2-methyl-1,4-naphthaquinone and all derivatives that exhibit qualitatively the biological activity of phytomenadione. The form of vitamin K present in foods is phytomenadione (vitamin K₁). The substances synthesized by bacteria are known as menaquinones (vitamin K₂). The parent compound of the vitamin K series is known as menadione (vitamin K₃); it is not natural substance and is not used in humans. Menadiol sodium phosphate is water-soluble derivative of menadione. Vitamin K is an essential co-factor for the hepatic synthesis of proteins involved in the regulation of blood clotting. These are: prothrombin (factor II), factors VII, IX, X and proteins C, S and Z. Vitamin K is responsible for the carboxylation of the bone protein, osteocalcin, to its active form. Osteocalcin regulates the function of calcium in bone turnover and mineralisation. Vitamin K is also required for the biosynthesis of some other proteins found in plasma and the kidney. It is reported to speed up resolution of bruising to decrease future bruising and correct aspects of photoaging.

Pseudo vitamins: Vitamin F was the designation originally given to essential fatty acids that the body cannot manufacture. They were “de-vitaminized” because they are fatty acids. Fatty acids are a major component of fats which, like water, are needed by the body in large quantities and thus do not fit the definition of vitamins which are needed only in trace amounts. Herbalists and naturopaths have named various therapedic chemicals “vitamins”, even though they are not, including vitamin T, S-Methylmethionine (vitamin U) and vitamin X. Some authorities say that ubiquinone, also called coenzyme Q10, is a vitamin. Ubiquinone is manufactured in small amounts by the body, like vitamin D. Pangamic acid, vitamin B₁₅; the related substance dimethylglycine is quite wrongly referred to as vitamin B₁₅ but also labeled B₁₆. The toxins laetrile and amygdaline are sometimes referred to as vitamin B₁₇. Both pangamic acid and laetrile were first proposed as vitamins by Ernst T. Krebs; neither are recognized by the medical community. Flavonoids are sometimes called vitamin P. Animal, bird, and bacterial growth factors have been designated vitamins such as para-aminobenzoic acid (PABA) vitamin B₁₀, the folacin (see folic acid) pteryl-heptaglutamic acid vitamin B₁₁ or vitamin Bc-conjugate and orotic acid as vitamin B₁₃. A few substances were once thought to be B-complex vitamins and are referred to as B-vitamins in older literature, including B₄ (adenine) and B₈ (adenylic acid), but are no longer recognized as such. An antitumor pterin phosphate named Vitamin B₁₄ and later abandoned.

Vitamins as anti oxidants. The antioxidant vitamins can be divided into those that are water-soluble and exist in aqueous solution—primarily vitamin C—and those that are fat-soluble and exist in membranes or lipoproteins—vitamin E and betacarotene. Lipid membranes are particularly vulnerable to oxidative breakdown by free radicals. Vitamin E protects cell membranes from destruction by undergoing preferential oxidation and destruction. Some quinones, such as ubiquinone (co-enzyme Q) also appear to have antioxidant properties. All these substances can act as free radical scavengers and can react directly with free radicals. Riboflavin also has a role as an antioxident.

They are believed to protect against certain diseases by preventing the deleterious effects of free-radical-mediated processes in cell membranes and by reducing the susceptibility of tissues to oxidative stress. An article by MP Ludo entitled “Antioxidants and Vitamins in Cosmetics” Clinics in Dermatology (2001): 19:467-473 discusses the benefits of vitamins and derivatives in cosmetics. Note that carotenoids and flavonoids also act as antioxidants

Synergism between vitamins is known, for example, synergism between vitamin A and vitamin E is described by Gallarate, Carlofti, Trotta, and Bovo in the International Journal of Pharmacuetics 188 (1999) 233-241 discussing a study on the stability of ascorbic acid. Any synergism known in the literature between vitamins to potentiate or facilitate their action can be used in the present invention. Details of the solubility of vitamins can be found for example in the Merck Index and other similar reference works and databases.

In one or more embodiment, the surface active agent can range from about less than 0.1% up to about 15% or up to about 20% by weight of composition depending on the surfactant selected or preferably is about 0.2% to about 0.5% by weight of composition.

In one or more embodiments, the vitamin is selected from the group consisting of vitamin A, B₁, B₂, B₃, B₅, B₆, B₇, B₉, B₁₂, PABA, C, D₁-D₄, E, K and F and a derivative thereof.

In another embodiment, the vitamin or a derivative thereof is susceptible to oxidation.

In a further embodiment, the vitamin or a derivative thereof is soluble in water.

In another embodiment, the vitamin is selected from the group consisting of vitamin B₁, B₂, B₃, B₅, B₆, B₇, B₉, B₁₂, PABA and C and a derivative thereof.

In an embodiment, the vitamin is vitamin B3 or a derivative thereof or combinations thereof.

In an embodiment, the vitamin is vitamin C or a derivative thereof or combinations thereof.

In an embodiment, the vitamin is the vitamin is vitamin K or a derivative thereof or combinations thereof.

In an embodiment, the vitamin is vitamin A or a derivative thereof or combinations thereof.

In an embodiment, the vitamin is vitamin E or a derivative thereof or combinations thereof.

In an embodiment, the vitamin is the vitamin is vitamin F or a derivative thereof or combinations thereof.

In one or more embodiments, the vitamin is a combination of two or more vitamins selected from the group comprising vitamin A, B₃, C, K, E, and F and a derivative thereof.

In an embodiment, the vitamin or a derivative thereof or combinations thereof comprises an antioxident.

In another embodiment, the vitamin or a derivative thereof or combinations thereof improves stimulates or promotes target site metabolism.

In a still further embodiment, the vitamin or a derivative thereof or combinations thereof alleviates, ameliorates, treats, prevents, retards or otherwise has a beneficial effect on a skin or boy cavity condition.

In an embodiment, the skin condition is selected from the group consisting of skin pigmentation, dry skin, a wound, acne, psoriasis and skin aging.

In one or more embodiments, the vitamin is a combination of two or more vitamins selected from the group comprising vitamin B₃, E and C and a derivative thereof.

In an embodiment, the therapeutic agent is a photodynamic therapy (PDT) agent. Suitable PDT agents include but are not limited to modified porphyrins, chlorins, bacteriochlorins, phthalocyanines, naphthalocyanines, pheophorbides, purpurins, m-THPC, mono-L-aspartyl chlorine6, bacteriochlorins, phthalocyanines, benzoporphyrin derivatives, as well as photosensitiser precursors, such as aminolevulinic acid and derivatives, esters, salts and mixtures thereof.

In an embodiment, the therapeutic agent is an antioxidant or a radical scavenger. Suitable antioxidants and radical scavengers agents include but are not limited to ascorbic acid, ascorbyl esters of fatty acids, magnesium ascorbyl phosphate, sodium ascorbyl phosphate, ascorbyl sorbate, tocopherol, tocopheryl sorbate, tocopheryl acetate, butylated hydroxy benzoic acid, 6-hydroxy-2,5,7,8-tetramethylchroman-2-carboxylic acid, gallic acid, propyl gallate, uric acid, sorbic acid, lipoic acid, diethylhydroxylamine, amino-guanidine, glutathione, dihydroxy fumaric acid, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and polyunsaturated oils, containing omega-3 and omega-6 fatty acids (e.g., linoleic and linolenic acid, gamma-linoleic acid, eicosapentaenoic acid and docosahexaenoic acid and derivatives, esters, salts and mixtures thereof.

In an embodiment, the therapeutic agent is a self-tanning agent, such as dihydroxyacetone.

In an embodiment, the therapeutic agent is an agent, capable of treating hyperhidrosis. Suitable hyperhidrosis agents include but are not limited to anticholinergic drugs, boric acid, tannic acid, resorcinol, potassium permanganate, formaldehyde, glutaraldehyde, methenamine, a Lewis acid, aluminum chloride, aluminum chlorohydrates, zirconium chlorohydrates, aluminum-zirconium-Glycine (AZG) complex, aluminum hydroxybromide, a glycopyrrolate compound, a 5-alpha-reductase inhibitor, finasteride, epristeride, flutamide, spironolactone, saw palmetto extract, cholestan-3-one, a mono- and dicarboxylic acid having 4 to 18 carbon atoms, botulinum toxin, a 5-HT2C receptor antagonist, a 5-HT2C receptor antagonist, ketanserin, ritanserin, mianserin, mesulergine, cyproheptadine, fluoxetine, mirtazapine, olanzapine and ziprasidone.

In an embodiment, the additional therapeutic agent is a sunscreen agent. Suitable sunscreen agents include but are not limited to titanium dioxide, zinc oxide, zirconium oxide, iron oxide, p-aminobenzoic acid and its derivatives (ethyl, isobutyl, glyceryl esters; p-dimethylaminobenzoic acid); anthranilic acid derivatives (i.e., o-amino-benzoates, methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid, o- and p-hydroxybiphenyldisulfonates, coumarin derivatives (7-hydroxy, 7-methyl, 3-phenyl), diazoles (2-acetyl-3-bromoindazole, phenyl benzoxazole, methyl naphthoxazole, quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzolesorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

In an embodiment, the additional therapeutic agent is a figure-forming agent and an agent, capable of treating cellulite. Suitable such agents include but are not limited to baldderwack extract, butcher's broom, cayenne, dandelion, red clover, ginkgo biloba, horse chestnut, witch hazel and borage oil, caffeic acid, nicotinic acid, theophiline and pentoxyphilline and salts and derivatives thereof.

Several disorders of the skin, body cavity or mucosal surface (e.g., the mucosa or the cavity of the nose, mouth, eye, ear, vagina or rectum) involve a combination of etiological factors. For example, fungal and bacterial infections and that are inflamed and have symptoms of redness and/or itching warrant therapy that combines an anti-infective agent and an anti-inflammatory agent. Thus, in several cases, combining at least two active agents that treat different etiological factors results in a synergistic effect and consequently higher success rate of the treatment.

In certain cases, the composition contains two active agents, where each of the active agents require a different pH environment in order to remain stable. For example, corticosteroids are typically stable at acidic pH values (they have a maximum stability at a pH of about 4-6) and of vitamin D analogues are typically stable at basic pH values (they have a maximum stability at pH values above about 8). In order to circumvent the problem of instability in such cases a dual applicator can be used, one canister with bmv at acidic pH and the other canister at alkali pH. Alternatively, one of the active ingredients could be provided as a precurser, which is more stable.

Whenever there is reference to an active agent and or derivatives the reference includes, derivatives, conjugates, analogues, prodrugs, chelates, complexes, ions, isomers, enantimers, and salts thereof.

### Hydrophobic Solvent

The foamable composition can be an emulsion, or microemulsion, including an aqueous phase and an organic carrier phase. The organic carrier is selected from a hydrophobic organic carrier (also termed herein “hydrophobic solvent”), an emollient, a polar solvent, and a mixture thereof.

A “hydrophobic organic carrier” as used herein, refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature. The identification of a hydrophobic organic carrier or “hydrophobic solvent”, as used herein, is not intended to characterize the solubilization capabilities of the solvent for any specific active agent or any other component of the foamable composition. Rather, such information is provided to aid in the identification of materials suitable for use as a hydrophobic carrier in the foamable compositions described herein.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C. The term “hydrophobic organic carrier” does not include thick or semi-solid materials, such as white petrolatum, also termed “Vaseline”, which, in certain compositions is disadvantageous due to its waxy nature and semi-solid texture.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also include polyunsaturated oils containing poly-unsaturated fatty acids. In one or more embodiments, the unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the present foamable composition. Thus, the hydrophobic solvent can include at least 6% of an oil selected from omega-3 oil, omega-6 oil, and mixtures thereof. In this context, oils that possess therapeutically-beneficial properties are termed “therapeutically active oils.”

Another class of hydrophobic solvents is the essential oils, which are also considered therapeutically active oil, which contain active biologically occurring molecules and, upon topical application, exert a therapeutic effect, which is conceivably synergistic to the beneficial effect of the steroid in the composition.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

In one or more embodiments, the hydrophobic carrier includes at least 2% by weight silicone oil or at least 5% by weight.

The solvent may be a mixture of two or more of the above hydrophobic solvents in any proportion.

A further class of solvents includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

According to one or more embodiments, the hydrophobic organic carrier includes a mixture of a hydrophobic solvent and an emollient. According to one or more embodiments, the foamable composition is a mixture of mineral oil and an emollient in a ratio between 2:8 and 8:2 on a weight basis.

### Polar Solvent

A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, l-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

### Polymeric Agent/Additive

The polymeric agent serves to stabilize the foam composition and to control drug residence in the target organ. Exemplary polymeric agents are classified below in a non-limiting manner. In certain cases, a given polymer can belong to more than one of the classes provided below.

In one or more embodiments, the composition includes at least one gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® R resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 alkyl groups for each sucrose molecule.

In one or more embodiment, the composition includes at least one polymeric agent, which is a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methyl hydroxyethylcellulose, methylhydroxypropylcellulose, hydroxyethylcarboxymethylcellulose, carboxymethylcellulose and carboxymethylhydroxyethylcellulose. More preferably, the water-soluble cellulose ether is selected from the group consisting of methylcellulose, hydroxypropyl cellulose and hydroxypropyl methylcellulose (Methocel). In one or more embodiments, the composition includes a combination of a water-soluble cellulose ether; and a naturally-occurring polymeric materials, selected from the group including xanthan gum, guar gum, carrageenan gum, locust bean gum and tragacanth gum.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having at least one acidic group per four repeating or monomeric subunit moieties, such as poly(acrylic)- and/or poly(methacrylic) acid (e.g., Carbopol®, Carbomer®), polycarpophil, poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure—[CH₂—CH(COOH)-]ₙ. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO₃H) or basic groups (NH₂, NRH, NR₂), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Yet, another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

The foam composition may contain a film forming component. The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the composition includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺).

Non-limiting examples of phase change polymers include poly(N-isopropylamide) and Poloxamer 407®.

The polymeric agent is present in an amount in the range of about 0.01% to about 5.0% by weight of the foam composition. In one or more embodiments, it is typically less than about 1 wt % of the foamable composition.

### Pseudoplastic or Semi-pseudoplasic Agents

Pseudoplastics or viscoelasic fluids are capable of displaying a decreased viscosity when shear stress is increased. This behavior is typical of suspensions made of different size particles some bigger and some smaller, which when moving at high speed become orientated in the direction of flow particles and their interaction decreases. The viscosity profile can be an important factor in determining what is an appropriate pseudoplastic or semi-pseudoplastic composition. For example, compositions displaying Newtonian flow profiles are unsuitable. Compositions displaying non-Newtonian flow or showing plastic flow are not ideal although in certain circumstances it may be possible to achieve usable foamable compositions. The preferable profile is that of a pseudoplastic or semi-pseudoplastic flow.

### Stoke's Equation

The velocity at which a sphere will rise or fall in a liquid varies as the square of its diameter:

$$V = \frac{{d}^{2} ( {ρ}_{d} - {ρ}_{c} ) r {∞}^{2}}{18 η}$$

where V=particle velocity
d=particle diameter
p_{d}=discrete phase density
p=continuous phase density
r=radius of axis of rotation
ω=angular velocity
η=continuous phase viscosity
Note: (r ω²) is replaced by g, the force of gravity when not in a centrifugal field

The physical stability of an emulsion can be improved according to Stokes' law by adding appropriate emulsifers/stabilizers to increase the viscosity of the continuous phase and or to reduce and or maintain the droplet size.

Emulsifier molecules (surfactants) have hydrophilic and hydrophobic parts. They adsorb at the interface between the disperse phase and the continuous phase thus forming a dense film around the droplets of the disperse phase. This film prevents the coalescence of the droplets. Thus they act to maintain droplet size. They can also encourage the formation of smaller droplets

$${V}_{St} = \frac{Dr \cdot g \cdot {x}^{2}}{18 \cdot {h}_{c}}$$

V_{St}=stokes sinking (or creaming) velocity
Dr=density differences between the phases of the emulsion
G=acceleration, due to gravity
x=droplet diameter
h_{c}=dynamic viscosity of the continuous phase

As can be seen from above, the droplet motion can be reduced by one or more of decreasing the droplet diameter, by decreasing the density difference and by increasing the viscosity of the continuous phase.

In some cases emulsions as they become sticky also have a certain yield point caused by a hydrocolloidal stabilizer preventing floating of the oil droplet.

Stabilizers are therefore selected for the purposes of improving resistance to creaming and are effectively carefully selected thickening agents and or emulsifiers. In one or more embodiments, appropriate effective amounts of thickening agents are dissolved in the continuous phase to increase its viscosity or create a yield point. This restricts the moveability of the droplets such that less collisions between the droplets occur thereby reducing also the risk of coalescence. In certain other embodiments appropriate effective amounts of emulsifiers are added to maintain or reduce droplet size. In other embodiments both thickening and emulsifying agents are added to provide a multi approach to improving resilience to creaming whilst maintaining or achieving the desired rheology, flowability and shakability as described in the specification.

It should be kept in mind that the stabilizing emulsifier or surfactant may be multifunctional in nature. It supports the separating film between the phases; it increases viscosity; it acts as a foam booster or adjuvant and it acts to stabilize, maintain or reduce droplet size. It may act on its own or in combination with one or more surfactants and it may be a complex emulgator. Combinations of emulsifiers/surfactants may be preferable to achieve a reduced or stabilized droplet size.

In one or more embodiments, the composition contains one or more polymeric or gelling agents in a concentration to provide viscosity of less than 12,000 CP's. Polymeric agents having molecular weight of more that about 500 possess gelling properties; i.e., they can increase the viscosity of a composition. Therefore, by combining polymeric agents with different molecular weights/melting points, one can attain varying levels of flowability and pseudoplasicity/semi pseudo plasticity as desirable for stabilizing the composition to be resilient to creaming. According to some embodiments, the concentration of the polymeric or gelling agent should be in a level that results in viscosity, prior to filling of the composition into aerosol canisters, of less than 12,000 CP's, and more preferably, less than 10,000 CP's. In one or more other embodiments, the concentration of the polymeric agent is selected such that the range of viscosity of the composition, prior to filling of the composition into aerosol canisters, is about 500 CP's to about less than 20,000 CPs, and preferably, is about 1000 CP's to about less than 12,000 CP's. And more preferably about 3000 CP's to about less than 9000 CP's, and more preferably about 4000 CP's to about 7000 CP's. In still other embodiments a more viscous pre foam formulation is desirable. For example where the application is body cavity use and the viscosity is a desirable consequence of the nature and amount of at least one bioadhesive agent being present. The bioadhesive agent may itself generate a high, medium or lower viscosity. In an embodiment it comprises a high viscosity methyl cellulose. In another embodiment it can be a lower viscosity methyl cellulose. In either case the methyl cellulose can be alone or in combination with one or more other polymeric agents such as carbopol or polycarbophil. The low viscosity methyl cellulose may nevertheless be used to generate foamable pre foam formulations with a higher viscosity For bioadhesive formulations a wide range of viscosity may be allowable from about 1000 CPs to about to about 100,000 CPs, preferably from about 2000 CPs to about to about 30,000 CPs.

Particle or droplet size of the emulsions can be a significant factor. The emulsions should primarily comprise of droplets with a size or an average size of about less than 30 microns, preferably less than about 20 microns, more preferably less than about 10 microns and more preferably still less than about 5 microns in size. In one or more embodiments the amount of surfactant, combination of surfactants, the complex emulgator, combination of surfactant and foam adjuvant and the like is selected such that the range of particle size or range of average particle size in the emulsion prior to filling of the emulsion into aerosol canisters, is about 1 micron to about less than 30 microns, preferably is about 1 micron to about less than 10 microns, more preferably is about 1 micron to about less than 5 microns. In certain embodiments, the particle size or average particle size can be reduced to below 1 micron to about 0.3 microns and can be a nanoemulsion.

Foams are very complex and sensitive systems and are not formed at will. Mere addition of basic ingredients like oil, water, surfactant and propellant is far from sufficient to produce homogenous emulsions that can be used to provide a shelf stable, shakable pharmaceutical or cosmetic compositions from which foams of quality that are homogenous, stable, breakable upon mechanical force can be generated. Small deviations may lead to emulsion separation or foam collapse. Much consideration needs to be given to facilitate the introduction of an active agent, such as examining compatibility and non reactivity with the various excipients and container and determining shelf life chemical stability

Conventional emulsions are not thermodynamically stable. API's can be unstable in an aqueous environment. One approach to is to form micro emulsions and nano emulsions, which can be thermodynamically stable, making them a favorable vehicle for pharmaceutical compositions, which have to maintain stability for long periods of time. However, the resultant emulsions are different, the properties such as viscosity and particle size are different, considerations for selection of ingredients and amounts to be used for micro and nano emulsions are different, the method of preparation is different and can be time consuming and expensive, Also making micro and nano emulsions with a polymeric agent present is quite different to achieving emulsion formulations without one incorporated and vice versa.

To achieve regular emulsions that are suitable and adapted for pharmaceutical use and are physically stable for long periods of time without phase separation, and chemically stable such that the ingredients and API are compatible and do not break down or react with each other or packaging materials in which they are stored careful selection of ingredients and materials and the proportions is required. For an aqueous foam emulsion system the surfactants and any foam adjuvants and the oil phase and the hydrophobic propellant should be selected to achieve good compatibility so that the ingredients complement each others function in achieving the multiple objectives of a storage stable emulsion; a chemically stable system and a resultant reproducible foam which itself is stable with an appropriate bubble size, satisfactory collapse time, non stinging and with a pleasant texture and sensation upon contact or spreading and delivery of an effective amount of the active agent to the target site.

Bubble size can be from a few microns to several hundreds of microns. In some situations two bubble size ranges can be seen a main range and a secondary range of larger bubbles. Preferably substantially all the bubbles are within a main range. In an embodiment the bubble size ranges from an average of about 30 to about 250 micron. In a preferred embodiment it ranges from about 50 to about 220 micron, in another preferred embodiment it ranges from about 70 t about 200 micron. In a more preferred embodiment average bubble size ranges from about 80 to about 150 micron.

Stabilizers for the purpose of anti creaming, in products of the present invention, are polymeric or gelling agents like polysaccharides and emulsifiers, which act as surfactants amongst other things to stabilize or reduce droplet size.

In principle, any polymeric or gelling agent may be suitable. See for example the comprehensive list of polymeric or gelling agents. Preferably, suitable polymeric or gelling agents such are cellulose derivatives, Xanthan gum, Sodium CMC, methylcellulose, hydroxylpropyl methyl cellulose and the like, Some polymeric or geling agents may be suitable from the point of view of rheology but are less desirable from a secondary aspect of say leaving a film on the skin, which may flake. So in one or more embodiments the polymeric agent selected does not form an undesirable film on the skin.

Polysaccharides are preferred stabilizing polymers for foams. Suitable polysaccharides can be natural polysaccharides, derivatives thereof and modified poysacharides or derivatives. Polysaccharides are complex carbohydrates, made up of multiple sugar molecules. This term is commonly used only for those containing more than ten monosaccharide residues. Examples of polysaccharides include starch, dextrin, glycogen, cellulose and chitin. Glycosaminoglycan (GAG) is the polysaccharide unit that makes up proteoglycans, a molecule made of saccharides and proteins. GAGs are extracellular matrix molecules that help give tissues like cartilage their rigid structure and may also be useful stabilizing molecules. GAGs include chondroitinsulphate, dermatan sulphate, keratan sulphate, heparan sulphate, heparin, and hyaluronan. Starches are polymers of glucose in which glucopyranose units are bonded by alpha-linkages. Starches can also be useful stabilizers. For example, amylose consists of a linear chain of several hundred glucose molecules and amylopectine is a branched molecule made of several thousand of glucose units.

Examples of polymeric agents and surfactants are described else where in detail in the specification. Foam adjuvants, described elsewhere, can also be suitable for stabilizing the formulations.

### Surface-Active Agent

The composition further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. As will be appreciated by a person skilled in the art which surfactant or surfactant system is more appropriate is related to the vehicle and intended purpose. In general terms a combination of surfactants is usually preferable where the vehicle is an emulsion. In an emulsion environment a combination of surfactants can be significant in producing breakable forms of good quality. It has been further discovered that the generally thought considerations for oil in water emulsions of using a surfactant or sufactant combination with preferably a HLB value or average in or towards the lipophilic side of the scale are not always binding for oil in water emulsions and that good quality foams can be produced with a surfactant or surfactant combination both where the HLB values are in or towards the lipophilic side of the scale and more surprisingly where the HLB values are in or towards the hydrophilic side of the scale.

According to one or more embodiments of the present invention, the one or more surfactants (surface active agents) of the present invention are compatible with one or more organic carriers, such as oils. By “compatible” is meant that the surfactant(s) is matched with the carrier, as is appreciated by a person skilled in the art.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments the composition contains a single surface active agent having an HLB value between about 7 and 14, or more than one surface active agent and the weighted average of their HLB values is between about 7 and about 14. Mid range HLB values may, in certain embodiments, be more suitable for oil in water emulsions.

According to one or more other embodiments the composition contains a single surface active agent having an HLB value between about 9 and about 19, or more than one surface active agent and the weighted average of their HLB values is between about 9 and about 19.

Preferably, the composition contains a non-ionic surfactant. Nonlimiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether). More exemplary stabilizing surfactants which may be suitable for use in the present invention are found below.

PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

PEG-Fatty Acid Diester Surfactants:

| Chemical name | Product example name | HLB |
|---|---|---|
| PEG-4 dilaurate | Mapeg .RTM. 200 DL (PPG), | 7 |
| | Kessco .RTM.PEG 200 DL | |
| | (Stepan), LIPOPEG 2-DL (Lipo | |
| | Chem.) | |
| PEG-4 | distearate Kessco .RTM. 200 | 5 |
| | DS (Stepan.sub) | |
| PEG-32 dioleate | Kessco .RTM. PEG 1540 DO | 15 |
| | (Stepan) | |
| PEG-400 dioleate) | Cithrol 4DO series (Croda | >10 |
| PEG-400 disterate | Cithrol 4DS series (Croda) | >10 |
| PEG-20 glyceryl oleate | Tagat .RTM. O (Goldschmidt) | >10 |

Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated | Cremophor RH 40 (BASF), | 13 |
| castor oil) | Croduret (Croda), Emulgin | |
| | HRE 40 (Henkel) | |

Polyglycerized Fatty Acids, such as:

| Chemical name | Product example name | LB |
|---|---|---|
| Polyglyceryl-6 dioleate | Caprol .RTM. 6G20 (ABITEC); | 8.5 |
| | PGO-62 (Calgene), PLUROL | |
| | OLEIQUE CC 497 | |
| | (Gattefosse)Hodag | |

PEG-Sorbitan Fatty Acid Esters

| Chemical name | Product example name | HLB |
|---|---|---|
| PEG-20 sorbitan | Tween 40 (Atlas/ICI), Crillet 2 | 16 |
| monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| | (Croda) | |
| PEG-20 sorbitan | Tween-80 (Atlas/ICI), Crillet 4 | 15 |
| | (Croda) | |

Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

Sugar Ester Surfactants

| Chemical name | Product example name | HLB |
|---|---|---|
| Sucrose distearate | Sisterna SP50, Surfope 1811 | 11 |

Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20 Span 80; Span 20 Span 60; combinations of sucrose esters and sorbitan esters, such as Surphope 1811 and Span 60; combinations of liquid polysorbate detergents and PEG compounds, such as Tween 80 PEG-40 stearate; methyl glucaso sequistearate; polymeric emulsifiers, such as Permulen (TR1 or TR2); liquid crystal systems, such as Arlatone (2121), Stepan (Mild RM1), Nikomulese (41) and Montanov (68) and the like.

In certain embodiments, the surfactant is preferably a combination of steareth-2 and steareth-21; in certain other embodiments the surfactant is a combination of polysorbate 80 and PEG-40 stearate. In certain other embodiments the surfactant is a combination of glyceryl monostearate/PEG 100 stearate.

In one or more embodiments the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 9 and about 14.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect. Non limiting examples of surfactants with postulated tendency to form interfacial liquid crystals are: phospholipids, alkyl glucosides, sucrose esters, sorbitan esters. In certain embodiments surfactants which tend to form liquid crystals may improve the quality of foams produced from compositions.

In one or more embodiments, the surfactant is a surfactant or surfactant combination is capable of or which tends to form liquid crystals.

In one or more embodiments, the at least one surface active agent is solid, semi solid or waxy.

In one or more embodiments, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone can provide formulations and foams of good or excellent quality in the carriers and compositions.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, surface active agent comprises a combination of a non-ionic surfactant and an ionic surfactant, at a ratio of between 1:1 and 20:1.

In one or more embodiments, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1. The resultant foam has a low specific gravity, e.g., less than 0.1 g/ml.

In selecting a suitable surfactant or combination thereof it should be borne in mind that the upper amount of surfactant that may be used may be limited by the shakability of the composition. In general terms, as the amount of non liquid surfactant is increased the shakability of the formulation reduces until a limitation point is reached where the formulation becomes non shakable and unsuitable. Thus in an embodiment any effective amount of surfactant may be used provided the formulation remains shakable.

In certain embodiments, the amount of surfactant or combination of surfactants is between about 0.05% to about 20%; between about 0.05% to about 15%. or between about 0.05% to about 10%. In a preferred embodiment the concentration of surface active agent is between about 0.2% and about 5%. In a more preferred embodiment the concentration of surface active agent is between about 1% and about 4%.

If the composition is formulated as a substantially non flowing composition for use as a gel, ointment or cream, then the above limitation of shakability does not apply. Suitable formulations include polyethylene glycol or derivatives or mixtures thereof. For example, comprising higher molecular weight polymers or polymer combinations which are usually waxy or solid at room temperature such as PEG 4000 alone or a Peg 4000/400 combination with significant amounts of say PEG 4000 of 5% of 10% of 15% of 20% of 25% of 30% of as a basis for gels, ointments or creams. Other similar combinations may be envisaged of say PEG6000/PEG200; PEG4000/PEG200; PEG4000/PEG400/PEG200; and the like.

As will be appreciated by someone skilled in the art, as the levels of PEG are increased the composition will become more suitable for a gel or ointment pharmaceutical or cosmetic composition.

### Modulating Agent

The term modulating agent is used to describe an agent which can improve the stability of or stabilize a foamable carrier or composition and or an active agent by modulating the effect of a substance or residue present in the carrier or composition. The substance or residue may for example be acidic or basic and potentially alter pH in an emulsion environment or it may be one or more metal ions which may act as a potential catalyst in an emulsion environment.

In one or more embodiments the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

In one or more further embodiments the modulating agent is used to describe an agent, which is a chelating or sequestering or complexing agent that is sufficiently soluble or functional in the solvent to enable it to “mop up” or “lock” metal ions.

In the embodiment modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basesity balance of an emulsion carrier, composition, foamable carrier or foamable composition or resultant foam.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another, stronger acid residues, then the pH in an emulsion environment should be lower. In contrast, if one residue was acid and the other basic, the net effect in the formulation maybe significantly reduced. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment sufficient modulating agent is added to achieve an artificial pH in which the active agent is preferably stable.

A buffer, as defined by Van Slyke [Van Slyke, J. Biol. Chem. 52, 525 (1922)], is “a substance which by its presence in solution increases the amount of acid or alkali that must be added to cause unit change in pH.”

A buffer solution is a solution of a definite pH made up in such a way that this pH alters only gradually with the addition of alkali or acid. Such a solution consists of a solution of a salt of the week acid in the presence of the three acid itself. The pH of the solution is determined by the dissociation equilibrium of the free acid.

An acid can be a strong acid or a weak acid. A strong acid is an acid, which is a virtually 100% ionized in solution. In contrast, a weak acid is one which does not ionize fully, when it is dissolved in water. The lower the value for pKa, the stronger is the acid and likewise, the higher the value for pKa, the weaker is the acid.

A base can be a strong base or a weak base. A strong base is a base, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more embodiments, the modulating agent comprises an organic compound.

In one or more preferred embodiments, the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

In one or more embodiments, a preferred non limiting example of the chelating agent is EDTA. Typically, the chelating and sequestering agent is present in the composition at a level of up to about 5.0%, preferably 1.0 percent, by weight, of the composition.

In one or more embodiments, the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. In one or more embodiments the modulating agent is a flavonoid. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

In one or more embodiments the formulations described herein may further contain a modulating agent.

### Humectant

A humectant, is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer, is a substance that helps retain moisture or add back moisture to the skin. Examples are allantoin, petrolatum, urea, lactic acid, sodium PCV, glycerin, shea buffer, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions may in one or more embodiments usefully comprise in addition a heumectant or a moisturizer or combinations thereof.

### Foam Adjuvant

Preferably, a therapeutically effective foam adjuvant is included in the foamable compositions, to increase the foaming capacity of surfactants and/or to stabilize the foam. In one or more embodiments, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition. In some embodiments, the fatty alcohol includes at least one unsaturated fatty alcohol.

In one or more embodiments, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

In one or more embodiments, a combination of a fatty acid and a fatty ester is employed.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

An important property of the fatty alcohols and fatty acids used in context of the composition is related to their therapeutic properties, per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and antiinflammatory properties (see, U.S. Pat. No. 4,874,794). Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Thus, in preferred embodiments, a combined and enhanced therapeutic effect is attained by including both a steroid and a therapeutically effective foam adjuvant in the same composition, thus providing a simultaneous anti-inflammatory and antiinfective effect from both components. Furthermore, in a further preferred embodiment, the composition concurrently comprises a steroid, a therapeutically effective foam adjuvant and a therapeutically active oil, as detailed above. Such combination provides an even more enhanced therapeutic benefit. Thus, the foamable carrier, containing the foam adjuvant provides an extra therapeutic benefit in comparison with currently used vehicles, which are inert and non-active.

The foam adjuvant according to one or more preferred embodiments includes a mixture of fatty alcohols, fatty acids and hydroxy fatty acids and derivatives thereof in any proportion, providing that the total amount is 0.1% to 5% (w/w) of the carrier mass. More preferably, the total amount is 0.4%-2.5% (w/w) of the carrier mass.

The therapeutic foam may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

Optionally, the composition further contains a penetration enhancer. This is particularly important when the steroid is supposed to reach deeper layers of the target tissue or when the active agent is intended for systemic administration, via the transdermal or transmucosal route. Non limiting examples of penetration enhancers include propylene glycol, butylene glycols, glycerol, pentaerythritol, sorbitol, mannitol, oligosaccharides, dimethyl isosorbide, monooleate of ethoxylated glycerides having about 8 to 10 ethylene oxide units, polyethylene glycol 200-600, transcutol, glycofurol and cyclodextrins.

### Propellants

Examples of suitable propellants include volatile hydrocarbons such as butane, propane, isobutane and fluorocarbon gases, or mixtures thereof.

In an embodiment the propellant is 1681, which is a mixture of propane, isobutene and butane. In another embodiment it is AP 70, which is a mixture of propane, isobutene and butane with a higher pressure.

The propellant makes up about 5-25 wt % of the foamable composition. In some circumstances the propellant may be up to 35%. The propellants are used to generate and administer the foamable composition as a foam. The total composition including propellant, foamable compositions and optional ingredients is referred to as the foamable composition.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. Such propellants include, but are not limited to, hydrofluorocarbon (HFC) propellants.

In one or more embodiments, the non inflammable propellants are used in combination with the more traditional hydrocarbon propellants.

In one or more embodiments foamable compositions comprise a combination of a HFC such as dimethyl ether and a hydrocarbon propellant such as n-butane or mixtures of hydrocarbon propellants such as propane, isobutane and butane.

Aerosol propellants are used to generate and administer the foamable composition as a foam. The total composition including propellant, foamable compositions and optional ingredients is referred to as the foamable carrier. The propellant makes up about 3% to about 25 wt % of the foamable carrier. Examples of suitable propellants include volatile hydrocarbons such as butane, propane, isobutane or mixtures thereof, and fluorocarbon gases.

### Composition and Foam Physical Characteristics

A pharmaceutical or cosmetic composition manufactured using the foam carrier according to one or more embodiments is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foam composition creates a stable emulsion having an acceptable shelf-life of at least one year, or at least two years at ambient temperature. A feature of a product for cosmetic or medical use is long term stability. Propellants, which are a mixture of low molecular weight hydrocarbons, tend to impair the stability of emulsions. It has been observed, however, that emulsion foam compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam. Compositions containing semi-solid hydrophobic solvents, e.g., white petrolatum, as the main ingredients of the oil phase of the emulsion, exhibit high viscosity and poor flowability and are inappropriate candidates for a foamable composition.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

A further aspect of the foam is breakability. The breakable foam is thermally stable, yet breaks under sheer force. Sheer-force breakability of the foam is clearly advantageous over thermally-induced breakability. Thermally sensitive foams immediately collapse upon exposure to skin temperature and, therefore, cannot be applied on the hand and afterwards delivered to the afflicted area.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. patent application Ser. No. 11/732,547, filed on Apr. 4, 2007, KERATOLYTIC ANTIFUNGAL FOAM; U.S. patent application Ser. No. 11/767,442, filed on Jun. 22, 2007, entitled FOAMABLE COMPOSITIONS AND KITS COMPRISING ONE OR MORE OF A CHANNEL AGENT, A CHOLINERGIC AGENT, A NITRIC OXIDE DONOR, AND RELATED AGENTS AND THEIR USES; U.S. patent application Ser. No. 11/825,406, filed on Jul. 5, 2007, entitled DICARBOXYLIC ACID FOAMABLE VEHICLE AND PHARMACEUTICAL COMPOSITIONS THEREOF; U.S. patent application Ser. No. 11/900,072, filed on Sep. 10, 2006, entitled FOAMABLE VEHICLE AND VITAMIN AND FLAVONOID PHARMACEUTICAL COMPOSITIONS THEREOF; and U.S. patent application Ser. No. 11/947,751, filed Nov. 29, 2007, entitled COMPOSITIONS WITH MODULATING AGENTS, all of which are incorporated herein by reference in their entirety. More particularly any of the active ingredients; the solvents; the surfactants; foam adjuvants; polymeric agents, penetration enhancers; preservatives, humectants; moisturizers; and other excipients as well as the propellants and methods listed therein can be applied herein and are incorporated by reference.

The invention is described with reference to the following examples. This invention is not limited to these examples and experiments. Many variations will suggest themselves and are within the full intended scope of the appended claims.

A “stable foam” is defined herein as a composition, which upon release from an aerosol can, creates a foam mass, which is sustained on a surface for at least one minute, more preferably at least two minutes, and yet more preferably for at least 5 minutes. A period of minutes is regarded as a short term, but nevertheless it allows a good and more than sufficient period of time for a subject to receive foam dispensed on a body surface and to spread it or to transfer it to another region and to spread it.

In terms of spreadability and absorption an acceptable foam is one, that does not readily collapse upon dispensing on the skin; spreads easily on a skin surface; at least partially absorbed following rubbing onto the skin, and more preferably, substantially absorbed following rubbing on the skin.

In terms of tactile properties, an acceptable foam is one, that: creates a pleasant feeling after application; leaves minimal oily residue; and leaves minimal shiny residual look.

Skin drying and skin barrier function. Short chain alcohols are known to dry the skin and impair the integrity of the skin barrier. By contrast, including a film forming agent in the composition of the present invention foes not cause unwanted skin barrier damage.

Irritability. Due to the lack of lower alcohols (C₁-C₅) and improvement in skin barrier function, skin irritability is eliminated.

### Fields of Pharmaceutical Applications

By including an appropriate steroid and optional active agents in the compositions, the composition are useful in treating a patient having any one of a variety of dermatological disorders, which include inflammation as one or their etiological factors (also termed “dermatoses”), such as classified in a non-limiting exemplary manner according to the following groups:
(i) Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;
(ii) Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;
(iii) Fungal Infections including dermatophyte infections, yeast Infections; parasitic Infections including scabies, pediculosis, creeping eruption;
(iv) Viral Infections;
(v) Disorders of hair follicles and sebaceous glands including acne, rosacea, perioral dermatitis, hypertrichosis (hirsutism), alopecia, including male pattern baldness, alopecia greata, alopecia universalis and alopecia totalis; pseudofolliculitis barbae, keratinous cyst;
(vi) Scaling papular diseases including psoriasis, pityriasis rosea, lichen planus, pityriasis rubra pilaris;
(vii) Benign tumors including moles, dysplastic nevi, skin tags, lipomas, angiomas, pyogenic granuloma, seborrheic keratoses, dermatofibroma, keratoacanthoma, keloid;
(viii) Malignant tumors including basal cell carcinoma, squamous cell carcinoma, malignant melanoma, paget's disease of the nipples, kaposi's sarcoma;
(ix) Reactions to sunlight, including sunburn, chronic effects of sunlight, photosensitivity;
(x) Bullous diseases including pemphigus, bullous pemphigoid, dermatitis herpetiformis, linear immunoglobulin A disease;
(xi) Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;
(xii) Disorders of cornification including ichthyosis, keratosis pilaris, calluses and corns, actinic keratosis;
(xiii) Pressure sores;
(xiv) Disorders of sweating; and

(xv) Inflammatory reactions including drug eruptions, toxic epidermal necrolysis; erythema multiforme, erythema nodosum, granuloma annulare.

The same advantage is expected when the composition, including a steroid, is topically applied to a body cavity or mucosal surfaces, including, but not limited to the cranial cavity, the thoratic cavity, the abdominal cavity, the venteral cavity, the vagina, the rectum and penile cavities, the urinary tract, the nasal cavity, the mouth, the eye, the ear the peritoneum, the large and small bowel, the caecum, bladder, and stomach, the cavity between the uterus and the fallopian tubes, the ovaries and other body areas, which may accept topically-applied products. The composition is suitable to treat conditions of a body cavity and a mucosal membrane, such as post-surgical adhesions, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

According to one or more embodiments, the compositions are also useful in the therapy of non-dermatological disorders by providing transdermal delivery of a steroid that is effective against non-dermatological disorders. The composition is further useful for the treatment of ear inflammation and/or infection, such as otitis externa and otitis media. Otitis is commonly treated by a combination of an antimicrobial agent and a steroid (e.g., Neomycin, polymyxin B, and hydrocortisone). The composition is additionally useful in the treatment of disores of the nasal cavity, such as rhinitis and sinusitis. Corticosteroids and estrogens, in combination with vitamin A and/or vitamin D are occasionally sprayed into the nose to treat the inflammation or to reduce crusting by promoting mucosal secretions in nasal cavity disorders.

In one or more embodiments, the disorder is a health abnormality that responds to treatment with a steroid hormone. A typical example of such abnormality is sexual dysfunction in men and women whereby androgen therapy is successfully used to restore sexual function. Other non-limiting examples of disorders/medical indications that are in the scope of treatment with a steroid hormone according to the present invention are androgen deficiency, estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, Hirsutism, Virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

More specific examples of body cavity applications of steroids on their own or in combination with one or more other active agents are presented below without derogating from the above fields of application. By including an appropriate active agent in the foamable composition, it is useful in the therapy and prevention of a variety of disorders of a body cavity or mucosal surfaces, including, but not limited to the cranial cavity, the thoracic cavity, the abdominal cavity, the ventral cavity, the vagina, the rectum and penile cavities, the urinary tract, bladder, the cavity between the uterus and the fallopian tubes, the ovaries, the nasal cavity, the mouth, the eye, the ear the peritoneum, the large and small bowel, the caecum, bladder, and stomach, and other body cavities or spaces, which may accept topically-applied products.

Exemplary treatable disorders are listed below.

Bacterial, Fungal and Viral Infections

Bacterial, fungal and viral infections: a variety of anti-infective, anti-bacterial, anti-fungal and anti-viral agents can be included in the foam, to be used for the treatment and/or prevention of diseases, such as chlamydia, gonorrhea, hepatitis B, herpes, HIV/AIDS, human papallomavirus (HPV) & genital warts; syphilis; bacterial vaginosis, candidiasis, chancroid, granuloma inguinale, lymphogranloma venereum, mucopurulent cervicitis (MPC), molluscum contagiosum, nongonococcal urethritis (NGU), trichomoniasis, and vulvar disorders.

A variety of active agents, known in the art, can be included in the foam to be used for the treatment and/or prevention of diseases such as vulvodynia (vulvar pain), yeast infections, genital warts (condyloma) vulvar dystrophy, vulvar intraepithelial neoplasia (VIN), invasive cancer of the vulva, contact dermatitis, pelvic inflammation, pelvic inflammatory disease (PID), genital cancer and cancer of the cervix, vulva or vagina.

Vaginal Dryness

Vaginal dryness is caused by a number of conditions and can be either an occasional hassle or a chronic problem. A variety of anti-inflammatory active agents, hormones, moisturizing, refatting and lubricating agents and local anesthetic agents can be included in the foam, to be used for the treatment and/or prevention of vaginal dryness.

Dyspareunia

Dysareunia is pain in the vagina or pelvis experienced during sexual intercourse. A variety of anti-inflammatory active agents, hormones, moisturizing, refatting and lubricating agents and local anesthetic agents can be included in the foam, to be used for the treatment and/or prevention of vaginal pain. Anal and rectal disease, such as anal abscess/fistula, anal cancer, anal fissure, anal warts, Crohn's disease, hemorrhoids, anal itch, also called pruritus ani, fecal incontinence, and polyps of the colon and rectum all may be treated using the foamable composition according to one or more embodiments.

The foam composition, comprising an active agent that is known to treat one of said anorectal disorders and administered rectally, expands effectively in the rectal cavity and provides optimal coverage of the cavity surface, for improved therapeutic results.

HIV and STD Treatment and Prevention

When comprising appropriate protective agents, the foam is active against HIV infection and other infections (bacterial and fungal), including sexually transmitted disease (STD) by creating a protective layer and/or decreasing the frequency of transmission. Non-binding examples of protective agents include:

Negatively charged sulfated polymers, which have been reported to have anti-HIV-1 activity and are being considered for development as topical microbicides.

Dextrin sulphate, a microbicide, which in various laboratory and pre-clinical studies, has been shown to block the transfer of HIV virus into mammalian cells while at the same time not causing injury to normal cell tissue.

Cellulose acetate phthalate, which inactivates HIV-1, herpesvirus types I (HSV-1) and 2 (HSV-2) and the major nonviral STD pathogens; and found effective in animal models for vaginal infection by HSV-2 and simian immunodeficiency virus.

Several polymers, such as hydroxypropyl methylcellulose phthalate, carrageenans, naphthalene sulfonate polymer, sodium alginate, and cationic polymer, such as chitosan, are insoluble in water and can be solubilized in water by adjusting the pH of the environment to about 6 or above, or by the use of appropriate organic solvents. Vaginal secretions from healthy, reproductive-age women are characteristically acidic (pH values of 3.4 to 6.0). Consequently, the topical application of a formulation in which such polymers would be soluble (i.e., pH>6) would be expected to contribute to a vaginal environment which is physiologically undesirable.

Thus, in one embodiment of present invention, there is an advantage to an oily foamable carrier, comprising solvents that solubilize water-insoluble polymers such as mentioned above. By way of non-limiting example, such solvents include polyethylene glycol, propylene glycol, hexylene glycol, benzyl alcohol, DMSO, isosorbide derivatives, such as dimethyl isosorbide, glycofurol and ethoxydiglycol (transcutol).

In one or more embodiments, the foam composition is useful in the therapy of disorders that respond to transmucosal delivery of an active agent. By way of example, such disorders include, which respond to hormone therapy, such as hormone replacement therapy, and other systemic disorders, known to be affected by drugs that are delivered transmucosally.

The goal of a mucosal vaccine is to induce antigen-specific immune responses (cellular and humoral) that are detectable at the mucosal surfaces of the host. Because many pathogens initiate infection at the mucosal surfaces, pathogen-specific mucosal immune responses may provide superior protection against infectious diseases than immune responses induced by parenteral vaccines because parenteral vaccines do not induce mucosal immunity.

In the context of the present invention, the term immunization or vaccination refers to administering a preparation that contains an infectious agent or its components, which is able to stimulate an immune response that will protect a person from illness due to that agent. Such vaccines are expected be capable of preventing the transmission or limiting the severity of sexually-transmitted infections, such as HIV and other infectious disease. Vaccines are usually administered in conjunction with an adjuvant—a substance that is used in a vaccine to improve the immune response so that less vaccine is needed to produce a non-specific stimulator of the immune response. There are several types of adjuvants, including, for example, minerals such as aluminum hydroxide, aluminum phosphate and calcium phosphate, oil emulsions, products from bacteria (their synthetic derivatives as well as liposomes) or gram-negative bacteria, endotoxins, cholesterol, fatty acids, aliphatic amines, paraffinic and vegetable oils.

The foam composition, comprising an immunizing agent, and optionally an adjuvant and administered onto the mucosal tissue of a body cavity, expands effectively in said cavity and provides optimal coverage of the cavity surface, for improved therapeutic results.

Post-Surgical Adhesions Treatment and Prevention

Adhesions are scars that form abnormal connections between tissue surfaces. Post-surgical adhesion formation is a natural consequence of surgery, resulting when tissue repairs itself following incision, cauterization, suturing, or other means of trauma. When comprising appropriate protective agents, the foam is suitable for the treatment or prevention of post surgical adhesions. The use of foam is particularly advantageous because foam can expand in the body cavity and penetrate into hidden areas that cannot be reached by any other alternative means of administration.

Hormonal Therapy

The foamable composition is suitable for administering a hormone to a mucosal membrane or a body cavity, in order to deliver the hormone into the tissue of the target organ, in any disorder that responds to treatment with a hormone. Topically applied hormones can also be useful in contraception, when administered in foam, using a metered dose unit.

The following examples exemplify the therapeutic kits and pharmacological compositions and methods and uses described herein. Both exemplary postulated examples and examples of actual formulations are provided. Formulations without physical or chemical data are to be considered as exemplary postulatd examples. The examples are for the purposes of illustration only and are not intended to be limiting. Many of the examples are formulations comprising a steroid and or an LCD extract. As would be appreciated by a man of the art they may be formulated with LCD on its own or with at least one steroid, or with other active agents described herein or with combinations thereof.

In one or more embodiments, the active agent may be consigned to a body which is suspended or contained within the formulation. In some embodiments, the active agent is carried by the body. In other embodiments, the active agent is captured by the body. In further embodiments, the active agent is contained in the body. In some embodiments, the body is a trapsome.

A “Trapsome” is an insoluble body that is insoluble in water and in the final formulations described herein that is capable of entrapping, incorporating or otherwise suspending an active agent within its structure or confines and releasing it upon a certain signal or stimulus, which can be a physical or a chemical or other signal. Non limiting examples of substances that can act as trapsomes are a) microsponges, b) silica, c) mineral bodies like (i) zeolite, (ii) bentonite, (iii) graphite, inclusive (iv) polymers, (v) dendrimers and (v) liposomes, etc. Trapsomes may be used to incorporate one or more active agents in a emulsion. For example, the trapsome may retain the at least one active agent during storage, yet release the at least one active agent at the delivery site responsive to a local temperature effect, a local pH effect, a local conductivity effect or any other local parameter of the delivery site, which is different from that of the storage conditions.

In one or more embodiments the trapsome is a micropsponge. Microsponges are macroporous beads, typically 10-25 microns in diameter, loaded with active agent. They consist of a copolymer, such as methyl methacrylate/glycol dimethacrylate crosspolymer. Depending on their chemical composition, microsponges may be biodegradable. Microsponges have the capacity to entrap a wide range of active ingredients such as emollients, fragrances, essential oils, sunscreens and anti-infective, etc. Examples of drugs that have been incorporated in microsponges include ibuprofen ketoprofen (non-steroidal anti-inflammatory agent), benzyl peroxide (an anti-acne agent), retinoids, such as retinoic acid and retinol, fluconazole (an antifungal agent) (Y Kawashima et al. 1992,D'Souza et al. 2005,Wster R C et al. 1991,Tansel et al. 2003).

Microsponges are used as a topical carrier system. When applied to the skin, the microsponges release the active ingredient on a time mode and also in response to other stimuli (rubbing, temperature, pH, etc). By delivering the active gradually to the skin, Microsponge-benzoyl peroxide formulations, for example, have excellent efficacy with minimal irritation. These are typical benefits from their use.

In addition to their drug entrapping properties, microsponges are capable of absorbing skin secretions, therefore reducing oiliness and shine from the skin. Clinical studies demonstrated that the use of microsponges in a lotion reduced perceived oiliness on the skin by 50%; shine was reduced by 20% microspheres.

Other embodiments of the present invention are directed to trapsomes other than microsponges. A person skilled in the art, will understand that one trapsome may be replaced by one or more other trapsomes in the compositions of the present invention.

“Silicon” containing solid matter includes silicon oxide, also termed “silica”, “fumed silica” and “silica gel”, a white or colorless insoluble solid (SiO₂); and talc, which is fine grained mineral consisting of hydrated magnesium silicate. Silicon may provide two different effects in emulsion compositions: a) a trapsome effect; and b) a sensation effect to the skin.

Other trapsomes include according to some embodiment, carbon, for example in the form of amorphous carbon or graphite; and/or oxidizing agents, such as benzoyl peroxide, calcium and magnesium hypochlorite.

According to some embodiments, the emulsion compositions may include metallic silver, in small particles, including nanocrystalline silver, which is used for antibacterial and wound healing purposes; other metal particles and mineral particles. Metallic particles may also provide a trapsome effect.

According to some embodiments, the emulsion compositions may further include cosmetic scrub materials, including, for example meals of strawberry seeds, raspberry seeds, apricot seeds, sweet almond, cranberry seeds; and pigments, which are insoluble in the bubbled-gel composition.

Silica” [Silicone, as used in the application is “silicone oil, like dimethicone, and silicone copolymers and surfactants] containing solid matter includes silicon oxide, also termed “silica”, “fumed silica” and “silica gel”, a white or colorless insoluble solid (SiO₂); and talc, which is fine grained mineral consisting of hydrated magnesium silicate. An example of entrapping silica is silica microcapsules, made by Sol-Gel Technologies, that can be fine-tuned to provide optimal release properties for numerous actives including liquids, hydrophobic materials, and solids with poor water-solubility, as described, for example, in U.S. Pat. No. 6,436,3759, 6,303,149, 6,238,650;

“Graphite intercalation compounds” are intercalation compounds with a graphite host. In this type of compound the graphite layers remain largely intact and the guest molecules or atoms are located in between. When the host and the guest interact by charge transfer, the in-plane electrical conductivity generally increases. When the guest forms covalent bonds with the graphite layers as in fluorides or oxides the conductivity decreases as the conjugated sp² system collapses. In a graphite intercalation compound not every layer is necessarily occupied by guests. In so-called stage 1 compounds graphite layers and intercalated layers alternate and in stage 2 compounds two graphite layers with no guest material in between alternate with an intercalated layer. The actual composition may vary and therefore these compounds are an example of non-stoichiometric compounds. It is customary to specify the composition together with the stage.

A “zeolite” is a mineral having a micro-porous structure. More than 150 zeolite types have been synthesized and 48 naturally occurring zeolites are known. They are basically hydrated alumino-silicate minerals with an “open” structure that can accommodate a wide variety of cations, such as Na⁺, K⁺, Ca²⁺ Mg²⁺ and others. These positive ions are rather loosely held and can readily be exchanged for others in a contact solution. Some of the more common mineral zeolites are: analcime, chabazite, heulandite, natrolite, phillipsite, and stilbite. An example mineral formula is: Na₂Al²Si₃O₁₀-2H₂O, the formula for natrolite. Zeolite products may be used in the bubbled-gel compositions of the present invention as inert carriers for one or more active agents.

Natural zeolites form where volcanic rocks and ash layers react with alkaline groundwater. Zeolites also crystallized in post-depositional environments over periods ranging from thousands to millions of years in shallow marine basins. Naturally occurring zeolites are rarely pure and are contaminated to varying degrees by other minerals, metals, quartz or other zeolites. For this reason, naturally occurring zeolites are excluded from many important commercial applications where uniformity and purity are essential.

Zeolites are the aluminosilicate members of the family of microporous solids known as “molecular sieves”. The term molecular sieve refers to a particular property of these materials, i.e. the ability to selectively sort molecules based primarily on a size exclusion process. This is due to a very regular pore structure of molecular dimensions. The maximum size of the molecular or ionic species that can enter the pores of a zeolite is controlled by the diameters of the tunnels. These are conventionally defined by the ring size of the aperture, where, for example, the term “8 ring” refers to a closed loop that is built from 8 tetrahedrally coordinated silicon (or aluminium) atoms and 8 oxygen atoms. These rings are not always perfectly flat and symmetrical due to a variety of effects, including strain induced by the bonding between units that are needed to produce the overall structure, or coordination of some of the oxygen atoms of the rings to cations within the structure. Therefore, the pore openings for all rings of one size are not identical. Thus, zeolites may serve to preferentially release one active agent, according to a small pore size opening and release another agent via larger pore size openings.

“Bentonite” is an absorbent aluminium phyllosilicate generally impure clay consisting mostly of montmorillonite. There are a few types of bentonites and their names depend on the dominant elements, such as K, Na, Ca, and Al. As noted in several places in the geologic literature, there are some nomenclatorial problems with the classification of bentonite clays. Bentonite usually forms from weathering of volcanic ash, most often in the presence of water. However, the term bentonite, as well as a similar clay called tonstein, have been used for clay beds of uncertain origin. For industrial purposes, two main classes of bentonite exist: sodium and calcium bentonite. Bentonite products may be used in the bubbled-gel compositions of the present invention as inert carriers for one or more active agents.

A “liposome” is a spherical vesicle composed of a bilayer membrane. In biology, this specifically refers to a membrane composed of a phospholipid and cholesterol bilayer (see on the right). Liposomes can be composed of naturally-derived phospholipids with mixed lipid chains (like egg phosphatidylethanolamine), or of pure surfactant components like DOPE (dioleoylphosphatidylethanolamine). Liposomes, usually but not by definition, contain a core of aqueous solution; lipid spheres that contain no aqueous material are called micelles, however, reverse micelles can be made to encompass an aqueous environment.

Liposomes are used for drug delivery due to their unique properties. A liposome encapsulates a region on aqueous solution inside a hydrophobic membrane; dissolved hydrophilic solutes can not readily pass through the lipids. Hydrophobic chemicals can be dissolved into the membrane, and in this way liposome can carry both hydrophobic molecules and hydrophilic molecules. To deliver the molecules to sites of action, the lipid bilayer can fuse with other bilayers such as the cell membrane, thus delivering the liposome contents.

Liposomes products may be used in the bubbled-gel compositions of the present invention as carriers for one or more active agents, for targeted delivery of one or more active agents to a delivery site.

“Microsponges” are rigid, porous and spongelike round microscopic particles of cross-linked polymer beads (e.g., polystyrene or copolymers thereof), each defining a substantially noncollapsible pore network. The Microsponges can be loaded with an active ingredient and can provide a controlled time release of the active ingredient to skin or to a mucosal membrane upon application of the formulation. The slow release is intended to reduce irritation by the active. Microsponge® delivery technology was developed by Advanced Polymer Systems.

Microsponges have a size range in between 5 to 300 μm depending upon the degree of smoothness or after feel required for the end formulations and can reduce perceived oiliness.

Wide ranges of uses for microsponges incorporated in foamable compositions are suggested aiming to provide increased efficacy for delivery of active agents topically and in a body cavity with enhanced control, spreadability, safety, stability and improved aesthetic properties. Microspheres can store an active agent until its release is triggered by application to the skin surface such as through rubbing and or higher than-ambient skin temperature. Microsponge do not pass through the skin but collect on the skin surface and slowly release the entrapped agent. The empty spheres are washed away with cleansing.

Microsponges may be incorporated in wide ranges of foam formulations. In one or more embodiments microsponges may be incorporated into the formulations exemplified and described herein.

In an embodiment, the amount of microsponges may be varied from about 1% to about 25% of the formulation, preferably about 5% to 15%.

In an embodiment, any active agent suitable for loading in microsponges may be used, such as benzyl peroxide (BPO), tretinoin, hydroquinone, kotoprofen, retinol, fluconazole, ibuprofen, trolamine and the like.

In an embodiment, the microsponges are loaded with one or more steroids.

In an embodiment, the microsponges are loaded with coal tar.

In an embodiment, the microsponges are loaded with one or more vitamins or with one or more flavonoids or combinations thereof. In another embodiment the vitamins and or flavonoids are fat soluble. In another embodiment they are water soluble.

In an embodiment, the vitamin is a retinoid, preferably a vitamin A, more preferably, retinoic acid or isoretinoic acid.

In an embodiment, the vitamin is preferably a vitamin D, a derivative or analogue thereof, more preferably calcipotriol or calcitriol or tacalcitol with or without a corticosteroid such as betmethasone or its esters (eg bmv), flucinonide, hydrocortisone or clobetasol proprionate.

In an embodiment, since retinoids and BPO can dry the skin, water soluble humectants, e.g., urea, sodium PCA, alpha-hydroxy acids, glycerin and other polyols may be added.

As can be noted from above and herein different types of active agents may be loaded into the microsponges. Accordingly the foam formulation selected in which to disperse the microsponges should be adapted so that the active agent remains substantially entrapped in the microsponges. In another embodiment the active agent is present both in the foam formulation and in the microsponges so that some of the active agent is available for immediate penetration on application of the foam and that other amounts of active agent are provided by slow or controlled release from the microsponges now sitting on the topical surface.

Where the active agent is oil soluble but not water soluble then formulations with minimal or no oil are preferred where the active agent is primarily to be located in the microsponges. So in an embodiment there is no oil in the formulations, only gels. In another embodiment, there are no significant amounts of true oils that solubilize the active ingredients and extract them from the microsponges.

In an embodiment, where the active ingredient is insoluble in water and is entrapped in the microsponges, there is provided true oil in water emulsion, where the active ingredient is only exposed to the external water phase and does not access the internal oil phase. This may be achieved in an embodiment by formulating an aqueous oil gel foam with substantial water content.

In an embodiment, the foam formulation comprises a large amount of hydrophobic surfactant or hydrophobic complex emulgators which can form a tight close surfactant layer surrounding the oil droplets sitting in the water phase to separate but still holding the water and oil phases together as an emulsion, thereby preventing or at least substantially reducing any leakage of active agent into the oil phase. In a further embodiment there is provided a foam, where the gelling agent is not fat soluble so as to prevent or at least substantially reduce leakage.

The methodology of loading microsponges with active agent and amounts that can be loaded are described in WO 01/85102, which is incorporated herein by way of reference. Where Drug Microsponge X % w/w is provided it refers to the microsponges including the trapped drug and any other ingredients incorporated when loading the microsponges.

### General Methodology

Emulsion Pre-Foamable Foam Formulation
1. Mix oily phase ingredients and heat to 75° C. to melt all ingredients and obtain homogeneous mixture.
2. Mix polymers in water with heating or cooling as appropriate for specific polymer.
3. Add all other water soluble ingredients to water-polymer solution and heat to 75° C.
4. Add slowly internal phase to external phase at 75° C. under vigorous mixing and homogenize to obtain fine emulsion.
5. Cool to below 40° C. and add sensitive ingredients with mild mixing.
6. Cool to room temperature.
Note: The formulation can the be stored and as required introduced into an aerosol canister with propellant.

### Production Under Vacuum

Optionally, the foamable formulation may be produced under nitrogen and under vacuum. Whilst the whole process can be carried out under an oxygen free environment, it can be sufficient to apply a vacuum after heating and mixing all the ingredients to obtain an emulsion or homogenous liquid. Preferably the production chamber is equipped to apply a vacuum but if not the formulation can be for example placed in a dessicator to remove oxygen prior to filing and crimping.

### Canisters Filling and Crimping

Each aerosol canister is filled with PFF and crimped with valve using vacuum crimping machine. The process of applying a vacuum will cause most of the oxygen present to be eliminated. Addition of hydrocarbon propellant may without being bound by any theory further help to reduce the likelihood of any remaining oxygen reacting with the active ingredient. It may do so, without being bound by any theory, by one or more of dissolving in the oil or hydrophobic phase of the formulation, by dissolving to a very limited extent in the aqueous phase, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing

### Propellant Filling

Pressurizing is carried out using a hydrocarbon gas or gas mixture. Canisters are filled and then warmed for 30 sec in a warm bath at 50° C. and well shaken immediately thereafter.

### Closure Integrity Test

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

### Analytical Methodology

### Foam Tests

By way of non limiting example the objectives of hardness, collapse time and FTC stability and other tests are briefly set out below as would be appreciated by a person of the art.

### Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can affect the degree of dermal penetration, efficacy, spreadability and acceptability to the user. The results can also be looked at as an indicator of softness. Note: the foam sample is dispensed into an aluminum sample holder and filled to the top of the holder.

### Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min. Thus foams which are structurally stable on the skin for at least one minute are termed “short term stable” compositions or foams.

### Drainage:

A simple indication of the rate of drainage or whether there is any significant drainage can be obtained from the collapse time method and results. In the collapse time method the foam is observed and filmed for 300 secs. The height of the foam is measured against a marked ruler and any changes recorded and plotted as a graph. Also the foam quality is observed. If during the measurement there is a change in quality it is noted. So simply if there is a reduction in quality say from Good to Fairly Good or from Excellent to Good then significant drainage is considered to have occurred. And the approximate time point when this change has been noted is said to be the drainage time. A slow drainage is a drainage of more than 300 seconds.

### Viscosity

Viscosity is measured with Brookfield LVDV-11+ PRO with spindle SC₄₋₂₅ at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude.

### FTC (Freeze Thaw Cycles)

To check the foam appearance under extreme conditions of repeated cycles of cooling, heating, (first cycle) cooling, heating (second cycle) etc., commencing with −100° C. (24 hours) followed by +400° C. (24 hours) measuring the appearance and again repeating the cycle for up to three times.

### Chemical Stability

The amount of active agent present is analyzed in foam expelled from various pressurized canisters containing foam formulations using HPLC. Analysis is carried out at zero time and at appropriate time intervals thereafter. The canisters are stored in controlled temperature incubators at 5° C., at 25° C., at, 40° C. and at 50° C. At appropriate time intervals canisters are removed and the amount of active agent in the foam sample is measured.

### Bubble Size

Foams are made of gas bubbles entrapped in liquid. The bubble size and distribution reflects in the visual texture and smoothness of the foam. Foam bubbles size is determined by dispensing a foam sample on a glass slide, taking a picture of the foam surface with a digital camera equipped with a macro lens. The diameter of about 30 bubbles is measured manually relatively to calibration standard template. Statistical parameters such as mean bubble diameter, standard deviation and quartiles are then determined. Measuring diameter may also be undertaken with image analysis software. The camera used was a Nikon D40X Camera (resolution 10 MP) equipped with Sigma Macro Lens (ref: APO MACRO 150 mm F2.8 EX DG HSM). Pictures obtained are cropped to keep a squared region of 400 pixels×400 pixels.

### Bioadhesion

Bioadhesion (adhesive force) is an important property which needs to be examined for the selection of the foam formulation prototype. Adhesiveness is defined as the force (g) needed to overcome attraction between two vaginal tissue-surfaces after being in contact. An animal model system for measuring adhesiveness using pig vaginal tissues was developed for this purpose. Measurements make use of the LFRA Brookfield Texture Analyzer. Two sections of pig vaginal tissue (2×2 cm) are used; one is positioned in the center of a Petri dish and the other is attached to the base of texture analyzer probe. A PFF sample is spread uniformly on the tissue section on the Petri dish. The probe is moved down and up, first bringing the two sections into contact, followed by separation. The Texture Analyzer enables the measurement of the force for separating the tissue sections. The adhesive force is expressed as a negative force with the force to bring the two sections in contact as a positive force.

Measurement of bioadhesion was performed on formulations which are considered appropriate for vaginal use, which showed suitable physical and chemical properties at T-0 and after storage. Adhesiveness of Replens Gel an existing product was measured to serve as a reference.

TABLE 6
Adhesiveness Test Results for Reference Product., Replens Gel.

| Formulation | Adhesive force (g)* |
|---|---|
| Replens Gel For vaginal | −12.13 |
| lubrication) | |

*results, average of three determinations

### Creaming by Centrifugation:

### 1. Principle of Test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

### 2. Procedure

2.1. Following preparation of the experimental formulation/s, allow to stand at room temperature for >24 h.
2.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
2.3. Transfer each mixture to 1.5 mL microtubes. Tap each microtube on the table surface to remove entrapped air bubbles.
2.4. Place visually balanced microtubes in the centrifuge rotor and operate the centrifuge at 3,000 rpm for 10 min or at 10,000 rpm for 10 min. The centrifuge can be a BHG HEMLE Z 231 M.
2.5. Visually inspect samples following centrifugation for creaming and for emulsion breakdown.

### Hardness

### 1. Scope

The LFRA100 instrument is used to characterize hardness.

### 2. Principle of Test

This principle of operation of the LFRA texture analyzer is to drive a probe into the test material. The resistance of the material to compression is measured by a calibrated load cell and reported in units of grams on the instrument display. At least 3 repeat test measurements are made for each sample. Operation and calibration is according instrument instructions and program. Note: the foam sample is dispensed into an aluminum sample holder and filled to the top of the holder.

Chemical stability at 25° C. and at 40° C.; API concentration; and assay of degradation product are all measured by an HPLC tests specific for each one of the API's as will be appreciated by a an of the art. For example, the HPLC method for quantitative analysis of Betamethasone 17 valerate and Betamethasone 21 valerate (BMV 17 and 21) is briefly set out below:

### Principle of Test

About 1 g of foam or pre-foam formulation is dissolved in 0.1% acetic acid in methanol (diluent), to which an internal standard (IS) solution is added. BMV, BMV-21 and IS are extracted by heating, cooling and centrifugation of the sample solution. The clear layer is analyzed by HPLC using a C-18 column; elution is performed with a mobile phase containing acetonitrile:water 60:40. The content of BMV and BMV-21 is calculated using the respective peak area ratio against average peak area ratio of BMV standard (0.2 mg/mL) to the IS present in the standard solution at the same concentration as in samples.

### EXAMPLES

### Example 1

### Oil in Water Anti-Inflammatory Steroid Foamable Emulsion Compositions with/without Additional Active Agents

| | Composition Code: | | | | |
|---|---|---|---|---|---|
| | HB1 | BV1 | BV2 | HB2 | HB3 |
| Ingredient | % | | | | |
| Hydrocortisone butyrate | 0.10 | | | | |
| (steroid) | | | | | |
| Betamethasone valerate | | 0.12 | 0.12 | | |
| (steroid) | | | | | |
| Haolbetasol (steroid) | | | | 0.02 | 0.01 |
| Alpha-Bisabolol | | 0.20 | | 0.20 | |
| (additional active agent) | | | | | |
| D-Panthenol 50P | | 10.00 | | 10.00 | |
| (additional active agent) | | | | | |
| Mineral oil | 12.00 | 12.00 | 12.00 | 12.00 | 12.00 |
| Isopropyl myristate | 12.00 | 12.00 | 12.00 | 12.00 | 12.00 |
| Dimeticone V100 | 3.00 | 3.00 | 3.00 | 3.00 | 3.00 |
| Glyceryl monostearate | 0.50 | 0.50 | 0.50 | 0.50 | 0.50 |
| MYRJ 52 | 3.00 | 3.00 | 3.00 | 3.00 | 3.00 |
| Microcrystalline | 2.00 | 1.00 | 2.00 | 2.00 | 2.00 |
| cellulose + | | | | | |
| carboxymethyl cellulose | | | | | |
| TWEEN 80 | 1.00 | 1.00 | 1.00 | 1.00 | 1.00 |
| Cocoamidopropyl betaine | 0.50 | 0.50 | 0.50 | 0.50 | 0.50 |
| Preservative | 0.30 | 0.30 | 0.30 | 0.30 | 0.30 |
| Purified water | To 100 | To 100 | To 100 | To 100 | To 100 |

### Example 2

### Oil in Water Steroid Hormone Foamable Emulsion Compositions

| | Composition Code: | | | |
|---|---|---|---|---|
| | HB1 | BV1 | BV2 | HB2 |
| Ingredient | % | | | |
| Testosterone propionate (steroid hormone) | 0.25 | 0.25 | 0.25 | 0.25 |
| Propylene glycol (penetration enhancer) | 2.00 | 4.00 | | |
| Diethylene glycol monoethyl ether | | | 6.00 | |
| (penetration enhancer) | | | | |
| Mineral oil | 12.00 | 12.00 | 12.00 | 12.00 |
| Isopropyl myristate | 12.00 | 12.00 | 12.00 | 12.00 |
| Dimeticone V100 | 3.00 | 3.00 | 3.00 | 3.00 |
| Glyceryl monostearate | 0.50 | 0.50 | 0.50 | 0.50 |
| MYRJ 52 | 3.00 | 3.00 | 3.00 | 3.00 |
| Microcrystalline cellulose + carboxymethyl | 2.00 | 1.00 | 2.00 | 2.00 |
| cellulose | | | | |
| TWEEN 80 | 1.00 | 1.00 | 1.00 | 1.00 |
| Cocoamidopropyl betaine | 0.50 | 0.50 | 0.50 | 0.50 |
| Preservative | 0.30 | 0.30 | 0.30 | 0.30 |
| Purified water | To | To | To | To |
| | 100 | 100 | 100 | 100 |

### Example 3

### Oil in Water Foamable Emulsion Compositions Comprising Steroids from Natural Sources

Avocado (Persea gratissima) oil and extracts are used as an example of plants that contain phytosteroids. The oil and extracts contain two important phytosteroids—stigmasterol and beta-sitosterol, which are known to provide a variety of beneficial effects, such as tissue regeneration, improvement of skin elasticity, moisturization, stimulation of collagen synthesis, inhibition of leukotriene and prostaglandin formation (anti-inflammatory effect), anti-bacterial effects, skin barrier repair, film-forming properties, improvement of trans-epidermal water loss. Lanolin oil contains high levels of cholesterol, iso-cholesterol and derivatives thereof. The following compositions include avocado oil and lanolin oil as part of the oil phase of the composition.

| Composition No: | | |
|---|---|---|
| LN1 | LN2 | AV1 |

| | |
|---|---|
| Ingredient | % |

| | | | |
|---|---|---|---|
| Lanolin oil | 2.50 | 2.50 | — |
| Avocado concentrate - Avocadin, | — | — | 5.60 |
| produced by Crodarom (steroid) | | | |
| Mineral oil | 5.60 | 5.60 | — |
| Isopropyl myristate | 5.60 | 5.60 | 5.60 |
| Glyceryl monostearate | 0.45 | 0.45 | 0.45 |
| Stearyl alcohol | 0.85 | 0.85 | 0.85 |
| Xanthan gum | 0.20 | 0.20 | 0.20 |
| Methocel K100M | 0.20 | 0.20 | 0.20 |
| Polysorbate 80 | 0.90 | 0.90 | 0.90 |
| PEG-40 stearate | 2.60 | 2.60 | 2.60 |
| EDTA disodium | 0.20 | 0.20 | 0.20 |
| Preservative | 0.25 | 0.25 | 0.25 |
| Propellant | 8.00 | 8.00 | 8.00 |
| Water | To 100 | To 100 | To 100 |

### Example 4

### An Emulsion Composition Containing a Steroid Solubilize in the Composition, but Insoluble Both in Water and in the Oil Phase of the Composition

| | Composition No: |
|---|---|
| | BV3 |
| Ingredient | % |
| Betamethasone valerate (steroid) | 0.12 |
| Mineral oil | 30.00 |
| Glyceryl stearate | 1.00 |
| Stearyl alcohol | 1.00 |
| PEG-40 stearate | 2.50 |
| Xanthan gum | 0.26 |
| Methocel K100 | 0.26 |
| Polysorbate 60 | 1.00 |
| Citric acid | 1.30 |
| Sodium citrate | 1.10 |
| Water | 61.46 |

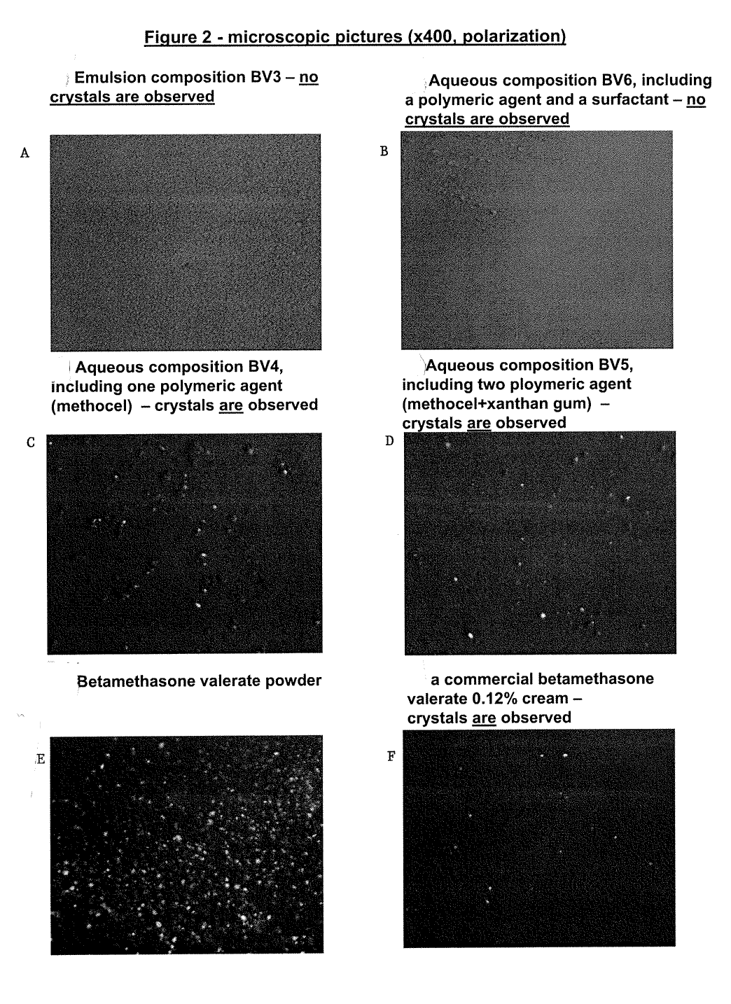

The emulsion composition of Example 4 was microscopically observed at x400 magnification with polarization. As demonstrated in FIG. 2A (Plate 1), no crystals were observed in the emulsion composition.

In an effort to identify the components that contribute to solubilization, the aqueous phase of the emulsion was prepared, with and without a surface active agent. See, FIGS. 2B and 2C. In this particular case, the surface active agent system consisted of a combination of one non-ionic surfactants PEG-40 stearate and Polysorbate 60, as shown in the following compositions:

| | Composition No: | | |
|---|---|---|---|
| | BV4 | BV5 | BV6 |
| | Water Phase, | Water Phase, | Water Phase, |
| Ingredient | No Surfactant | With Surfactant | With Surfactant |
| Betamethasone | 0.12 gr. | 0.12 gr. | 0.12 gr. |
| valerate (steroid) | | | |
| PEG-40 stearate | — | — | 2.50 gr. |
| (surface active agent) | | | |
| Polysorbate 60 | — | — | 1.00 gr. |
| (surface active agent) | | | |
| Xanthan gum | — | 0.26 gr. | 0.26 gr. |
| (polymeric agent) | | | |
| Methocel K100 | 0.26 gr. | 0.26 gr. | 0.26 gr. |
| (polymeric agent) | | | |
| Citric acid | 1.30 gr. | 1.30 gr. | 1.30 gr. |
| Sodium citrate | 1.10 gr. | 1.10 gr. | 1.10 gr. |
| Water | 61.46 gr. | 61.46 gr. | 61.46 gr. |

As shown in FIG. 2B-2D, composition BV4 and BV5, prior to the addition of a surface active agent exhibited high crystal content (Plate 3 and 4), while in composition BV6 including a surface active agent system, no crystals were observed (Plate 2). For reference purposes in FIGS. 2E and 2F, Plates 5 and 6 illustrate the microscopic pictures of Betamethasone valerate powder and a commercial betamethasone valerate 0.12% cream (Betnovate, GlaxoSmithkline).

Thus, in one or ore embodiments there is provided a composition containing a steroid solubilized or substantially solubilized in the composition, but insoluble both in water and in the oil phase of the composition.

### Example 5

This monocenter, single Investigator study was conducted to determine the skin irritation, and sensitivity of a series of foamable compositions in subjects with normal and sensitive skin. The objectives of the study were:

To assess the skin irritation potential of Foamix Products and two controls: purified water as the negative control and SLS as the positive control, by means of a single 48-hour patch application to the skin of healthy volunteers, having either “normal skin” or “sensitive skin”.

A total of forty subjects were screened. Thirty subjects with “normal skin” and ten subjects with “sensitive skin”, who satisfied the inclusion and exclusion criteria, were able to accept the prohibitions and restrictions and provided written informed consent were enrolled onto the study and participated in the Irritation and sensitivity tests study.

### Results

Irritation Potential in Subjects with Normal Skin:

Thirty subjects with normal skin successfully completed the skin Irritation study.

30 subjects received Foamix Product No. 7 and 2 controls one positive and one negative. The patches remained in place for 48 hours.

Foamix tested article No. 7 did not reveal any sign of irritation following application, in any of the study subjects. Thus, under the current study protocol and conditions, Foamix tested article, Betamethasone valerate 0.12% can be regarded as non-irritating to normal skin.

Irritation Potential in Subjects with Sensitive Skin:

Ten subjects with sensitive skin successfully completed the Irritation Test.

Each received Foamix Products No. 7 at a dose of about 0.1 mL under occlusive patches. The patches remained in place for 48 hours.

Foamix tested article No. 7 did not reveal any sign of irritation following application, in any of the study subjects. Thus, under the current study protocol and conditions, Foamix tested article Betamethasone valerate 0.12% can be regarded as non-irritating to sensitive skin.

### Safety

No adverse events was recorded.

List of tested Products ID numbers

| Product | Product ID Number |
|---|---|
| Betamethasone Valerate 0.12% | 7 |
| Purified water | 12 |
| detergent solution 2% Sodium lauryl sulphate | 13 |

### Tested Article Formulation

Test Article No 7: Betamethasone valerate 0.12%

| Ingredients | % w/w |
|---|---|
| Betamethasone valerate | 0.12 |
| Isopropyl myristate | 10.00 |
| Octyldodecanol | 12.00 |
| Stearyl alcohol | 1.00 |
| Glyceryl stearate | 1.00 |
| PEG-40 stearate | 2.60 |
| Methylcellulose K100M | 0.28 |
| Xanthan gum | 0.28 |
| Polysorbate 60 | 1.00 |
| Purified water | 68.32 |
| Parabens in phenoxyethanol | 1.00 |
| Citric acid | 1.30 |
| Sodium citrate | 1.10 |
| Total product: | 100.00 |

### Example 6

### Hydrocortisone and LCD Matrix of Compositions Showing Resilience to Creaming a) with Carboxymethylcellulose Sodium and b) with Hypromellose and Xanthan Gum

6(a) Matrix with Carboxymethylcellulose sodium

| USAN name | W/W % | W/W % | W/W % |
|---|---|---|---|
| Mineral oil, heavy | 0.00 | 0.00 | 5.00 |
| PPG-15 Stearyl ether | 0.00 | 3.00 | 3.00 |
| Petrolatum, white | 1.25 | 2.50 | 0.00 |
| Lanolin | 0.00 | 0.00 | 0.00 |
| Octyl dodecanol | 3.00 | 6.00 | 0.00 |
| Cetearyl octanoate | 0.75 | 1.50 | 0.00 |
| Glyceryl monostearate | 0.00 | 1.00 | 0.00 |
| Cetearyl alcohol | 1.00 | 1.00 | 1.00 |
| Ceteareth-20 | 0.00 | 3.00 | 3.00 |
| Polyoxyl 40 stearate | 2.50 | 0.00 | 0.00 |
| Dimethicone 350 | 0.00 | 0.00 | 1.00 |
| Benzyl alcohol | 0.60 | 0.60 | 0.60 |
| Carboxymethylcellulose | 0.50 | 0.50 | 0.50 |
| Sodium | | | |
| Polysorbate 80 | 1.00 | 0.00 | 0.00 |
| Dimethicone copolyol | 1.00 | 0.00 | 0.00 |
| Cyclomethicone | 0.00 | 1.00 | 0.00 |
| Purified water | 72.30 | 63.80 | 69.80 |
| Glycerin | 5.00 | 5.00 | 5.00 |
| LCD | 10.00 | 10.00 | 10.00 |
| Peppermint oil | 1.00 | 1.00 | 1.00 |
| Hydrocortisone | 0.10 | 0.10 | 0.10 |
| Total | 100.00 | 100.00 | 100.00 |

| | |
|---|---|
| NaOH (18% Solution) | to pH 6.0 |

Results of testing

| | | | |
|---|---|---|---|
| Appearance: | | | |
| Quality | E | E | E |
| Color | Yellow | Yellow | Yellow |
| Odor | Faint | Faint | Faint |
| Collapse time, sec | >120 | >120 | >120 |
| Density, g/mL | 0.037 | 0.039 | 0.036 |
| Centrifugation 3K rpm × 10 min | Stable* | Stable* | Stable* |
| Centrifugation 10K rpm × 10 min | 90 | 95 | 95 |

*Stable = no phase separation or creaming wereobserved.

6(b). Matrix with Hypromellose and Xanthan gum

| USAN name | W/W % | W/W % | W/W % |
|---|---|---|---|
| Mineral oil, heavy | 10.00 | 10.00 | 0.00 |
| PPG-15 Stearyl ether | 3.00 | 0.00 | 0.00 |
| Caprylic/Capric triglyceride | 0.00 | 0.00 | 5.00 |
| Isopropyl myristate | 0.00 | 0.00 | 5.00 |
| Lanolin | 0.00 | 2.00 | 0.00 |
| Cetearyl alcohol | 0.00 | 1.00 | 0.00 |
| Ceteareth-20 | 0.00 | 0.00 | 3.00 |
| Stearic acid | 1.00 | 0.00 | 1.00 |
| Polyoxyl 40 stearate | 2.50 | 2.50 | 0.00 |
| Dimethicone 350 | 1.00 | 0.00 | 0.00 |
| Benzyl alcohol | 0.60 | 0.60 | 0.60 |
| Hypromellose K100M | 0.25 | 0.25 | 0.25 |
| Xanthan gum | 0.25 | 0.25 | 0.25 |
| Polysorbate 80 | 1.00 | 1.00 | 0.00 |
| Dimethicone copolyol | 1.00 | 0.00 | 1.00 |
| Cyclomethicone | 0.00 | 1.00 | 0.00 |
| Purified water | 63.30 | 65.30 | 67.80 |
| Glycerin | 5.00 | 5.00 | 5.00 |
| LCD | 10.00 | 10.00 | 10.00 |
| Peppermint oil | 1.00 | 1.00 | 1.00 |
| Hydrocortisone | 0.10 | 0.10 | 0.10 |
| Total | 100.00 | 100.00 | 100.00 |

| | |
|---|---|
| NaOH (18% Solution) | to pH 6.0 |

Results of testing

| | | | |
|---|---|---|---|
| rance: | | | |
| Quality | E | E | E |
| Color | Yellow | Yellow | Yellow |
| Odor | Faint | Faint | Faint |
| Collapse time, sec | >120 | >120 | >120 |
| Density, g/mL | 0.038 | 0.044 | 0.036 |
| Centrifugation 3K RPM 10 min | Stable* | Stable* | Stable* |
| 10K rpm × 10 min, % of creaming | 30 | 20 | 20 |

*Stable = no phase separation or creaming were observed.

Comments: Foams comprising LCD in combination with Hydrocortisone were prepared with exemplary ingredients, mineral oil, PPG-15 stearyl ether, capric/caprylic triglyceride, octyl dodecanol and isopropyl myristate, stabilized with ceteareth 20, or PEG-40 stearate, and polysorbate 80 and co-stabilized with glyceryl monostearate, stearic acid, and stabilizing resilient polymers, methylcellulose, xanthan gum or sodium carboxymethyl cellulose, vaseline and lanoline, glycerin and silicone as skin feeling modifiers, and benzyl alcohol as microbial preservative.

All the foamable compositions produced excellent foams with a reasonable collapse time during which the expanded foam remains substantially intact. Both of the resilient agents a) methylcellulose and Xanthan gum or b) Sodium Carboxymethyl cellulose were successfully applied to produce a pseudoplastic or semi-pseudoplastic formulation which was resistant to creaming/phase separation when subjected to both significant centrifugal force. No separation of oily phase ingredients and no creaming were observed following centrifugation at 3000 rpm. It can be extrapolated that compositions, which can withstand such a g-force are probably capable of remaining physically stable at room temperature during the expected two years shelf life of a pharmaceutical product or for a lesser reasonable period. The three compositions comprising sodium carboxymethyl cellulose were also substantially resilient to harsh centrifugation at 10,000 rpm. It may be possible to further increase the resilience of the compositions for example by raising the level of the resilient agents.

### Example 7

### A matrix of Compositions Comprising Fluocinonide and LCD as Active Agents, which are Resilient to Creaming

7(a) Matrix with Carboxymethylcellulose sodium

| USAN name | W/W % | W/W % | W/W % |
|---|---|---|---|
| Mineral oil, heavy | 0.00 | 0.00 | 5.00 |
| PPG-15 Stearyl ether | 0.00 | 3.00 | 3.00 |
| Petrolatum, white | 1.25 | 2.50 | 0.00 |
| Lanolin | 0.00 | 0.00 | 0.00 |
| Octyl dodecanol | 3.00 | 6.00 | 0.00 |
| Cetearyl octanoate | 0.75 | 1.50 | 0.00 |
| Glyceryl monostearate | 0.00 | 1.00 | 0.00 |
| Cetearyl alcohol | 1.00 | 1.00 | 1.00 |
| Ceteareth-20 | 0.00 | 3.00 | 3.00 |
| Polyoxyl 40 stearate | 2.50 | 0.00 | 0.00 |
| Dimethicone 350 | 0.00 | 0.00 | 1.00 |
| Benzyl alcohol | 0.60 | 0.60 | 0.60 |
| Carboxymethylcellulose | 0.50 | 0.50 | 0.50 |
| Sodium | | | |
| Polysorbate 80 | 1.00 | 0.00 | 0.00 |
| Dimethicone copolyol | 1.00 | 0.00 | 0.00 |
| Cyclomethicone | 0.00 | 1.00 | 0.00 |
| Purified water | 72.30 | 63.80 | 69.80 |
| Glycerin | 5.00 | 5.00 | 5.00 |
| LCD | 10.00 | 10.00 | 10.00 |
| Peppermint oil | 1.00 | 1.00 | 1.00 |
| Fluocinonide | 0.10 | 0.10 | 0.10 |
| Total | 100.00 | 100.00 | 100.00 |

| | |
|---|---|
| NaOH (18% Solution) | to pH 6.0 |

Results of testing

| | | | |
|---|---|---|---|
| Appearance: | | | |
| Quality | E | E | E |
| Color | Yellow | Yellow | Yellow |
| Odor | Faint | Faint | Faint |
| Collapse time, sec | >120 | >120 | >120 |
| Density, g/Ml | 0.037 | 0.039 | 0.036 |
| Centrifugation 3K rpm × 10 min | Stable* | Stable* | Stable* |
| Centrifugation 10K rpm × 10 min | 90 | 95 | 95 |

E = excellent;
*Stable = no phase separation or creaming was observed.

7(2) Matrix with Hypromellose and Xanthan gum

| USAN name | W/W % | W/W % | W/W % |
|---|---|---|---|
| Mineral oil, heavy | 10.00 | 10.00 | 0.00 |
| PPG-15 Stearyl ether | 3.00 | 0.00 | 0.00 |
| Caprylic/Capric triglyceride | 0.00 | 0.00 | 5.00 |
| Isopropyl myristate | 0.00 | 0.00 | 5.00 |
| Lanolin | 0.00 | 2.00 | 0.00 |
| Cetearyl alcohol | 0.00 | 1.00 | 0.00 |
| Ceteareth-20 | 0.00 | 0.00 | 3.00 |
| Stearic acid | 1.00 | 0.00 | 1.00 |
| Polyoxyl 40 stearate | 2.50 | 2.50 | 0.00 |
| Dimethicone 350 | 1.00 | 0.00 | 0.00 |
| Benzyl alcohol | 0.60 | 0.60 | 0.60 |
| Hypromellose K100M | 0.25 | 0.25 | 0.25 |
| Xanthan gum | 0.25 | 0.25 | 0.25 |
| Polysorbate 80 | 1.00 | 1.00 | 0.00 |
| Dimethicone copolyol | 1.00 | 0.00 | 1.00 |
| Cyclomethicone | 0.00 | 1.00 | 0.00 |
| Purified water | 63.30 | 65.30 | 67.80 |
| Glycerin | 5.00 | 5.00 | 5.00 |
| LCD | 10.00 | 10.00 | 10.00 |
| Peppermint oil | 1.00 | 1.00 | 1.00 |
| Fluocinonide | 0.10 | 0.10 | 0.10 |
| Total | 100.00 | 100.00 | 100.00 |

| | |
|---|---|
| NaOH (18% Solution) | to pH 6.0 |

Results of testing

| | | | |
|---|---|---|---|
| Appearance: | | | |
| Quality | E | E | E |
| Color | Yellow | Yellow | Yellow |
| Odor | Faint | Faint | Faint |
| Collapse time, sec | >120 | >120 | >120 |
| Density, g/mL | 0.038 | 0.044 | 0.036 |
| Centrifugation 3K RPM 10 min | Stable* | Stable* | Stable* |
| 10K rpm × 10 min, % of creaming | 30 | 20 | 20 |

E = excellent;
*Stable = no phase separation or creaming were observed.

Comments: Foams comprising LCD in combination with flucocinide were prepared with exemplatory ingredients, mineral oil, PPG-15 stearyl ether, capric/caprylic triglyceride, octyl dodecanol and isopropyl myristate, stabilized with ceteareth 20, or PEG-40 stearate, and polysorbate 80 and co-stabilized with glyceryl monostearate, stearic acid, and stabilizing resilient polymers, methylcellulose, xanthan gum or sodium carboxymethyl cellulose, vaseline and lanoline, glycerin and silicone as skin feeling modifiers, and benzyl alcohol as microbial preservative.

All the foamable compositions produced excellent foams with a reasonable collapse time of over two minutes during which the expanded foam remains substantially intact. Both of the resilient agents a) methylcellulose and Xanthan gum or b) sodium carboxymethyl cellulose were successfully applied to produce a pseudoplastic or semi-pseudoplastic formulation which was resistant to creaming/phase separation when subjected to a significant centrifugal force. No separation of oily phase ingredients and no creaming were observed following centrifugation at 3000 rpm. It can be extrapolated that compositions, which can withstand such a g-force are probably capable of remaining physically stable at room temperature during the expected two years shelf life of a pharmaceutical product or for a lesser reasonable period. The three compositions comprising sodium carboxymethyl cellulose were also resilient to harsh centrifugation at 10,000 rpm.

### Example 8

### Formulation of Betamethasone Valerate which is Physically Stable and in which the Active Pharmaceutical Ingredient is also Stable Chemically for a) Two Years at Room Temperature and b) Six Months at 40° C.

| Ingredient | % w/w |
|---|---|
| Isopropyl myristate | 10.00 |
| Octyl dodecanol | 12.00 |
| Stearyl alcohol | 1.00 |
| Glyceryl stearate | 1.00 |
| PEG-40 stearate | |
| Hypromellose | 0.28 |
| Xanthan gum | 0.28 |
| Polysorbate 60 | 1.00 |
| Water, purified | 68.314 |
| Mixture of Propyl, Ethyl and Methyl | 1.00 |
| Paraben in | |
| 2-phenoxyethanol | |
| Citric acid | 1.30 |
| Sodium citrate | 1.10 |
| Betamethasone Valerate | 0.126 |
| Total: | 100.00 |
| Citric acid or/and sodium citrate to pH = 4.0-4.4 | |
| Propellant (butane, isobutane, propane) | 8.00 |
| Foam quality | Excellent |
| Color | white |
| Odor | no odor |
| Density | 0.034 |
| Collapse time | >300 |
| Hardness | 15.3 |
| Centrifugation 3,000 RPM | Stable |
| Centrifugation: 10,000 RPM | 30% |

Stability of BMV-formulation at 40° C.

| | Specification | | | | | |
|---|---|---|---|---|---|---|
| Test parameter | for Release | T-0 | T-1 | T-2 | T-3 | T-6 |
| API assay: | | | | | | |
| concentration(% | 0.120-0.132 | 0.128 | 0.125 | 0.123 | 0.124 | 0.120 |
| w/w) | | | | | | |
| % of label claim: | 100.0-110.0 | 106.7 | 104.0 | 102.5 | 103.3 | 100.0 |
| Assay of | NMT 2.5 | ND | 0.653 | 1.4 | 2.1 | 5.30 |
| degradation | | | | | | |
| product | | | | | | |
| (BM21V as % of | | | | | | |
| BM17V peak) | | | | | | |
| Appearance: | | | NA | NA | | |
| Quality | Good (G)- | E | | | E | E |
| | excellent (E) | | | | | |
| Color | White to off | White | | | White | White |
| | white | | | | | |
| Odor | Very faint to no | No Odor | | | Very faint | Very faint |
| | odor | | | | | odor |
| Density (g/mL) | 0.030 to 0.070 | 0.034 | NA | NA | 0.037 | 0.041 |
| Expansion time | <300 | 48 | NA | NA | 50 | 45 |
| (sec) | | | | | | |
| Collapse time (sec) | ≧120 | >300 | NA | NA | >300 | >300 |
| pH (diluted 1:5 in | 3.90-4.40 | 3.94 | NA | NA | 3.99 | 4.02 |
| water) | | | | | | |
| Texture, hardness | For Information | 15.3 | NA | NA | 16.1 | 16.5 |
| (g) | only | | | | | |

T = time in months,
NA = not applicable;
N/D: not determined.

Stability of BMV-formulation at 25° C.

| | Specification | | | |
|---|---|---|---|---|
| Test parameter | for Release | T-0 | T-6 | T-12 |
| API assay: | | | | |
| concentration(% | 0.120-0.132 | 0.128 | 0.126 | 0.125 |
| w/w) | | | | |
| % of label claim: | 100.0-110.0 | 106.7 | 105.8 | 104.2 |
| Assay of | | | | |
| degradation | | | | |
| product | | | | |
| (BM21V as % of | NMT 2.5 | ND | 0.46 | 0.74 |
| BM17V peak) | | | | |
| Content uniformity, | NMT 5.0 | 1.6 | NA | 3.8 |
| intra-canister (%): | | | | |
| Appearance: | | | | |
| Quality | Good (G)- | E | E | E |
| | excellent (E) | | | |
| Color | White to off | White | White | White |
| | white | | | |
| Odor | Very faint to | No odor | Very faint | Very faint |
| | no odor | | | odor |
| Density (g/mL) | 0.030 to 0.070 | 0.034 | 0.034 | 0.039 |
| Expansion time | <300 | 48 | 60 | 48 |
| (sec) | | | | |
| Collapse time (sec) | ≧120 | >300 | >300 | >300 |
| pH (diluted 1:5 in | 3.90-4.40 | 3.94 | 3.98 | 4.01 |
| water) | | | | |
| Texture, hardness | For | 15.3 | 14.4 | 15.0 |
| (g) | Information | | | |
| | only | | | |

Comments: The results show that betamethsone 17 valerate was stabile in the formulation with negligible degradation to betamethasone 21 valerate at room temperature and an also under accelerated conditions of 40C.

### Example 9

### Betamethasone Diproprionate Foamable Emulsion Compositions Betamethasone Dipropionate Matrix

| Chemical name | W/W % | W/W % |
|---|---|---|
| Isopropyl myristate | 2.00 | 12.00 |
| Glyceryl stearate | 1.00 | 1.00 |
| Stearyl alcohol | 1.00 | — |
| PEG-40 stearate | 2.50 | 2.50 |
| Xanthan gum | 0.26 | — |
| Hydroxypropyl methyl cellulose K100 | 0.26 | — |
| Carboxymethyl cellulose | — | 0.50 |
| Polysorbate 60 | 1.00 | — |
| Polysorbate 80 | — | 1.00 |
| Citric acid | 1.30 | 0.70 |
| Sodium citrate | 1.10 | 0.60 |
| Water | 87.53 | 81.65 |
| Betamethasone dipropionate | 0.05 | 0.05 |
| Total | 89.98 | 100.00 |
| Propellant | 8.00 | 8.00 |
| Results | | |
| Appearance: | | |
| Quality | Excellent | Excellent |
| Color | White | White |
| Odor | No odor | No odor |
| Collapse time | >120 | >120 |
| Hardness | 14.28 | 18.59 |
| Density | 0.024 | 0.035 |

Betamethasone diropionate was formulated in two exemplary composition, the first low oil concentration and the second medium oils concentration. In these examples, the major emollient oil is isopropyl myristate and the stabilizers are common surfactants and polymers. The foams are of excellent quality and withstand at least one cycle of stress freeze and thaw.

### Example 10

### PPG o/w Emulsion: with Fluocinonide in Combination with Clindamycin Phosphate

| Chemical name | W/W % | W/W % |
|---|---|---|
| PPG-15 stearyl ether | 6.00 | 15.00 |
| Octyldodecanol | 6.00 | |
| Brij721 | 1.60 | 1.60 |
| Laureth-4 | 2.20 | 2.20 |
| Ceteth-20 | 1.50 | 1.50 |
| Methyl cellulose | 0.25 | 0.25 |
| K100M | | |
| Xanthan gum | 0.25 | 0.25 |
| Water, purified | 76.90 | 73.90 |
| Benzyl alcohol | 1.00 | 1.00 |
| Propylene Glycol | 3.00 | 3.00 |
| Clindamycin Phosphate | 1.2 | 1.2 |
| Fluocinonide | 0.10 | 0.10 |
| Sodium Hydroxide | To pH 4.7 | To pH 4.7 |
| Solution 18% | | |
| Results | | |
| Appearance: | | |
| Quality | Excellent | Excellent |
| Color | White | White |
| Odor | No odor | No odor |
| Collapse time | >120 | >120 |
| Hardness | 12.51 | 12.46 |
| Density | 0.038 | 0.037 |
| Centrifugation 3K 10 min | Stable* | Stable* |
| Centrifugation 10K 10 min | 35% | 35% |
| | creaming | creaming |

*Stable = no phase separation or creaming were observed.

Comments: Fluocinonide in combination with clindamycin phosphate were formulated in two emollient oil-in-water emulsions, one based on octyl dodecanol in combination with PPG-15 stearyl ether and the other solely on PPG-15 sterayl ether. Both formulations produced foams of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 11

### Two Compositions Comprising Hydrocortisone and Clindamycin Phosphate (CDM) as Active Agents, which are Resilient to Creaming

| Chemical name | W/W % | W/W % |
|---|---|---|
| PPG-15 stearyl ether | 6.00 | 15.00 |
| Octyldodecanol | 6.00 | 0 |
| Brij721 | 1.60 | 1.60 |
| Laureth-4 | 2.20 | 2.20 |
| Ceteth-20 | 1.50 | 1.50 |
| Methyl cellulose | 0.25 | 0.25 |
| K100M | | |
| Xanthan gum | 0.25 | 0.25 |
| Water, purified | 76.00 | 73.00 |
| Benzyl alcohol | 1.00 | 1.00 |
| Propylene Glycol | 3.00 | 3.00 |
| Clindamycin Phosphate | 1.2 | 1.2 |
| Hydrocortisone | 1.00 | 1.00 |
| Sodium Hydroxide | To pH 4.7 | To pH 4.7 |
| Solution 18% | | |
| Results | | |
| Appearance: | | |
| Quality | Excellent | Excellent |
| Color | White | White |
| Odor | No odor | No odor |
| Collapse time | >120 | >120 |
| Hardness | 12.51 | 12.46 |
| Density | 0.038 | 0.037 |
| Centrifugation 3K 10 min | Stable* | Stable* |
| Centrifugation 10K 10 min | 35% | 35% |
| | creaming | creaming |

*Stable = no phase separation or creaming were observed.

Comments: Hydrocortisone in combination with clindamycin phosphate were formulated in two emollient oil-in-water emulsions, one based on octyl dodecanol in combination with PPG-15 stearyl ether and the other solely on PPG-15 sterayl ether. Both formulations produced foams of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 12

### A matrix of Compositions Comprising Hydrocortisone and Azelaic Acid (AZL) as Active Agents, which are Resilient to Creaming

| Chemical name | W/W % | W/W % | W/W % | W/W % |
|---|---|---|---|---|
| Caprylic/Capric | 5.00 | 10.00 | 10.00 | 10.00 |
| Triglyceride | | | | |
| Cetearyl alcohol | 0.90 | 1.00 | 1.00 | 1.00 |
| Glyceryl stearate | 0.45 | 0.50 | 0.50 | 0.50 |
| Cholesterol | 1.00 | 1.00 | | |
| Benzyl alcohol | 1.00 | | | 1.00 |
| Benzoic acid | | 0.20 | 0.20 | |
| Butylated | 0.10 | 0.10 | 0.10 | 0.10 |
| hydroxytoluene | | | | |
| PEG-40 Stearate | 2.60 | 2.60 | 2.60 | 2.60 |
| Methylcellulose | 0.10 | | 0.10 | 0.10 |
| K100M | | | | |
| Methylcellulose A4M | | 0.10 | | |
| Xanthan gum | 0.25 | 0.25 | 0.25 | 0.25 |
| Polysorbate 80 | 0.90 | 0.90 | 0.90 | 0.90 |
| EDTA disodium | 0.10 | | | |
| Sodium Hydroxide | | | | 0.50 |
| Water purified | 55.60 | 57.35 | 53.35 | 67.05 |
| Dimethyl isosorbode | 10.00 | | 5.00 | |
| Propylene glycol | 6.00 | 5.00 | 10.00 | |
| PEG-400 | | 5.00 | | |
| Azelaic Acid | 15.00 | 15.00 | 15.00 | 15.00 |
| Hydrocortisone | 1.00 | 1.00 | 1.00 | 1.00 |
| Total | 100 | 100 | 100 | 100 |
| Results | | | | |
| Appearance: | | | | |
| Quality | E | E | E | E |
| Color | O.W | O.W | O.W | O.W |
| Odor | V.F.O | V.F.O | V.F.O | V.F.O |
| Collapse time | >120 | >120 | >120 | >120 |
| Hardness | 22.3 | 21.3 | 39.0 | 14.5 |
| Density | 0.038 | 0.030 | 0.032 | 0.044 |
| Centrifugation 3K | Stable* | Stable* | Stable* | Stable* |
| 10 min | | | | |
| Centrifugation 10K | 10% | 40% | 30% | 20% |
| 10 min | Creaming | Creaming | Creaming | Creaming |

*Stable = no phase separation or creaming were observed; E = excellent; OW = off white; and V.F.O = very faint odor.

Comments: It is very difficult to stabilize such compositions since azaleic acid is a solid and significant quantities are required. Hydrocortisone, in combination with azaleic acid, were successfully formulated in oil-in-water emulsions. The formulations produced foams of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period. These compositions were rugged and resilient to harsh centrifugation of 10,000 rpm.

### Example 13

### A matrix of Compositions Comprising Fluocinonide and Azelaic Acid (AZL) as Active Agents, which are Resilient to Creaming

| | W/W % | W/W % | W/W % | W/W % |
|---|---|---|---|---|
| Chemical name | | | | |
| Caprylic/Capric | 5.00 | 10.00 | 10.00 | 10.00 |
| Triglyceride | | | | |
| Cetearyl alcohol | 0.90 | 1.00 | 1.00 | 1.00 |
| Gleceryl stearate | 0.45 | 0.50 | 0.50 | 0.50 |
| Cholesterol | 1.00 | 1.00 | | |
| Benzyl alcohol | 1.00 | | | 1.00 |
| Benzoic acid | | 0.20 | 0.20 | |
| Butylated | 0.10 | 0.10 | 0.10 | 0.10 |
| hydroxytoluene | | | | |
| PEG-40 Stearate | 2.60 | 2.60 | 2.60 | 2.60 |
| Methylcellulose K100M | 0.10 | | 0.10 | 0.10 |
| Methylcellulose A4M | | 0.10 | | |
| Xanthan gum | 0.25 | 0.25 | 0.25 | 0.25 |
| Polysorbate 80 | 0.90 | 0.90 | 0.90 | 0.90 |
| EDTA disodium | 0.10 | | | |
| Sodium Hydroxide | | | | 0.50 |
| Water purified | 56.50 | 58.25 | 54.25 | 67.95 |
| Dimethyl isosorbode | 10.00 | | 5.00 | |
| Propylene glycol | 6.00 | 5.00 | 10.00 | |
| PEG-400 | | 5.00 | | |
| Azelaic Acid | 15.00 | 15.00 | 15.00 | 15.00 |
| Fluocinonide | 0.10 | 0.10 | 0.10 | 0.10 |
| Total | 100 | 100 | 100 | 100 |
| Results | A | B | C | D |
| Appearance: | | | | |
| Quality | E | E | E | E |
| Color | Off white | Off white | Off white | Off white |
| Odor | V.F.O* | V.F.O* | V.F.O* | V.F.O* |
| Collapse time | >120 | >120 | >120 | >120 |
| Hardness | 22.3 | 21.3 | 39.0 | 14.5 |
| Density | 0.038 | 0.030 | 0.032 | 0.044 |
| Centrifugation 3K | Stable** | Stable** | Stable** | Stable** |
| 10 min | | | | |
| Centrifugation 10K | 10% | 40% | 30% | 20% |
| 10 min | Creaming | Creaming | Creaming | Creaming |

*Stable = no phase separation or creaming; E = excellent; V.F.O = very faint odor.

Comments: fluocinonide in combination with azelaic acid were formulated in oil-in-water emulsions based on caprylic/capric triglyceride. The 15% azelaic acid suspension with fluocinonide shows minimal settling and azelaic acid particles in the emollient emulsion are readily re-suspendable upon slight hand shaking. The formulations produced foams of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 14

### A Composition Comprising Hydrocortisone Butyrate, which is Resilient to Creaming

| | W/W % |
|---|---|
| Chemical name | |
| Mineral oil | 12.00 |
| Isopropyl myristate | 12.00 |
| Dimeticone V100 | 3.00 |
| Glyceryl monostearate | 0.50 |
| MYRJ 52 | 3.00 |
| Microcrystalline cellulose + | 2.00 |
| carboxymethyl cellulose) | |
| TWEEN 80 | 1.00 |
| Cocoamidopropyl betaine | 0.50 |
| Citric acid buffer | 1.0 |
| Preservative | 0.30 |
| Purified water | 64.60 |
| Hydrocortisone butyrate | 0.10 |
| Total | 100.00 |
| Propellant | 8.00 |

results

| | |
|---|---|
| Appearance: | |
| Quality | Excellent |
| Color | White |
| Odor | No odor |
| Collapse time | >120 |
| Hardness | 16.87 |
| Density | 0.038 |
| Centrifugation 3K 10 min | Stable* |

*Stable = no phase separation or creaming were observed.

Hydrocortisone butyrate was formulated in an emollient oil-in-water emulsion with silicone to provide an enhanced silky skin feeling. The formulation produced a foam of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 15

### A Composition Comprising Fluocinolone Acetonide, which is Resilient to Creaming

| | W/W % |
|---|---|
| Chemical name | |
| Mineral oil | 12.00 |
| Glyceryl stearate | 1.00 |
| PEG-40 stearate | 2.50 |
| Carboxymethyl cellulose | 0.50 |
| Polysorbate 80 | 1.00 |
| Citric acid buffer | 1.0 |
| Water | 81.99 |
| Fluocinolone acetonide | 0.01 |
| Total | 100.00 |
| Propellant | 8.00 |

Results

| | |
|---|---|
| Appearance: | |
| Quality | Excellent |
| Color | White |
| Odor | No odor |
| Collapse time | >120 |
| Hardness | 14.99 |
| Density | 0.035 |
| Centrifugation 3K 10 min | Stable* |
| pH | 4.2-4.4 |

*Stable = no phase separation or creaming

Comments: Fluocinolone acetonide was stabilized at pH 4.5 by the aid of a citrate buffer in a mineral oil in water foamable emollient emulsion stabilized with a complex emulgator PEG-40 stearate and between 80 and with co-surfactant glyceryl stearate and with carboxymethyl cellulose. The formulation produced a foam of excellent quality with beneficial physical properties such as desired hardness, a reasonable collapse time of over two minutes during which the expanded foam remains substantially intact, and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 16

### Two Compositions Comprising Clobetasol-17-Propionate as Active Agents, which is Resilient to Creaming

| | W/W % | W/W % |
|---|---|---|
| Chemical name | | |
| Mineral oil | 2.00 | 12.00 |
| Isopropyl myristate | 2.00 | — |
| Glyceryl stearate | 1.00 | 1.00 |
| Stearyl alcohol | 1.00 | — |
| PEG-40 stearate | 2.50 | 2.50 |
| Xanthan gum | 0.26 | — |
| Methocel K100 | 0.26 | — |
| Carboxymethyl cellulose | — | 0.50 |
| Polysorbate 60 | 1.00 | — |
| Polysorbate 80 | — | 1.00 |
| Citric acid | 1.30 | 0.7 |
| Sodium citrate | 1.10 | 0.6 |
| Purified Water | 87.53 | 81.65 |
| Clobetasol-17-propionate | 0.05 | 0.05 |
| Total | 100.00 | 100.00 |
| Propellant | 8.00 | 8.00 |

Results

| | | |
|---|---|---|
| Appearance: | | |
| Quality | Excellent | Excellent |
| Color | White | White |
| Odor | No odor | No odor |
| Collapse time | >120 | >120 |
| Hardness | 22.2 | 18.44 |
| Density | 0.038 | 0.042 |
| Centrifugation 3k 10 min | stable* | stable* |

*Stable = no phase separation or creaming; N/M = not measured.

Clobetasole proprionate was formulated with low and medium oil content and stabilized to a desired pH with the aid of a citric buffer. Both formulations produced a foam of excellent quality and resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period. It is observed that the variations in the types of polysorbate, polymers and oils and in the citric acid concentration, do not appear to affect the physical stability.

### Example 17

### Hydrocortisone and C₁₋Clopiroxolamine (CPO) with and without Various Oils including Silicone and Different Excipient Options

17(a) Matrix with three different oils.

| | W/W % | W/W % | W/W % |
|---|---|---|---|
| USAN name | | | |
| Mineral oil, light | 5.00 | 0.00 | 0.00 |
| Caprylic/Capric triglyceride | 0.00 | 10.00 | 0.00 |
| Isopropyl myristate | 5.00 | 0.00 | 0.00 |
| Octyldodecanol | 0.00 | 0.00 | 10.00 |
| Cyclomethicone | 2.00 | 0.00 | 0.00 |
| Glyceryl stearate | 0.45 | 0.45 | 0.45 |
| Stearyl alcohol | 0.85 | 0.85 | 0.85 |
| Sorbitan stearate | 0.60 | 0.60 | 0.60 |
| Steareth-21 | 0.00 | 0.00 | 2.00 |
| Laureth-4 | 2.00 | 2.00 | 0.00 |
| PEG-40 Stearate | 2.60 | 2.60 | 2.60 |
| Benzyl Alcohol | 2.00 | 2.00 | 0.00 |
| Methocel A15C | 0.30 | 0.30 | 0.30 |
| Xanthan gum | 0.26 | 0.26 | 0.26 |
| Polysorbate 80 | 0.90 | 0.90 | 0.90 |
| Glycerin | 0.00 | 0.00 | 0.00 |
| Water, purified | 76.04 | 78.04 | 80.04 |
| Ciclopiroxolamine | 1.00 | 1.00 | 1.00 |
| Hydrocortisone | 1.00 | 1.00 | 1.00 |
| Total | 100.00 | 100.00 | 100.00 |
| Lactic acid | to pH 7.0 | to pH 7.0 | To pH 7.0 |
| Propellant (Propane/Butane/ | 8.0 | 8.0 | 1.0 |
| Isobutane) | | | |
| Dymel 134 ® | — | — | 7.0 |

Results

| | | | |
|---|---|---|---|
| Appearance: | | | |
| Quality | Excellent | Excellent | Good |
| Color | White | White | White |
| Odor | None | None | None |
| Centrifugation 3,000 RPM 10 min | 100 | 100 | N/M |
| Centrifugation 10,000 RPM 10 min | 25 | 40 | N/M |
| Hardness (g) | 12 | 13 | N/M |
| Collapse time at 36° C. (s) | 120 | >300 | >300 |

18(b) Matrix without oils

| | W/W % | W/W % | W/W % |
|---|---|---|---|
| USAN name | | | |
| PPG-15 Stearyl ether | 0.00 | 15.00 | 0.00 |
| Glyceryl stearate | 0.50 | 0.00 | 0.50 |
| Stearyl alcohol | 0.92 | 0.00 | 0.92 |
| Behenyl alcohol | 0.00 | 1.00 | 0.00 |
| Sorbitan stearate | 0.65 | 0.00 | 0.65 |
| Ceteth-2 | 0.00 | 2.00 | 0.00 |
| Ceteareth-20 | 0.00 | 1.60 | 0.00 |
| Steareth-21 | 2.20 | 0.00 | 2.20 |
| PEG-40 Stearate | 2.85 | 0.00 | 2.85 |
| Methocel A4M | 0.00 | 0.16 | 0.00 |
| Methocel A15C | 0.35 | 0.00 | 0.35 |
| Xanthan gum | 0.30 | 0.00 | 0.30 |
| Pemulen TR2 | 0.00 | 0.05 | 0.00 |
| Polysorbate 80 | 1.00 | 0.00 | 1.00 |
| Glycerin | 0.00 | 3.00 | 0.00 |
| Dimethyl isosorbide | 0.00 | 0.00 | 10.00 |
| Propylene glycol | 0.00 | 5.40 | 0.00 |
| Water, purified | 89.23 | 69.79 | 79.23 |
| Ciclopiroxolamine | 1.00 | 1.00 | 1.00 |
| Hydrocortisone | 1.00 | 1.00 | 1.00 |
| Total | 100.00 | 100.00 | 100.00 |
| Lactic acid | to pH | to pH | to pH |
| | 7.5 ± 0.2 | 7.5 ± 0.2 | 7.5 ± 0.2 |
| Propellant (Propane/Butane/ | 8.0 | 8.0 | 8.0 |
| Isobutane) | | | |

Results

| | | | |
|---|---|---|---|
| Appearance: | | | |
| Ouality | Excellent | Excellent | Excellent |
| Color | White | White | White |
| Odor | None | None | None |
| Creaming (centrifugation) 3,000 rpm | 70 | 30 | N/M |
| (%) | | | |
| Creaming (centrifugation) 10,000 | 70 | 50 | N/M |
| rpm (%) | | | |
| Hardness (g) | N/M | 11 | N/M |
| Collapse time at 36° C. (s) | 120 | >300 | N/M |

Hydrocortisone was formulated with ciclopiroxolamine (CPO) with and without various oils including silicone and different excipient options. All foams were of excellent quality.

A different propellant type, perfluorocarbon (Dymel 134®) was tested and also found to be appropriate for propelling the steroid foamable formulation.

Resilience to creaming was significantly reduced at 3000 rpm in the absence of oils. Increasing the centrifugal force in the absence of oils did not substantially change the resilence.

### Example 19

### Example 19(a)

### Foamable Pharmaceutical Emulsion Compositions with 25% Petrolatum as Unctuous Emollient, Steareth-2/Steareth-21/ASOS, and Betamethasone 17 Valerate Micronized, as API

| | Betamethasone |
|---|---|
| | 17 |
| | valerate |
| | micronized |
| Water | 63.43 |
| Carbomer 941 | 0.12 |
| sodium Lauryl sulfate | 0.10 |
| Disodium EDTA | 0.05 |
| Methylparaben | 0.20 |
| Petrolatum | 25.00 |
| Stearath-2 | 1.73 |
| Steareth-20 | 0.58 |
| Propylparaben | 0.15 |
| Cetyl alcohol | 0.10 |
| Cyclomethicone | 5.00 |
| Aluminum starch | 3.00 |
| octenylsuccinate | |
| Imidazolidinyl urea | 0.30 |
| Triethanolamine | 0.12 |
| API | 0.12 |
| Total | 100.00 |
| Propellant | 8.00 |
| (Propane/Butane/Isobutane) | |
| FOAM QUALITY | Excellent |
| ODOR | White |
| COLOR | V.F.O |
| SHAKABILITY | GOOD |
| HARDNESS | 9.77 |
| COLLAPSE TIME AT | 180 SEC. |
| 36° C. | |

### Example 19(b)

### Foamable Pharmaceutical Emulsion Compositions with 12.5% Petrolatum as Unctuous Emollient, Steareth-2/Steareth-21/ASOS and Betamethasone 17 Valerate Micronized, as API

| | Betamethasone 17 |
|---|---|
| | valerate |
| | micronized |
| Water | 75.93 |
| Carbomer 941 | 0.12 |
| sodium Lauryl | 0.1 |
| sulfate | |
| Disodium EDTA | 0.05 |
| Methylparaben | 0.2 |
| Petrolatum | 12.5 |
| Stearath-2 | 1.73 |
| Steareth-20 | 0.58 |
| Propylparaben | 0.15 |
| Cetyl alcohol | 0.1 |
| Cyclomethicone | 5 |
| Aluminum starch | 3 |
| octenylsuccinate | |
| Imidazolidinyl urea | 0.3 |
| Triethanolamine | 0.12 |
| API | 0.12 |
| Total | 100 |
| Propellant | 8 |
| (Propane/Butane/Isobutane) | |
| FOAM QUALITY | Excellent |
| ODOR | White |
| COLOR | V.F.O |
| SHAKABILITY | GOOD |
| HARDNESS | 10.47 |
| COLLAPSE TIME | >300 SEC |
| AT 36° C. | |

Comments: Excellent steroid foams with good physical properties and an elegant skin feeling were produced using petrolatum as unctuous emollient.

### Example 20

### Estradiol Emulsion Foamable Compositions for Metered Dose

| | A W/W % | B W/W % | C W/W % |
|---|---|---|---|
| Ingredient | | | |
| Liquid Paraffin | 10.1 | 10.2 | |
| Octyldodecanol | | | 10.0 |
| Peg-100 stearate | 2.0 | 2.0 | 2.0 |
| Glyceryl monostearate | | | 2.0 |
| Polysorbate 60 | 0.6 | 0.6 | |
| Sorbitan Monostearate | 0.4 | 0.4 | |
| Hydroxypropyl | 0.3 | | |
| methyl cellulose | | | |
| Methyl cellulose | | | 0.3 |
| Carbomer 934P | 0.2 | 0.3 | |
| Triethanol amine | 0.2 | 0.3 | |
| Hydroxyethyl cellulose | | | 0.3 |
| Cetearyl alcohol | 1.0 | 1.0 | |
| Propylene Glycol | 3.0 | 3.0 | 3 |
| Propyl 4-hydroxybenzoate | 0.1 | 0.1 | 0.1 |
| Methyl 4-hydroxybenzoate | 0.2 | 0.2 | 0.2 |
| water purified | 81.9 | 81.9 | 81.5 |
| Sodium citrate | | | 0.2 |
| Citric Acid | | | 0.4 |
| Estradiol hemihydrate | 0.0051 | 0.0051 | 0.0051 |
| Propellant | 8.0 | 8.0 | 8 |
| Results | | | |
| Centrifugation 3,000 rpm 10 min | Stable* | Stable* | 90% |
| | | | creaming |
| Centrifugation 10,000 rpm | 70% creaming | 90% | 30% |
| 10 min | | creaming | creaming |
| Appearance | Good | Good | Good |
| Color | White | White | White |
| Odor | Very faint odor | no odor | no odor |
| Density (g) | 0.056 | 0.086 | 0.057 |
| Hardness (g) | 14.23 | 23.782 | 5.86 |
| Collapse time (sec) | >300 | >300 | >300 |
| Ph | 5.92 | 5.45 | 3.55 |

*Stable = no phase separation or creaming

Comments: Estradiol foam designed to deliver a dose of 25 micrograms of Estradiol in each dose of 0.5 ml of PFF. The foamable composition was packaged in pressurized aluminum canisters fitted with metered dose valves of various dosage volumes: 100, 200, 500 and 1,000 microlitters. The Estradiol PFF was filled and pressurized using metered dose valves of the “dose retention chamber” type and “rapid fill/drain” type. The metered dose valves or metering valve are available commercially from Valois pharmaceutical division, France, Lablabo, France and Seaquist-perfect, France or USA.

The formulations produced foams of good quality with beneficial physical properties such as desired hardness, a stable collapse time of over five minutes during which the expanded foam remains substantially intact, and in the presence of liquid paraffin a resistance to creaming/phase separation when subjected to centrifugation at 3000 rpm. It can be extrapolated that compositions, which can withstand such a g-force are probably capable of remaining physically stable at room temperature during the expected two years shelf life of a pharmaceutical product or for a lesser reasonable period.

### Example 21

### Progesterone

Progesterone formulation

| | W/W % |
|---|---|
| Chemical name | |
| Mineral Oil | 8.0 |
| Carbomer 934P | 0.3 |
| Cetearyl Alcohol | 1.0 |
| Sorbitan Monostearate | 0.4 |
| Hypromellose | 0.3 |
| Polysorbate 60 | 0.6 |
| Tri ethanol amine | 0.2 |
| Water purified | 79.4 |
| Propyl 4-hydroxybenzoate | 0.1 |
| Methyl 4-hydroxybenzoate | 0.2 |
| Propylene Glycol | 3.0 |
| PEG-100 Stearate | 2.0 |
| Progesterone | 4.0 |
| Control | 100 |
| Propellant | 8.0 |
| Results | |
| Centrifugation 3,000 rpm, 10 min | Stable* |
| Centrifugation 10,000 rpm, 10 min | 70% creaming |
| Appearance | Good |
| Color | White |
| Odor | Very faint |
| | odor |

Stable* = no phase separation or creaming

Comments: Note the composition can be delivered for example as a metered dose of say a 4.0% vaginal application with each dose being of (1) one ml comprising 40 mg progesterone.

### Example 22

### Prophetic Example of Corticosteroid Foam Compositions For Mucosal Or Body Cavity Application

| | % w/w | % w/w | % w/w | % w/w | % w/w |
|---|---|---|---|---|---|
| Mineral oil | 5.60 | 5.60 | 5.60 | 5.60 | 5.60 |
| Isopropyl myristate | 5.60 | 5.60 | 5.60 | 5.60 | 5.60 |
| Glyceryl monostearate | 0.45 | 0.45 | 1.00 | 1.00 | 1.00 |
| Stearyl alcohol | 0.85 | 0.85 | 0.85 | 0.85 | 0.85 |
| Myrj 52 | 2.60 | 2.60 | 2.60 | 2.60 | 2.60 |
| Xanthan gum | 0.26 | — | — | — | — |
| Methocel K100M | 0.26 | — | — | — | — |
| Chitosan | — | — | 1.00 | — | — |
| Hyaluronic acid | — | — | — | 0.50 | 0.50 |
| Avicel CL611 | — | 2.00 | 2.00 | 2.00 | 2.00 |
| TWEEN 80 | 0.90 | 0.90 | 0.90 | 0.90 | 0.90 |
| Cocoamidopropyl betaine | 0.41 | 0.41 | 0.41 | 0.41 | 0.41 |
| Betametasone valerate | 0.12 | — | 0.12 | 0.12 | — |
| Hydrocortisone butyrate | — | 0.10 | — | — | 0.10 |
| Propylene glycol | 3.00 | 3.00 | 3.00 | 3.00 | 3.00 |
| Parabens (phenoxy | 0.8 | 0.8 | 0.8 | 0.8 | 0.8 |
| ethanol and methyl, ethyl | | | | | |
| and propyl hydroxy | | | | | |
| benzoate mixture) | | | | | |
| Propellant | 12.00 | 12.00 | 12.00 | 12.00 | 12.00 |
| Propane/butane* | | | | | |
| Water | To 100 | To 100 | To 100 | To 100 | To 100 |

The liquefied or gas propellant can be added at a concentration of about 3% to about 25%. The foams of this proposed example have a non-ionic surfactant to ionic surfactant ratio ranging from about 20:1 to about 14:1. Total surface active agent, foam adjuvant and polymeric agent ranges from about 2% to about 3.5% (w/w).

### Example 23

### Prophetic Example of Composition Comprising a Corticosteroid for Mucosal or Body Cavity Application

| Ingredient | % w/w |
|---|---|
| Betamethasone valerate | 0.1 |
| Caprylic/Capric Triglycerides | 60.9 |
| Propylene glycol | 10.0 |
| Potent solvent | — |
| Lecithin | 10.0 |
| Stearyl alcohol | 5.0 |
| Glyceryl monostearate | 2.0 |
| PVP K90 | 2.0 |
| Preservative | 0.3 |
| Propellant | 10.0 |
| Propane/butane* | |
| Purified water** | T0 100 |

The liquefied or gas propellant can be added at a concentration of about 3% to about 25%. Water content is about 10%

### Example 24

### Calcipitriol Foamable Compositions, Physical and Chemical Stability

a) Formulations

| | Formulation: | 1 | 2 | 3 | 4 | 5 | 6 |
|---|---|---|---|---|---|---|---|
| Phase | Ingredient | % w/w | % w/w | % w/w | % w/w | % w/w | % w/w |
| API Solution | Calcipotriol | 0.005 | 0.005 | 0.005 | 0.005 | 0.005 | 0.005 |
| | hydrate | | | | | | |
| | Propylene glycol | 5.00 | — | — | — | — | 5.00 |
| | PEG-400 | — | 5.00 | 5.00 | 5.00 | 5.00 | — |
| Oil Phase | PPG-15 stearyl | 15 | 15 | 3 | 10 | 3 | 3 |
| | ether | | | | | | |
| | Octyldodecanol | — | — | 12 | — | 12 | 12 |
| | Isopropyl | — | — | — | 5 | 5 | 5 |
| | myristate | | | | | | |
| | Lanolin | — | 2 | 2 | 2 | 2 | — |
| | Behenyl alcohol | 1 | 1 | 1 | 1 | 1 | 1 |
| | Steareth-21 | 1.5 | — | — | — | — | 1.5 |
| | Ceteareth-20 | — | 1.5 | 1.5 | 1.5 | 1.5 | — |
| | Ceteth-20 | 1.5 | 1.5 | — | — | — | — |
| | Laureth-4 | 2 | 2 | 2 | 2 | 2 | 2 |
| | Dichlorobenzyl | 0.1 | 0.1 | 0.1 | 0.1 | 0.1 | 0.1 |
| | alcohol | | | | | | |
| Water Phase | Carboxymethyl | — | 0.5 | 0.5 | 0.5 | 0.5 | — |
| | cellulose sodium | | | | | | |
| | Methyl cellulose | 0.15 | — | — | — | — | 0.15 |
| | Xanthan gum | 0.15 | — | — | — | — | 0.15 |
| Buffering | Tromethamine | 0.5 | — | — | — | — | 0.5 |
| Agents Phase | Disodium | — | 0.25 | 0.25 | 0.25 | 0.25 | — |
| | phosphate | | | | | | |
| | dehydrate | | | | | | |
| | Disodium | 0.05 | 0.05 | 0.05 | 0.05 | 0.05 | 0.05 |
| | edentate | | | | | | |
| | dehydrate | | | | | | |
| Preservative | Diazolidinyl urea | 0.3 | 0.3 | 0.3 | 0.3 | 0.3 | 0.3 |
| Phase | Glycerin | 3 | 3 | 3 | 3 | 3 | 3 |
| pH Adjustment | NaOH, 18% | to pH | to pH | to pH | to pH | To pH | To pH |
| | solution | 8.5 ± 0.5 | 8.5 ± 0.5 | 8.5 ± 0.5 | 8.5 ± 0.5 | 8.5 ± 0.5 | 8.5 ± 0.5 |
| | Propellant | 8.00 | 8.00 | 8.00 | 8.00 | 8.00 | 8.00 |
| | Purified water | To 100 | To 100 | To 100 | To 100 | To 100 | To 100 |

b) Creaming resilience

| | Pre-Foam Formulation | | | | | | | | | | |
|---|---|---|---|---|---|---|---|---|---|---|---|
| | (Serum Bottles) | | | | | Foam Formulations in Canister | | | | | |
| | Creaming | Creaming | | pH | Assay | Appearance | | | | | Collapse |
| Formulation | (centrifugation) | (centrifugation) | pH | 1:5 diluted | of API | [Scoring] | | | Density | pH 1:5 diluted | time |
| Name | 3,000 rpm (%) | 10,000 rpm (%) | undiluted | with water | (% w/w) | Quality | Color | Odor | [g/mL] | with water | at 36° C. [s] |
| 1 | 100 | 40 | 8.67 | 8.50 | 0.0045 | 6 | 1 | 1 | 0.032 | 8.60 | >300 |
| 2 | 100 | 100 | 8.11 | 7.86 | 0.0050 | 6 | 1 | 1 | 0.038 | 7.77 | >300 |
| 3 | 100 | 70 | 8.37 | 8.15 | 0.0048 | 6 | 1 | 1 | 0.038 | 7.92 | >300 |
| 4 | 100 | 75 | 8.50 | 8.32 | 0.0049 | 6 | 1 | 1 | 0.037 | 8.00 | >300 |
| 5 | 100 | 85 | 8.54 | 8.20 | 0.0050 | 6 | 1 | 1 | 0.037 | 8.07 | >300 |
| 6 | 100 | 50 | 8.79 | 8.60 | 0.0044 | 6 | 1 | 1 | 0.043 | 8.50 | >300 |

6 = Excellent;
1 = white or off white;
1 = no odor or faint odor.

c) Density and Foam Quality Test on subjection to freeze thaw cycles.

| | Foam Quality | | | | | | | Density | | Density | |
|---|---|---|---|---|---|---|---|---|---|---|---|
| | [Scoring] | | | | Density | Density | | [g/mL] | % ΔDensity | [g/mL] | % ΔDensity |
| Formulation | | FTC | 3 wk | 3 wk | [g/mL] | [g/mL] | % ΔDensity | 3 wk | 3 wk | 3 wk | 3 wk |
| Name | T-0 | 4 cycles | 30° C. | 40° C. | T-0 | FTC | FTC | 30° C. | 30° C. | 40° C. | 40° C. |
| 1 | 6 | 6 | 6 | 6 | 0.032 | 0.031 | −3 | 0.032 | 0 | 0.030 | −6 |
| 2 | 6 | 6 | 6 | 6 | 0.038 | 0.034 | −11 | 0.038 | 0 | 0.035 | −8 |
| 3 | 6 | 6 | 6 | 6 | 0.038 | 0.032 | −16 | 0.041 | 8 | 0.043 | 13 |
| 4 | 6 | 6 | 6 | 6 | 0.037 | 0.039 | 5 | 0.037 | 0 | 0.037 | 0 |
| 5 | 6 | 6 | 6 | 6 | 0.037 | 0.038 | 3 | 0.039 | 5 | 0.047 | 27 |
| 6 | 6 | 6 | 6 | 6 | 0.043 | 0.038 | −12 | 0.037 | −14 | 0.038 | −12 |

6 = Excellent

d) pH & Stability on subjection to freeze thaw cycles

| | | | | | | | | Shakability | | |
|---|---|---|---|---|---|---|---|---|---|---|
| | | | | pH diluted 1:5 | | pH diluted 1:5 | | [Scoring | | |
| | pH diluted 1:5 | pH diluted 1:5 | | with water | Δ pH | with water | Δ pH | 1 = Shakable] | | |
| Formulation | with water | with water | Δ pH | 3 wk | 3 wk | 3 wk | 3 wk | | 3 wk | 3 wk |
| Name | T-0 | FTC | FTC | 30° C. | 30° C. | 40° C. | 40° C. | T-0 | 30° C. | 40° C. |
| 1 | 8.60 | 8.41 | −0.19 | 8.66 | 0.06 | 8.52 | −0.08 | ND* | 1 | 1 |
| 2 | 7.77 | 7.66 | −0.11 | 7.77 | 0 | 7.75 | −0.02 | ND* | 1 | 1 |
| 3 | 7.92 | 7.74 | −0.18 | 7.90 | −0.02 | 7.85 | −0.07 | ND* | 1 | 1 |
| 4 | 8.00 | 7.78 | −0.22 | 7.99 | −0.01 | 7.87 | −0.13 | ND* | 1 | 1 |
| 5 | 8.07 | 7.71 | −0.36 | 8.13 | 0.06 | 7.92 | −0.15 | ND* | 1 | 1 |
| 6 | 8.50 | 8.54 | 0.04 | 8.65 | 0.15 | 8.63 | 0.13 | ND* | 1 | 1 |

ND: not determined

### Chemical Stability Results for Pre-Foam Formulations

Each pre-foam formulation was tested for chemical stability at baseline (T-0), following the FTC procedure and after 3 weeks of storage at various temperatures. Stability data results were compared to T-0, Stability test results are summarized in Tables a-b.

a: Chemical Stability Results of Pre-Foam Formulations

| | Assay of API (% w/w) | | | | |
|---|---|---|---|---|---|
| | | | | Pre-Foam Formulations | |
| | Pre-Foam Formulations | | | in Canisters | |
| | in Serum Bottles | | | (without gas) | |
| Formulation | | 3 wk | | | 3 wk |
| Name | T-0 | 30° C. | 3 wk 50° C. | 3 wk 40° C. | 50° C. |
| 1 | 0.0045 | 0.0047 | 0.0047 | 0.0048 | 0.0048 |
| 2 | 0.0050 | 0.0052 | 0.0047 | 0.0059 | 0.0047 |
| 3 | 0.0048 | 0.0048 | 0.0041 | 0.0053 | 0.0043 |
| 4 | 0.0049 | 0.0049 | 0.0045 | 0.0050 | 0.0048 |
| 5 | 0.0050 | 0.0051 | 0.0045 | 0.0053 | 0.0043 |
| 6 | 0.0044 | 0.0044 | 0.0043 | 0.0046 | 0.0043 |

b: Chemical Stability Results of Foam Formulation

| | Assay of API (% w/w) | |
|---|---|---|
| | Pre-Foam | |
| | Formulation | Foam |
| Formulation | (Serum Bottles) | Formulation in Canisters |

| Name | T-0 | 3 wk 30° C. | 3 wk 40° C. |
|---|---|---|---|
| 1 | 0.0045 | 0.0046 | 0.0049 |
| 2 | 0.0050 | 0.0050 | 0.0052 |
| 3 | 0.0048 | 0.0046 | 0.0047 |
| 4 | 0.0049 | 0.0049 | 0.0049 |
| 5 | 0.0050 | 0.0049 | 0.0051 |
| 6 | 0.0044 | 0.0045 | 0.0047 |

The two tables hereinabove demonstrate that the foamable emulsion compositions of the present invention are suitable for storing, together with at least one API without losing any significant API activity. These compositions are deemed “storable compositions”, wherein less than 100 ppm of the API activity is lost during the storage period of at least one week.

### Example 25

O/W Emulsion Carrier Formulations with Light Mineral Oil with and without Petrolatum

A) with Xanthan Gum and Methocel as Polymeric Agent

| | HLB | RHLB | 001 | 002 | 006 |
|---|---|---|---|---|---|
| light mineral oil | | 10.5 | 11.00 | 11.00 | 6.00 |
| cetostearyl alcohol | | 15.5 | 2.00 | 2.00 | 2.00 |
| white petrolatum | | 7 | | | 14.00 |
| Ceteth 10 | 12.9 | | | | 2.50 |
| Sorbitane laurate | 8.6 | | | | 3.00 |
| steareth-2 | 4.9 | | 4.30 | | |
| steareth-21 | 15.5 | | 2.90 | | |
| glyceryl stearate | 3.8 | | | 2.90 | 2.00 |
| polysorbate 80 | 15 | | | 1.50 | |
| peg 40 stearate | 16.9 | | | 2.80 | |
| xanthan gum | | | 0.30 | 0.30 | 0.30 |
| methocel k100M | | | 0.30 | 0.30 | 0.30 |
| purified water | | | 79.20 | 79.20 | 69.90 |
| Total: | | | 100.00 | 100.00 | 100.00 |
| Propellant (AP-70) | | | 8.00 | 8.00 | 8.00 |
| Cent. 3K | | | Stable | Stable | Stable |
| Cent. 10K | | | Stable | Stable | Stable |
| Viscosity. | | | 17676.23 | 11181.61 | 19531.83 |
| foam quality | | | G-E | G-E | G-E |
| color | | | White | White | White |
| odor | | | No odor | No odor | No odor |
| Shakability | | | moderate | moderate | poor |
| Density | | | 0.031 | 0.035 | 0.043 |
| Collapse time | | | >300/G | >300/FG | >300/G |
| BUBBLE SIZE | | | 119 | 110 | 116 |
| (μm) | | | | | |
| BUBBLE SIZE | | | 0 | 0 | 0 |
| (above 500 μm) | | | | | |

Comments: The above carriers all generated foams of good to excellent quality in the presence and absence of petrolatum. The foamable formulations all showed good resistance to accelerated aging and were found to be stable even when subjected to centrifugation at 10,000 rpm for 10 mins. They all had good bubble size averages and a collapse time in excess of 300 secs. These formulations are suitable as foamable vehicles for active agents including coal tar and or steroids. The stability of the emulsions is at least to an extent a reflection of careful selection of compatible surfactants, and matching with the oily phase.

B) Without Polymeric Agent

| | | | QFC008- | QFC012- |
|---|---|---|---|---|
| | HLB | RHLB | 071220 | 080106 |
| light mineral oil | | 10.5 | 11.00 | 15.00 |
| Isopropyl myristate | | 11.5 | | 15.00 |
| cetostearyl alcohol | | 15.5 | 3.00 | 3.00 |
| glyceryl stearate | 3.8 | | 3.20 | 4.50 |
| polysorbate 80 | 15 | | 2.20 | 3.70 |
| peg 40 stearate | 16.9 | | 3.30 | 3.80 |
| purified water | | | 77.30 | 55.00 |
| Total: | | | 100.00 | 100.00 |
| Propellant (AP-70) | | | 8.00 | 8.00 |
| Cent. 3K | | | Stable | Stable |
| Cent. 10K | | | 90% Cream. | Stable |
| Viscosity. | | | 13725.07 | 4303.08 |
| foam quality | | | G-E | G |
| color | | | White | White |
| odor | | | No odor | v.f.odor |
| Shakability | | | poor | 1 |
| Density | | | 0.063 | 0.039 |
| Collapse time | | | >300/G | >300/G |
| BUBBLE SIZE (μm) | | | 123 | 69 |
| BUBBLE SIZE (above | | | 0 | 0 |
| 500 μm) | | | | |

Comments: The above carriers all generated foams of good to excellent quality. The foamable formulations all showed good resistance to accelerated aging. When subjected to centrifugation at 10,000 rpm for 10 mins both showed resistance to creaming one being stable. They all had good bubble size averages and a collapse time in excess of 300 secs. These formulations are suitable as foamable vehicles for active agents including coal tar and or steroids. The stability of the emulsions is at least to an extent a reflection of careful selection of compatible surfactants, and matching with the oily phase.

### Example 26

### Fatty Alcohol Carrier Formulation

| | HLB | RHLB | 011 |
|---|---|---|---|
| stearyl alcohol | | 15.5 | 6.00 |
| Oleyl alcohol | | 14 | 6.00 |
| cetyl alcohol | | 15.5 | 6.00 |
| polysorbate 65 | 10.5 | | 2.70 |
| polysorbate 20 | 16.7 | | 3.60 |
| peg 40 stearate | 16.9 | | 3.10 |
| xanthan gum | | | 0.25 |
| methocel k100M | | | 0.25 |
| purified water | | | 72.10 |
| Total: | | | 100.00 |
| Propellent (AP-70) | | | 8.00 |
| Cent. 3K | | | Stable |
| Cent. 10K | | | Stable |
| viscosity. | | | 1 rpm-348885.53 |
| foam quality | | | G |
| color | | | White |
| odor | | | no odor |
| Shakability | | | 0 |
| Density | | | 0.045 |
| Collapse time | | | >300/G |
| BUBBLE SIZE (μm) | | | 79 |
| BUBBLE SIZE (above 500 μm) | | | 0 |

Comments: These unique fatty alcohol carrier formulations are stable to centrifugation and generate good quality foam and good bubble size with a collapse time in excess of 300 secs. These formulations are suitable as foamable vehicles for active agents including coal tar and or steroids

### Example 27

### PPG 15 Stearyl Ether or Isoprpyl Myristate/Avocardo Oil Emulsion Carrier Formulations

| | HLB | RHLB | 004 | 005 |
|---|---|---|---|---|
| PPG 15-stearyl ether | | 7 | 18.00 | |
| Isopropyl myristate | | 11.5 | | 6.00 |
| cetostearyl alcohol | | 15.5 | 2.00 | |
| stearyl alcohol | | 15.5 | | 1.00 |
| Avocado oil | | 7 | | 6.00 |
| steareth-2 | 4.9 | | 6.50 | |
| steareth-21 | 15.5 | | 2.50 | |
| glyceryl stearate | 3.8 | | | 3.30 |
| polysorbate 80 | 15 | | | 1.00 |
| peg 40 stearate | 16.9 | | | 2.00 |
| xanthan gum | | | 0.30 | 0.30 |
| methocel k100M | | | 0.30 | 0.30 |
| purified water | | | 70.40 | 80.10 |
| Total: | | | 100.00 | 100.00 |
| Propellent (AP-70) | | | 8.00 | 8.00 |
| Cent. 3K | | | Stable | Stable |
| Cent. 10K | | | Stable | Stable |
| viscosity. | | | 27962.03 | 15196.76 |
| foam quality | | | G-E | G-E |
| color | | | White | White |
| odor | | | No odor | No odor |
| Shakability | | | moderate | moderate |
| Density | | | 0.067 | 0.042 |
| Collapse time | | | >300/G | >300/FG |
| BUBBLE SIZE (μm) | | | 194 | 158 |
| BUBBLE SIZE (above 500 μm) | | | 0 | 0 |

Comments: These carrier formulations are also stable to centrifugation and generate good quality foam and good bubble size with a collapse time in excess of 300 secs. These formulations are suitable as foamable vehicles for active agents including coal tar and or steroids

### Example 28

### LCD Carrier Formulations

Part A —O/w Emulsion Formulations with Heavy Mineral Oil and Hydrocortisone

| | | | 009 | 010 |
|---|---|---|---|---|
| cf | HLB | RHLB | Ex. 6b | Ex. 6b |
| Mineral oil, heavy | | 10.5 | 10.00 | 10.00 |
| Lanolin | | 10 | 2.00 | 2.00 |
| cetostearyl alcohol | | 15.5 | 2.00 | 2.00 |
| Cyclomethicone | | 7.75 | 1.00 | 1.00 |
| glyceryl stearate | 3.8 | | 1.90 | 2.50 |
| polysorbate 80 | 15 | | 1.00 | 1.40 |
| peg 40 stearate | 16.9 | | 1.60 | 2.00 |
| xanthan gum | | | 0.25 | 0.25 |
| methocel k100M | | | 0.25 | 0.25 |
| purified water | | | 63.30 | 61.90 |
| Benzyl alcohol | | | 0.60 | 0.60 |
| Glycerin | | | 5.00 | 5.00 |
| LCD | | | 10.00 | 10.00 |
| Peppermint oil | | | 1.00 | 1.00 |
| Hydrocortisone | | | 0.10 | 0.10 |
| Total: | | | 100.00 | 100.00 |
| Propellent (AP-70) | | | 8.00 | 8.00 |
| Cent. 3K | | | Stable | Stable |
| Cent. 10K | | | 60% | 85% Cream. |
| | | | Cream. | |
| Viscosity. | | | | 13213.18 |
| foam quality | | | FG | G |
| color | | | | Yellow |
| odor | | | | charact.odor |
| Shakability | | | | 1 |
| Density | | | | 0.042 |
| Collapse time | | | | >300/G |
| BUBBLE SIZE (μm) | | | | 197 |
| BUBBLE SIZE (above 500 μm) | | | | 6.3 |

Comments: By increasing the surfactant mix the foam quality of the carrier formulation was improved. The formulations are stable to centrifugation at 3000K and do not phase separate at 10,000 rpm for 10 mins. These formulations are suitable as foamable vehicles for active agents including coal tar and or steroids

### Example 29

### Bioadhesive Estradiol Formulations with Carbopol or Polycarbophil and High or Low Viscosity Hypromellose

### Part A High Viscosity Hypromellose Formulations and Carbopol

Part A High Viscosity Hypromellose Formulations and Carbopol

| | 1 | 2 | 3 | 4 | 5 | 6 |
|---|---|---|---|---|---|---|
| | % w/w | % w/w | % w/w | % w/w | % w/w | % w/w |
| HV- | | | | | | |
| Octyldodecanol | 13.5 | 5 | 13.5 | 5 | 5 | 13.5 |
| Isopropyl | 5 | 5 | 5 | 5 | 5 | 5 |
| myristate | | | | | | |
| Sorbitan | 1 | — | — | 1 | — | — |
| stearate | | | | | | |
| Polysorbate 60 | — | 1 | 0.5 | — | — | 1 |
| Polysorbate 80 | — | | — | — | 0.5 | — |
| Stearic acid | 3 | 2 | — | — | — | — |
| Stearyl alcohol | — | — | 3 | 3 | — | — |
| Cetearyl alcohol | | | | — | 3 | 3 |
| Polyoxyl 100 | 2.5 | 2.5 | 2.5 | 2.5 | 2.5 | 2.5 |
| stearate | | | | | | |
| Carbopol 934P | 1 | 0.4 | 0.4 | 0.4 | 0.4 | 0.4 |
| Polycarbophil | — | — | — | — | — | — |
| Hypromellose | 0.2 | 0.2 | 0.2 | 0.2 | 0.2 | 0.2 |
| (Methocel | | | | | | |
| K100M) | | | | | | |
| Triethanolamine | 0.2 | 0.2 | 0.2 | 0.2 | 0.2 | 0.2 |
| Lactic Acid | to pH | to pH | to pH | to pH | to pH | to pH |
| | 4.0-4.5 | 4.0-4.5 | 4.0-4.5 | 4.0-4.5 | 4.0-4.5 | 4.0-4.5 |
| Propylene | 2 | 3.33 | 3.33 | 3.34 | 3.34 | 3.34 |
| glycol | | | | | | |
| Propyl paraben | 0.1 | 0.1 | 0.1 | 0.1 | 0.1 | 0.1 |
| Methyl paraben | 0.15 | 0.15 | 0.15 | 0.15 | 0.15 | 0.15 |
| Estradiol | 0.005 | 0.005 | 0.005 | 0.005 | 0.005 | 0.005 |
| hemihydrate | | | | | | |
| Purified water | to 100.00 | to 100.00 | to 100.00 | to 100.00 | to 100.00 | to 100.00 |
| Propellant | 8% | 8% | 8% | 8% | 8% | 8% |
| (propane, | | | | | | |
| butane, | | | | | | |
| isobutene mix) | | | | | | |
| Results | | | | | | |
| pH (undiluted) | 4.05 | 4.41 | 4.57 | canisters | 4.56 | 4.38 |
| | | | | were | | |
| | | | | blocked | | |
| pH (dil. 1:5 with | 4.2 | 4.69 | 4.97 | | 4.85 | 4.63 |
| water) | | | | | | |
| Centrifugation | | | | | | |
| at: | | | | | | |
| 1) 3,000 rpm: | stable | 90% | stable | | stable | stable |
| (% creaming) | | creaming | | | | |
| 2) 10,000 rpm | stable | 10% | 90% | | 90% | 90% |
| (% creaming): | | creaming | creaming | | creaming | creaming |
| Viscosity (cPs) | 106164 | 7673 | 23570 | | 37417 | 18105 |
| Microscopy | no crystals | no crystals | no crystals | | no crystals | no crystals |
| (x200) | observed | observed | observed | | observed | observed |
| Homogeneity of | homogeneous | homogeneous | homogeneous | | homogeneous | homogeneous |
| contents | | | | | | |
| Foam quality at | G | G | FG | | FG | G |
| T-0 | | | | | | |
| Foam quality | G | FG | FG | | FG | G |
| after 3 weeks | | | | | | |
| storage at 25° C. | | | | | | |

Comments: These high viscous formulations were found to be physically stable, homogenous and resistant to aging as seen from the centrifugation tests. The high viscosity hypermellose formulations proved to be more resistant to creaming at 10,000 rpm for about 10 mins than there low viscosity counterparts seen below. The difficulties in achieving good formulations at high viscosity can be seen when 0.5% polysorbate was replaced by 1% sorbitam stearate and the formulation did not expel from the canister. Formulations 1 and 6 produced good quality foam which remained unchanged after 3 weeks storage.

### Part B Low Viscosity Hypromellose Formulations and Carbopol

Part B Low Viscosity Hypromellose Formulations and Carbopol

| | 1 | 2 | 3 | 4 | 5 | 6 |
|---|---|---|---|---|---|---|
| | % w/w | % w/w | % w/w | % w/w | % w/w | % w/w |
| LV- | | | | | | |
| Octyldodecanol | 13.5 | 5 | 13.5 | 5 | 5 | 13.5 |
| Isopropyl | 5 | 5 | 5 | 5 | 5 | 5 |
| myristate | | | | | | |
| Sorbitan stearate | 1 | — | — | 1 | — | — |
| Polysorbate 60 | — | 1 | — | — | — | — |
| Polysorbate 80 | — | — | 0.5 | — | 0.5 | 1 |
| Stearic Acid | 3 | 2 | — | — | — | — |
| Stearyl alcohol | — | — | 3 | 3 | — | — |
| Cetearyl alcohol | — | — | — | | 3 | 3 |
| Polyoxyl 100 | 2.5 | 2.5 | 2.5 | 2.5 | 2.5 | 2.5 |
| stearate | | | | | | |
| Carbopol 934 | 1 | 0.4 | 0.4 | 0.4 | 0.4 | 0.4 |
| Polycarbophil | — | — | — | — | — | — |
| Hypromellose | 0.2 | 0.2 | 0.2 | 0.2 | 0.2 | 0.2 |
| (Methocel K100LV) | | | | | | |
| Triethanolamine | 0.2 | 0.2 | 0.2 | 0.2 | 0.2 | 0.2 |
| Lactic Acid for pH | q.s | q.s | q.s | q.s | q.s | q.s |
| for pH adjustment | | | | | | |
| to 4.0-4.5 | | | | | | |
| Propylene glycol | 2 | 2 | 2 | 2 | 2 | 2 |
| Propyl paraben | 0.1 | 0.1 | 0.1 | 0.1 | 0.1 | 0.1 |
| Methyl paraben | 0.15 | 0.15 | 0.15 | 0.15 | 0.15 | 0.15 |
| Estradiol | 0.005 | 0.005 | 0.005 | 0.005 | 0.005 | 0.005 |
| hemihydrate | | | | | | |
| Purified water | to 100.00 | to 100.00 | to 100.00 | to 100.00 | to 100.00 | to 100.00 |
| Propellant | 8% | 8% | 8% | 8% | 8% | 8% |
| (propane, butane, | | | | | | |
| isobutene mix) | | | | | | |
| Results | | | | | | |
| pH (undiluted) | 4.16 | 4.28 | 4.03 | 4.14 | 4.37 | 4.46 |
| pH (dil. 1:5 with | Not | 4.63 | 4.43 | 4.42 | 4.72 | 4.9 |
| water) | Recorded | | | | | |
| Centrifugation at: | | | | | | |
| 1) 3,000 rpm: | stable | 20% | stable | stable | 90% | stable |
| (% creaming) | | creaming | | | creaming | |
| 2) 10,000 rpm | 80% | 10% | 30% | 10% | 20% | 20% |
| (% creaming): | creaming | creaming | creaming | creaming | creaming | creaming |
| Viscosity (cPs) | 30664 | 2242 | 23661 | 15808 | 19712 | 15739 |
| Microscopy | no crystals | no crystals | no crystals | no crystals | no crystals | no crystals |
| (x200) | observed | observed | observed | observed | observed | observed |
| Homogeneity of | homogeneous | homogeneous | homogeneous | homogeneous | homogeneous | homogeneous |
| contents | | | | | | |
| Bioadhesiveness | −17.66 | −8.5 | −8.75 | −14.41 | −14.05 | −5.32 |
| (g): | | | | | | |
| Foam quality at | E | E | E | E | E | E |
| T-0 | | | | | | |
| Foam quality | Not tested | FG | E | E | E | E |
| after 3 weeks at | | | | | | |
| 25° C. | | | | | | |

Comments: These formulations made with lower viscous hypromellose were found to be stable, homogenous and resistant to aging as seen from the centrifugation tests. When blocked formulation 4 was repeated with low viscosity hypromellose the formulation was shakable and homogeneous. All these formulations displayed bioadhesive properties. When the level of carbopol was increased form 0.4% to 1% the bioadhesive strength of the formulation increased substantially. Some of the bioadhesion may be due to the presence of waxy fatty acids such as sorbitan stearate. The foams made with lower viscosity hypromellose were all of excellent quality. Thus, in a preferred embodiment the hypromellose is low viscosity. In an embodiment the level of bioadhesiveness is about −3 g to about −30.g

Part C Low Viscosity Hypromellose Formulations and Polycarbophil with and without Carbopol

| | 7 | 8 | 9 |
|---|---|---|---|
| | % w/w | % w/w | % w/w |
| LV- | | | |
| Octyldodecanol | 5 | 5 | 5 |
| Isopropyl | 5 | 5 | 5 |
| myristate | | | |
| Sorbitan stearate | 1 | — | — |
| Polysorbate 60 | — | — | — |
| Polysorbate 80 | — | 0.5 | 0.5 |
| Stearic Acid | 3 | — | — |
| Stearyl alcohol | — | 1.5 | 1.5 |
| Cetearyl alcohol | — | — | — |
| Polyoxyl 100 | 2.5 | 2.5 | 2.5 |
| stearate | | | |
| Carbopol 934 | 0.4 | — | — |
| Polycarbophil | 2 | 0.2 | 0.2 |
| Hypromellose | 0.2 | — | 0.2 |
| (Methocel K100LV) | | | |
| Triethanolamine | 0.2 | 0.2 | 0.2 |
| Lactic Acid for pH | q.s | q.s | q.s |
| for pH adjustment | | | |
| to 4.0-4.5 | | | |
| Propylene glycol | 2 | 2 | 2 |
| Propyl paraben | 0.1 | 0.1 | 0.1 |
| Methyl paraben | 0.15 | 0.15 | 0.15 |
| Estradiol | 0.005 | 0.005 | 0.005 |
| hemihydrate | | | |
| Purified water | to 100.00 | to 100.00 | to 100.00 |
| Propellant | 8% | 8% | 8% |
| (propane, butane, | | | |
| isobutene mix) | | | |
| Results | | | |
| pH (undiluted) | canisters were | 3.93 | 4.26 |
| pH (dil. 1:5 with | blocked | 4.36 | 4.62 |
| water) | | | |
| Centrifugation at: | | | |
| 1) 3,000 rpm: | | 25% creaming. | 25% creaming. |
| (% creaming) | | | |
| 2) 10,000 rpm | | 20% creaming | 20% creaming |
| (% creaming): | | | |
| Viscosity (cPs) | | 35.8 | 76.6 |
| Microscopy (x200) | | crystals | no crystals |
| | | observed | observed |
| Homogeneity of | | creaming re- | creaming re- |
| contents | | dispersible on | dispersible on |
| | | light shaking | light shaking |
| Foam quality at T-0 | | E | E |
| Foam quality after | | E | E |
| 3 weeks at 25° C. | | | |

Comments: The formulations with polycarbophil showed creaming but not phase separation on standing but were easily re-dispersed on light shaking. The canister became blocked when higher levels of polycarbophil are used with stearic acid. Upon reducing the amount of polycarbophil from 2% to about 0.2% and eliminating stearic acid excellent foam was observed, which remained unchanged after 3 weeks storage.

### Note

1. Viscosity of Methocel 100LV is-110 cPs (low viscosity); whereas for “Methocel 100M” viscosity is 103,510 cPs (high viscosity) being almost 1000 times greater.
2. Stearyl alcohol, sorbitan stearate, cetostearyl alcohol are powders (solid).
3. Polysorbate is a viscous liquid.

The references cited herein teach many principles of treatment of disorders that are applicable to the present invention. Therefore the full contents of these publications are incorporated by reference herein where appropriate for teachings of additional or alternative details, features and/or technical background.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art.

What is claimed is:

1. A storable foamable emulsion composition adapted for delivery of an active pharmaceutical ingredient (API) to a delivery site in a subject, the composition comprising:
a) at least one organic carrier selected from the group consisting of a hydrophobic organic carrier, an organic polar solvent, an emollient and mixtures thereof, at a concentration of about 2% to about 50% by weight;
b) at least one surface-active agent at a concentration of about 0.01% to about 5% by weight;
c) at least one polymeric agent selected from the group consisting of a bioadhesive agent, a gelling agent, a film forming agent and a phase change agent, each in a concentration of about 0.01% to about 5% by weight;
d) water;
e) an effective amount of at least one API selected from the group consisting of a steroid, a steroid derivative, and combinations thereof;
f) optionally, a further active agent; and
g) a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least about one month;
wherein the at least one API remains chemically stable for at least about one month; and
wherein the composition is stored in an aerosol container and upon release expands to form a breakable foam having an average bubble size range of about 30 to about 250 micron.

2. The storable foamable emulsion composition of claim 1, wherein when compressed between two surfaces a negative adhesion force is required to effect their separation.

3. The storable foamable emulsion composition of claim 1, wherein the at least one API is contained in a Trapsome.

4. The storable foamable emulsion composition of claims 1, 2 and 3, wherein the API is selected from the group consisting of:
i. a steroid compound containing a cyclopenta[a]phenanthrene skeleton;
ii. a steroid compound containing a cyclopenta[a]phenanthrene skeleton carrying one or more functional groups selected from halogens, alkyl groups, aryl groups, benzyl groups, carboxy groups and alkoxy groups;
iii. a steroid compound selected from the families of (a) cardanolides, (b) bufanolides, (c) spirostans, (d) furostans, (e) steroid alkaloids, (f) steroid lactones, (g) oxo-steroids, (h) steroid-alcohols and (i) steroid-amines;
iv. a steroid compound, where one or more of the cyclopenta[a]phenanthrene rings is contracted by loss of an unsubstituted methylene group;
v. a steroid compound, where one or more of the cyclopenta[a]phenanthrene rings is expanded by inclusion of a methylene group;
vi. a steroid compound containing a cyclopenta[a]phenanthrene skeleton and a carbocyclic or heterocyclic ring component fused to it;
vii. a compound, wherein two or more steroid molecules are linked together covalently;
viii. a compound selected from the group consisting of 5-pregnane, 5β-pregnane, 5α-cholane (allocholane), 5β-cholane, 5β-cholestane, 5β-cholestane, 5α-ergostane, 5-ergostane, 5α-campestane, 5-campestane, 5α-poriferastane, 5β-poriferastane, 5α-stigmastane, 5-stigmastane, 5α-gorgostaneacrihellin, actodigin, alfacalcidol, aldosterone, androsterone, betamethasone, brassinolide, calcidiol, calciol, calcitriol, canrenone, clomegestone, cholesterol, cholic acid, corticosterone, cortisol, cortisol acetate, cortisone, cortisone acetate, cyproterone, deoxycorticosterone, dexamethasone, disogluside, ecdysone, ercalciol, ergosterol, estradiol, estriol, estrone, ethinylestradiol, fluazacort, fluocortin, fusidic acid, gestrinone, gonane, halometasone, hydrocortisone, lanosterol, lithocholic acid, mebolazine, medroxyprogesterone, meproscillarin, mespirenone, mestranol, naflocort, norenthisterone, norgesterone, norgestrel, oxandrolone, oxymetholone, pancuronium bromide, prednisolone, prednisone, progesterone, proscillardin, pseudotigogenin, roxibolone, sarsasapogenin, smilagenin, spironolactone, timobesone, testosterone, tigogenin triamcinolone, ursodeoxycholic acid;
ix. an anti-inflammatory steroid;
x. a steroid possessing immunomodulating and/or anti-inflammatory properties;
xi. a steroid, selected from the group of low-potency anti-inflammatory steroids, medium potency anti-inflammatory steroids and high potency anti-inflammatory steroids;
xii. an anti-inflammatory steroid, selected from the group consisting of hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol, clobetasol-17-propionate;
xiii. a steroid that positively affects the McKenzie vasoconstrictor assay;
xiv. a steroid hormone;
xv. a steroid hormone, selected from the group consisting of an androgen, an estrogen and a progestogen;
xvi. an androgen, selected from the group consisting of testosterone, testosterone cipionate, testosterone decanoate, testosterone enantate, testosterone isocaproate, testosterone phenylpropionate, testosterone propionate, testosterone undecylate, 5α-dihydrotestosterone, dehydroepiandrosterone (also termed prasterone and DHEA), androstenedione, androstanediol, androsterone, androstenolone, prasterone enantate, prasterone sodium sulfate, ormeloxifene, mesterolone, fluoxymesterone, methyltestosterone, gestrinone, delmadinone, delmadinone acetate, chlormadinone, chlormadinone acetate, danazol and testolactone;
xvii. an estrogen selected from the group consisting of estradiol, estradiol benzoate, estradiol cipionate, estradiol dipropionate, estradiol enantate, estradiol hexahydrobenzoate, estradiol phenylpropionate, estradiol valerate, polyestradiol phosphate, estriol, estriol sodium succinate, estriol succinate, polyestriol phosphate, quinestradol, ethinylestradiol, estrapronicate, mestranol, estrapronicate and equilin;
xviii. a progestogen, selected from the group consisting of progesterone, norethisterone, norethisterone acetate, norethisterone enantate, medroxyprogesterone acetate, delmadinone acetate, flugestone acetate, dydrogesterone, desogestrel, norgestrel, levonorgestrel, dydrogesterone, gestodene, chlormadinone acetate, dienogest, drospirenone, lynestrenol, tybolone, cyproterone acetate, megestrol acetate, nomegestrol acetate;
xix. an inhibitor of a steroid hormone;
xx. an inhibitor of a steroid hormone selected from the group consisting of finasteride, dutasteride and spironolactone;
xxi. a vitamin D;
xxii. a steroid that exhibits qualitatively the biological activity of calciol;
xxiii. a vitamin D selected from the group consisting of cholecalciferol, 25-hydroxycholecalciferol, 1α,25-dihydroxycholecalciferol, ergocalciferol, 1α,25-dihydroxyergocalciferol, 22,23-dihydroergocalciferol, 1,24,25-trihydroxycholecalciferol, previtamin D3, tachysterol3 (also termed tacalciol);
xxiv. a vitamin D3 analogue;
xxv. isovitamin D3, dihydrotachysterol3, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol;
xxvi. a vitamin D3 analogue selected from the group consisting of calcipotriol, tacalcitol, maxacalcitol, and calcitriol;
xxvii. a phytosteroid or a phytosterol;
xxviii. a steroid derived or extracted from one of the families of phytosteroids, phytosterols, phytostanols, ecdysones, withanolids, sterines, steroid saponins and soflavonoids;
xxix. a steroid selected from the group consisting of alpha-sitosterol, beta-sitosterol, stigmastanol, campesterol, alpha-sitostanol, beta-sitostanol, stigmastanol, campestanol, avenosterol, brassicasterol, desmosterol, chalinosterol, beta-ecdysone, whithaferin A, beta-sitosterine, stigmasterine, campesterine, ergosterine, diosgenin, daidzein, glycitein, genistein, muristerone, poriferasterol, clionasterol, campestanol, and cycloartenol;
xxx. a plant oil or a plant extract, which contains a steroid; and
xxxi. a plant oil or a plant extract, selected from the group consisting of nuts seeds, sprouted seeds and grains (such as alfalfa), St. Mary's thistle, ginkgo biloba, saw palmetto, panax, siberian ginseng, foeniculum vulgare, cimicifuga racemosa, licorice root, red clover, sage, sarsaparilla, sassafras, angelica sinensis achillea millefolium, anemone pratensis, angelica sinensis, glycyrrhiza glabra, hypericum perforatum, larrea, panax, piscidia erythrina, plantago psyllium, serenoa repens, symphytum, taraxacum officinale, trifolium pratense, turnera spp., tussilago farfara, valeriana officinalis, viburnum prunifolium, and calendula officinalis.

5. The storable foamable emulsion composition of claim 4, further comprising at least one quiescence agent selected from the group consisting of a stabilizing agent, a polymeric agent, a gelling agent, a viscoelastic agent, a pseuedoplastic or semi-pseudoplastic agent, a resilient agent and a modulating agent and mixtures thereof.

6. The storable foamable emulsion composition of claim 1, wherein the foam demonstrates at least seven of the following properties:
(a) a foam quality of 5-6;
(b) a bioadhesivity to said delivery site, requiring a force of about −3 g to −30 g to dislodge said foam from said delivery site;
(c) a bioadhesivity to said delivery site, requiring a force of about −8 g to about −25 g to dislodge said foam from said delivery site;
(d) a color of white to off-white; or a yellowish color:
(e) no odor or faint odor; or substantially masked odor;
(f) a foam quality of 5-6 after at least one freeze-thaw cycle;
(g) a foam quality of 5-6 after about 3 weeks' storage at 30° C.;
(h) a foam quality of 5-6 after about 3 weeks' storage at 40° C.; and
(i) a foam quality of 5-6 after about 3 weeks' storage at 50° C.;
(j) a collapse time of more than 50 seconds;
(k) a collapse time of more than 120 seconds; and
(l) a collapse time of more than 180 seconds.

7. The storable foamable emulsion composition of claim 6, wherein the foam demonstrates all of said properties.

8. The storable foamable emulsion composition of claim 5, wherein the resilient agent is a viscoelastic material capable of forming a pseudoplastic or semi-pseudoplastic composition.

9. The storable foamable emulsion composition of claim 8, wherein the viscoelastic material comprises an effective amount of at least one polymeric additive selected to form a foamable carrier or composition which is resilient to creaming and or phase separation.

10. The storable foamable emulsion composition of claim 8, wherein the viscoelastic material comprises an effective amount of at least one polymeric additive selected to form a foamable carrier or composition which is shakable and flowable to a sufficient amount to be expelled by a propellant from a canister to form a foam of appropriate quality for topical or mucosal application.

11. The storable foamable emulsion composition of claim 8, wherein the viscoelastic material is at least one polymeric additive selected from the group consisting of polysaccharides, natural polysaccharides, derivatives thereof, modified poysacharides, derivatives thereof, starch, dextrin, glycogen, cellulose and chitin, glycosaminoglycans (GAG's), chondroitin sulphate, dermatan sulphate, keratan sulphate, heparan sulphate, heparin, and hyaluronan, amylose and amylopectine, cellulose derivatives, xanthan gum, sodium CMC, methylcellulose, hydroxyl propyl methyl cellulose, carbomer, carbopol, and polycarbophil.

12. The storable foamable emulsion composition of claim 1, wherein the delivery site is a body cavity.

13. The storable foamable emulsion composition of claim 12, wherein said composition is suitable for spreading substantially throughout the body cavity surface, effectively such that the active agent reaches a target area.

14. The storable foamable emulsion composition of claim 13, wherein the body cavity is selected from the group consisting of vagina, rectum, colon, penile urethra, nasal cavity and ear cavity.

15. The storable foamable emulsion composition of claim 14, wherein the body cavity is a vagina, and wherein the foam is adapted to spread substantially throughout the vaginal cavity surface, effectively such that the API reaches the cervix.

16. The storable foamable emulsion composition of claim 15, wherein the foam has a bioadhesivity of at least to said delivery site, requiring a force of about −3 g to about −30 g to dislodge said foam from said delivery site.

17. The storable foamable emulsion composition of claim 1, further comprising at least one additional non-steroidal therapeutic agent selected from the group consisting of an anti-infective, an antibiotic, an antibacterial agent, an antifungal agent, an antiviral agent, an antiparasitic agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, benzoyl chloride, calcium hypochlorite, magnesium hypochlorite, an anti-wrinkle agent, a radical scavenger, a metal, silver, a metal oxide, titanium dioxide, zinc oxide, zirconium oxide, iron oxide, silicone oxide, talc, carbon, an anti wrinkle agent, a skin whitening agent, a skin protective agent, a masking agent, an anti-wart agent, a refatting agent, a lubricating agent and mixtures thereof.

18. The storable foamable emulsion composition of claim 1, wherein the at least one polymeric agent is selected from the group consisting of a water-soluble cellulose ether and naturally-occurring polymeric material.

19. The storable foamable emulsion composition of claim 18, wherein the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose, carboxymethylhydroxyethylcellulose, xanthan gum, guar gum, carrageenin gum, locust bean gum and tragacanth gum.

20. The storable foamable emulsion composition of claim 1, comprising:
i. a steroid;
ii. at least one organic carrier selected from a hydrophobic organic carrier, an emollient and mixtures thereof, at a concentration of about 2% to about 50% by weight;
iii. a surface-active agent;
iv. about 0.01% to about 5% by weight of at least one polymeric agent selected from the group consisting of a bioadhesive agent, a gelling agent, a film forming agent and a phase change agent;
v. water; and
vi. liquefied or compressed gas propellant at a concentration of about 3% to about 25% by weight of the total composition,
wherein the composition is an emulsion, which is flowable to a sufficient extent to be expelled by the propellant from a canister to form a bioadhesive foam of appropriate quality for topical, body cavity or mucosal application having a bioadhesivity to said delivery site, requiring a force of about −3 g to about −30 g to dislodge said foam from said delivery site.

21. A composition according to claim 1 wherein:
(a) the at least one organic carrier comprises at least one carrier selected from the group consisting of ocytyldodecanol, isopropyl myristate, mineral oil, capric/caprylic triglyceride, silicone, lanolin and glycerin;
(b) the at least one a surface-active agent comprises at least one of, Polysorbate 60, Polysorbate 80, Polyoxyl 100 stearate, PEG 40 stearate, PPG 15 Stearyl Ether, and Ceteareth 20,
(c) the at least one polymeric additive comprises at least one of hypromellose, polycarbophil, carbopol and additionally optionally propylene glycol; and
(d) optionally further comprising a foam adjuvant comprising at least one of cetearyl alcohol, oleyl alcohol, stearyl alcohol and stearic acid.

22. A storable foamable emulsion composition adapted for delivery of an active pharmaceutical ingredient (API) to a delivery site in a subject, the composition comprising:
a) at least one fatty alcohol, at a concentration of about 2% to about 50% by weight;
b) at least one surface-active agent at a concentration of about 0.01% to about 5% by weight;
c) at least one polymeric agent selected from the group consisting of a bioadhesive agent, a gelling agent, a film forming agent and a phase change agent, each in a concentration of about 0.01% to about 5% by weight;
d) water;
e) an effective amount of at least one API selected from the group consisting of a steroid, a steroid derivative, a further active agent together with the steroid or derivative thereof and combinations thereof; and
f) a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least about one month;
wherein the at least one API remains chemically stable for about at least about one month; and
wherein the composition is stored in an aerosol container and upon release expands to form a breakable foam having an average bubble size range of about 30 to about 250 micron.

23. The storable foamable emulsion composition of claim 22, wherein the fatty alcohol includes at least one unsaturated fatty alcohol.

24. A method of treating, alleviating or preventing a disorder of the skin, a body cavity or a mucosal surface, wherein the disorder involves inflammation as one of its etiological factors, which method comprise administering topically to a subject having the disorder in need thereof a therapeutically effective amount of a storable foamable emulsion composition comprising:
a. at least one organic carrier selected from the group consisting of a hydrophobic organic carrier, an organic polar solvent, an emollient and mixtures thereof, at a concentration of about 2% to about 50% by weight;
b. at least one surface-active agent at a concentration of about 0.01% to about 5% by weight;
c. at least one polymeric additive selected from the group consisting of a bioadhesive agent, a gelling agent, a film forming agent and a phase change agent, each in a concentration of about 0.01% to about 5% by weight;
d. water;
e. an effective amount of at least one API selected from the group consisting of a steroid, a steroid derivative, a further active agent together with the steroid or derivative thereof and combinations thereof; and
f. a propellant at a concentration of about 3% to about 25% by weight of the total foamable composition,
wherein, at ambient temperature, the storable foamable emulsion composition is shakable, is resistant to centrifugation at about 3000 rpm for about 10 min, is substantially devoid of crystals, is resistant to at least one freeze-thaw cycle and does not phase separate within at least about one month;
wherein the at least one API remains chemically stable for at least about one month; and
wherein the composition is stored in an aerosol container and upon release expands to form a breakable bioadhesive foam having an average bubble size range of about 30 to about 250 micron.
